(19) United States
(12) Patent Application Publication
(10) Pub. No.: US 2025/0043349 A1
(43) Pub. Date: Feb. 6, 2025

(54) DEORPHANIZING ODORS USING SINGLE CELL SEQUENCING

(71) Applicant: PRESIDENT AND FELLOWS OF HARVARD COLLEGE, Cambridge, MA (US)

(72) Inventors: Sandeep DATTA, Cambridge, MA (US); Tatsuya TSUKAHARA, Cambridge, MA (US); David BRANN, Cambridge, MA (US)

(73) Assignee: PRESIDENT AND FELLOWS OF HARVARD COLLEGE, Cambridge, MA (US)

(21) Appl. No.: 18/714,885

(22) PCT Filed: Dec. 2, 2022
(86) PCT No.: PCT/US2022/051618

§ 371 Date: May 30, 2024

Related U.S. Application Data
(60) Provisional application No. 63/285,307, filed on Dec. 2, 2021.

(51) Int. Cl. C12Q 1/6883; G01N 33/50

(52) U.S. Cl. CPC C12Q 1/6883; G01N 33/5023; G01N 33/5058; C12Q 2600/158; G01N 2333/11; G01N 2333/165; G01N 2800/2821; G01N 2800/2835

(57) ABSTRACT

Scents are perceived by the olfactory sensory neurons (OSNs) that line the upper nasal cavity. Each OSN expresses one odorant receptor, and these odorant receptors contact the scent molecules. Methods for determining which odorant receptor(s) are activated by a scent are lacking, making it difficult to replicate or improve scents. The technology as disclosed herein refers to methods relating to activating odor response genes found in olfactory sensory neurons after the neurons are exposed to at least one volatilized chemical compound.

### CROSS-REFERENCE TO RELATED APPLICATIONS

This application claims benefit under 35 U.S.C. 119 (e) of U.S. Provisional Application No. 63/285,307, filed Dec. 2, 2021, the contents of which are incorporated herein by reference in their entirety.

### GOVERNMENT SUPPORT

This invention was made with government support under Grant Nos. DC016222 and NS112953 awarded by the National Institutes of Health (NIH). The government has certain rights in the invention.

### TECHNICAL FIELD

The technology described herein relates to sensory neurons and altering odor responses.

### BACKGROUND

Scents are perceived by the olfactory sensory neurons (OSNs) that line the upper nasal cavity. Each OSN expresses one odorant receptor, and these odorant receptors contact the scent molecules. Methods for determining which odorant receptor(s) are activated by a scent are lacking, making it difficult to replicate or improve scents.

### SUMMARY

The inventors have identified a suite of genes whose expression is altered when an odorant receptor is activated by a scent (e.g., one or more volatilized chemical compounds). This discovery has been applied to provide methods of identifying which odorant receptors perceive a scent. The method can be further used to screen scents for those that activate a given odorant receptor or set of odorant receptors, e.g., to provide a mimic of a benchmark scent. The ability to match odors to their receptors is groundbreaking for scent and fragrance use, as it permits the rational design of odors for foods and fragrances.

In one aspect of any of the embodiments, described herein is a method comprising measuring the expression of at least one odor response gene in a first olfactory sensory neuron (OSN) of a first subtype that has been contacted with at least one volatilized chemical compound.

In one embodiment in any of the aspects, the method further comprises measuring the expression of at least one odor response gene in a second OSN of the first subtype that has not been contacted with the at least one volatilized chemical compound. In one embodiment of any of the aspects described herein, the method further comprises determining that the first subtype expresses an odorant receptor that binds to the at least one volatilized chemical compound if the expression of the at least one odor response gene in the first OSN is different from a reference level of expression in a second OSN of the first subtype that has not been contacted with the at least one volatilized chemical compound.

In one aspect of any of the embodiments, described herein is a method comprising: measuring the expression of at least one odor response gene in a first OSN of a first subtype that has been contacted with at least one volatilized chemical compound; measuring expression of at least one odor response gene in a second OSN of the first subtype that has not been contacted with the at least one volatilized chemical compound; and determining that the first subtype expresses an odorant receptor that binds to the at least one volatilized chemical compound if the expression of the at least one odor response gene in the first OSN and the second OSN are different.

In one embodiment in any of the aspects, the at least one odor response gene is selected from the group consisting of: Tsc22d1, Rcan1, Fosb, Pcdh10, Dusp14, Srxn1, Gm13889, Golga4, Heg1, Cdkn1a, Gpr137c, Zfp655, Ublcp1, Efr3b, Etf1, Srrm4, Epcam, Trp53inp2, Arhgap17, Ric8b, 9530034E10Rik, Oaz2, Ctxn3, Slc38a2, Exoc6b, Nfatc1, Junb, Tle3, Siah2, Fos, Ago2, Arhgef28, Ddx3x, Hspa8, Hnrnph3, Erf, Ak2, Pnrc1, Mafg, D1Ertd622c, Arid5b, Scn3b, Phtf2, Hsph1, Cpeb4, Ubc, Sema7a, Dleu2, Rc3h1, Pcnx4, Btg2, 6430548M08Rik, Selenok, Psmd8, Map9, Cry2, Hspa41, Mcl1, Dnaja1, Fth1, Fam208a, Bmpr2, Zfp608, BC005561, Rab11fip4, Smim13, Ppp2ca, Sod1, Neat1, Brinp2, Tuba4a, Cdc14a, Kdm6b, Uckl1os, Stk40, Dnajb2, Dnajb9, Spsb3, Sub1, Rheb, Manea, Fzd3, Zfand5, Uhrf1bp11, Cebpb, Wdfy3, Mfgc8, Inpp4a, Nab1, Tceal9, Ube2g1, Pcdh7, Eef1a1, Tnrc6c, Cd82, BC005537, Sdcbp, Ddx5, Olfm1, Srpk1, M6pr, Braf, Ahi1, Clcn3, Eef2, Rbm26, Arfgef3, Eif5, Tmem150c, Acbp2, Snrk, Bex3, Epb4112, Samd4b, Stard10, Pappa, Grsf1, Ralgds, Arih1, Micu3, Ptges3, Kras, Morf412, Rtp2, Wdr26, Hspa5, Zmynd8, Sv2c, Paip2, Rab39b, Ywhaz, Gpat3, Psen2, Pde4a, Atp2b1, Irs2, Cfap69, Hspa9, Tob1, Hspa14, Pard6a, Mapk4, Nr4a1, Sik3, Epha7, Hagh, Kalrn, Prkar1a, Lmbrd1, Erich3, Sf3b1, Ksr2, Ybx1, Nfix, Hivep1, Mfn2, Elof1, Wdfy2, Emd, Azin1, Wbp1, Nop53, Syt7, Ncoa1, Smarca5, Aes, Rnf150, Plxnb2, Wdsub1, Dyrk4, Epb4111, Pcdh8, Daam1, Prpf4b, Tnrc6a, Rufy3, Ptdss2, Tmem230, Scn3a, Rock1, Dph3, Zbtb7b, Yipf4, Ift74, Ebf1, Wsb2, Rnf182, Arglu1, Ppm11, Ccdc189, Ptov1, Ccdc157, Ppa1, Pnpla8, Rack1, Rsrp1, Ttc9, Hon2, Ccdc40, Kat2b, Cngb1, Ndufa3, Eefla2, Fmn2, Macrod1, Polr2i, 1700016K19Rik, Jade1, Slcla2, Ralbp1, Eif4c3, Morn2, Trappc21, Scrn1, Rtp1, Phyh, Fetub, Mapre3, Dnajb13, Coa3, Spef1, 1810058124Rik, Cep290, Bbs4, Nin, D430042009Rik, Tex9, Sdc3, Ebf4, Oaz1, Napa, Ttc8, Sdhaf4, Cuta, Commd6, Sfr1, Hcfc1r1, Nxn12, Kcnh3, Trpm7, Glb112, Slc25a39, Nsmf, Pifo, Oscp1, 1110008P14Rik, Egr1, Ttll6, Sem1, Aplg1, Fkbp2, Suclg1, Cidea, Dynlrb2, Ndufa2, Map1a, Sun1, Rufy2, Rnh1, Dtna, Anapc16, Pdhb, A430035B10Rik, Faim2, Ebf2, Ccdc28b, Ttll3, Drc1, Sys1, Prdx5, Palm, Slu7, Dnajc15, Elmod1, Paqr9, Tubb3, Ascc1, Ndufc1, Pon2, Fkbp4, Trp1, Cldn3, Cby1, Tctex1d2, Slc48a1, Nme5, 1110004E09Rik, Cnppd1, Naa38, Vdac3, Puf60, Cers5, Cbr1, Ift43, Fam217a, Ankrd10, Ubc2n, Pfdn6, Mpc2, Ncbp2, Timm13, Lamtor5, Uqer10, Slc27a2, Arpc51, Adh5, Gkap1, Ndufaf4, Psip1, Omp, Bcl11a, Ift81, 2010107E04Rik, Ubxn1, Ech1, Tnrc6b, Tmpo, Ssbp4, 2410015M20Rik, Mapk8ip2, Naxc, Gm13589, Hdgfl3, Slc22a23, Cacna1h, Slc25a5, Ndufa13, Anp32a, Ndufb10, Lhx2, Cenpx, Rnf220, Ankrd54, Ppdpf, Dtd1, Auh, Mrp151, Psmd13, Lamtor4, Hist2h2bc, Mapkap1, Arhgdig, Ndufv3, Ilkap, Ctsa, Ift27, Lpcat4, Atp50, Ypel3, Gad11, Msln1, Hint2, Acbd7, Manf, Lamtor2, Ak1, Arl6, Uxs1, Sri, Tomm7, Ubxn6, Cd47, Snrpd3, Ppid, Rsph9, Rbfox2, Pigp, C1qbp, Fmc1, Bcl7a, Krtcap2, Flrt1, Sdhd, Bphl, Sod2, Ndufb7, Prrc2b, Rex1bd, Rab36, Pdrg1, Pfn2, Rarg, B230118H07Rik, Elavl3, Mlf1, Eif1, Mettl26, Kmt5a, Ndufa5, Ndufb3, Emc8, Bad, Smc3, Dpm3, Ndufa11, Cisd1, Mrpl27, Cldn9, Sec61b, Tpd5212, Polr2c, Ndufa7, Atf5, Gabarap, Srsf3, Psmb3, Bax, Atox1, 2410004P03Rik, Atpla1, Ptprs, Cib1, Hist1h2bc, Atp5k, Hmox2, Fam92b, Chd4, Elob, Kif5a, Tbcb, Ndufa4, Uqer11, Hras, Atp6vlf, Pam16, Pim3, Lrriq1, Nfu1, Supt4a, Swi5, Mtx2, Faim, Taldo1, Ccdc34, Rbm39, 2210016L21Rik, Dpy30, Tomm22, Pygo1, Mat2b, Mrp128, Eid1, Snrpe, Atp5j, Ndufs7, Park7, Ndufs4, Cirbp, Polr2f, Fbx09, Mgst3, Gtf2h5, Mtch1, Mrps24, Actr1b, Sh3bgrl3, Bmpr1a, Pfn1, Tesc, Tmem258, Mrps14, Abhd16a, Vdac2, Nxph3, Polr2g, Them6, Cfap126, Pitpnc1, Kctd1, and Dmkn. In one embodiment of any of the aspects, at least one odor response gene is selected from the group consisting of: Tsc22d1, Rcan1, Fosb, Pcdh10, Dusp14, Srxn1, Nr4a2, Btg2, Egr1, Fos, Fosb, Gm13889, and Junb. In one embodiment of any of the aspects, the at least one odor response gene comprises at least one odor response gene, at least two odor response genes, at least three odor response genes, at least five odor response genes, at least ten odor response genes, at least fifty odor response genes, or at least one hundred odor response genes.

In one embodiment of any of the aspects, the method further comprises measuring the expression of at least one odorant receptor gene.

In one embodiment of any of the aspects, the expression of the at least one odor response gene is measured 2 or more hours after contacting the OSN with the at least one volatilized chemical compound. In one embodiment of any of the aspects, the expression of the at least one odor response gene is measured 3 days or more after contacting the OSN with the at least one volatilized chemical compound.

In one embodiment of any of the aspects, the first subtype is an odorant receptor subtype. In one embodiment of any of the aspects, the first subtype expresses one odorant receptor. In one embodiment of any of the aspects, the odorant receptor is a human odorant receptor. In one embodiment of any of the aspects, the odorant receptor is a murine odorant receptor. In one embodiment of any of the aspects, the human odorant receptor is selected from the group consisting of: human OR family 1, human OR family 2, human OR family 3, human OR family 4, human OR family 5, human OR family 6, human OR family 7, human OR family 8, human OR family 9, human OR family 10, human OR family 11, human OR family 12, human OR family 13, human OR family 14, human OR family 51, human OR family 52, human OR family 55, human OR family 56, and any variant thereof having an amino acid sequence having at least 80% identity with the amino acid sequence of one of said human odorant receptors. In one embodiment of any of the aspects, the odorant receptor is selected from the group consisting of: Olfr 545, Olfr 160, Olfr 17, Olfr 727, Olfr 728, and Olfr 729. In one embodiment of any of the aspects, the odorant receptor is selected from the group consisting of: Olfr 545, Olfr 160, and Olfr 17. In one embodiment of any of the aspects, the murine odorant receptor is selected from the group consisting of: murine OR family 1, murine OR family 2, murine OR family 3, murine OR family 4, murine OR family 5, murine OR family 6, murine OR family 7, murine OR family 8, murine OR family 9, murine OR family 10-15, murine OR family 16-34.

In one embodiment of any of the aspects, the first OSN and/or the second OSN expresses at least one olfactory receptor. In one embodiment of any of the aspects, the first OSN and/or the second OSN is in vitro during the contacting.

In one embodiment of any of the aspects, the first OSN and/or the second OSN is in vivo during the contacting. In one embodiment of any of the aspects, the contacting the first OSN comprises contacting a subject comprising the first OSN with air comprising the at least one volatilized chemical compound.

In one embodiment of any of the aspects, the not contacting the second OSN comprises: occluding the nostrils of a subject comprising the second OSN, or contacting a subject comprising the second OSN with air not comprising the at least one volatilized chemical compound. In one embodiment of any of the aspects, the second OSN is not contacted with the at least one volatilized chemical compound for at least two hours prior to the measuring.

In one embodiment of any of the aspects, measuring the expression comprises measuring the level of one or more mRNAs. In one embodiment of any of the aspects, measuring the level of one or more mRNAs comprises measuring the level of the one or more mRNAs in a single cell. In one embodiment of any of the aspects, measuring the level of one or more mRNAs comprises single-cell sequencing.

In one embodiment of any of the aspects, the method further comprises measuring the expression of at least one odor response gene in a plurality of OSNs contacted with at least one volatilized chemical compound, each of the plurality of OSNs expressing a different odorant receptor. In one embodiment of any of the aspects, the method further comprises measuring the expression of at least one odor response gene in a plurality of OSNs, each of the plurality of OSNs having been contacted with a different at least one volatilized chemical compound. In one embodiment of any of the aspects, each of the plurality of OSNs is cultured in different wells of a multi-well plate or different cell culture containers. In one embodiment of any of the aspects, the plurality of OSNs are cultured in the same well of a multi-well plate or the same cell culture container. In one embodiment of any of the aspects, each well of a multi-well plate or each cell culture container is contacted with the same at least one volatilized chemical compound. In one embodiment of any of the aspects, each well of a multi-well plate or cell culture container is contacted with a different at least one volatilized chemical compound.

In one embodiment of any of the aspects, each of the plurality of OSNs is present in a different subject during the contacting. In one embodiment of any of the aspects, the plurality of OSNs are present in the same subject during the contacting. In one embodiment of any of the aspects, each subject is contacted with the same at least one volatilized chemical compound. In one embodiment of any of the aspects, each subject is contacted with a different at least one volatilized chemical compound.

In one embodiment of any of the aspects, the method further comprises obtaining a sample comprising the first OSN and/or second OSN from the nose of a subject after the contacting and before the measuring. In one embodiment of any of the aspects, sample is obtained from the olfactory bulb, the olfactory epithelium, the nerve endings, and/or the nasal cavity. In one embodiment of any of the aspects, the sample comprises epithelial cells, endothelial cells, olfactory sensory neurons, goblet cells, cilia, and/or mast cells.

In one embodiment of any of the aspects, the subject is a mammal. In one embodiment of any of the aspects, the subject is a mouse or human.

In one embodiment of any of the aspects, the method further comprises comparing the expression of the at least one odor response gene in the first OSN to the expression of the at least one odor response genes in an OSN of the first subtype that has been contacted with a benchmark scent.

In one aspect of any of the embodiments, described herein is a method of identifying an odorant receptor ligand, or measuring the strength of an odorant receptor ligand's binding, the method comprising measuring the expression of at least one odor response gene in a first olfactory sensory neuron (OSN) expressing a first odorant receptor that has been contacted with a candidate odorant receptor ligand; and determining that the candidate odorant receptor ligand binds the first odorant receptor if the expression of the at least one odor response gene in the first OSN is different from a reference level of expression in OSN expressing the first odorant receptor that has not been contacted with the candidate odorant receptor ligand.

In one embodiment of any of the aspects, the method further comprises measuring the expression of at least odor response gene in a plurality of OSNs, each of the plurality of OSNs having been contacted with a different candidate odorant receptor ligand. In one embodiment of any of the aspects, the magnitude of the difference in expression of the at least one odor response gene in the first OSN from the reference level of expression in an OSN expressing the first odorant receptor that has not been contacted with the candidate odorant receptor ligand correlates to the strength of the binding of the candidate odorant receptor ligand and the odorant receptor.

In one aspect of any of the embodiments, described herein is a method comprising measuring the expression of at least one odor response gene in a first olfactory sensory neuron (OSN) that has been contacted with at least one volatilized chemical compound, wherein the first OSN is obtained from or present in a subject; and determining that the subject has a loss of smell if the expression of the at least one odor response gene in the first OSN is different from the expression in a second OSN of the first subtype obtained from or present in normal, healthy individual. In one embodiment of any of the aspects, the loss of smell indicates the subject has or is at risk of having Parkinson's Disease. Alzheimer's Disease COVID-19, SARS-COV, MERS-COV, coronavirus, influenza, staphalococcus, and streptococcus. In one embodiment of any of the aspects, the method further comprises administering a treatment for Parkinson's Disease, Alzheimer's Disease, COVID-19, SARS-COV, MERS-COV, coronavirus, influenza, staphalococcus, and streptococcus if the subject is determined to have a loss of smell.

### BRIEF DESCRIPTION OF THE DRAWINGS

FIGS. 1A-1J examine how each OR is associated with a distinct OSN transcriptome.

FIG. 1A depicts a schematic of single cell RNA-seq (scSeq) experiments. Odorant receptor (OR) genes, which identify each OSN subtype, were excluded from the highly variable genes used in downstream analyses of OSN gene expression. FIG. 1B shows UMAP plots visualizing gene expression in mature OSNs, with normalized expression of known identity-related marker genes (38.345 mature OSNs from 6 mice depicted). FIG. 1C examines a UMAP plot depicting OSN subtypes expressing the indicated ORs. FIG. 1D shows a (left) transcriptome distances between individual OSNs expressing Olfr727, Olfr728 or Olfr729 (bars indicate individual mice). FIG. 1D shows (right) distributions of distances between cells expressing the same OR (within-OR, mean and 2.5-97.5th percentile across 1,000 restarts indicated) or different ORs (between-OR, mean and interquartile range across OSN subtypes of the pairwise distances between a given OSN subtype and all others indicated). FIG. 1E examines the accuracy of pairwise linear classifiers at predicting via OSN transcriptomes which OR is expressed by a given OSN (black dashed line indicates median, boxes represent 25^{th}/75^{th} percentile. 12.5^{th}/87.5^{th} percentile, etc. across 831C2=344,865 pairs). Classification performance is at chance levels upon shuffling OR labels across OSNs. FIG. 1F examines the accuracy of linear classifiers predicting which OR (out of 831) is expressed, at varying levels of prediction accuracy, from perfect (0.0%) to the rate at which the correct OR was within the top 2.5% of predicted ORs. Distributions depict the mean accuracy across the 831 OR-defined OSN subtypes (each averaged across 1,000 restarts). Performance is at chance levels upon shuffling OR labels across OSNs. FIG. 1G examines the accuracy of linear classifiers predicting which OR is expressed in an OSN from a held-out mouse, with training data provided from 5 separate mice. Both perfect (dark blue) and the top 1% (light blue) of predictions are shown. FIG. 1H shows a schematic of the genomic loci for the OR-swap and P2-IRES-GFP mouse lines. FIG. 1I shows UMAP plots of mature OSNs from wild-type and the mice shown in H, highlighting OSNs expressing M72, S50, or P2 receptors. FIG. 1J examines the accuracy of linear classifiers predicting OR identity for OSNs expressing M72, S50, or P2, trained and tested as indicated. See also FIGS. 8A-8P.

FIGS. 2A-2H depict OSN transcriptional variation can be decomposed into identity and activity gene expression programs. FIG. 2A displays the consensus non-negative matrix factorization (cNMF) identifies coherent gene expression programs (GEPs). Gene expression in each cell can be decomposed into a set of usages across a small number of GEPs, each of which are defined by their loadings for each gene. FIG. 2B shows UMAP plots showing the usage of each functionally annotated GEP in mature OSNs from wild-type mice housed in the home cage. FIG. 2C examines GEPHigh and GEPLow usages by the OSNs shown in B. Each point indicates the mean GEP usage for all OSNs expressing the same OR for each of the 831 ORs detected in at least 10 OSNs. FIG. 2D shows environmental state (ES) scores for each OSN are calculated by taking the difference between GEPHigh and GEPLow. FIG. 2E shows the mean ES score for each OSN subtype. ES scores were shuffled 1,000 times across cells, and the mean interquartile range across shuffles is depicted in gray for each OSN subtype, with the mean across sorted shuffles depicted by the dashed line. FIG. 2F is similar as in FIG. 2B, but for ES scores. FIG. 2G shows between-mouse correlation coefficients of the mean ES score for each OSN subtype. FIG. 2H examines the accuracy of pairwise linear classifiers (as in FIG. 1E) predicting OR identity using only the usages of GEPHigh and GEPLow. See also FIG. 9A-9L.

FIGS. 3A-3R depict OSN transcriptomes are shaped by the odor environment. FIG. 3A shows a schematic of the chronic naris occlusion experiment. FIG. 3B examines UMAP plots of mature OSNs depicting the nostril of origin. ES scores, and OSNs expressing example ORs. FIG. 3C depicts mean occlusion-dependent changes in GEP usage across OSN subtypes, as defined by OR expression. FIG. 3D shows the mean ES score for each OSN subtype for each nostril. ES scores decreased by occlusion for 770 out of 797 OSN subtypes (FDR≤0.01). FIG. 3E examines the correlation of the mean ES scores for each OSN subtype from the indicated nostril pairs (p=1.68×10⁻⁵, Mann-Whitney U test, for both within-vs. between-nostril comparisons). FIG. 3F depicts a schematic of the chronic optogenetic stimulation experiment. FIG. 3G depicts a mean ES score for each dorsal OSN subtype from stimulated and control (no-light) mice. ES scores increased following optogenetic stimulation for 56 out of 90 subtypes (FDR≤0.01). FIG. 3H shows the distribution of mean ES scores across OSN subtypes from each condition. ES scores following optogenetic stimulation are right-shifted (p=1.33×10⁻³⁴, KS-test). FIG. 3I shows a schematic of the environment switch experiment. FIG. 3J examines UMAP plots highlighting (left) OSNs expressing example ORs from mice housed in each of the three environments and (right) OSN ES scores. FIG. 3K depicts pairwise correlations of the mean ES scores for each OSN subtype, across mice from the indicated environments. FIG. 3L depicts pairwise correlations from FIG. 3K separated by the type of comparison. Correlations are higher for mice housed within the same environment (p=7.17×10⁻⁷, Mann-Whitney U test). FIG. 3M shows the percent of OSN subtypes whose ES scores change significantly (FDR≤0.01), for animals from the same or different environments. FIG. 3N shows the distribution of ES score changes, for OSN subtypes from the same or different environments. ES score changes are larger between environments (p=5.16×10⁻¹⁴⁰, KS-test). FIG. 3O depicts the observed occlusion-induced change in ES scores for each OSN subtype, as a function of ES scores from open nostrils. FIG. 3P shows the distribution of the mean ES scores across OSN subtypes from each nostril. ES scores following occlusion are left-shifted (p=3.74×10⁻²¹⁷, KS-test). FIG. 3Q shows the accuracy of a minimum distance classification procedure (see Methods) to predict in which environment a mouse was housed, based upon ES scores for each OSN subtype (observed or shuffled environment as labeled, curves depict the mean and interquartile range of classification accuracy for each environment across 1000 restarts). FIG. 3R examines the cumulative distribution of ES scores in a given reference environment for OSN subtypes whose ES scores were significantly increased, decreased, or unchanged across environments. Those OSNs whose ES scores decreased tended to have higher ES scores in the reference environment (p=4.03×10⁻⁸, KS-test); the opposite was true for those OSNs whose scores increased (p=2.32×10⁻¹⁴). See also FIGS. 10A-10T.

FIG. 4A-4F depicts large-scale transcriptional variation is organized into an environment-dependent rheostat. FIG. 4A examines the categorization of ES neuronal genes with high loadings in either GEPHigh or GEPLow, including “functional” genes that may regulate sensory responses. A subset of the 117 genes used in FIGS. 4B, 4E, and 4F are shown. FIG. 4B shows a heatmap of the z-scored expression across OSN subtypes of 117 ES neuronal genes (including 73 functional genes). FIG. 4C examines the expression (normalized by the total number of transcripts per cell and averaged across cells for each OSN subtype) of example functional genes associated with cither GEPHigh or GEPLow, as a function of ES scores. FIG. 4D depicts the expression of 73 functional genes as a function of OSN ES scores (binned into 50 quantiles); plots depict the mean and standard deviation of the z-scored expression of functional genes associated with GEPHigh and GEPLow. FIG. 4E shows (top) a heatmap of the chronic occlusion-dependent change in z-scored expression of ES neuronal genes for each OSN subtype. (bottom) Similar to FIG. 4D, but for changes in both functional gene expression and ES scores. FIG. 4F is similar to FIG. 4E, but for environment-dependent changes in functional gene expression. See also FIGS. 11A-11K.

FIGS. 5A-5L shows that Act-Seq identifies odor-responsive receptors and reveals that OSN transcriptomes determine acute in vivo odor responses. FIG. 5A shows a schematic of the Act-Seq experiment. FIG. 5B examines z-scored expression of 10 immediate early genes (IEGs, listed in Methods) for each OSN subtype from control or odor conditions (acetophenone (ACE) or octanal (OCT)), sorted for each odor. IEG expression was higher in the odor conditions (p<1×10⁻¹⁸ for each odor via Wilcoxon signed-rank test). FIG. 5C depicts odor-evoked activation score for each OSN subtype, as a function of that evoked by control solvent (dipropylene glycol. DPG). Odor-evoked activation scores are higher for responsive OSN subtypes (p<1×10⁻¹⁴, one-sided Wilcoxon signed-rank test for each odor). FIG. 5D shows a schematic of the acute (two hour) optogenetic stimulation experiment. FIG. 5E examines the distribution of mean activation scores for OSN subtypes expressing dorsal ORs in optogenetically-stimulated and control conditions. Activation scores increase with stimulation frequency (p<1×10⁻⁵, one-sided Jonckheere-Terpstra trend test). FIG. 5F depicts the percent of cells activated by acetophenone, for OSN subtypes that were responsive at 10% (and colored dark to light by the number of concentrations that activated each OSN subtype), increase with concentration (p<1×10⁻⁵, one-sided Jonckheere-Terpstra trend test). FIG. 5G shows activation scores, for OSN subtypes that were responsive at 10%, increase with increasing concentrations of acetophenone. (p<1×10⁻⁵, one-sided Jonckheere-Terpstra trend test). FIG. 5H depicts activation scores for OSNs expressing either M72 (Olfr160) or Olfr923 to either acetophenone or 2-hydroxy acetophenone (2HA) at the indicated concentrations. FIG. 5I shows activation scores for each odor as a function of control ES scores, for odor-responsive OSN subtypes. FIG. 5J examines the mean-squared error of predicted activation scores via linear regression models fit on either ES scores or expression of 73 functional genes, for odor-responsive OSN subtypes. Error bars represent mean and SD across 1,000 restarts, normalized to the models fit on ES scores. FIG. 5K shows activation scores for acetophenone-responsive OSN subtypes whose ORs have known in vitro EC50 values (Jiang et al., 2015; von der Weid et al., 2015). FIG. 5L is similar to FIG. 5I but for optogenetically-activated dorsal OSN subtypes (Medium intensity in FIG. 5D). See also FIGS. 12A-12S.

FIGS. 6A-6Q shows changes in OSN transcriptomes predict changes in acute odor responses, and acute odor responses predict long-term transcriptome changes. FIG. 6A displays a model depicting how occlusion- or environment-dependent changes in ES scores could affect acute odor responses. FIG. 6B shows a schematic of transient naris occlusion followed by Act-Seq. FIG. 6C displays the ES score, for the 745 OSN subtypes identified in transiently-occluded (but not unplugged) mice. Occlusion decreased ES scores for 726 OSN subtypes (554 significantly at FDR≤ 0.01). FIG. 6D examines the activation scores for acetophenone-responsive OSN subtypes are higher in unplugged than open nostrils (p=2.29×10⁻⁷. Wilcoxon signed-rank test). FIG. 6E shows changes in activation scores between acetophenone-responsive OSN subtypes from open or unplugged nostrils, as a function of the occlusion-dependent change for each subtype (as measured in the data in FIG. 6C). FIG. 6F examines correlation of the activation scores for acetophenone-responsive OSN subtypes, using data from either the same or different environments. Activation scores are more consistent within a given environment (p=0.014 Mann-Whitney U test). FIG. 6G depicts changes in activation scores across environments, as a function of changes in ES scores across environments, for acetophenone-responsive OSN subtypes. FIG. 6H shows a model depicting how activation upon a shift to a novel odor environment like environment A (envA) could subsequently change ES scores. FIG. 6I examines activation scores (relative to the mean across all OSN subtypes) two hours after the indicated environment shift. Compared to shifts between the same environment, home-envA and envA-home shifts induced opposing changes in activation scores, specifically for OSN subtypes with either significant increases or decreases in ES scores after two weeks (p<1×10⁻⁵ one-sided Jonckheere-Terpstra trend test for both: p=p=0.662 for OSN subtypes that remained constant after two weeks). FIG. 6J depicts changes in ES scores as a function of activation scores observed two hours after a shift to envA, for each OSN subtype (relative to their mean across all OSN subtypes). FIG. 6K shows accuracy of classifiers predicting whether ES scores would rise or fall after two weeks, based upon the activation scores observed two hours after a shift to envA. For 1,000 restarts, classifiers were fit using only OSN subtypes with significant ES scores changes after two weeks in envA, and the mean accuracy was summarized across OSN subtypes (error bars depict the mean and SD across restarts, accuracy is at chance levels after permuting the signs of the ES score changes).

FIG. 6L examines mean change in ES scores after five days of chronic exposure (relative to the mean across all OSN subtypes) to the indicated concentration of acetophenone for OSN subtypes that were acutely responsive to 10% odor (as identified in the experiments depicted in FIG. 5A-5K). Subtypes were separated into those responding to either 10% alone or at least two concentrations (significant effect of OR responsivity (10% only vs. at least two concentrations. F(1.270)=43.04, p=2.72×10⁻¹⁰) and chronic acetophenone concentration (F(2.270)=4.48, p=0.012) on ES score changes, but the interaction was not significant (F(2.270)=0.0085, p=0.99), via two-way ANOVA). FIG. 6M shows a change in ES scores (relative to the mean across all OSN subtypes) after chronic exposure to acetophenone, as a function of the acute activation score, for all acetophenone-responsive OSN subtypes identified at each concentration. FIG. 6N is the same as FIG. 6M, but for ES score changes as a function of activation scores after acute optogenetic stimulation (Medium intensity, see FIG. 5D). FIG. 6O shows a change in ES scores (relative to the mean across all OSN subtypes), at various timepoints after a home-envA shift, for OSN subtypes categorized based on the sign and significance of their two-week ES score changes. FIG. 6P is similar to FIG. 6M, but for changes in ES scores (relative to the mean across all OSN subtypes) induced by chronic exposure to acetophenone as a function of ES scores from mice housed in the home-cage. FIG. 6Q is similar to FIG. 6P, but for optogenetic stimulation and plotting changes in ES scores. See also FIGS. 13A-13S.

FIGS. 7A-7L depict the ambient odor environment determines functional odor responses. FIG. 7A shows a schematic of the olfactory bulb imaging experiment. Scale bar=200 μm. Data shown in FIGS. 7A-7D are from mouse 1 (see FIG. 14B). FIG. 7B examines mean z-scored dF/F responses to pentanal for example glomeruli and the mean fluorescence image (in gray) for each environment. Data from glomeruli marked a-c are depicted in FIG. 7C and FIG. 7D. Scale bars=200 μm. FIG. 7C depicts Z-scored dF/F responses for example glomeruli. Heatmaps depict average across trials within each session, and the mean±SEM across sessions for each environment are shown above. FIG. 7D examines glomerular responses to pentanal for each environment (mean±SEM across sessions), normalized to the home-cage (home1) response, for pentanal-responsive glomeruli. Asterisks indicate glomeruli whose responses in environment A (envA) differ from those of home1 (FDR≤0.01, permutation test). FIG. 7E shows the distance between the population response to pentanal in home1 and the response in cither envA or home2, as a function of time (see Methods). Shaded error bars depict the mean and SD across 1000 restarts. FIG. 7F examines the accuracy of pairwise linear classifiers predicting in which environment (home-cage or envA) a mouse was housed from the mean odor responses of increasing populations of glomeruli for each odor (as colored in H.).

FIG. 7G shows the accuracy of linear classifiers predicting odor identity from mean glomerular responses, to each of 16 odors, using increasing populations of glomeruli. Classification in which training and test data are from either the same or different environments, and from data with shuffled environment labels, are shown separately. FIG. 7H depicts a correlation matrix summarizing pairwise relationships between mean odor responses across the population of glomeruli from mouse 5 (see FIG. 14B). FIG. 7I examines the difference between the correlation matrices shown in FIG. 7H. FIG. 7J shows the procedure for evaluating changes in odor codes across environments, by assessing how well a given pairwise odor classifier generalizes, across all other odor pairs, to test data from the same or different environments. FIG. 7K depicts the classification accuracy for each of the test×train pairs (120×119 pairs), sorted by the accuracy when training and test data are from the same environment (within-environment). Colors depict whether training and test data are either from same or different environments, or from data with shuffled environment labels. FIG. 7L is a summary of FIG. 7K, showing the cumulative fraction of the absolute change in generalization accuracy, relative to within-environment accuracy. Sec also FIGS. 14A-14J.

FIGS. 8A-8Q examine OSN transcriptomes are predictable across individuals, and variation is distributed across hundreds of genes, related to FIGS. 1A-1J. FIG. 8A depicts a histogram of the number of cells expressing each OR, from wild-type mice housed in the home cage. FIG. 8B shows the cumulative percent of ORs observed in OSNs (sorted by how often each OR is observed) as a function of the cumulative fraction of OSNs that express any given OR. The top 200 ORs (of the 1016 detected) account for >50% of OSNs. FIG. 8C examines the number of OSN subtypes identified, thresholded by the number of OSNs expressing a given OR; colors represent numbers of OSNs used for this analysis: a threshold of 10 OSNs yielded 831 OSN subtypes. FIG. 8D determines the percent of OSNs expressing 0, 1 or >1 ORs thresholded by the number of transcripts (unique molecular identifiers, UMIs) required for an OR gene to be considered “detected”. Many of the rare 2 OR cells were clearly doublets and contained higher than average total numbers of UMIs compared to a typical 1 OR cell. Therefore, downstream analyses used a threshold of 3 UMIs and only considered cells in which only one OR was detected. FIG. 8E shows UMAP plots highlighting OSNs expressing the indicated ORs. FIG. 8F examines the mean fraction of all OSNs expressing a given OR (averaged across 1,000 restarts) as a function of the rank-ordered transcriptome distance from each cell expressing that OR. Shaded error indicates the interquartile range across ORs. FIG. 8G examines the distribution of within-OR transcriptome distances across OSN subtypes using cells from the same or different mice, or after shuffling OR labels. FIG. 8H shows the distribution of within-OR transcriptome distances as a function of the number of OSNs expressing each OR. FIG. 8I depicts the separability of the distributions of within-OR and between-OR transcriptome distances, as measured by the Jensen-Shannon Divergence (JSD), for various number of genes and increasing number of principal components. JSD was normalized to the maximum JSD value across all conditions. Highly variable genes (HVGs, used to compute the principal components) were selected based on their variability across OSNs expressing different ORs. Stars represent the number of HVGs used in the analyses shown in the paper. The JSD is low, and separability is at chance levels, when OR labels are shuffled. FIG. 8J shows the distribution of accuracies for linear classifiers predicting which OR is expressed in a given OSN, as a function of the number of OSNs expressing each OR. FIG. 8K examines the mean accuracy across OSN subtypes of linear classifiers predicting which OR is expressed as a function of the number of HVGs included (sorted by their informativeness, see Methods). Shading indicates classification thresholds, and error bars depict the mean and SD across 10 restarts. FIG. 8L is similar to FIG. 8K, but as a function of the number of genes removed. FIG. 8M (left) shows the accuracy of linear classifiers predicting which OR (out of 831) is expressed, after excluding all transcription factor (TF) and axon guidance genes. Distributions depict the mean accuracy across the 831 OR-defined OSN subtypes (each averaged across 1,000 restarts). FIG. 8M (right) is similar to left, but for various gene sets, including axon guidance genes and TF genes (which both contain HVGs and non-HVGs), and the 100 most predictive or all HVGs (reproduced from FIG. 1F). Classifier hyperparameters were optimized for each gene set. FIG. 8N depicts the accuracy of pairwise linear classifiers predicting which OR is expressed, with respect to (left) anatomical identity or (middle) OR class. FIG. 8N (right) depicts the accuracy of linear classifiers to predict which of dorsal class I, dorsal class II or ventral class II OR is expressed. FIG. 8O is similar to left and middle of FIG. 8N, but with respect to CD36-positivity. FIG. 8P is similar to left and middle of FIG. 8N, but with respect to genomic distance (distance between transcriptional start sites (TSS), in base pairs). FIG. 8Q is similar to left and middle of FIG. 8N, but with respect to the phylogenetic distance.

FIGS. 9A-9L examines how GEPHigh and GEPLow are aligned with the main axis of transcriptional variation across OSNs, related to FIGS. 2A-2H. FIG. 9A depicts consensus non-negative matrix factorization (cNMF) identifies 10 OR-specific gene expression programs (GEPs) whose usages are correlated among OSNs that express the same OR. OSNs expressing the same OR were randomly split into two populations and the correlation in GEP usage between the two populations was computed for each GEP. Error bars depict the mean correlation with SD across 10,000 restarts. 6 GEPs (including one with many genes related to the unfolded protein response (UPR)) only showed weak correlation, indicating that the usages for such GEPs are not specific to ORs in mature OSNs. FIG. 9B shows UMAP plots showing the usages of identified GEPs, together with the expression of genes with high loadings for each GEP. FIG. 9C examines gene loadings for the subset of HVGs whose loadings was higher than 2/1350 (total number of HVGs) in any of the 10 OR-specific GEPs; genes were hierarchically clustered by their loadings. FIG. 9D depicts the accuracy of linear classifiers predicting OR identity using GEPs across (left) OR pairs or (right) across all ORs. Performance upon shuffling OR labels is at chance=1/831. FIG. 9E shows the correlation coefficients between mean GEP usages for each OSN subtype. Strong anticorrelation between Dorsal/Ventral (p=−0.85). Anterior/Posterior (p=−0.70) and High/Low GEPs (p=−0.87) is observed. FIG. 9F examines mean usages of (left) GEPDorsal and GEPVentral or (right) GEPAnterior and GEPPosterior for each OSN subtype, from wild-type mice housed in the home cage. FIG. 9G examines the accuracy of pairwise linear classifiers predicting OR identity using only the usages of either the Dorsal/Ventral (D/V) or the Anterior/Posterior (A/P) GEPs. FIG. 9H depicts correlation coefficients of environmental state (ES), Dorsal/Ventral (DV), and Anterior/Posterior (AP) scores. DV and AP scores are calculated by GEPDorsal-GEPVentral and GEP Anterior-GEPPosterior, respectively. Individual mice are indicated as colored on top. FIG. 9I depicts correlation coefficients, computed across OSN subtypes, between the GEP usages and PC scores (calculated from the top 1.350 HVGs). FIG. 9J shows absolute values of the correlation coefficient, computed across OSN subtypes, between the ES score and the PC score for each of top 10 PCs. The ES score is highly correlated with the first PC (p=0.89), indicating that the ES score is a major axis of transcriptional variation across OSNs derived from mice housed in the home cage. Error bars depict the mean and SD across 1,000 restarts. FIG. 9K examines mean z-scored expression of immature (Gng8, Gap43) and mature (Gna1, Omp) OSN marker genes as a function of the developmental pseudotime from the onset of OR expression in late-immature OSNs (see Methods). FIG. 9L shows (left) GEPLow. (middle) GEPHigh, and (right) ES scores as a function of the developmental pseudotime from the onset of OR expression in late-immature OSNs. OSN subtypes are fractionated by their ES scores in mature OSNs.

FIGS. 10A-10T determines the dependence of OSN transcriptomes on long-term interactions with odors and air, related to FIGS. 3A-3R. FIG. 10A shows the accuracy of linear classifiers predicting OR identity based on HVG expression (see FIGS. 1E-1F) from open or occluded nostrils across OR pairs (left) or across all ORs (right). FIG. 10B examines the accuracy of linear classifiers predicting whether a given cell for each OSN subtype is from the open or closed nostril, using the usage of either (left) all 10 OR-specific GEPs or (right) only GEPHigh/GEPLow. FIG. 10C shows gene loadings for each GEP for HVGs that are differentially expressed (DE), or are unchanged (non-DE, in gray) by chronic naris occlusion. HVGs that decrease upon occlusion (in blue) have higher than average loadings compared to non-DE genes for GEPHigh (p<1×10⁻⁵⁷, KS-test, adjusted by the Holm-Bonferroni method) and lower than average loadings for GEPLow, and vice versa for genes that increased (in red, p<1×10⁻⁴⁸. KS-test, adjusted by the Holm-Bonferroni method). FIG. 10D depicts mean usages of GEPHigh and GEPLow for 797 OSN subtypes for each nostril, using data from all mice. FIG. 10E depicts the mean ES score for each OSN subtype for each nostril. Data from each mouse are shown separately. ES scores decreased by occlusion for 314/341. 356/391, and 320/340 of OSN subtypes, respectively (FDR≤0.01). FIG. 10F shows the mean ES scores for each OSN subtype for mouse pairs from the same or different environments. FIG. 10G depicts the cumulative percent of OSN subtypes whose ES scores differ significantly (FDR≤0.01) in different numbers of environment pairs. FIG. 10H shows the mean environment-dependent changes in GEP usage across OSN subtypes, categorized based on the sign and significance of their ES score changes across environments. FIG. 10I examines the accuracy of pairwise linear classifiers predicting OR identity using only the usages of the two ES GEPs (GEPHigh and GEPLow) of OSNs from each nostril in the chronic occlusion data. FIG. 10J shows a UMAP plot highlighting OSNs expressing the indicated ORs, using only cells from occluded nostrils. FIG. 10K shows a UMAP plot depicting the z-scored ES scores, using only cells from occluded nostrils. FIG. 10L examines the accuracy of pairwise linear classifiers predicting OR identity using only the usages of the two ES GEPs of OSNs from each of the three environments. FIG. 10M examines the accuracy of pairwise linear classifiers predicting in which environment a mouse was housed, based upon usages of either all GEPs or ES GEPs. Classification was performed for each OSN subtype, and distributions of the mean accuracies (across 1,000 restarts) across OSN subtypes with or without significant ES score changes across environments are shown (shuffled=environment labels permuted). FIG. 10N shows OR expression levels (as quantified by the number of transcripts of the expressed OR normalized by the total number of transcripts per cell) in each nostril, for each OSN subtype. FIG. 10O is similar to FIG. 10N, but for optogenetically-stimulated (12h) and control (no light) mice. FIG. 10P depicts pairwise correlations across all environments of the (left) mean OR expression levels or (right) the number of cells in which each OR was detected. FIG. 10Q shows, as in the right panel of FIG. 10F, but for Dorsal/Ventral (DV), and Anterior/Posterior (AP) scores. FIG. 10R examines ES scores for each OSN subtype from wild-type (WT) and β-Arresting mutant mice housed in the home cage. FIG. 10S is similar to FIG. 10R, but for OR expression levels. FIG. 10T shows (left) mean change in ES scores (relative to the mean across all OSN subtypes) 5 days after a shift to environment A (envA), for OSN subtypes from β-Arresting mutant mice. OSN subtypes whose ES score rose or fell after 2 week in envA in WT data show increase or decrease in ES scores in mutants (p<1×10⁻¹⁵ via Wilcoxon signed-rank test). FIG. 10T shows (right) correlation coefficient of changes in ES score for each OSN subtype between WT and β-Arresting mutant mice (p<1×10⁻⁴ via permutation test across 10,000 shuffles).

FIGS. 11A-11K. Functional gene expression exhibits organized correlation structure, related to FIGS. 4A-4F. FIG. 11A shows a heatmap showing pairwise correlations between the expression levels, across OSN subtypes, of the 117 ES neuronal genes, in data from mice housed in the home cage. FIG. 11B depicts a summary of the correlations between pairs of the 73 functional genes (see FIG. 4A), separated by pairs from either GEPHigh alone. GEPLow alone, or both GEPHigh and GEPLow. FIG. 11C shows correlation of the expression of individual functional genes and the ES score, computed across OSN subtypes, compared to correlations observed upon shuffling ES scores across OSN subtypes. FIG. 11D examines mean ES score and mean “functional” ES score (using GEPHigh and GEPLow usages calculated from the loadings of the 73 functional genes) for each OSN subtype from mice housed in the home cage. FIG. 11E depicts the accuracy of pairwise linear classifiers trained to predict OR identity using the GEPHigh and GEPLow usages calculated from the loadings of the 73 functional genes. FIG. 11F shows the mean occlusion-dependent change in ES score and “functional” ES score for each OSN subtype. FIG. 11G is similar to FIG. 11B, but for occlusion-dependent changes in functional gene expression. FIG. 11H is similar to FIG. 11C, but for occlusion-dependent changes in ES scores and functional gene expression. FIG. 11I shows optogenetic stimulation-dependent changes in expression of 73 functional genes as a function of changes in ES scores (binned into 10 quantiles); plots depict the mean and SD of the z-scored expression of functional genes associated with GEPHigh and GEPLow. FIG. 11J is similar to FIG. 11B, but for environment-dependent changes in functional gene expression. FIG. 11K is similar to FIG. 11C, but for environment-dependent changes in ES scores and functional gene expression.

FIGS. 12A-12S depict Act-Seq identified odor-activated OSNs and quantified the degree of transcriptional odor response, related to FIGS. 5A-5K. FIG. 12A shows (top left) UMAP plot showing mean z-scored expression of 10 immediate early genes (IEGs, see Methods for list of IEGs). FIG. 12A shows (top right) fraction of cells per OSN subtype that were activated by acetophenone (ACE) or octacal (OCT) based on IEG expression. OSN subtypes were defined as odor-responsive when at least 70 percent of cells expressing the given OR for that subtype were activated (dashed line), and these ORs were considered as activated. FIG. 12A shows (middle left) UMAP plot highlighting activated cells identified by unsupervised Leiden clustering (see Methods). FIG. 12A shows (middle right) fraction of cells per OSN subtype that were activated based on Leiden clustering, colored by odor-responsiveness as assessed by IEG expression. FIG. 12A shows (bottom left) UMAP plot depicting odor-driven activation as quantified by unsupervised tensor component analysis (TCA). FIG. 12A shows (bottom right) the distribution of TCA loadings of a factor representing odor activation (with 11-times higher weight on the odor source compared to the control condition) for OSN subtypes for mice exposed to ACE and OCT, colored by odor-responsiveness as assessed by IEG expression. FIG. 12B depicts the distribution of the mean expression of 10 IEGs across OSN subtypes, separated by odor-responsiveness. FIG. 12C shows a Venn diagram depicting the overlap between ORs identified as acetophenone receptors via Act-seq, the DREAM method, and phospho-S6 immunoprecipitation (from von der Wied et al. 2015. Jiang et al. 2015). For the 8 ORs identified by only the phospho-S6 method, 3 of them were not activated in any cells via Act-Seq and the remaining 5 of them were partially activated (activated in 43.8% of cells per OR on average). This analysis only includes the 717 ORs which were present in four or more OSNs in these odor-activation experiments. Although Act-Seq captured nearly all such receptors that were also captured by DREAM and phospho-S6 (and many missed by both of these techniques), the 717 observed ORs included only 16/22 DREAM and 25/49 phospho-S6 ACE ORs; these “missed” receptors possibly reflect ORs that are expressed in particularly small numbers of OSNs. FIG. 12D shows a Venn diagram depicting the overlap between ORs activated by ACE and OCT. The overlap is at chance levels (odds ratio=1.42, p=0.29 Fisher's exact test). FIG. 12E examines comparisons of acute gene expression changes induced by ACE or OCT compared to solvent-only (DPG) condition for 996 genes with significant changes (FDR≤1×10⁻³) in at least one odor. FIG. 12F shows the mean percent of ORs activated by the indicated concentration of acetophenone. FIG. 12G shows the mean percent of ORs activated by 10% acetophenone and its analogs (2-hydroxyacetophenone (2HA), 4-methyl acetophenone (4MA), methyl salicylate (MS)). FIG. 12H depicts a heatmap depicting percent overlap of activated ORs among four acetophenone-related odors at 10% concentration. ORs activated by acetophenone analogs are also consistently activated by acetophenone (odds ratio>22, p<1×10⁻¹¹ via Fisher's exact test for each of three analogs). FIG. 12I examines correlation coefficients across individual mice of activation scores for OSN subtypes responsive to either ACE or OCT (p≥0.89 between mice exposed to the same odor). FIG. 12J shows correlation coefficients comparing activation scores for OSN subtypes activated by acetophenone-related odors, shown for pairs of mice exposed to the same or different odors. The correlation is higher in mice exposed to the same odor (p=0.002. Mann-Whitney U test). FIG. 12K depicts activation scores for four acetophenone-related odors, for OSN subtypes responsive to each odor. FIG. 12L shows the accuracy of pairwise linear classifiers trained to distinguish pairs of acetophenone-related odors based upon activation scores from odor-responsive OSN subtypes (using only OSN subtypes responsive to both odors in each pair). Observed and shuffled (odor labels are shuffled 1,000 times) classification accuracies are shown. FIG. 12M examines the accuracy of a minimum distance classification procedure to predict odor identity of the four acetophenone-related odors using OSN subtype-specific activation scores (see Methods), as a function of the number of OSN subtypes (from the set of OSN subtypes responsive to any of the four odors). The curves depict the mean and interquartile range of classifier accuracy for each odor across 1,000 restarts for the observed data, and for data in which odor labels were shuffled for each OSN subtype. FIG. 12N depicts the correlation coefficient between the activation score and the control ES score for each OSN subtype, evaluated at various thresholds used for identifying activated cells based on IEG expression and for identifying responsive ORs (fraction of cells activated). The threshold used in FIG. 5I (70% of cells per OR and IEG expression>0.2) is highlighted with stars. FIG. 12O shows the correlation coefficient between the activation score and the control ES score for each OSN subtype, within and between individual mice. For each odor, the observed negative correlation between activation and control ES score is higher (p<1×10⁻⁴ via permutation test) than any value in the distribution of correlation coefficients measured after permuting the activation score across OSN subtypes for 10,000 shuffles. FIG. 12P is similar to FIG. 12O, but for four acetophenone-related odors (p<8×10⁻³ via permutation test for each odor). FIG. 12Q shows mean activation scores at the indicated concentration for each of the acetophenone-responsive OSN subtypes, as a function of the in vitro EC50 for each OR, as reported in Jiang et al. 2015 and Saito et al. 2009 (see also FIG. 5K). FIG. 12R examines normalized error for predictions (see Methods) of activation scores (both observed and shuffled) for OSN subtypes responsive to ACE at 10%, based on control ES scores, the in vitro EC50 for each OR (from Jiang et al. 2015 and Saito et al. 2009), or both. Error bars represent mean and SD across 1,000 restarts. FIG. 12S is similar to FIG. 12O, but for data derived from the acute optogenetic stimulation experiment (p<1×10⁻⁴ via permutation test for each stimulation frequency).

FIGS. 13A-13S depict odor responses are determined by OSN Environmental State (ES) scores and changes in ES score are influenced by acute odor responses, related to FIGS. 6A-6Q. FIG. 13A shows a summary of the correlations between pairs of the 73 functional genes, separated by pairs from either GEPHigh alone. GEPLow alone, or both GEPHigh and GEPLow, using data from transiently-occluded (but not unplugged) nostrils (see FIG. 6B). FIG. 13B depicts mean transient occlusion-dependent changes in GEP usage across OSN subtypes. FIG. 13C shows the fraction of OSN subtypes responsive to acetophenone (ACE) from unplugged nostrils, for OSN subtypes responsive to 10% ACE (as shown in the top right of FIG. 12A). FIG. 13D examines mean changes in ES (left) or activation scores (right) between acetophenone-responsive OSN subtypes from open or unplugged nostrils, as a function of the difference in ES scores (binned into quartiles), as measured in control mice that were transiently-occluded (but not unplugged). OSN subtypes whose ES scores fell more showed increased activation (p=2.63×10⁻⁴, Mann-Whitney U test). FIG. 13E shows normalized mean-squared error for predictions of the occlusion-dependent change in activation scores for OSN subtypes responsive to acetophenone, with predictions based upon the occlusion-dependent changes in functional gene expression. Model errors increase if the nostril type (open vs. transiently occluded) of the control mice was shuffled, or if the activation scores are shuffled across OSN subtype. Errors were normalized to the value from the observed data, and the error bars depict the mean and SD across 1,000 restarts. FIG. 13F depicts a Venn diagram, showing that ORs identified as activated by acetophenone in each environment are largely consistent but not fully overlapped; modulation of odor responses across environments may contribute to the changes in activated ORs identified in each environment via our relatively conservative thresholds (see FIG. 13G). FIG. 13G shows an activation score distributions for OSN subtypes that were responsive to acetophenone in different numbers of environments (0-3). OSN subtypes that responded to acetophenone in 1-2 environments were still activated more in the other environments in which they were not considered as responsive (shown in gray) than those that were not responsive in any environment, consistent with the possibility that these receptors were activated to some extent in all environments. FIG. 13H examines the overlap in identified OSN subtypes responsive to acetophenone between pairs of mice housed within the same or different olfactory environments. Pairs of mice exposed to acetophenone exhibit activation in overlapping sets of OSN subtypes (positive log-odds), but the overlap is greater between mice that were housed within the same olfactory environment (p=0.026. Mann-Whitney U test). The overlap and log-odds are at chance levels after permuting the identity of ACE ORs across 1,000 restarts for each mouse pair. FIG. 13I shows the cumulative percent of acetophenone-responsive OSN subtypes identified in each environment, colored by the number of environments in which each subtype responded. FIG. 13J examines the mean activation scores for each acetophenone-responsive OSN subtype, sorted by each environment, for OSN subtypes responsive in all three environments. FIG. 13K depicts the correlation coefficient between the activation score and the control ES score for each acetophenone-responsive OSN subtypes, using data from mice housed in the indicated environments. The observed value is stronger than any value in the distribution of correlation coefficients measured after permuting the activation score across OSN subtypes for 10,000 shuffles (p<1×10⁻⁴ via permutation test for each environment). FIG. 13L examines the classification accuracy for a pairwise linear classifier predicting the environment from which a given set of OSNs derived. Classification was performed for each acetophenone-responsive OSN subtype with significant ES score changes across environments, using the activation scores in each environment for that OSN subtype. Shuffle=permuted environment. FIG. 13M shows the mean environment-dependent changes in activation scores, for acetophenone-responsive OSN subtypes, separated in OSN subtypes whose ES scores rose or fell across environments. OSNs whose ES scores increased in one environment relative to another exhibited lower acetophenone-driven activation scores, while those whose ES scores decreased showed the opposite trend (p=1.77×10⁻¹¹. Mann-Whitney U test). FIG. 13N is similar to FIG. 13E, but for the environment-dependent changes in activation scores. FIG. 13O shows OR expression levels after chronic exposure to the indicated concentration of acetophenone for 5 days, normalized to the control condition (exposure to DPG for 5 days). ORs identified as activated in none of the concentrations, at 10% alone, or at 2 or more concentrations by acute (two-hour) Act-Seq are shown separately. FIG. 13P examines the correlation between environment-dependent changes in ES scores (relative to their mean across all OSN subtypes) after 24 hours and 2 weeks, for individual OSN subtypes. FIG. 13Q depicts the mean change in z-scored expression of 73 functional genes (relative to the mean change observed across all OSN subtypes and show separately for genes associated with GEPHigh and GEPLow), at various timepoints after a shift to envA, for OSN subtypes categorized based on the sign and significance of their two-week ES score changes. FIG. 13R examines mean ES scores for OSN subtypes from mice exposed to acetophenone or DPG for 2 h and housed in home cages for 22h. Acetophenone-induced changes in the ES scores were not significantly different for acetophenone-responsive OSN subtypes compared to those that did not respond to acetophenone (Mann-Whitney U test, p=0.214). FIG. 13S examines mean expression of Nr4a1 and Fos normalized by the total number of transcripts per cell for OSN subtypes, as a function of the ES scores (binned by steps of 50) for each OSN subtype in mice housed in the home cage, compared to that observed in acetophenone-responsive OSN subtypes following two-hour exposure to acetophenone.

FIGS. 14A-14J examine odor environments determine acute functional odor responses in the olfactory bulb, related to FIGS. 7A-7L. FIG. 14A shows max-normalized mean fluorescence for individual sessions from a single mouse (mouse 1 in FIG. 14B), imaged for five weeks across environments (rows). Scale bars=200 μm. FIG. 14B examines pairwise enhanced correlation coefficients for the entire FOV for all imaging sessions from each mouse (after registering FOVs across sessions, see Methods). FIG. 14C shows the percent of odor-responsive glomeruli (see Methods), for the three indicated odors at two separate concentrations. Each circle indicates a single mouse. FIG. 14D shows the percent of environment-sensitive glomeruli (of the glomeruli responsive to each odor) whose odor responses significantly differed (FDR≤0.01 via permutation test), when mice were housed in home1 compared to environment A (envA). Odors were tested at two concentrations; pentanal-high corresponds to the concentration used in FIGS. 7B-7E. Each point represents a single mouse. FIG. 14E examines the fold-change in the number of glomeruli that differ significantly when comparing odor responses between home1/envA and home1/home2; each circle indicates a single mouse. A larger number of glomeruli change significantly across environments (see also FIG. 14F). FIG. 14F depicts the percent of glomeruli whose difference in odor response between home1/envA has the opposite sign as that observed between envA/home2, for all glomeruli significantly different between home1/envA (dots refer to individual mice); upon return to the home cage after experiencing envA, the odor responses of nearly all glomeruli are at least partially reverted. FIG. 14G shows the percent of odor-responsive glomeruli for the mice used for decoding analysis (mouse 5 and 6 in FIG. 14B). Each circle indicates an odor, and colors correspond to those in FIG. 7H. FIG. 14H examines the percent of environment-sensitive glomeruli (of the glomeruli responsive to each odor, colored as in FIG. 7H) whose odor responses significantly differed (FDR≤0.01 via permutation test), when mice were housed in home1 compared to envA. FIG. 14I shows absolute changes in pairwise correlations for mean odor responses (see FIGS. 7H-7I). Observed values and distribution of means after permuting environment labels across 10,000 shuffles are shown for each mouse (p<1×10⁻⁴ for each mouse). FIG. 14J is similar to FIG. 7K. Classification accuracy for each of the test×train pairs (120×119 pairs), colored by whether training and test data are from either the same or different environments, or from data with shuffled environment labels (and sorted independently for each of the three conditions).

FIG. 15A-15D show mouse Act-Seq for various odors and concentrations. FIG. 15A shows the percent of odorant receptors (OR) activated by each of the odor-concentration pairs. 22 odors from each of the four different odor classes are shown: aldehyde, ketone, ester, and acid, and the lightness of the color represents chain length, lighter colors=shorter chain length. Most of the odors were tested in at least two concentrations. Act-seq can identify activated ORs for both very narrowly tuned odors (short chain and acids) and broadly tuned ones (long chain). For each odor-concentration pair, we had ˜800 ORs that were detected in at least 4 cells. FIG. 15B examines the percent of ORs activated as a function of odor concentration. Each color indicates each odor as shown in FIG. 15A. FIG. 15C depicts a UMAP plot showing two dimensional representation of the patterns of Activation score for 22 odors, across 969 ORs. Colors indicate the clusters identified based on Activation scores. One concertation was used for each odor based on the vapor pressure. FIG. 15D shows a heatmap showing Activation scores across 969 ORs and 22 odors. Colors on the left represents the OR clusters shown in FIG. 15C, and colors on top represents odors as in FIG. 15A.

FIG. 16A-16D show in vivo human act-seq data. FIG. 16A depicts a UMAP plot showing olfactory cells obtained from a human subject using a nasal swab after 1 hour exposure to 2-phenylethanol, which smells like rose oil. 1 hour exposure is performed by asking the subject to sniff the odor vial every 2-5 minutes. All major olfactory cell types were identified. OSN: olfactory sensory neuron lineage (progenitors and immature or mature neurons). SUS: sustentacular cells. HBC: horizontal basal cell. MV: microvillar cells. BG: Bowman's gland cells. FIG. 16B examines total count normalized expression of GFY gene (a mature OSN marker) in the UMAP plot. FIG. 16C examines total count normalized expression of immediate early genes (IEG) in OSNs that expressed odorant receptors (OR). FIG. 16D shows mean total count normalized expression of 9 IEGs in each of the OSNs expressing OR10A6 or other ORs. Horizontal bars indicate mean across cells. OSNs expressing OR10A6 have much higher IEG expression, which is a hallmark of neuronal activation. OR10A6 was shown to be activated by 2-phenylethanol in vitro in a previous study (doi.org/10.3390/molecules25204743).

### DETAILED DESCRIPTION

In mammals, the sense of smell is controlled by olfactory sensory neurons (OSNs). Each OSN expresses an odorant receptor that binds to a volatizlied chemical compound (e.g., a scent molecule or a molecule that is part of a scent). The binding of the odorant receptor to the volatilized chemical compound is then transmitted to the central nervous system by the OSN, and the mammal perceives a scent. Importantly, each OSN expresses only one odorant receptor and there are approximately 1,000 different odorant receptors. It has therefore proven extremely difficult to determine which odorant receptor binds to which volatilized chemical compound(s), particularly in vivo.

The inventors have now discovered downstream transcriptional targets of odorant receptor activation. Combined with single cell sensitivity in expression assays (e.g., sequencing), this makes it possible to identify the individual OSNs that are responsive to any given volatilized chemical compound and thereby identify the odorant receptor that binds that volatilized chemical compound. This ability to dissect the chemical and genetic basis of scent perception permits improved methods of replicating or modulating scents.

In one aspect of any of the embodiments, described herein is a method comprising: measuring the expression of at least one odor response gene in a first olfactory sensory neuron (OSN) of a first subtype that has been contacted with at least one volatilized chemical compound. In some embodiments of any of the aspects, the method further comprises measuring the expression of at least one odor response gene in a second OSN that has not been contacted with the at least one volatilized chemical compound. In some embodiments of any of the aspects, the method further comprises determining that the first subtype expresses an odorant receptor that binds to the at least one volatilized chemical compound if the expression of the at least one odor response gene in the first OSN is different from a reference level of expression in a second OSN that has not been contacted with the at least one volatilized chemical compound. In some embodiments of any of the aspects, the method further comprises determining that the first subtype expresses an odorant receptor that binds to the at least one volatilized chemical compound if the expression of the at least one odor response gene in the first OSN is increased relative to a reference level of expression in a second OSN that has not been contacted with the at least one volatilized chemical compound.

In some embodiments of any of the aspects, the method further comprises measuring the expression of at least one odor response gene in a second OSN of the first subtype that has not been contacted with the at least one volatilized chemical compound. In some embodiments of any of the aspects, the method further comprises determining that the first subtype expresses an odorant receptor that binds to the at least one volatilized chemical compound if the expression of the at least one odor response gene in the first OSN is different from a reference level of expression in a second OSN of the first subtype that has not been contacted with the at least one volatilized chemical compound. In some embodiments of any of the aspects, the method further comprises determining that the first subtype expresses an odorant receptor that binds to the at least one volatilized chemical compound if the expression of the at least one odor response gene in the first OSN is increased relative to a reference level of expression in a second OSN of the first subtype that has not been contacted with the at least one volatilized chemical compound.

In one aspect of any of the embodiments, described herein is a method comprising: measuring the expression of at least one odor response gene in a first OSN of a first subtype that has been contacted with at least one volatilized chemical compound; measuring expression of at least one odor response gene in a second OSN that has not been contacted with the at least one volatilized chemical compound; and determining that the first subtype expresses an odorant receptor that binds to the at least one volatilized chemical compound if the expression of the at least one odor response gene in the first OSN and the second OSN are different. In one aspect of any of the embodiments, described herein is a method comprising: measuring the expression of at least one odor response gene in a first OSN of a first subtype that has been contacted with at least one volatilized chemical compound; measuring expression of at least one odor response gene in a second OSN of the first subtype that has not been contacted with the at least one volatilized chemical compound; and determining that the first subtype expresses an odorant receptor that binds to the at least one volatilized chemical compound if the expression of the at least one odor response gene in the first OSN and the second OSN are different.

In one aspect of any of the embodiments, described herein is a method comprising: measuring the expression of at least one odor response gene in a first OSN of a first subtype that has been contacted with at least one volatilized chemical compound; measuring expression of at least one odor response gene in a second OSN that has not been contacted with the at least one volatilized chemical compound; and determining that the first subtype expresses an odorant receptor that binds to the at least one volatilized chemical compound if the expression of the at least one odor response gene in the first OSN is increased relative to the expression of the at least one odor response gene in the second OSN. In one aspect of any of the embodiments, described herein is a method comprising: measuring the expression of at least one odor response gene in a first OSN of a first subtype that has been contacted with at least one volatilized chemical compound; measuring expression of at least one odor response gene in a second OSN of the first subtype that has not been contacted with the at least one volatilized chemical compound; and determining that the first subtype expresses an odorant receptor that binds to the at least one volatilized chemical compound if the expression of the at least one odor response gene in the first OSN is increased relative to the expression of the at least one odor response gene in the second OSN.

As used herein, an “odor response gene” is defined as a gene whose expression alters when a chemoreceptor binds its cognate scent molecule. These chemoreceptors, which are also known as odorant receptors, are expressed in the cell membranes of olfactory receptor neurons and are responsible for the detection of odorants (for example, compounds that have an odor) which give rise to the sense of smell. Activated odorant receptors trigger nerve impulses which transmit information about odor to the brain. Described herein are at least 800 human odor response genes at least 1400 murine odor response genes.

Examples of odor response genes include, but are not limited to Tsc22d1, Rcan1, Fosb, Pcdh10, Dusp14, Srxn1, Gm13889, Golga4, Heg1, Cdkn1a, Gpr137c, Zfp655, Ublcp1, Efr3b, Etf1, Srrm4, Epcam, Trp53inp2, Arhgap17, Ric8b, 9530034E10Rik, Oaz2, Ctxn3, Slc38a2, Exoc6b, Nfatc1, Junb, Tle3, Siah2, Fos, Ago2, Arhgef28, Ddx3x, Hspa8, Hnmnph3, Erf, Ak2, Pnrc1, Mafg, D1Ertd622c, Arid5b, Scn3b, Phtf2, Hsph1, Cpeb4, Ubc, Sema7a, Dleu2, Rc3h1, Pcnx4, Btg2, 6430548M08Rik, Selenok, Psmd8, Map9, Cry2, Hspa41, Mcl1, Dnaja1, Fth1, Fam208a, Bmpr2, Zfp608, BC005561, Rab11fip4, Smim13, Ppp2ca, Sod1, Neat1, Brinp2, Tuba4a, Cdc14a, Kdm6b, Uckl1os, Stk40, Dnajb2, Dnajb9, Spsb3, Sub1, Rheb, Manea, Fzd3, Zfand5, Uhrflbp1l, Cebpb, Wdfy3, Mfgc8, Inpp4a, Nab1, Tccal9, Ubc2g1, Pcdh7, Eefla1, Tnrc6c, Cd82, BC005537, Sdcbp, Ddx5, Olfm1, Srpk1, M6pr, Braf, Ahi1, Clcn3, Eef2, Rbm26, Arfgef3, Eif5, Tmem150c, Acbp2, Snrk, Bcx3, Epb4112, Samd4b, Stard10, Pappa, Grsf1, Ralgds, Arih1, Micu3, Ptges3, Kras, Morf412, Rtp2, Wdr26, Hspa5, Zmynd8, Sv2c, Paip2, Rab39b, Ywhaz, Gpat3, Psen2, Pde4a, Atp2b1, Irs2, Cfap69, Hspa9, Tob1, Hspa14, Pard6a, Mapk4, Nr4a1, Sik3, Epha7, Hagh, Kalrn, Prkar1a, Lmbrd1, Erich3, Sf3b1, Ksr2, Ybx1, Nfix, Hivep1, Mfn2, Elof1, Wdfy2, Emd, Azin1, Wbp1, Nop53, Syt7, Ncoa1, Smarca5, Aes, Rnf150, Plxnb2, Wdsub1, Dyrk4, Epb4111, Pcdh8, Daam1, Prpf4b, Tnrc6a, Rufy3, Ptdss2, Tmem230, Scn3a, Rock 1, Dph3, Zbtb7b, Yipf4, Ift74, Ebf1, Wsb2, Rnf182, Arglu1, Ppm11, Ccdc189, Ptov1, Ccdc157, Ppa1, Pnpla8, Rack1, Rsrp1, Ttc9, Hcn2, Ccdc40, Kat2b, Cngb1, Ndufa3, Eefla2, Fmn2, Macrod1, Polr2i, 1700016K19Rik, Jade1, Slcla2, Ralbp1, Eif4c3, Morn2, Trappc21, Scrn1, Rtp1, Phyh, Fetub, Mapre3, Dnajb13, Coa3, Spef1, 1810058124Rik, Ccp290, Bbs4, Nin, D430042009Rik, Tex9, Sdc3, Ebf4, Oaz1, Napa, Ttc8, Sdhaf4, Cuta, Commd6, Sfr1, Hcfclr1, Nxn12, Kcnh3, Trpm7, Glb112, Slc25a39, Nsmf, Pifo, Oscp1, 1110008P14Rik, Egr1, Ttll6, Sem1, Aplg1, Fkbp2, Suclg1, Cidea, Dynlrb2, Ndufa2, Map1a, Sun1, Rufy2, Rnh1, Dtna, Anapc16, Pdhb, A430035B10Rik, Faim2, Ebf2, Ccdc28b, Ttll3, Drc1, Sys1, Prdx5, Palm, Slu7, Dnajc15, Elmod1, Paqr9, Tubb3, Ascc1, Ndufc1, Pon2, Fkbp4, Trp1, Cldn3, Cby1, Tctex1d2, Slc48a1, Nme5, 1110004E09Rik, Cnppd1, Naa38, Vdac3, Puf60, Cers5, Cbr1, Ift43, Fam217a, Ankrd10, Ubc2n, Pfdn6, Mpc2, Ncbp2, Timm13, Lamtor5, Uqcr10, Slc27a2, Arpc51, Adh5, Gkap1, Ndufaf4, Psip1, Omp, Bcl11a, Ift81, 2010107E04Rik, Ubxn1, Ech1, Tnrc6b, Tmpo, Ssbp4, 2410015M20Rik, Mapk8ip2, Naxc, Gm13589, Hdgf13, Slc22a23, Cacna1h, Slc25a5, Ndufa13, Anp32a, Ndufb10, Lhx2, Cenpx, Rnf220, Ankrd54, Ppdpf, Dtd1, Auh, Mrpl51, Psmd13, Lamtor4, Hist2h2be, Mapkap1, Arhgdig, Ndufv3, Ilkap, Ctsa, Ift27, Lpcat4, Atp50, Ypel3, Gadl1, Msln1, Hint2, Acbd7, Manf, Lamtor2, Ak1, Arl6, Uxs1, Sri, Tomm7, Ubxn6, Cd47, Snrpd3, Ppid, Rsph9, Rbfox2, Pigp, C1qbp, Fmc1, Bcl7a, Krtcap2, Flrt1, Sdhd, Bphl, Sod2, Ndufb7, Prrc2b, Rex1bd, Rab36, Pdrg1, Pfn2, Rarg, B230118H07Rik, Elavl3, Mlf1, Eif1, Mett126, Kmt5a, Ndufa5, Ndufb3, Emc8, Bad, Smc3, Dpm3, Ndufa11, Cisd1, Mrpl27, Cldn9, Sec61b, Tpd5212, Polr2c, Ndufa7, Atf5, Gabarap, Srsf3, Psmb3, Bax, Atox1, 2410004P03Rik, Atpla1, Ptprs, Cib1, Hist1h2bc, Atp5k, Hmox2, Fam92b, Chd4, Elob, Kif5a, Tbcb, Ndufa4, Uqcr11, Hras, Atp6vlf, Pam16, Pim3, Lrriq1, Nfu1, Supt4a, Swi5, Mtx2, Faim, Taldo1, Ccdc34, Rbm39, 2210016L21Rik, Dpy30, Tomm22, Pygo1, Mat2b, Mrpl28, Eid1, Snrpe, Atp5j, Ndufs7, Park7, Ndufs4, Cirbp, Polr2f, Fbxo9, Mgst3, Gtf2h5, Mtch1, Mrps24, Actr1b, Sh3bgrl3, Bmpr1a, Pfn1, Tesc, Tmem258, Mrps14, Abhd16a, Vdac2, Nxph3, Polr2g, Them6, Cfap126, Pitpnc1, Kctd1, and Dmkn. In some embodiments, the at least one odor response gene is selected from Tsc22d1, Rcan1, Fosb, Pcdh10, Dusp14, Srxn1, Btg2, Egr1, Fos, Fosb, Gm13889, Junb, and Nr4a1. In some embodiments, the at least one odor response gene is selected from the group consisting of: Tsc22d1, Rcan1, Fosb, Pcdh10, Dusp14, Srxn1, Fos, and Nr4a1. In some embodiments, the at least one odor response gene is selected from the at least one odor response gene is selected from the group consisting of: Tsc22d1, Rcan1, Fosb, Pcdh10, Dusp14, and Srxn1. In some embodiments, the at least one odor response gene is selected from the group consisting of: Btg2, Egr1, Fos, Fosb, Gm13889, Junb, Nr4a1, Nr4a2, Pcdh10, and Srxn1. In some embodiments, the at least one odor response gene is selected from the group consisting of: Btg2, Egr1, Fos, Fosb, Gm13889, Junb, Nr4a1, Nr4a2, Pcdh10, Srxn1, Tsc22d1, Rcan1, and Dusp14.

The sequences of the odor response genes are known in the art for a number of species. For example, the sequences of the odor response genes can be the sequences available in the NCBI database for the Gene ID Nos, in Table 1, e.g., as of Dec. 2, 2022. The NCBI database entries provide genomic and mRNA sequences, including splice variants and homologs and orthologs for a number of species. As a further example, the sequences of the odor response genes can be the sequences available in the ENSEMBL database for the ESEMBL Gene ID Nos, in Table 1, e.g., as of Dec. 2, 2022 (ENSEMBL release 108). The ENSEMBL database entries provide genomic and mRNA sequences, including splice variants and homologs and orthologs for a number of species.

TABLE 1
Odor Response Genes

| | | | | start_ | end_ | NCBI |
|---|---|---|---|---|---|---|
| gene | kind | ensembl_gene_id | chr | position | position | Gene ID |
| Btg2 | ieg | ENSMUSG00000020423 | 1 | 134075170 | 134079120 | 12227 |
| Egr1 | ieg | ENSMUSG00000038418 | 18 | 34859823 | 34864984 | 13653 |
| Fos | ieg | ENSMUSG00000021250 | 12 | 85473890 | 85477273 | 14281 |
| Fosb | ieg | ENSMUSG00000003545 | 7 | 19302696 | 19310051 | 14282 |
| Gm13889 | ieg | ENSMUSG00000087006 | 2 | 93955810 | 93957201 | 620695 |
| Junb | ieg | ENSMUSG00000052837 | 8 | 84974484 | 84978718 | 16477 |
| Nr4a1 | ieg | ENSMUSG00000023034 | 15 | 101254269 | 101274795 | 15370 |
| Nr4a2 | ieg | ENSMUSG00000026826 | 2 | 57106830 | 57124003 | 18227 |
| Pcdh10 | ieg | ENSMUSG00000049100 | 3 | 45378398 | 45435623 | 18526 |
| Srxn1 | ieg | ENSMUSG00000032802 | 2 | 152105516 | 152111376 | 76650 |
| Tsc22d1 | activation | ENSMUSG00000022010 | 14 | 76414961 | 76507765 | 21807 |
| Rcan1 | activation | ENSMUSG00000022951 | 16 | 92391953 | 92470867 | 54720 |
| Fosb | activation | ENSMUSG00000003545 | 7 | 19302696 | 19310051 | 14282 |
| Pcdh10 | activation | ENSMUSG00000049100 | 3 | 45378398 | 45435623 | 18526 |
| Dusp14 | activation | ENSMUSG00000018648 | 11 | 84048041 | 84069261 | 56405 |
| Srxn1 | activation | ENSMUSG00000032802 | 2 | 152105516 | 152111376 | 76650 |
| Gm13889 | activation | ENSMUSG00000087006 | 2 | 93955810 | 93957201 | 620695 |
| Golga4 | activation | ENSMUSG00000038708 | 9 | 118506267 | 118582519 | 54214 |
| Heg1 | activation | ENSMUSG00000075254 | 16 | 33684370 | 33771576 | 77446 |
| Cdkn1a | activation | ENSMUSG00000023067 | 17 | 29090979 | 29100722 | 12575 |
| Gpr137c | activation | ENSMUSG00000049092 | 14 | 45219717 | 45282725 | 70713 |
| Zfp655 | activation | ENSMUSG00000007812 | 5 | 145231715 | 145247302 | 72611 |
| Ublcp1 | activation | ENSMUSG00000041231 | 11 | 44454571 | 44470498 | 79560 |
| Efr3b | activation | ENSMUSG00000020658 | 12 | 3962554 | 4038915 | 668212 |
| Etf1 | activation | ENSMUSG00000024360 | 18 | 34902785 | 34932007 | 225363 |
| Srrm4 | activation | ENSMUSG00000063919 | 5 | 116439275 | 116591817 | 68955 |
| Epcam | activation | ENSMUSG00000045394 | 17 | 87635979 | 87651106 | 17075 |
| Trp53inp2 | activation | ENSMUSG00000038375 | 2 | 155381059 | 155389850 | 68728 |
| Arhgap17 | activation | ENSMUSG00000030766 | 7 | 123279218 | 123369915 | 70497 |
| Ric8b | activation | ENSMUSG00000035620 | 10 | 84917616 | 85018337 | 237422 |
| 9530034E10Rik | activation | ENSMUSG00000086554 | 4 | 117975036 | 117993972 | 78615 |
| Oaz2 | activation | ENSMUSG00000040652 | 9 | 65668001 | 65690300 | 18247 |
| Ctxn3 | activation | ENSMUSG00000069372 | 18 | 57468516 | 57478133 | 629147 |
| Slc38a2 | activation | ENSMUSG00000022462 | 15 | 96687392 | 96699730 | 67760 |
| Exoc6b | activation | ENSMUSG00000033769 | 6 | 84618487 | 85069513 | 75914 |
| Nfatc1 | activation | ENSMUSG00000033016 | 18 | 80606205 | 80713071 | 18018 |
| Junb | activation | ENSMUSG00000052837 | 8 | 84974484 | 84978718 | 16477 |
| Tle3 | activation | ENSMUSG00000032280 | 9 | 61372366 | 61418497 | 21887 |
| Siah2 | activation | ENSMUSG00000036432 | 3 | 58674938 | 58692400 | 20439 |
| Fos | activation | ENSMUSG00000021250 | 12 | 85473890 | 85477273 | 14281 |
| 7-Mar | activation | ENSMUSG00000026977 | 2 | 60209887 | 60250676 | 57438 |
| Ago2 | activation | ENSMUSG00000036698 | 15 | 73095844 | 73184935 | 239528 |
| Arhgef28 | activation | ENSMUSG00000021662 | 13 | 97899469 | 98206439 | 110596 |
| Ddx3x | activation | ENSMUSG00000000787 | X | 13280970 | 13294052 | 13205 |
| Hspa8 | activation | ENSMUSG00000015656 | 9 | 40800984 | 40810087 | 15481 |
| Hnrnph3 | activation | ENSMUSG00000020069 | 10 | 63014664 | 63024217 | 432467 |
| Erf | activation | ENSMUSG00000040857 | 7 | 25242561 | 25250761 | 13875 |
| Ak2 | activation | ENSMUSG00000028792 | 4 | 128991958 | 129011529 | 11637 |
| Pnrc1 | activation | ENSMUSG00000040128 | 4 | 33245423 | 33290163 | 108767 |
| Mafg | activation | ENSMUSG00000051510 | 11 | 120625117 | 120633600 | 17134 |
| D1Ertd622e | activation | ENSMUSG00000044768 | 1 | 97606318 | 97662074 | 52392 |
| Arid5b | activation | ENSMUSG00000019947 | 10 | 68092520 | 68278740 | 71371 |
| Scn3b | activation | ENSMUSG00000049281 | 9 | 40269217 | 40291618 | 235281 |
| Phtf2 | activation | ENSMUSG00000039987 | 5 | 20758663 | 20882124 | 68770 |
| Hsph1 | activation | ENSMUSG00000029657 | 5 | 149614287 | 149636376 | 15505 |
| Cpeb4 | activation | ENSMUSG00000020300 | 11 | 31872211 | 31935634 | 67579 |
| Ubc | activation | ENSMUSG00000008348 | 5 | 125385965 | 125390202 | 22190 |
| Sema7a | activation | ENSMUSG00000038264 | 9 | 57940112 | 57962865 | 20361 |
| Dleu2 | activation | ENSMUSG00000097589 | 14 | 61602839 | 61682373 | 668253 |
| Rc3h1 | activation | ENSMUSG00000040423 | 1 | 160906418 | 160974978 | 381305 |
| Pcnx4 | activation | ENSMUSG00000034501 | 12 | 72536383 | 72580119 | 67708 |
| Btg2 | activation | ENSMUSG00000020423 | 1 | 134075170 | 134079120 | 12227 |
| 6430548M08Rik | activation | ENSMUSG00000031824 | 8 | 120114152 | 120165306 | 234797 |
| Selenok | activation | ENSMUSG00000042682 | 14 | 29968308 | 29975662 | 80795 |
| Psmd8 | activation | ENSMUSG00000030591 | 7 | 29174188 | 29180701 | 57296 |
| Map9 | activation | ENSMUSG00000033900 | 3 | 82358044 | 82395268 | 213582 |
| Cry2 | activation | ENSMUSG00000068742 | 2 | 92403646 | 92434043 | 12953 |
| Hspa4l | activation | ENSMUSG00000025757 | 3 | 40744495 | 40796103 | 18415 |
| Mcl1 | activation | ENSMUSG00000038612 | 3 | 95658788 | 95663176 | 17210 |
| Dnaja1 | activation | ENSMUSG00000028410 | 4 | 40722150 | 40737149 | 15502 |
| Fth1 | activation | ENSMUSG00000024661 | 19 | 9982703 | 9985092 | 14319 |
| Fam208a | activation | ENSMUSG00000040651 | 14 | 27428834 | 27483555 | 218850 |
| Bmpr2 | activation | ENSMUSG00000067336 | 1 | 59763400 | 59879014 | 12168 |
| Zfp608 | activation | ENSMUSG00000052713 | 18 | 54888045 | 54990180 | 269023 |
| BC005561 | activation | ENSMUSG00000079065 | 5 | 104508352 | 104522611 | 100042165 |
| Rab11fip4 | activation | ENSMUSG00000017639 | 11 | 79591212 | 79698023 | 268451 |
| Smim13 | activation | ENSMUSG00000091264 | 13 | 41249844 | 41276577 | 108934 |
| Ppp2ca | activation | ENSMUSG00000020349 | 11 | 52098681 | 52127778 | 19052 |
| Sod1 | activation | ENSMUSG00000022982 | 16 | 90220754 | 90226329 | 20655 |
| Neat1 | activation | ENSMUSG00000092274 | 19 | 5824708 | 5845478 | 66961 |
| Brinp2 | activation | ENSMUSG00000004031 | 1 | 158245269 | 158356326 | 240843 |
| Tuba4a | activation | ENSMUSG00000026202 | 1 | 75214228 | 75219865 | 22145 |
| Cdc14a | activation | ENSMUSG00000033502 | 3 | 116272553 | 116424032 | 229776 |
| Kdm6b | activation | ENSMUSG00000018476 | 11 | 69398508 | 69413675 | 216850 |
| Uckl1os | activation | ENSMUSG00000010492 | 2 | 181578480 | 181585170 | 100271841 |
| Stk40 | activation | ENSMUSG00000042608 | 4 | 126103957 | 126141029 | 74178 |
| Dnajb2 | activation | ENSMUSG00000026203 | 1 | 75236406 | 75245692 | 56812 |
| Dnajb9 | activation | ENSMUSG00000014905 | 12 | 44205559 | 44210326 | 27362 |
| Spsb3 | activation | ENSMUSG00000024160 | 17 | 24886643 | 24892152 | 79043 |
| Sub1 | activation | ENSMUSG00000022205 | 15 | 11981339 | 11996983 | 20024 |
| Rheb | activation | ENSMUSG00000028945 | 5 | 24802823 | 24842624 | 19744 |
| Manea | activation | ENSMUSG00000040520 | 4 | 26324506 | 26346891 | 242362 |
| Fzd3 | activation | ENSMUSG00000007989 | 14 | 65192449 | 65262463 | 14365 |
| Zfand5 | activation | ENSMUSG00000024750 | 19 | 21272278 | 21282289 | 22682 |
| Uhrf1bp1l | activation | ENSMUSG00000019951 | 10 | 89744991 | 89819871 | 75089 |
| Cebpb | activation | ENSMUSG00000056501 | 2 | 167688915 | 167690418 | 12608 |
| Wdfy3 | activation | ENSMUSG00000043940 | 5 | 101832956 | 102069921 | 72145 |
| Mfge8 | activation | ENSMUSG00000030605 | 7 | 79133768 | 79149060 | 17304 |
| Inpp4a | activation | ENSMUSG00000026113 | 1 | 37299865 | 37410736 | 269180 |
| Nab1 | activation | ENSMUSG00000002881 | 1 | 52457294 | 52500679 | 17936 |
| Tceal9 | activation | ENSMUSG00000042712 | X | 136245079 | 136247139 | 22381 |
| Ube2g1 | activation | ENSMUSG00000020794 | 11 | 72607283 | 72686481 | 67128 |
| Pcdh7 | activation | ENSMUSG00000029108 | 5 | 57717967 | 58133230 | 54216 |
| Eef1a1 | activation | ENSMUSG00000037742 | 9 | 78478449 | 78489151 | 13627 |
| Tnrc6c | activation | ENSMUSG00000025571 | 11 | 117654289 | 117763439 | 217351 |
| Cd82 | activation | ENSMUSG00000027215 | 2 | 93419111 | 93463140 | 12521 |
| BC005537 | activation | ENSMUSG00000019132 | 13 | 24801657 | 24816197 | 79555 |
| Sdcbp | activation | ENSMUSG00000028249 | 4 | 6365650 | 6408423 | 53378 |
| Ddx5 | activation | ENSMUSG00000020719 | 11 | 106780355 | 106789185 | 13207 |
| Olfm1 | activation | ENSMUSG00000026833 | 2 | 28192992 | 28230736 | 56177 |
| Srpk1 | activation | ENSMUSG00000004865 | 17 | 28587648 | 28622521 | 20815 |
| M6pr | activation | ENSMUSG00000007458 | 6 | 122308720 | 122317680 | 17113 |
| Braf | activation | ENSMUSG00000002413 | 6 | 39603237 | 39725463 | 109880 |
| Ahi1 | activation | ENSMUSG00000019986 | 10 | 20952547 | 21080429 | 52906 |
| Clcn3 | activation | ENSMUSG00000004319 | 8 | 60910389 | 60983300 | 12725 |
| Eef2 | activation | ENSMUSG00000034994 | 10 | 81176631 | 81182498 | 13629 |
| Rbm26 | activation | ENSMUSG00000022119 | 14 | 105106751 | 105177327 | 74213 |
| Arfgef3 | activation | ENSMUSG00000019852 | 10 | 18581839 | 18743949 | 215821 |
| Eif5 | activation | ENSMUSG00000021282 | 12 | 111538016 | 111546752 | 217869 |
| Tmem150c | activation | ENSMUSG00000050640 | 5 | 100077872 | 100159808 | 231503 |
| Aebp2 | activation | ENSMUSG00000030232 | 6 | 140622663 | 140678472 | 11569 |
| Snrk | activation | ENSMUSG00000038145 | 9 | 122117266 | 122169702 | 20623 |
| Bex3 | activation | ENSMUSG00000046432 | X | 136270253 | 136271978 | 12070 |
| Epb41l2 | activation | ENSMUSG00000019978 | 10 | 25359798 | 25523519 | 13822 |
| Samd4b | activation | ENSMUSG00000109336 | 7 | 28399522 | 28598144 | 233033 |
| Stard10 | activation | ENSMUSG00000030688 | 7 | 101317086 | 101346626 | 56018 |
| Pappa | activation | ENSMUSG00000028370 | 4 | 65124174 | 65357509 | 18491 |
| Grsf1 | activation | ENSMUSG00000044221 | 5 | 88659448 | 88676171 | 231413 |
| Ralgds | activation | ENSMUSG00000026821 | 2 | 28513125 | 28553081 | 19730 |
| Arih1 | activation | ENSMUSG00000025234 | 9 | 59388258 | 59486618 | 23806 |
| Micu3 | activation | ENSMUSG00000039478 | 8 | 40307458 | 40386308 | 78506 |
| Ptges3 | activation | ENSMUSG00000071072 | 10 | 128058954 | 128077272 | 56351 |
| Kras | activation | ENSMUSG00000030265 | 6 | 145216699 | 145250239 | 16653 |
| Morf4l2 | activation | ENSMUSG00000031422 | X | 136732942 | 136743690 | 56397 |
| Rtp2 | activation | ENSMUSG00000047531 | 16 | 23925548 | 23932394 | 224055 |
| Wdr26 | activation | ENSMUSG00000038733 | 1 | 181173228 | 181211552 | 226757 |
| Hspa5 | activation | ENSMUSG00000026864 | 2 | 34771970 | 34777547 | 14828 |
| Zmynd8 | activation | ENSMUSG00000039671 | 2 | 165784152 | 165899016 | 228880 |
| Sv2c | activation | ENSMUSG00000051111 | 13 | 95954594 | 96132577 | 75209 |
| Paip2 | activation | ENSMUSG00000037058 | 18 | 35598617 | 35617186 | 67869 |
| Rab39b | activation | ENSMUSG00000031202 | X | 75572046 | 75578231 | 67790 |
| Ywhaz | activation | ENSMUSG00000022285 | 15 | 36770770 | 36796929 | 22631 |
| Gpat3 | activation | ENSMUSG00000029314 | 5 | 100845713 | 100899102 | 231510 |
| Psen2 | activation | ENSMUSG00000010609 | 1 | 180227004 | 180263438 | 19165 |
| Pde4a | activation | ENSMUSG00000032177 | 9 | 21165714 | 21213248 | 18577 |
| Atp2b1 | activation | ENSMUSG00000019943 | 10 | 98914406 | 99026143 | 67972 |
| Irs2 | activation | ENSMUSG00000038894 | 8 | 10984681 | 11008458 | 384783 |
| Cfap69 | activation | ENSMUSG00000040473 | 5 | 5579278 | 5664239 | 207686 |
| Hspa9 | activation | ENSMUSG00000024359 | 18 | 34937414 | 34954357 | 15526 |
| Tob1 | activation | ENSMUSG00000037573 | 11 | 94211454 | 94215495 | 22057 |
| Hspa14 | activation | ENSMUSG00000109865 | 2 | 3488850 | 3512814 | 50497 |
| Pard6a | activation | ENSMUSG00000005699 | 8 | 105701148 | 105703496 | 56513 |
| Mapk4 | activation | ENSMUSG00000024558 | 18 | 73928486 | 74065359 | 225724 |
| Nr4a1 | activation | ENSMUSG00000023034 | 15 | 101254269 | 101274795 | 15370 |
| Sik3 | activation | ENSMUSG00000034135 | 9 | 46012820 | 46224194 | 70661 |
| Epha7 | activation | ENSMUSG00000028289 | 4 | 28813131 | 28967499 | 13841 |
| Hagh | activation | ENSMUSG00000024158 | 17 | 24840143 | 24864450 | 14651 |
| Kalrn | activation | ENSMUSG00000061751 | 16 | 33969073 | 34573532 | 545156 |
| Prkar1a | activation | ENSMUSG00000020612 | 11 | 109649405 | 109669656 | 19084 |
| Lmbrd1 | activation | ENSMUSG00000073725 | 1 | 24678630 | 24766301 | 68421 |
| Erich3 | activation | ENSMUSG00000078161 | 3 | 154663859 | 154767790 | 209601 |
| Sf3b1 | activation | ENSMUSG00000025982 | 1 | 54985169 | 55027481 | 81898 |
| Ksr2 | activation | ENSMUSG00000061578 | 5 | 117414000 | 117775003 | 333050 |
| Ybx1 | activation | ENSMUSG00000028639 | 4 | 119277981 | 119294604 | 22608 |
| Nfix | activation | ENSMUSG00000001911 | 8 | 84699876 | 84800344 | 18032 |
| Hivep1 | activation | ENSMUSG00000021366 | 13 | 42052021 | 42192537 | 110521 |
| Mfn2 | activation | ENSMUSG00000029020 | 4 | 147873599 | 147904704 | 170731 |
| Elof1 | activation | ENSMUSG00000013822 | 9 | 22112989 | 22117172 | 66126 |
| Wdfy2 | activation | ENSMUSG00000014547 | 14 | 62837678 | 62961509 | 268752 |
| Emd | activation | ENSMUSG00000001964 | X | 74254687 | 74261548 | 13726 |
| Azin1 | activation | ENSMUSG00000037458 | 15 | 38487427 | 38519266 | 54375 |
| Wbp1 | activation | ENSMUSG00000030035 | 6 | 83119044 | 83121559 | 22377 |
| Nop53 | activation | ENSMUSG00000041560 | 7 | 15936183 | 15946074 | 68077 |
| Syt7 | activation | ENSMUSG00000024743 | 19 | 10389090 | 10453181 | 54525 |
| Ncoa1 | activation | ENSMUSG00000020647 | 12 | 4247362 | 4477182 | 17977 |
| Smarca5 | activation | ENSMUSG00000031715 | 8 | 80698507 | 80739497 | 93762 |
| Aes | activation | ENSMUSG00000054452 | 10 | 81559488 | 81566362 | 14797 |
| Rnf150 | activation | ENSMUSG00000047747 | 8 | 82863356 | 83091268 | 330812 |
| Plxnb2 | activation | ENSMUSG00000036606 | 15 | 89155549 | 89180788 | 140570 |
| Wdsub1 | activation | ENSMUSG00000026988 | 2 | 59852364 | 59882591 | 72137 |
| Dyrk4 | activation | ENSMUSG00000030345 | 6 | 126876020 | 126921839 | 101320 |
| Epb41l1 | activation | ENSMUSG00000027624 | 2 | 156420909 | 156543214 | 13821 |
| Pcdh8 | activation | ENSMUSG00000036422 | 14 | 79766775 | 79771312 | 18530 |
| Daam1 | activation | ENSMUSG00000034574 | 12 | 71831078 | 71992333 | 208846 |
| Prpf4b | activation | ENSMUSG00000021413 | 13 | 34875302 | 34906064 | 19134 |
| Tnrc6a | activation | ENSMUSG00000052707 | 7 | 123123885 | 123195296 | 233833 |
| Rufy3 | activation | ENSMUSG00000029291 | 5 | 88565040 | 88651392 | 52822 |
| Ptdss2 | activation | ENSMUSG00000025495 | 7 | 141122382 | 141157606 | 27388 |
| Tmem230 | activation | ENSMUSG00000027341 | 2 | 132239492 | 132247807 | 70612 |
| Scn3a | activation | ENSMUSG00000057182 | 2 | 65457118 | 65567627 | 20269 |
| Rock1 | activation | ENSMUSG00000024290 | 18 | 10064401 | 10181792 | 19877 |
| Dph3 | activation | ENSMUSG00000021905 | 14 | 32080566 | 32085729 | 105638 |
| 3-Sep | activation | ENSMUSG00000022456 | 15 | 82274893 | 82294574 | 24050 |
| Zbtb7b | activation | ENSMUSG00000028042 | 3 | 89377644 | 89394776 | 22724 |
| Yipf4 | activation | ENSMUSG00000024072 | 17 | 74489493 | 74500277 | 67864 |
| Ift74 | activation | ENSMUSG00000028576 | 4 | 94614491 | 94693229 | 67694 |
| Ebf1 | activation | ENSMUSG00000057098 | 11 | 44617317 | 45008091 | 13591 |
| Wsb2 | activation | ENSMUSG00000029364 | 5 | 117357304 | 117378601 | 59043 |
| Rnf182 | activation | ENSMUSG00000044164 | 13 | 43615710 | 43670945 | 328234 |
| Arglu1 | activation | ENSMUSG00000040459 | 8 | 8665075 | 8690521 | 234023 |
| Ppm1l | activation | ENSMUSG00000027784 | 3 | 69316861 | 69560802 | 242083 |
| Ccdc189 | activation | ENSMUSG00000057176 | 7 | 127582383 | 127588595 | 233899 |
| Ptov1 | activation | ENSMUSG00000038502 | 7 | 44863067 | 44869788 | 84113 |
| Ccdc157 | activation | ENSMUSG00000051427 | 11 | 4141123 | 4160293 | 216516 |
| Ppa1 | activation | ENSMUSG00000020089 | 10 | 61648552 | 61674168 | 67895 |
| Pnpla8 | activation | ENSMUSG00000036257 | 12 | 44221370 | 44322532 | 67452 |
| Rack1 | activation | ENSMUSG00000020372 | 11 | 48800332 | 48806434 | 14694 |
| Rsrp1 | activation | ENSMUSG00000037266 | 4 | 134923592 | 134927671 | 27981 |
| Ttc9 | activation | ENSMUSG00000042734 | 12 | 81631249 | 81667557 | 69480 |
| Hcn2 | activation | ENSMUSG00000020331 | 10 | 79716634 | 79736108 | 15166 |
| Ccdc40 | activation | ENSMUSG00000039963 | 11 | 119228572 | 119265236 | 207607 |
| Kat2b | activation | ENSMUSG00000000708 | 17 | 53566861 | 53672720 | 18519 |
| Cngb1 | activation | ENSMUSG00000031789 | 8 | 95239045 | 95306585 | 333329 |
| Ndufa3 | activation | ENSMUSG00000035674 | 7 | 3617373 | 3620327 | 66091 |
| Eef1a2 | activation | ENSMUSG00000016349 | 2 | 181147653 | 181157014 | 13628 |
| Fmn2 | activation | ENSMUSG00000028354 | 1 | 174501825 | 174822729 | 54418 |
| Macrod1 | activation | ENSMUSG00000036278 | 19 | 7056768 | 7198061 | 107227 |
| Polr2i | activation | ENSMUSG00000019738 | 7 | 30231948 | 30233390 | 69920 |
| 1700016K19Rik | activation | ENSMUSG00000053783 | 11 | 75999912 | 76003569 | 74230 |
| Jade1 | activation | ENSMUSG00000025764 | 3 | 41555731 | 41616864 | 269424 |
| Slc1a2 | activation | ENSMUSG00000005089 | 2 | 102658659 | 102790784 | 20511 |
| Ralbp1 | activation | ENSMUSG00000024096 | 17 | 65848433 | 65885755 | 19765 |
| Eif4e3 | activation | ENSMUSG00000093661 | 6 | 99625135 | 99666771 | 66892 |
| Morn2 | activation | ENSMUSG00000045257 | 17 | 80290203 | 80297476 | 378462 |
| Trappc2l | activation | ENSMUSG00000015013 | 8 | 122611640 | 122616660 | 59005 |
| Scrn1 | activation | ENSMUSG00000019124 | 6 | 54501173 | 54566489 | 69938 |
| Rtp1 | activation | ENSMUSG00000033383 | 16 | 23429133 | 23433960 | 239766 |
| Phyh | activation | ENSMUSG00000026664 | 2 | 4919019 | 4938730 | 16922 |
| Fetub | activation | ENSMUSG00000022871 | 16 | 22918334 | 22939778 | 59083 |
| Mapre3 | activation | ENSMUSG00000029166 | 5 | 30814641 | 30866106 | 100732 |
| Dnajb13 | activation | ENSMUSG00000030708 | 7 | 100501716 | 100514960 | 69387 |
| Coa3 | activation | ENSMUSG00000017188 | 11 | 101277968 | 101279114 | 52469 |
| Spef1 | activation | ENSMUSG00000027329 | 2 | 131170261 | 131187282 | 70997 |
| 1810058l24Rik | activation | ENSMUSG00000073155 | 6 | 35252654 | 35263496 | 67705 |
| Cep290 | activation | ENSMUSG00000019971 | 10 | 100487558 | 100574840 | 216274 |
| Bbs4 | activation | ENSMUSG00000025235 | 9 | 59321990 | 59353508 | 102774 |
| Nin | activation | ENSMUSG00000021068 | 12 | 70011435 | 70113717 | 18080 |
| D430042O09Rik | activation | ENSMUSG00000032743 | 7 | 125707888 | 125874793 | 233865 |
| Tex9 | activation | ENSMUSG00000090626 | 9 | 72450394 | 72492212 | 21778 |
| Sdc3 | activation | ENSMUSG00000025743 | 4 | 130792537 | 130826319 | 20970 |
| Ebf4 | activation | ENSMUSG00000053552 | 2 | 130295169 | 130370481 | 228598 |
| Oaz1 | activation | ENSMUSG00000035242 | 10 | 80826656 | 80829290 | 18245 |
| Napa | activation | ENSMUSG00000006024 | 7 | 16098458 | 16117975 | 108124 |
| Ttc8 | activation | ENSMUSG00000021013 | 12 | 98920574 | 98983238 | 76260 |
| Sdhaf4 | activation | ENSMUSG00000026154 | 1 | 23995939 | 24005656 | 68002 |
| Cuta | activation | ENSMUSG00000024194 | 17 | 26937973 | 26939478 | 67675 |
| Commd6 | activation | ENSMUSG00000075486 | 14 | 101632981 | 101640686 | 66200 |
| Sfr1 | activation | ENSMUSG00000025066 | 19 | 47731756 | 47735588 | 67788 |
| Hcfc1r1 | activation | ENSMUSG00000023904 | 17 | 23673596 | 23675227 | 353502 |
| Nxnl2 | activation | ENSMUSG00000021396 | 13 | 51171022 | 51175188 | 75124 |
| Kcnh3 | activation | ENSMUSG00000037579 | 15 | 99224861 | 99242817 | 16512 |
| Trpm7 | activation | ENSMUSG00000027365 | 2 | 126791565 | 126876230 | 58800 |
| Glb1l2 | activation | ENSMUSG00000036395 | 9 | 26763044 | 26806468 | 244757 |
| Slc25a39 | activation | ENSMUSG00000018677 | 11 | 102402985 | 102407946 | 68066 |
| Nsmf | activation | ENSMUSG00000006476 | 2 | 25054355 | 25062881 | 56876 |
| Pifo | activation | ENSMUSG00000010136 | 3 | 105996957 | 106014646 | 100503311 |
| Oscp1 | activation | ENSMUSG00000042616 | 4 | 126058565 | 126092195 | 230751 |
| 1110008P14Rik | activation | ENSMUSG00000039195 | 2 | 32377097 | 32381938 | 73737 |
| Egr1 | activation | ENSMUSG00000038418 | 18 | 34859823 | 34864984 | 13653 |
| Ttll6 | activation | ENSMUSG00000038756 | 11 | 96133786 | 96165451 | 237930 |
| Sem1 | activation | ENSMUSG00000042541 | 6 | 6557294 | 6578663 | 20422 |
| Ap1g1 | activation | ENSMUSG00000031731 | 8 | 109778554 | 109864204 | 11765 |
| Fkbp2 | activation | ENSMUSG00000056629 | 19 | 6977741 | 6980461 | 14227 |
| Suclg1 | activation | ENSMUSG00000052738 | 6 | 73248382 | 73276911 | 56451 |
| Cidea | activation | ENSMUSG00000024526 | 18 | 67343564 | 67367794 | 12683 |
| Dynlrb2 | activation | ENSMUSG00000034467 | 8 | 116504974 | 116515915 | 75465 |
| Ndufa2 | activation | ENSMUSG00000014294 | 18 | 36742332 | 36744557 | 17991 |
| Map1a | activation | ENSMUSG00000027254 | 2 | 121289600 | 121310832 | 17754 |
| Sun1 | activation | ENSMUSG00000036817 | 5 | 139200637 | 139249840 | 77053 |
| Rufy2 | activation | ENSMUSG00000020070 | 10 | 62980223 | 63017210 | 70432 |
| Rnh1 | activation | ENSMUSG00000038650 | 7 | 141160326 | 141172857 | 107702 |
| Dtna | activation | ENSMUSG00000024302 | 18 | 23415135 | 23659715 | 13527 |
| Anapc16 | activation | ENSMUSG00000020107 | 10 | 59987409 | 60003135 | 52717 |
| Pdhb | activation | ENSMUSG00000021748 | 14 | 8165996 | 8173025 | 68263 |
| A430035B10Rik | activation | ENSMUSG00000087305 | 6 | 8488795 | 8510430 | 320312 |
| Faim2 | activation | ENSMUSG00000023011 | 15 | 99497012 | 99528165 | 72393 |
| Ebf2 | activation | ENSMUSG00000022053 | 14 | 67233292 | 67430918 | 13592 |
| Ccdc28b | activation | ENSMUSG00000028795 | 4 | 129619274 | 129623947 | 66264 |
| Ttll3 | activation | ENSMUSG00000030276 | 6 | 113389260 | 113414587 | 101100 |
| Drc1 | activation | ENSMUSG00000073102 | 5 | 30281388 | 30366695 | 381738 |
| Sys1 | activation | ENSMUSG00000045503 | 2 | 164456964 | 164479638 | 66460 |
| Prdx5 | activation | ENSMUSG00000024953 | 19 | 6906697 | 6910106 | 54683 |
| Palm | activation | ENSMUSG00000035863 | 10 | 79793572 | 79820896 | 18483 |
| Slu7 | activation | ENSMUSG00000020409 | 11 | 43433744 | 43447981 | 193116 |
| Dnajc15 | activation | ENSMUSG00000022013 | 14 | 77820681 | 77875125 | 66148 |
| Elmod1 | activation | ENSMUSG00000041986 | 9 | 53911457 | 53975301 | 270162 |
| Paqr9 | activation | ENSMUSG00000064225 | 9 | 95559657 | 95568023 | 75552 |
| Tubb3 | activation | ENSMUSG00000062380 | 8 | 123411424 | 123422015 | 22152 |
| Ascc1 | activation | ENSMUSG00000044475 | 10 | 60002805 | 60099988 | 69090 |
| Ndufc1 | activation | ENSMUSG00000037152 | 3 | 51404677 | 51408988 | 66377 |
| Pon2 | activation | ENSMUSG00000032667 | 6 | 5264147 | 5298455 | 330260 |
| Fkbp4 | activation | ENSMUSG00000030357 | 6 | 128429735 | 128438677 | 14228 |
| Trnp1 | activation | ENSMUSG00000056596 | 4 | 133491100 | 133498550 | 69539 |
| Cldn3 | activation | ENSMUSG00000070473 | 5 | 134986214 | 134987472 | 12739 |
| Cby1 | activation | ENSMUSG00000022428 | 15 | 79659199 | 79667660 | 73739 |
| Tctex1d2 | activation | ENSMUSG00000014075 | 16 | 32419702 | 32429099 | 66061 |
| Slc48a1 | activation | ENSMUSG00000081534 | 15 | 97778520 | 97792692 | 67739 |
| Nme5 | activation | ENSMUSG00000035984 | 18 | 34562634 | 34579115 | 75533 |
| 1110004E09Rik | activation | ENSMUSG00000022972 | 16 | 90925809 | 90935114 | 68001 |
| Cnppd1 | activation | ENSMUSG00000033159 | 1 | 75134554 | 75142711 | 69171 |
| Naa38 | activation | ENSMUSG00000059278 | 11 | 69395487 | 69396680 | 78304 |
| Vdac3 | activation | ENSMUSG00000008892 | 8 | 22577075 | 22593813 | 22335 |
| Puf60 | activation | ENSMUSG00000002524 | 15 | 76070182 | 76080924 | 67959 |
| Cers5 | activation | ENSMUSG00000023021 | 15 | 99734881 | 99772885 | 71949 |
| Cbr1 | activation | ENSMUSG00000051483 | 16 | 93605853 | 93610505 | 12408 |
| Ift43 | activation | ENSMUSG00000007867 | 12 | 86082541 | 86162459 | 76411 |
| Fam217a | activation | ENSMUSG00000021414 | 13 | 34909960 | 34924310 | 71864 |
| Ankrd10 | activation | ENSMUSG00000031508 | 8 | 11611583 | 11635757 | 102334 |
| Ube2n | activation | ENSMUSG00000074781 | 10 | 95515145 | 95545657 | 93765 |
| Pfdn6 | activation | ENSMUSG00000024309 | 17 | 33938821 | 33940343 | 14976 |
| Mpc2 | activation | ENSMUSG00000026568 | 1 | 165460637 | 165481214 | 70456 |
| Ncbp2 | activation | ENSMUSG00000022774 | 16 | 31948513 | 31961781 | 68092 |
| Timm13 | activation | ENSMUSG00000020219 | 10 | 80899450 | 80900969 | 30055 |
| Lamtor5 | activation | ENSMUSG00000087260 | 3 | 107278858 | 107284082 | 68576 |
| Uqcr10 | activation | ENSMUSG00000059534 | 11 | 4701973 | 4704342 | 66152 |
| Slc27a2 | activation | ENSMUSG00000027359 | 2 | 126552407 | 126588243 | 26458 |
| Arpc5l | activation | ENSMUSG00000026755 | 2 | 39005348 | 39015877 | 74192 |
| Adh5 | activation | ENSMUSG00000028138 | 3 | 138443093 | 138455499 | 11532 |
| Gkap1 | activation | ENSMUSG00000021552 | 13 | 58233346 | 58275699 | 56278 |
| Ndufaf4 | activation | ENSMUSG00000028261 | 4 | 24898083 | 24905001 | 68493 |
| Psip1 | activation | ENSMUSG00000028484 | 4 | 83455680 | 83486459 | 101739 |
| Omp | activation | ENSMUSG00000074006 | 7 | 98143359 | 98145502 | 18378 |
| Bcl11a | activation | ENSMUSG00000000861 | 11 | 24078056 | 24174123 | 14025 |
| Ift81 | activation | ENSMUSG00000029469 | 5 | 122550204 | 122614518 | 12589 |
| 2010107E04Rik | activation | ENSMUSG00000021290 | 12 | 111961375 | 111966977 | 70257 |
| Ubxn1 | activation | ENSMUSG00000071655 | 19 | 8871559 | 8875663 | 225896 |
| Ech1 | activation | ENSMUSG00000053898 | 7 | 28825217 | 28832247 | 51798 |
| Tnrc6b | activation | ENSMUSG00000047888 | 15 | 80711313 | 80941085 | 213988 |
| Tmpo | activation | ENSMUSG00000019961 | 10 | 91147571 | 91181315 | 21917 |
| Ssbp4 | activation | ENSMUSG00000070003 | 8 | 70597490 | 70608872 | 76900 |
| 2410015M20Rik | activation | ENSMUSG00000049760 | 17 | 56607452 | 56609771 | 224904 |
| Mapk8ip2 | activation | ENSMUSG00000022619 | 15 | 89453913 | 89464468 | 60597 |
| Naxe | activation | ENSMUSG00000028070 | 3 | 88056520 | 88058495 | 246703 |
| Gm13589 | activation | ENSMUSG00000085950 | 2 | 38424002 | 38455851 | |
| Hdgfl3 | activation | ENSMUSG00000025104 | 7 | 81881251 | 81934473 | 29877 |
| Slc22a23 | activation | ENSMUSG00000038267 | 13 | 34179158 | 34345182 | 73102 |
| Cacna1h | activation | ENSMUSG00000024112 | 17 | 25374285 | 25433783 | 58226 |
| Slc25a5 | activation | ENSMUSG00000016319 | X | 36795651 | 36798807 | 11740 |
| Ndufa13 | activation | ENSMUSG00000036199 | 8 | 69894180 | 69902558 | 67184 |
| Anp32a | activation | ENSMUSG00000032249 | 9 | 62341293 | 62378812 | 11737 |
| Ndufb10 | activation | ENSMUSG00000040048 | 17 | 24722060 | 24724478 | 68342 |
| Lhx2 | activation | ENSMUSG00000000247 | 2 | 38339281 | 38369733 | 16870 |
| Cenpx | activation | ENSMUSG00000025144 | 11 | 120710942 | 120713738 | 20892 |
| Rnf220 | activation | ENSMUSG00000028677 | 4 | 117271463 | 117497052 | 66743 |
| Ankrd54 | activation | ENSMUSG00000033055 | 15 | 79053094 | 79062893 | 223690 |
| Ppdpf | activation | ENSMUSG00000016344 | 2 | 181187247 | 181188771 | 66496 |
| Dtd1 | activation | ENSMUSG00000027430 | 2 | 144599897 | 144768758 | 66044 |
| Auh | activation | ENSMUSG00000021460 | 13 | 52835119 | 52929681 | 11992 |
| Mrpl51 | activation | ENSMUSG00000030335 | 6 | 125191801 | 125196269 | 66493 |
| Psmd13 | activation | ENSMUSG00000025487 | 7 | 140881968 | 140898643 | 23997 |
| Lamtor4 | activation | ENSMUSG00000050552 | 5 | 138255482 | 138259398 | 66096 |
| Hist2h2be | activation | ENSMUSG00000068854 | 3 | 96221119 | 96223738 | 319190 |
| Mapkap1 | activation | ENSMUSG00000038696 | 2 | 34406771 | 34624950 | 227743 |
| Arhgdig | activation | ENSMUSG00000073433 | 17 | 26196226 | 26207786 | 14570 |
| Ndufv3 | activation | ENSMUSG00000024038 | 17 | 31520115 | 31531326 | 78330 |
| Ilkap | activation | ENSMUSG00000026309 | 1 | 91373861 | 91398815 | 67444 |
| Ctsa | activation | ENSMUSG00000017760 | 2 | 164832873 | 164841032 | 19025 |
| Ift27 | activation | ENSMUSG00000016637 | 15 | 78159463 | 78174107 | 67042 |
| Lpcat4 | activation | ENSMUSG00000027134 | 2 | 112239468 | 112247111 | 99010 |
| Atp50 | activation | ENSMUSG00000116933 | 16 | 91684398 | 91926982 | 28080 |
| Ypel3 | activation | ENSMUSG00000042675 | 7 | 126776955 | 126780514 | 66090 |
| Gadl1 | activation | ENSMUSG00000056880 | 9 | 115909455 | 116076176 | 73748 |
| Mslnl | activation | ENSMUSG00000041062 | 17 | 25736040 | 25748330 | 328783 |
| Hint2 | activation | ENSMUSG00000028470 | 4 | 43654227 | 43656466 | 68917 |
| Acbd7 | activation | ENSMUSG00000026644 | 2 | 3336168 | 3340993 | 78245 |
| Manf | activation | ENSMUSG00000032575 | 9 | 106838312 | 106891979 | 74840 |
| Lamtor2 | activation | ENSMUSG00000028062 | 3 | 88549819 | 88553074 | 83409 |
| Ak1 | activation | ENSMUSG00000026817 | 2 | 32621758 | 32635058 | 11636 |
| Arl6 | activation | ENSMUSG00000022722 | 16 | 59612949 | 59639391 | 56297 |
| Uxs1 | activation | ENSMUSG00000057363 | 1 | 43746966 | 43827800 | 67883 |
| Sri | activation | ENSMUSG00000003161 | 5 | 8046078 | 8069379 | 109552 |
| Tomm7 | activation | ENSMUSG00000028998 | 5 | 23838944 | 23844161 | 66169 |
| Ubxn6 | activation | ENSMUSG00000019578 | 17 | 56067045 | 56075028 | 66530 |
| Cd47 | activation | ENSMUSG00000055447 | 16 | 49800533 | 49915010 | 16423 |
| Snrpd3 | activation | ENSMUSG00000020180 | 10 | 75517551 | 75537381 | 67332 |
| Ppid | activation | ENSMUSG00000027804 | 3 | 79591342 | 79603650 | 67738 |
| Rsph9 | activation | ENSMUSG00000023966 | 17 | 46122035 | 46144216 | 75564 |
| Rbfox2 | activation | ENSMUSG00000033565 | 15 | 77078990 | 77307004 | 93686 |
| Pigp | activation | ENSMUSG00000022940 | 16 | 94358763 | 94371842 | 56176 |
| C1qbp | activation | ENSMUSG00000018446 | 11 | 70977836 | 70983026 | 12261 |
| Fmc1 | activation | ENSMUSG00000019689 | 6 | 38533502 | 38539449 | 66117 |
| Bcl7a | activation | ENSMUSG00000029438 | 5 | 123343834 | 123374992 | 77045 |
| Krtcap2 | activation | ENSMUSG00000042747 | 3 | 89245966 | 89249906 | 66059 |
| Flrt1 | activation | ENSMUSG00000047787 | 19 | 7092014 | 7105729 | 396184 |
| Sdhd | activation | ENSMUSG00000000171 | 9 | 50596357 | 50603812 | 66925 |
| Bphl | activation | ENSMUSG00000038286 | 13 | 34037597 | 34074074 | 68021 |
| Sod2 | activation | ENSMUSG00000006818 | 17 | 13007839 | 13018119 | 20656 |
| Ndufb7 | activation | ENSMUSG00000033938 | 8 | 83566671 | 83571626 | 66916 |
| Prrc2b | activation | ENSMUSG00000039262 | 2 | 32151082 | 32234537 | 227723 |
| Rex1bd | activation | ENSMUSG00000058833 | 8 | 70504292 | 70506752 | 66462 |
| Rab36 | activation | ENSMUSG00000020175 | 10 | 75037058 | 75054748 | 76877 |
| Pdrg1 | activation | ENSMUSG00000027472 | 2 | 153008890 | 153015427 | 68559 |
| Pfn2 | activation | ENSMUSG00000027805 | 3 | 57841895 | 57848079 | 18645 |
| Rarg | activation | ENSMUSG00000001288 | 15 | 102234938 | 102257517 | 19411 |
| B230118H07Rik | activation | ENSMUSG00000027165 | 2 | 101560781 | 101649532 | 68170 |
| Elavl3 | activation | ENSMUSG00000003410 | 9 | 22015005 | 22052023 | 15571 |
| Mlf1 | activation | ENSMUSG00000048416 | 3 | 67374097 | 67400003 | 17349 |
| Eif1 | activation | ENSMUSG00000035530 | 11 | 100319885 | 100322096 | 20918 |
| Mettl26 | activation | ENSMUSG00000025731 | 17 | 25875464 | 25886007 | 68347 |
| Kmt5a | activation | ENSMUSG00000049327 | 5 | 124439930 | 124462308 | 67956 |
| Ndufa5 | activation | ENSMUSG00000023089 | 6 | 24518666 | 24528013 | 68202 |
| Ndufb3 | activation | ENSMUSG00000026032 | 1 | 58586384 | 58595964 | 66495 |
| Emc8 | activation | ENSMUSG00000031819 | 8 | 120653914 | 120668573 | 18117 |
| Bad | activation | ENSMUSG00000024959 | 19 | 6941861 | 6951898 | 12015 |
| Smc3 | activation | ENSMUSG00000024974 | 19 | 53600398 | 53645833 | 13006 |
| Dpm3 | activation | ENSMUSG00000042737 | 3 | 89259358 | 89267079 | 68563 |
| Ndufa11 | activation | ENSMUSG00000002379 | 17 | 56717762 | 56724248 | 69875 |
| Cisd1 | activation | ENSMUSG00000037710 | 10 | 71330480 | 71344954 | 52637 |
| Mrpl27 | activation | ENSMUSG00000024414 | 11 | 94653767 | 94660089 | 94064 |
| Cldn9 | activation | ENSMUSG00000066720 | 17 | 23682584 | 23684018 | 56863 |
| Sec61b | activation | ENSMUSG00000053317 | 4 | 47474658 | 47483242 | 66212 |
| Tpd52l2 | activation | ENSMUSG00000000827 | 2 | 181497142 | 181517966 | 66314 |
| Polr2c | activation | ENSMUSG00000031783 | 8 | 94857450 | 94873535 | 20021 |
| Ndufa7 | activation | ENSMUSG00000041881 | 17 | 33824591 | 33838316 | 66416 |
| Atf5 | activation | ENSMUSG00000038539 | 7 | 44812256 | 44816658 | 107503 |
| Gabarap | activation | ENSMUSG00000018567 | 11 | 69991143 | 69994951 | 56486 |
| Srsf3 | activation | ENSMUSG00000071172 | 17 | 29032673 | 29043366 | 20383 |
| Psmb3 | activation | ENSMUSG00000069744 | 11 | 97703399 | 97713500 | 26446 |
| Bax | activation | ENSMUSG00000003873 | 7 | 45461697 | 45466898 | 12028 |
| Atox1 | activation | ENSMUSG00000018585 | 11 | 55446641 | 55461239 | 11927 |
| 2410004P03Rik | activation | ENSMUSG00000071398 | 12 | 17003319 | 17011727 | 73667 |
| Atp1a1 | activation | ENSMUSG00000033161 | 3 | 101576219 | 101604684 | 11928 |
| Ptprs | activation | ENSMUSG00000013236 | 17 | 56412426 | 56476483 | 19280 |
| Cib1 | activation | ENSMUSG00000030538 | 7 | 80227147 | 80232813 | 23991 |
| Hist1h2bc | activation | ENSMUSG00000018102 | 13 | 23684199 | 23692908 | 68024 |
| Atp5k | activation | ENSMUSG00000050856 | 5 | 108433244 | 108434448 | 11958 |
| Hmox2 | activation | ENSMUSG00000004070 | 16 | 4726361 | 4766742 | 15369 |
| Fam92b | activation | ENSMUSG00000042269 | 8 | 120166397 | 120177466 | 436062 |
| Chd4 | activation | ENSMUSG00000063870 | 6 | 125095981 | 125130591 | 107932 |
| Elob | activation | ENSMUSG00000055839 | 17 | 23824740 | 23829109 | 67673 |
| Kif5a | activation | ENSMUSG00000074657 | 10 | 127225696 | 127263348 | 16572 |
| Tbcb | activation | ENSMUSG00000006095 | 7 | 30224131 | 30232272 | 66411 |
| Ndufa4 | activation | ENSMUSG00000029632 | 6 | 11900292 | 11907497 | 17992 |
| Uqcr11 | activation | ENSMUSG00000020163 | 10 | 80402997 | 80406830 | 66594 |
| Hras | activation | ENSMUSG00000025499 | 7 | 141189105 | 141194005 | 15461 |
| Atp6v1f | activation | ENSMUSG00000004285 | 6 | 29467718 | 29470512 | 66144 |
| Pam16 | activation | ENSMUSG00000014301 | 16 | 4616464 | 4624988 | 66449 |
| Pim3 | activation | ENSMUSG00000035828 | 15 | 88862186 | 88865726 | 223775 |
| Lrriq1 | activation | ENSMUSG00000019892 | 10 | 103046031 | 103236322 | 74978 |
| Nfu1 | activation | ENSMUSG00000029993 | 6 | 87009236 | 87028461 | 56748 |
| Supt4a | activation | ENSMUSG00000020485 | 11 | 87737552 | 87743623 | 20922 |
| Swi5 | activation | ENSMUSG00000044627 | 2 | 32278816 | 32288075 | 72931 |
| Mtx2 | activation | ENSMUSG00000027099 | 2 | 74825803 | 74878431 | 53375 |
| Faim | activation | ENSMUSG00000032463 | 9 | 98986373 | 99002021 | 23873 |
| Taldo1 | activation | ENSMUSG00000025503 | 7 | 141392199 | 141402968 | 21351 |
| Ccdc34 | activation | ENSMUSG00000027160 | 2 | 110017817 | 110173360 | 68201 |
| Rbm39 | activation | ENSMUSG00000027620 | 2 | 156147239 | 156180238 | 170791 |
| 2210016L21Rik | activation | ENSMUSG00000029559 | 5 | 114942158 | 114949783 | 72357 |
| Dpy30 | activation | ENSMUSG00000024067 | 17 | 74299474 | 74323944 | 66310 |
| Tomm22 | activation | ENSMUSG00000022427 | 15 | 79670861 | 79673400 | 223696 |
| Pygo1 | activation | ENSMUSG00000034910 | 9 | 72925645 | 72952181 | 72135 |
| Mat2b | activation | ENSMUSG00000042032 | 11 | 40679314 | 40695203 | 108645 |
| Mrpl28 | activation | ENSMUSG00000024181 | 17 | 26123520 | 26126613 | 68611 |
| Eid1 | activation | ENSMUSG00000091337 | 2 | 125673095 | 125674785 | 58521 |
| Snrpe | activation | ENSMUSG00000090553 | 1 | 133603871 | 133610291 | 20643 |
| Atp5j | activation | ENSMUSG00000022890 | 16 | 84827866 | 84835625 | 11957 |
| Ndufs7 | activation | ENSMUSG00000020153 | 10 | 80249121 | 80256794 | 75406 |
| Park7 | activation | ENSMUSG00000028964 | 4 | 150897133 | 150914437 | 57320 |
| Ndufs4 | activation | ENSMUSG00000021764 | 13 | 114287795 | 114388258 | 17993 |
| Cirbp | activation | ENSMUSG00000045193 | 10 | 80165985 | 80172786 | 12696 |
| Polr2f | activation | ENSMUSG00000033020 | 15 | 79141009 | 79151774 | 69833 |
| Fbxo9 | activation | ENSMUSG00000001366 | 9 | 78081499 | 78109065 | 71538 |
| Mgst3 | activation | ENSMUSG00000026688 | 1 | 167371966 | 167393841 | 66447 |
| Gtf2h5 | activation | ENSMUSG00000034345 | 17 | 6079786 | 6086517 | 66467 |
| Mtch1 | activation | ENSMUSG00000024012 | 17 | 29332072 | 29347904 | 56462 |
| Mrps24 | activation | ENSMUSG00000020477 | 11 | 5703983 | 5715680 | 64660 |
| Actr1b | activation | ENSMUSG00000037351 | 1 | 36698114 | 36714422 | 226977 |
| Sh3bgrl3 | activation | ENSMUSG00000028843 | 4 | 134127406 | 134128789 | 73723 |
| Bmpr1a | activation | ENSMUSG00000021796 | 14 | 34410734 | 34503341 | 12166 |
| Pfn1 | activation | ENSMUSG00000018293 | 11 | 70651850 | 70654644 | 18643 |
| Tesc | activation | ENSMUSG00000029359 | 5 | 118027743 | 118061878 | 57816 |
| Tmem258 | activation | ENSMUSG00000036372 | 19 | 10204014 | 10207824 | 69038 |
| Mrps14 | activation | ENSMUSG00000058267 | 1 | 160195215 | 160202170 | 64659 |
| Abhd16a | activation | ENSMUSG00000007036 | 17 | 35089263 | 35102987 | 193742 |
| Vdac2 | activation | ENSMUSG00000021771 | 14 | 21825238 | 21845879 | 22334 |
| Nxph3 | activation | ENSMUSG00000046719 | 11 | 95509845 | 95514570 | 104079 |
| Polr2g | activation | ENSMUSG00000071662 | 19 | 8793129 | 8798557 | 67710 |
| Them6 | activation | ENSMUSG00000056665 | 15 | 74721204 | 74724639 | 223626 |
| Cfap126 | activation | ENSMUSG00000026649 | 1 | 171113918 | 171126967 | 75472 |
| Pitpnc1 | activation | ENSMUSG00000040430 | 11 | 107207892 | 107470699 | 71795 |
| Kctd1 | activation | ENSMUSG00000036225 | 18 | 14968685 | 15151446 | 106931 |
| Dmkn | activation | ENSMUSG00000060962 | 7 | 30763756 | 30781063 | 73712 |

In some embodiments of any of the aspects, the odor response genes comprise at least 1 of the genes of Table 1. In some embodiments of any of the aspects, the odor response genes comprise at least 2 of the genes of Table 1. In some embodiments of any of the aspects, the odor response genes comprise at least 3 of the genes of Table 1. In some embodiments of any of the aspects, the odor response genes comprise at least 4 of the genes of Table 1. In some embodiments of any of the aspects, the odor response genes comprise at least 5 of the genes of Table 1. In some embodiments of any of the aspects, the odor response genes comprise at least 6 of the genes of Table 1. In some embodiments of any of the aspects, the odor response genes comprise at least 10 of the genes of Table 1. In some embodiments of any of the aspects, the odor response genes comprise at least 20 of the genes of Table 1. In some embodiments of any of the aspects, the odor response genes comprise at least 30 of the genes of Table 1. In some embodiments of any of the aspects, the odor response genes comprise at least 40 of the genes of Table 1. In some embodiments of any of the aspects, the odor response genes comprise at least 50 of the genes of Table 1. In some embodiments of any of the aspects, the odor response genes comprise at least 100 of the genes of Table 1. In some embodiments of any of the aspects, the odor response genes comprise each of the genes of Table 1.

In some embodiments of any of the aspects, the odor response genes comprise at least 1 human ortholog of the genes of Table 1. In some embodiments of any of the aspects, the odor response genes comprise at least 2 human orthologs of the genes of Table 1. In some embodiments of any of the aspects, the odor response genes comprise at least 3 human orthologs of the genes of Table 1. In some embodiments of any of the aspects, the odor response genes comprise at least 4 human orthologs of the genes of Table 1. In some embodiments of any of the aspects, the odor response genes comprise at least 5 human orthologs of the genes of Table 1. In some embodiments of any of the aspects, the odor response genes comprise at least 6 human orthologs of the genes of Table 1. In some embodiments of any of the aspects, the odor response genes comprise at least 10 human orthologs of the genes of Table 1. In some embodiments of any of the aspects, the odor response genes comprise at least 20 human orthologs of the genes of Table 1. In some embodiments of any of the aspects, the odor response genes comprise at least 30 human orthologs of the genes of Table 1. In some embodiments of any of the aspects, the odor response genes comprise at least 40 human orthologs of the genes of Table 1. In some embodiments of any of the aspects, the odor response genes comprise at least 50 human orthologs of the genes of Table 1. In some embodiments of any of the aspects, the odor response genes comprise at least 100 human orthologs of the genes of Table 1. In some embodiments of any of the aspects, the odor response genes comprise human orthologs each of the genes of Table 1.

In some embodiments, measuring the expression of at least one odor response gene can comprise measuring the expression of at least one odor response gene, at least two odor response genes, at least three odor response genes, at least four response genes, at least five odor response genes, at least six odor response genes, at least seven odor response genes, at least eight odor response genes, at least nine odor response genes, at least ten odor response genes, at least 11 odor response genes, at least 12 odor response genes, at least 13 odor response genes, at least 14 odor response genes, at least 15 odor response genes, at least 16 odor response genes, at least 17 odor response genes, at least 18 odor response genes, at least 19 odor response genes, at least 20 odor response genes, at least 21 odor response genes, at least 22 odor response genes, at least 23 odor response genes, at least 24 odor response genes, at least 25 odor response genes, at least 26 odor response genes, at least 27 odor response genes, at least 28 odor response genes, at least 29 odor response genes, at least 30 odor response genes, at least 31 odor response genes, at least 32 odor response genes, at least 33 odor response genes, at least 34 odor response genes, at least 35 odor response genes, at least 36 odor response genes, at least 37 odor response genes, at least 38 odor response genes, at least 39 odor response genes, at least 40 odor response genes, at least 41 odor response genes, at least 42 odor response genes, at least 43 odor response genes, at least 44 odor response genes, at least 45 odor response genes, at least 46 odor response genes, at least 47 odor response genes, at least 48 odor response genes, at least 49 odor response genes, at least fifty odor response genes, at least 51 odor response genes, at least 52 odor response genes, at least 53 odor response genes, at least 54 odor response genes, at least 55 odor response genes, at least 55 odor response genes, at least 56 odor response genes, at least 57 odor response genes, at least 58 odor response genes, at least 59 odor response genes, at least 60 odor response genes, at least 61 odor response genes, at least 62 odor response genes, at least 63 odor response genes, at least 64 odor response genes, at least 65 odor response genes, at least 66 odor response genes, at least 67 odor response genes, at least 68 odor response genes, at least 69 odor response genes, at least 70 odor response genes, at least 71 odor response genes, at least 72 odor response genes, at least 73 odor response genes, at least 74 odor response genes, at least 75 odor response genes, at least 76 odor response genes, at least 77 odor response genes, at least 78 odor response genes, at least 79 odor response genes, at least 80 odor response genes, at least 81 odor response genes, at least 82 odor response genes, at least 83 odor response genes, at least 84 odor response genes, at least 85 odor response genes, at least 86 odor response genes, at least 87 odor response genes, at least 88 odor response genes, at least 89 odor response genes, at least 90 odor response genes, at least 91 odor response genes, at least 92 odor response genes, at least 93 odor response genes, at least 94 odor response genes, at least 95 odor response genes, at least 96 odor response genes, at least 97 odor response genes, at least 98 odor response genes, at least 99 odor response genes, at least one hundred odor response genes or more.

In some embodiments of any of the aspects, the odor response genes comprise Tsc22d1, Rcan1, Fosb, Pcdh10, Dusp14, Srxn1, Fos, and Nr4a1. In some embodiments of any of the aspects, the odor response genes comprise Tsc22d1. In some embodiments of any of the aspects, the odor response genes comprise Rcan1. In some embodiments of any of the aspects, the odor response genes comprise Fosb. In some embodiments of any of the aspects, the odor response genes comprise Pcdh10. In some embodiments of any of the aspects, the odor response genes comprise Dusp14. In some embodiments of any of the aspects, the odor response genes comprise Srxn1. In some embodiments of any of the aspects, the odor response genes comprise Fos. In some embodiments of any of the aspects, the odor response genes comprise Nr4a1. In some embodiments of any of the aspects, the odor response genes comprise Tsc22d1 and Rcan1. In some embodiments of any of the aspects, the odor response genes comprise Tsc22d1 and Fosb. In some embodiments of any of the aspects, the odor response genes comprise Tsc22d1 and Pcdh10. In some embodiments of any of the aspects, the odor response genes comprise Tsc22d1 and Dusp14. In some embodiments of any of the aspects, the odor response genes comprise Tsc22d1 and Srxn1. In some embodiments of any of the aspects, the odor response genes comprise Tsc22d1 and Fos. In some embodiments of any of the aspects, the odor response genes comprise Tsc22d1 and Nr4a1 In some embodiments of any of the aspects, the odor response genes comprise Rcan1 and Fosb. In some embodiments of any of the aspects, the odor response genes comprise Rcan1 and Pcdh10. In some embodiments of any of the aspects, the odor response genes comprise Rcan1 and Dsup14. In some embodiments of any of the aspects, the odor response genes comprise Rcan1 and Srxn1. In some embodiments of any of the aspects, the odor response genes comprise Rcan1 and Fos. In some embodiments of any of the aspects, the odor response genes comprise Rcan1 and Nr4a1. In some embodiments of any of the aspects, the odor response genes comprise Pcdh10 and Dusp14. In some embodiments of any of the aspects, the odor response genes comprise Pcdh10 and Srxn1. In some embodiments of any of the aspects, the odor response genes comprise Pcdh10 and Fos. In some embodiments of any of the aspects, the odor response genes comprise Pcdh10 and Nr4a1. In some embodiments of any of the aspects, the odor response genes comprise Dusp14 and Srxn1. In some embodiments of any of the aspects, the odor response genes comprise Dusp14 and Fos. In some embodiments of any of the aspects, the odor response genes comprise Dusp14 and Nr4a1. In some embodiments of any of the aspects, the odor response genes comprise Srxn1 and Fos. In some embodiments of any of the aspects, the odor response genes comprise Srxn1 and Nr4a1. In some embodiments of any of the aspects, the odor response genes comprise Fos and Nr4a1.

In some embodiments of any of the aspects, the odor response genes comprise Tsc22d1, Rcan1, Fosb, Pcdh10, Dusp14, Srxn1, Fos, and Nr4a1, In some embodiments of any of the aspects, the odor response genes comprise at least 1 of Tsc22d1, Rcan1, Fosb, Pcdh10, Dusp14, Srxn1, Fos, and Nr4a1. In some embodiments of any of the aspects, the odor response genes comprise at least 2 of Tsc22d1, Rcan1, Fosb, Pcdh10, Dusp14, Srxn1, Fos, and Nr4a1. In some embodiments of any of the aspects, the odor response genes comprise at least 3 of Tsc22d1, Rcan1, Fosb, Pcdh10, Dusp14, Srxn1, Fos, and Nr4a1. In some embodiments of any of the aspects, the odor response genes comprise at least 4 of Tsc22d1, Rcan1, Fosb, Pcdh10, Dusp14, Srxn1, Fos, and Nr4a1. In some embodiments of any of the aspects, the odor response genes comprise at least 5 of Tsc22d1, Rcan1, Fosb, Pcdh10, Dusp14, Srxn1, Fos, and Nr4a1. In some embodiments of any of the aspects, the odor response genes comprise at least 6 of Tsc22d1, Rcan1, Fosb, Pcdh10, Dusp14, Srxn1, Fos, and Nr4a1. In some embodiments of any of the aspects, the odor response genes comprise at least 7 of Tsc22d1, Rcan1, Fosb, Pcdh10, Dusp14, Srxn1, Fos, and Nr4a1.

In some embodiments of any of the aspects, the odor response genes comprise Btg2, Egr1, Fos, Fosb, Gm13889, and Junb. In some embodiments of any of the aspects, the odor response genes comprise at least 1 of Btg2, Egr1, Fos, Fosb, Gm13889, and Junb. In some embodiments of any of the aspects, the odor response genes comprise at least 2 of Btg2, Egr1, Fos, Fosb, Gm13889, and Junb. In some embodiments of any of the aspects, the odor response genes comprise at least 3 of Btg2, Egr1, Fos, Fosb, Gm13889, and Junb. In some embodiments of any of the aspects, the odor response genes comprise at least 4 of Btg2, Egr1, Fos, Fosb, Gm13889, and Junb. In some embodiments of any of the aspects, the odor response genes comprise at least 5 of Btg2. Egr1. Fos. Fosb. Gm13889, and Junb.

In some embodiments of any of the aspects, the odor response genes comprise Btg2 and Egr1. In some embodiments of any of the aspects, the odor response genes comprise Btg2 and Fos. In some embodiments of any of the aspects, the odor response genes comprise Btg2 and Fosb. In some embodiments of any of the aspects, the odor response genes comprise Btg2 and Gm13889. In some embodiments of any of the aspects, the odor response genes comprise Btg2 and Junb. In some embodiments of any of the aspects, the odor response genes comprise Egr1 and Fos. In some embodiments of any of the aspects, the odor response genes comprise Egr1 and Fosb. In some embodiments of any of the aspects, the odor response genes comprise Egr1 and Gm13889. In some embodiments of any of the aspects, the odor response genes comprise Egr1 and Junb. In some embodiments of any of the aspects, the odor response genes comprise Fos and Fosb. In some embodiments of any of the aspects, the odor response genes comprise Fos and Gm13889. In some embodiments of any of the aspects, the odor response genes comprise Fos and Junb. In some embodiments of any of the aspects, the odor response genes comprise Fosb and Gm13889. In some embodiments of any of the aspects, the odor response genes comprise Fosb and Junb. In some embodiments of any of the aspects, the odor response genes comprise Gm13889 and Junb.

In some embodiments of any of the aspects, the odor response genes comprise Tsc22d1, Rcan1, Fosb, Pcdh10, Dusp14, and Srxn1. In some embodiments of any of the aspects, the odor response genes comprise at least 1 of Tsc22d1, Rcan1, Fosb, Pcdh10, Dusp14, and Srxn1. In some embodiments of any of the aspects, the odor response genes comprise at least 2 of Tsc22d1, Rcan1, Fosb, Pcdh10, Dusp14, and Srxn1. In some embodiments of any of the aspects, the odor response genes comprise at least 3 of Tsc22d1, Rcan1, Fosb, Pcdh10, Dusp14, and Srxn1. In some embodiments of any of the aspects, the odor response genes comprise at least 4 of Tsc22d1, Rcan1, Fosb, Pcdh10, Dusp14, and Srxn1. In some embodiments of any of the aspects, the odor response genes comprise at least 5 of Tsc22d1, Rcan1, Fosb, Pcdh10, Dusp14, and Srxn1.

In some embodiments of any of the aspects, the odor response genes comprise Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 1 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 2 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 3 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 4 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 5 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 6 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 7 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 8 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 9 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 10 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 11 of Btg2, Egr1, Fos, Fosb, Gm13889, Junb, Nr4a1, Nr4a2, Pcdh10, Srxn1, Tsc22d1, Rcan1, and Dusp14. In some embodiments of any of the aspects, the odor response genes comprise at least 12 of Btg2, Egr1, Fos, Fosb, Gm13889, Junb, Nr4a1, Nr4a2, Pcdh10, Srxn1, Tsc22d1, Rcan1, and Dusp14.

An “olfactory sensory neuron (OSN)” is defined as a bipolar neuron with dendrites facing the external surface of the cribriform plate with axons that pass through the cribriform foramina with terminal end at olfactory bulbs. An OSN can also be referred to as an olfactory receptor neuron (ORN). Humans have between 10 and 20 million OSNs that are located in the olfactory epithelium in the nasal cavity. The cell bodies of the OSNs are distributed among all three of the stratified layers of the olfactory epithelium.

As used herein, a “subtype” of an OSN refers to an OSN or group of OSN expressing a specific odorant receptor. When a first OSN and a second OSN are of the same subtype, the first OSN and second OSN are characterized by expressing the same odorant receptor. In some embodiments of any of the aspects, the first subtype expresses one odorant receptor. In some embodiments of any of the aspects, the first subtype expresses a first odorant receptor.

An odor receptor, olfactory receptor, or “odorant receptor”, is a chemoreceptor expressed in the cell membranes of olfactory sensory neurons which is responsible for the detection of one or more odorants (for example, compounds that have an odor) which give rise to the sense of smell. Odorant receptors are located on the membranes of the cilia of the OSNs have been classified as a complex type of ligand-gated metabotropic channels. An odorant will dissolve into the mucus of the olfactory epithelium and then bind to an odor receptor. Odor receptors can bind to a variety of odor molecules, with varying affinities. The difference in affinities causes differences in activation patterns resulting in unique odorant profiles. The activated odor receptor in turn activates the intracellular G-protein. GOLF (GNAL), adenylate cyclase and production of cyclic AMP (CAMP) opens ion channels in the cell membrane, resulting in an influx of sodium and calcium ions into the cell, and an efflux of chloride ions. This influx of positive ions and efflux of negative ions causes the neuron to depolarize, generating an action potential. As described herein, the inventors have identified odor response genes, the expression of which in an OSN is modulated by activation of the odorant receptor.

In some embodiments of any of the aspects, the odorant receptor is a human odorant receptor. In some embodiments of any of the aspects, the odorant receptor is a murine odorant receptor. Odorant receptors and their sequences and relationships are known in the art. For further discussion of odorant receptor sequences and relationships, see. e.g., Glusman G. Yanai I. Rubin I. Lancet D (May 2001). “The complete human olfactory subgenome”. Genome Research. 11 (5); 685-702. doi: 10.1101/gr.171001. PMID 11337468 and Godfrey. P et al. (2004). “The mouse olfactory receptor gene family”. PNAS. 101 (7): 2156-61; each of which are incorporated by reference herein in their entireties.).

In some embodiments of any of the aspects, the human odorant receptor is selected from the group consisting of: human OR family 1, human OR family 2, human OR family 3, human OR family 4, human OR family 5, human OR family 6, human OR family 7, human OR family 8, human OR family 9, human OR family 10, human OR family 11, human OR family 12, human OR family 13, human OR family 14, human OR family 51, human OR family 52, human OR family 55, human OR family 56, and any variant thereof having an amino acid sequence having at least 80% identity with the amino acid sequence of one of said human odorant receptors.

In some embodiments of any of the aspects, the odorant receptor is selected from the group consisting of: Olfr 545, Olfr 160, Olfr 17, Olfr 727, Olfr 728, and Olfr 729.

In some embodiments of any of the aspects, the odorant receptor is selected from the group consisting of: Olfr 545, Olfr 160, and Olfr 17.

In some embodiments of any of the aspects, the murine odorant receptor is selected from the group consisting of: murine OR family 1, murine OR family 2, murine OR family 3, murine OR family 4, murine OR family 5, murine OR family 6, murine OR family 7, murine OR family 8, murine OR family 9, murine OR family 10-15, murine OR family 16-34.

In some embodiments of any of the aspects, the first OSN expresses at least one olfactory receptor. In some embodiments of any of the aspects, the second OSN expresses at least one olfactory receptor.

In some embodiments of any of the aspects, the method further comprises measuring the expression of at least one odorant receptor gene. An OSN expresses one odorant receptor gene, and thus the subtype of the OSN can be identified by detecting which odorant receptor gene is most highly expressed, e.g., in parallel or in the same assay as the measurement of the expression of the at least one odor response gene.

In some embodiments of any of the aspects, a first OSN and a second OSN are isogenic. In some embodiments of any of the aspects, a first OSN and a second OSN are derived from the same cell line. In some embodiments of any of the aspects, a first OSN and a second OSN are clonal. In some embodiments of any of the aspects, a first OSN and a second OSN are obtained from the same subject. In some embodiments of any of the aspects, a first OSN and a second OSN are of the same species.

In some embodiments of any of the aspects, a first OSN and a second OSN of the same subtype are isogenic. In some embodiments of any of the aspects, a first OSN and a second OSN of the same subtype are derived from the same cell line. In some embodiments of any of the aspects, a first OSN and a second OSN of the same subtype are clonal. In some embodiments of any of the aspects, a first OSN and a second OSN of the same subtype are obtained from the same subject. In some embodiments of any of the aspects, a first OSN and a second OSN of the same subtype are of the same species.

An “odor” or “odorant” is caused by one or more volatilized chemical compounds that are generally found in low concentrations that humans and animals can perceive via their sense of smell. An odor can also be referred to as a “smell” or a “scent”, which can refer to either a pleasant or an unpleasant odor. Volatile organic compounds (VOCs) are organic chemicals that have a high vapour pressure at room temperature. High vapor pressure correlates with a low boiling point, which relates to the number of the sample's molecules in the surrounding air, a trait known as volatility.

A volatilized chemical compound refers to a chemical compound that is or can be evaporated or dispersed in vapor. For an individual chemical or class of chemical compounds to be perceived as an odor, it must be sufficiently volatile for transmission via the air to the olfactory system in the upper part of the nose. A scent or odor can comprise one or more volatilized chemical compounds. In the methods described herein, an OSN can be contacted (or not contacted) with at least volatilized chemical compound, e.g., 1 volatilized chemical compound, multiple volatilized chemical compounds, or a scent or odor. Examples of volatilized chemical compounds include but are not limited to isoprene, terpenes, pinene isomers, sesquiterpenes, and methanol. Volatilized chemical compounds that are perceived by humans or mice, and/or which are common in scents and odors are known in the art. For example, see, Fahlbusch, Karl-Georg; Hammerschmidt, Franz-Josef; Panten, Johannes; Pickenhagen, Wilhelm; Schatkowski, Dietmar; Bauer, Kurt; Garbe, Dorothea; Surburg, Horst. “Flavors and fragrances”. Ullmann's Encyclopedia of Industrial Chemistry. Weinheim: Wiley-VCH, doi: 10.1002/14356007.a11_141, which is incorporated by reference herein in its entirety.

In some embodiments of any of the aspects, the olfactory sensory neuron has been contacted with or exposed to at least one volatilized chemical compound (e.g., odorant). In some embodiments, an OSN is in vitro during the contacting. In other embodiments, an OSN is in vivo during the contacting.

Contacting an OSN with at least one volatilized chemical compound can comprise contacting with air containing the at least one volatilized chemical compound, piping air comprising the at least one volatilized chemical compound towards the OSN/subject or into a space holding the OSN/subject, placing a source of the at least one volatilized chemical compound in a room or enclosure with the subject or OSN, or placing the at least one volatilized chemical compound on an object that is brought in close proximity to the subject's nose. In some embodiments of any of the aspects, the contacting an OSN with at least one volatilized chemical compound comprises contacting a subject comprising the OSN with air comprising the at least one volatilized chemical compound.

In other embodiments, the olfactory sensory neuron has not been contacted with or exposed to an odorant. In some embodiments, an OSN is in vitro during the not contacting. In other embodiments, an OSN is in vivo during the not contacting.

Not contacting an OSN with at least one volatilized chemical compound can comprise occluding the nostrils of a subject comprising the second OSN or contacting a subject comprising the second OSN with air not comprising the at least one volatilized chemical compound. Occluding the nostrils of a subject can comprise the use of a physical device (e.g., nose plug or mask) that blocks the nostrils, physical action (e.g., holding the subject's nose using fingers), providing instructions to the subject to breathe through their mouth, exposing the subject to an allergen or infectious agent that results in increased mucosal production and/or inflammatory cytokine production in the nostrils, exposing the subject to a physical or chemical irritant of the epithelial lining of the nostril (e.g., cigarette or cigar smoke), or selecting a human subject that is over the age of 60.

As described above herein, the methods of the instant technology relate to determining or measuring the expression of at least one odor response gene and/or odorant receptor (which are collectively referred to herein as “targets”). In some embodiments, the first subtype expresses an odorant receptor that binds to at least one volatilized chemical compound. The binding to at least one volatilized chemical compound activates odor response genes and initiates transcription of those genes. The expression level of the odor response genes in a first OSN is compared to the expression level of odor response genes in a second OSN (optionally of the same subtype (e.g., expressing the same odorant receptor as the first OSN)) that has not been contacted with the at least least one volatilized chemical compound. In some embodiments, if the expression level of the odor response genes in the first OSN is log 2 fold-change of −0.5 or greater relative to the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is log 2 fold-change of −1 or greater relative to the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is log 2 fold-change of −2 or greater relative to the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is log 2 fold-change of −3 or greater relative to the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is log 2 fold-change of −4 or greater relative to the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is log 2 fold-change of −5 or greater relative to the expression level of the odor response genes in the second OSN (optionally of the same subtype (e.g., expressing the same odorant receptor as the first OSN)), then the odorant receptor of the OSNs binds specifically with the at least one volatilized chemical compound. If the expression level of the odor response genes in the first OSN is the same or less than the expression level of the odor response genes in the second OSN (optionally of the same subtype (e.g., expressing the same odorant receptor as the first OSN)), then it is considered to be that the odorant receptor does not bind specifically to the at least one volatilized chemical compound.

In some embodiments, if the expression level of the odor response genes in the first OSN is at least 1.5× the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is at least 2.5× the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is at least 2.5× the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is at least 3× the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is at least 4× the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is at least 5× the expression level of the odor response genes in the second OSN (optionally of the same subtype (e.g., expressing the same odorant receptor as the first OSN)), then the odorant receptor of the OSNs binds specifically with the at least one volatilized chemical compound. In some embodiments, if the expression level of the odor response genes in the first OSN is at least 10× the expression level of the odor response genes in the second OSN (optionally of the same subtype (e.g., expressing the same odorant receptor as the first OSN)), then the odorant receptor of the OSNs binds specifically with the at least one volatilized chemical compound.

Alternatively, a plurality of first OSNs and a plurality of second OSNs (optionally of the same subtype (e.g., expressing the same odorant receptor as the first OSN)) can be used. In such cases, an odorant receptor can be considered to bind specifically to an at least one volatilized chemical compound if at least 50% of the first OSNs exhibit an increased in the expression level of the at least one odor response genes. In some embodiments, an odorant receptor can be considered to bind specifically to an at least one volatilized chemical compound if at least 60% of the first OSNs exhibit an increased in the expression level of the at least one odor response genes. In such cases, an odorant receptor can be considered to bind specifically to an at least one volatilized chemical compound if at least 70% of the first OSNs exhibit an increased in the expression level of the at least one odor response genes.

In some embodiments, an odorant receptor can be considered to bind specifically to an at least one volatilized chemical compound if at least 50% of the first OSNs exhibit an increased in the expression level of at least 4 odor response genes. In some embodiments, an odorant receptor can be considered to bind specifically to an at least one volatilized chemical compound if at least 60% of the first OSNs exhibit an increased in the expression level of at least 4 odor response genes. In such cases, an odorant receptor can be considered to bind specifically to an at least one volatilized chemical compound if at least 70% of the first OSNs exhibit an increased in the expression level of at least 4 odor response genes.

In some embodiments of any of the aspects, the first OSN and/or the second OSN is in vitro during the contacting. In some embodiments of any of the aspects, the first OSN and/or the second OSN is in vivo during the contacting.

In some embodiments of any of the aspects, the contacting the first OSN comprises contacting a subject comprising the first OSN with air comprising the at least one volatilized chemical compound.

In some embodiments of any of the aspects, the not contacting the second OSN comprises: occluding the nostrils of a subject comprising the second OSN, or contacting a subject comprising the second OSN with air not comprising the at least one volatilized chemical compound.

In some embodiments of any of the aspects, the second OSN is not contacted with the at least one volatilized chemical compound for at least two hours prior to the measuring.

In some embodiments of any of the aspects, measurement of the level of a target and/or detection of the level or presence of a target, e.g. of an expression product (nucleic acid or polypeptide of one of the genes described herein) can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments of any of the aspects, a DNA sample can undergo enzymatic replication, e.g., by polymerase chain reaction (PCR).

Transformation, measurement, and/or detection of a target molecule, e.g. a odor response gene and/or odorant receptor mRNA or polypeptide can comprise contacting a sample (or OSN) with a reagent (e.g. a detection reagent) which is specific for the target, e.g., a target-specific reagent. In some embodiments of any of the aspects, the target-specific reagent is detectably labeled. In some embodiments of any of the aspects, the target-specific reagent is capable of generating a detectable signal. In some embodiments of any of the aspects, the target-specific reagent generates a detectable signal when the target molecule is present.

In certain embodiments, the gene expression products as described herein can be measured by determining the level of messenger RNA (mRNA) expression of the genes (e.g., targets) described herein. Such molecules can be isolated, derived, or amplified from a biological sample, such as an epithelialsample or OSN. Techniques for the detection of mRNA expression are known by persons skilled in the art, and can include but are not limited to, PCR procedures, RT-PCR, quantitative RT-PCR Northern blot analysis, differential gene expression. RNAse protection assay, microarray based analysis, next-generation sequencing: hybridization methods, etc.

In general, a PCR procedure relates a method of gene amplification which is comprised of (i) sequence-specific hybridization of primers to specific genes or sequences within a nucleic acid sample or library. (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization. i.e. each primer is specifically designed to be complementary to a strand of the genomic locus to be amplified. In an alternative embodiment. mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments of any of the aspects, the level of an mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing. SOLID sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning; A Laboratory Manual (4 ed.). Cold Spring Harbor Laboratory Press. Cold Spring Harbor, N.Y., USA (2012); which are incorporated by reference herein in their entireties.

In some embodiments of any of the aspects, measuring the level of one or more mRNAs can be performed using single cell sequencing, q-RT-PCR, MERFISH and related multiplexed in situ detection methods. MERFISH is an imaging method capable of simultaneously measuring the copy number and spatial distribution of hundreds to thousands of RNA species in single cells (See e.g.; Moffit, J. M. et al. Chapter One-RNA Imaging with Multiplexed Error-Robus Fluorescence In Situ Hybridization (MERFISH). Methods in Enzymology. 572. 1-49, which is incorporated by reference in its entirety). One who is skilled in the art will be able to perform these techniques.

In some embodiments of any of the aspects, measuring the expression comprises measuring the level of one or more mRNAs. In some embodiments of any of the aspects, measuring the expression comprises measuring the level of the mRNA(s) of the at least one odor response gene.

In some embodiments of any of the aspects, measuring the level of one or more mRNAs comprises measuring the level of the one or more mRNAs in a single cell. In some embodiments of any of the aspects, measuring the level of one or more mRNAs comprises single-cell sequencing.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff. A et al. PCR: Clinical Diagnostics and Research. Springer (1994)).

In some embodiments of any of the aspects, measuring the expression of a target can comprise measuring the level of a polypeptide gene expression product. Such methods to measure gene expression products, e.g., protein level, include immunocytochemistry. ELISA (enzyme linked immunosorbent assay), western blot, immunoprecipitation, LFIA, immunoprecipitation: enzyme-linked immunosorbent assay (ELISA); radioimmunological assay (RIA); sandwich assay; fluorescence in situ hybridization (FISH); immunohistological staining: radioimmunometric assay; immunofluorescence assay: mass spectroscopy: immunoelectrophoresis assay; and/or immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

In some embodiments of any of the aspects, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluorescence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycocrythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycocrythrin, AMCA, CyDyes™, 6-carboxyfhiorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J). N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes. e.g. Cy3, Cy5 and Cy7 dyes: coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments of any of the aspects, a detectable label can be a radiolabel including, but not limited to ³H. ¹²⁵I, ³⁵S, ¹⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urcase, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin, Other detection systems can also be used, for example, a biotin-streptavidin system. In this system, the antibodies immunoreactive (i.e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e.g. from DAKO; Carpinteria, CA. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can be a level of the target molecule in a OSN or subject not contacted with the at least one volatilized chemical compound. In some embodiments of any of the aspects, the reference can also be a level of expression of the target molecule in a control sample, a pooled sample of control individuals or a numeric value or range of values based on the same. In some embodiments of any of the aspects, the reference can be the level of a target molecule in a sample obtained from the same subject at an earlier point in time. e.g., the methods described herein can be used to determine if a subject's sensitivity or response is changing over time.

In some embodiments of any of the aspects, the reference can be a level of the target molecule in a population of subjects who do not have or are not diagnosed as having, and/or do not exhibit signs or symptoms of Parkington's Disease, Alzheimer's Disease, COVID-19, SARS-COV, MERS-COV, related coronavirus infections, and infections from common viral and bacterial agents like influenza, staphalococcus, and streptococcus. In some embodiments of any of the aspects, the reference can also be a level of expression of the target molecule in a control sample, a pooled sample of control individuals or a numeric value or range of values based on the same. In some embodiments of any of the aspects, the reference can be the level of a target molecule in a sample obtained from the same subject at an earlier point in time, e.g., the methods described herein can be used to determine if a subject's sensitivity or response to a given therapy is changing over time.

In some embodiments of any of the aspects, the expression of a target described herein (e.g. the at least one odor response gene and/or the odorant receptor) is measured after contacting the OSN with the at least one volatilized chemical compound. In some embodiments of any of the aspects, the expression of a target described herein (e.g, the at least one odor response gene and/or the odorant receptor) is measured 10 minutes or more after contacting the OSN with the at least one volatilized chemical compound. In some embodiments of any of the aspects, the expression of a target described herein (e.g, the at least one odor response gene and/or the odorant receptor) is measured 20 minutes or more after contacting the OSN with the at least one volatilized chemical compound. In some embodiments of any of the aspects, the expression of the a target described herein (e.g, the at least one odor response gene and/or the odorant receptor) is measured 30 minutes or more after contacting the OSN with the at least one volatilized chemical compound. In some embodiments of any of the aspects, the expression of the a target described herein (e.g, the at least one odor response gene and/or the odorant receptor) is measured 1 hour or more after contacting the OSN with the at least one volatilized chemical compound. In some embodiments of any of the aspects, the expression of the a target described herein (e.g, the at least one odor response gene and/or the odorant receptor) is measured 2 or more hours after contacting the OSN with the at least one volatilized chemical compound. In some embodiments of any of the aspects, the expression of the a target described herein (e.g, the at least one odor response gene and/or the odorant receptor) is measured 3 days or more after contacting the OSN with the at least one volatilized chemical compound.

In some embodiments, the expression of a target described herein (e.g, the at least one odor response gene and/or the odorant receptor is measured between 0.5 hours and 6 weeks, measured between 0.5 hours and 5.5 weeks, measured between 0.5 hours and 5 weeks, measured between 0.5 hours and 4.5 weeks, measured between 0.5 hours and 4 weeks, measured between 0.5 hours and 3.5 weeks, measured between 0.5 hours and 3 weeks, measured between 0.5 hours and 2.5 weeks, measured between 0.5 hours and 2 weeks, measured between 0.5 hours and 1.5 weeks, measured between 0.5 hours and 1 week, measured between 0.5 hours and 6.5 days, measured between 0.5 hours and 6 days, measured between 0.5 hours and 5.5 days, measured between 0.5 hours and 5 days, measured between 0.5 hours and 4.5 days, measured between 0.5 hours and 4 days, measured between 0.5 hours and 3.5 days, measured between 0.5 hours and 3 days, measured between 0.5 hours and 2.5 days, measured between 0.5 hours and 2 days, measured between 0.5 hours and 1.5 days, measured between 0.5 hours and 24 hours, measured between 0.5 hours and 23.5 hours, measured between 0.5 hours, and 23 hours, measured between 0.5 hours and 22.5 hours, measured between 0.5 hours and 22 hours, measured between 0.5 hours and 21.5 hours, measured between 0.5 hours and 21 hours, measured between 0.5 hours and 20.5 hours, measured between 0.5 hours and 20.5 hours, measured between 0.5 hours and 20 hours, measured between 0.5 hours and 20 hours, measured between 0.5 hours and 19.5 hours, measured between 0.5 hours and 19 hours, measured between 0.5 hours and 18.5 hours, measured between 0.5 hours and 18 hours, measured between 0.5 hours and 17.5 hours, measured between 0.5 hours and 17 hours, measured between 0.5 hours and 16.5 hours, measured between 0.5 hours and 16 hours, measured between 0.5 hours and 15.5 hours, measured between 0.5 hours and 15 hours, measured between 0.5 hours and 14.5 hours, measured between 0.5 hours and 14 hours, measured between 0.5 hours and 13.5 hours, measured between 0.5 hours and 13 hours, measured between 0.5 hours and 12.5 hours, measured between 0.5 hours and 12 hours, measured between 0.5 hours and 11.5 hours, measured between 0.5 hours and 11 hours, measured between 0.5 hours and 10.5 hours, measured between 0.5 hours and 10 hours, measured between 0.5 hours and 9.5 hours, measured between 0.5 hours and 9 hours, measured between 0.5 hours and 8.5 hours, measured between 0.5 hours and 8 hours, measured between 0.5 hours and 7.5 hours, measured between 0.5 hours and 7.0 hours, measured between 0.5 hours and 6.5 hours, measured between 0.5 hours and 6 hours, measured between 0.5 hours and 6 hours, measured between 0.5 hours and 5.5 hours, measured between 0.5 hours and 5 hours, measured between 0.5 hours and 4.5 hours, measured between 0.5 hours and 4 hours, measured between 0.5 hours and 3.5 hours, measured between 0.5 hours and 3 hours, measured between 0.5 hours and 2.5 hours, measured between 0.5 hours and 2 hours, measured between 0.5 hours and 1.5 hours, or measured between 0.5 hours and 1 hour.

In some embodiments, the expression of a target described herein (e.g., the at least one odor response gene and/or the odorant receptor) is measured after at least 2 hours after contacting an OSN with at least one volatilized chemical compound, after at least 3 hours, after at least 4 hours, after at least 5 hours, after at least 6 hours, after at least 7 hours, after at least 8 hours, after at least 9 hours, after at least 10 hours, after at least 11 hours, after at least 12 hours, after at least 13 hours, after at least 14 hours, after at least 15 hours, after at least 16 hours, after at least 17 hours, after at least 18 hours, after at least 19 hours, after at least 20 hours, after at least 21 hours, after at least 22 hours, after at least 23 hours, after at least 24 hours, after at least 25 hours, after at least 26 hours, after at least 27 hours, after at least 28 hours, after at least 29 hours, after at least 30 hours, after at least 31 hours, after at least 32 hours, after at least 33 hours, after at least 34 hours, after at least 35 hours, after at least 36 hours, after at least 37 hours, after at least 38 hours, after at least 39 hours, after at least 40 hours, after at least 41 hours, after at least 42 hours, after at least 43 hours, after at least 44 hours, after at least 45 hours, after at least 46 hours, after at least 47 hours, after at least 48 hours, after at least 49 hours, after at least 50 hours, after at least 51 hours, after at least 52 hours, after at least 53 hours, after at least 54 hours, after at least 55 hours, after at least 56 hours, after at least 57 hours, after at least 58 hours, after at least 59 hours, after at least 60 hours, after at least 61 hours, after at least 62 hours, after at least 63 hours, after at least 64 hours, after at least 65 hours, after at least 66 hours, after at least 67 hours, after at least 68 hours, after at least 69 hours, after at least 70 hours, after at least 71 hours, after at least 72 hours, after at least 73 hours, after at least 74 hours, after at least 75 hours, after at least 76 hours, after at least 77 hours, after at least 78 hours, after at least 79 hours, after at least 80 hours, after at least 81 hours, after at least 82 hours, after at least 83 hours, after at least 84 hours, after at least 85 hours, after at least 86 hours, after at least 87 hours, after at least 88 hours, after at least 89 hours, after at least 90 hours, after at least 91 hours, after at least 92 hours, after at least 92 hours, after at least 93 hours, after at least 94 hours, after at least 95 hours, after at least 96 hours, after at least 97 hours, after at least 98 hours, after at least 99 hours, after at least 100 hours, after at least 101 hours, after at least 102 hours, after at least 103 hours, after at least 104 hours, after at least 105 hours, after at least 106 hours, after at least 107 hours, after at least 108 hours, after at least 109 hours, after at least 110 hours, after at least 111 hours, after at least 112 hours, after at least 113 hours, after at least 114 hours, after at least 115 hours, after at least 116 hours, after at least 117 hours, after at least 118 hours, after at least 119 hours, or after at least 120 hours or more.

The methods described herein also relate to screens, e.g., for a volatilized chemical compound or mixture of volatilized chemical compounds that activates a particular OSN subtype, or activates a plurality of OSN subtypes in a specific pattern, or which activates or or more OSN subtypes in a way that most closely resembles a reference volatilized chemical compound or benchmark scent.

As used herein, the term “benchmark scent” is a selected scent that one is trying to mimic or improve. e.g. by using it as a benchmark to compare a library of compounds/scents to.

As used herein. “candidate” refers to an element, e.g., a ligand, volatilized chemical compounds, or odorant receptor, that are to be screened and/or analyzed for the strength of their interaction with one or more other elements. A “candidate” element can be known to have the relevant activity or structure, but be a candidate in the sense of being a candidate for binding of a particular level of avidity or specificity, a candidate for binding in particular physical conditions, or a candidate for binding as compared to or in direct competition with other candidates. Where a “candidate” element is referred to herein, the reference includes known elements of that category. For example, where “candidate ligand” is used herein, it encompasses volatilized chemical compounds known in the art. For the methods described herein, candidates may be screened individually, or in groups. Group screening is particularly useful where hit rates for effective candidates are expected to be low such that one would not expect more than one positive result for a given group.

As used herein, the term “ligand” refers to a molecule that binds specifically with an odorant receptor to. The ligand can be a volatilized chemical compound. When the ligand is bound to the odorant receptor, odor response genes are modulated as described elsewhere herein.

In some embodiments of any of the aspects, the method further comprises measuring the expression of at least one odor response gene in a plurality of OSNs contacted with at least one volatilized chemical compound, each of the plurality of OSNs expressing a different odorant receptor. Such methods permit identification of which odorant receptors an at least one volatilized chemical compound activates, both qualitatively and quantitatively. Such methods can be used to identify volatilized chemical compounds that activate a set of odorant receptors in a pattern that mimics a benchmark scent.

In some embodiments of any of the aspects, the method further comprises measuring the expression of at least one odor response gene in a plurality of OSNs, each of the plurality of OSNs having been contacted with a different at least one volatilized chemical compound. Such methods permit identification of which compounds activate a particular odorant receptor, either quantitatively or qualitatively. Such methods can be used to identify volatilized chemical compounds that activate a particular odorant receptor to a degree that mimics a benchmark scent.

In some embodiments of any of the aspects, each of the plurality of OSNs is cultured in different wells of a multi-well plate or different cell culture containers. In some embodiments of any of the aspects, the plurality of OSNs are cultured in the same well of a multi-well plate or the same cell culture container. In some embodiments of any of the aspects, each well of a multi-well plate or each cell culture container is contacted with the same at least one volatilized chemical compound. In some embodiments of any of the aspects, each well of a multi-well plate or cell culture container is contacted with a different at least one volatilized chemical compound.

In some embodiments of any of the aspects, each of the plurality of OSNs is present in a different subject during the contacting. Such approaches can permit a larger sample pool, accounting for individual differences in scent perception. In some embodiments of any of the aspects, the plurality of OSNs are present in the same subject during the contacting, e.g., to reflect the complexity of scent perception, which typically involves multiple odorant receptors in multiple OSNs at one time. In some embodiments of any of the aspects, each subject is contacted with the same at least one volatilized chemical compound. In some embodiments of any of the aspects, each subject is contacted with a different at least one volatilized chemical compound.

In some embodiments of any of the aspects, the method further comprising obtaining a sample comprising the first OSN and/or second OSN from the nose of a subject after the contacting and before the measuring, e.g., the contacting occurs in vivo and the measuring is done ex vivo. The obtaining of the sample can be done noninvasively, e.g., with nasal swabbing or scrapping. In some embodiments of any of the aspects, the obtaining of the sample does not comprise surgical methods.

In some embodiments of any of the aspects, the method further comprises comparing the expression of the at least one odor response gene in the first OSN to the expression of the at least one odor response genes in an OSN of the first subtype that has been contacted with a benchmark scent. In some embodiments of any of the aspects, a method of identifying an odorant receptor ligand, or measuring the strength of an odorant receptor ligand's binding comprises: measuring the expression of at least one odor response gene in a first olfactory sensory neuron (OSN) expressing a first odorant receptor that has been contacted with a candidate odorant receptor ligand; and determining that the candidate odorant receptor ligand binds the first odorant receptor if the expression of the at least one odor response gene in the first OSN is different from a reference level of expression in OSN expressing the first odorant receptor that has not been contacted with the candidate odorant receptor ligand. In some embodiments of any of the aspects, the method further comprises measuring the expression of at least odor response gene in a plurality of OSNs, each of the plurality of OSNs having been contacted with a different candidate odorant receptor ligand. In some embodiments of any of the aspects, the magnitude of the difference in expression of the at least one odor response gene in the first OSN from the reference level of expression in an OSN expressing the first odorant receptor that has not been contacted with the candidate odorant receptor ligand correlates to the strength of the binding of the candidate odorant receptor ligand and the odorant receptor.

In some embodiments of any of the aspects, a method of identifying an odorant receptor ligand, or measuring the strength of an odorant receptor ligand's binding comprises: measuring the expression of at least one odor response gene in a first olfactory sensory neuron (OSN) expressing a first odorant receptor that has been contacted with a candidate odorant receptor ligand; and determining that the candidate odorant receptor ligand binds the first odorant receptor if the expression of the at least one odor response gene in the first OSN is increased relative to a reference level of expression in OSN expressing the first odorant receptor that has not been contacted with the candidate odorant receptor ligand. In some embodiments of any of the aspects, the method further comprises measuring the expression of at least odor response gene in a plurality of OSNs, each of the plurality of OSNs having been contacted with a different candidate odorant receptor ligand. In some embodiments of any of the aspects, the magnitude of the increase in expression of the at least one odor response gene in the first OSN relative to the reference level of expression in an OSN expressing the first odorant receptor that has not been contacted with the candidate odorant receptor ligand correlates to the strength of the binding of the candidate odorant receptor ligand and the odorant receptor.

In some embodiments of any of the aspects, the level of expression products of no more than 1,000 other genes is determined. In some embodiments of any of the aspects, the level of expression products of no more than 500 other genes is determined. In some embodiments of any of the aspects, the level of expression products of no more than 200 other genes is determined. In some embodiments of any of the aspects, the level of expression products of no more than 100 other genes is determined. In some embodiments of any of the aspects, the level of expression products of no more than 20 other genes is determined. In some embodiments of any of the aspects, the level of expression products of no more than 10 other genes is determined. In some embodiments of any of the aspects, the level of expression of no more than 6 other genes is determined.

In some embodiments of the foregoing aspects, the expression level of a given gene can be normalized relative to the expression level of one or more reference genes or reference proteins.

In some embodiments, the reference level can be the level in a sample of similar cell type, sample type, sample processing, and/or obtained from a subject of similar age, sex and other demographic parameters as the sample/subject for which the level of odor response gene activity is to be determined. In some embodiments, the test sample and control reference sample are of the same type, that is, obtained from the same biological source, and comprising the same composition, e.g, the same number and type of cells.

In some embodiments of any of the aspects, the sample is obtained from the olfactory bulb, the olfactory epithelium, the nerve endings, and/or the nasal cavity. In some embodiments of any of the aspects, the sample is obtained from the nasal cavity. In some embodiments of any of the aspects, the sample is obtained from the olfactory bulb. In some embodiments of any of the aspects, the sample is obtained from the olfactory epithelium. In some embodiments of any of the aspects, the sample comprises epithelial cells, endothelial cells, olfactory sensory neurons, goblet cells, cilia, and/or mast cells. In some embodiments of any of the aspects, a biological sample can comprise epithelial cells, endothelial cells, olfactory sensory neurons, goblet cells, cilia, and/or mast cells that originate from the nostril of a nose of a subject.

The term “sample” or “test sample” as used herein denotes a sample taken or isolated from a biological organism. e.g., an epithelial or tissue sample from a subject. In some embodiments of any of the aspects, the present invention encompasses several examples of a biological sample. In some embodiments of any of the aspects, the biological sample is cells, or tissue, or bodily fluid. Exemplary biological samples include, but are not limited to, a biopsy, biofluid sample mucus; tissue biopsy; organ biopsy; mucosal secretion; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject. In some embodiments of any of the aspects, the test sample can be epithelial cells, endothelial cells, olfactory sensory neurons, goblet cells, cilia, and/or mast cells that originate from the nostril of a nose of a subject.

The sample can be obtained by removing a sample from a subject, but can also be accomplished by using a previously isolated sample (e.g. isolated at a prior timepoint and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample. e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods, assays, and systems described herein can further comprise a step of obtaining or having obtained a test sample from a subject. In some embodiments of any of the aspects, the subject is a mammal. In some embodiments of any of the aspects, the subject is a mouse. In some embodiments of any of the aspects, the subject is a mammal. In some embodiments of any of the aspects, the subject is a human. In some embodiments of any of the aspects, the subject can be a subject in need of treatment for (e.g. having or diagnosed as having) Parkinson's Disease, Alzheimer's Disease, COVID-19, SARS-COV, MERS-COV, and related coronavirus infections, as well as infections from common viral and bacterial agents such as influenza, staphalococcus, streptococcus or a subject at risk of or at increased risk of developing Parkinson's Disease, Alzheimer's Disease, COVID-19, SARS-COV, MERS-COV, and related coronavirus infections, as well as infections from common viral and bacterial agents such as influenza, staphalococcus, streptococcus as described elsewhere herein.

In one aspect of any of the embodiments, described herein is a kit comprising primers that hybridize to and permit amplification of at least one odor response gene mRNA. The kit can optionally include primers that hybridize and permit amplication of at least one odorant receptor mRNA, one more more volatilized chemical compounds, a benchmark scent, or an OSN as described herein. The primers and other elements of the kit can be present in separate formulations of the kit, e.g., for separate administration or for mixing prior to administration.

A kit is any manufacture (e.g., a package or container) comprising at least one reagent, e.g., primers that hybridize with at least one odor response gene, the manufacture being promoted, distributed, or sold as a unit for performing the methods described herein. The kits described herein can optionally comprise additional components useful for performing the methods described herein. By way of example, the kit can comprise fluids and compositions (e.g., buffers, needles, syringes etc.) suitable for performing one or more of the administrations according to the methods described herein, an instructional material which describes performance of a method as described herein, and the like. Additionally, the kit may comprise an instruction leaflet.

Several diseases are characterized by a loss or reduction in the sense of smell. Accordingly, the quantitative measures of scent perception used herein can be used to detect or diagnose such diseases or symptoms. In some embodiments of any of the aspects, the method comprises measuring the expression of at least one odor response gene in a first olfactory sensory neuron (OSN) that has been contacted with at least one volatilized chemical compound, wherein the first OSN is obtained from or present in a subject; and determining that the subject has a loss of smell if the expression of the at least one odor response gene in the first OSN is different from the expression in a second OSN (optionally of the same subtype (e.g., expressing the same odorant receptor as the first OSN)) that has been contacted with the at least one volatilized chemical compound and obtained from or is present in normal, healthy individual. In some embodiments of any of the aspects, the method comprises measuring the expression of at least one odor response gene in a first olfactory sensory neuron (OSN) that has been contacted with at least one volatilized chemical compound, wherein the first OSN is obtained from or present in a subject; and determining that the subject has a loss of smell if the expression of the at least one odor response gene in the first OSN is lower than from the expression in a second OSN (optionally of the same subtype (e.g., expressing the same odorant receptor as the first OSN)) that has been contacted with the at least one volatilized chemical compound and obtained from or is present in normal, healthy individual. In some embodiments of any of the aspects, the loss of smell indicates the subject has or is at risk of having Parkinson's Disease, Alzheimer's Disease, COVID-19, SARS-COV, MERS-COV, and related coronavirus infections, as well as infections from common viral and bacterial agents such as influenza, staphalococcus, streptococcus. In some embodiments of any of the aspects, the method further comprises administering a treatment for Parkinson's Disease, Alzheimer's Disease, COVID-19, SARS-COV, MERS-COV, and related coronavirus infections, as well as infections from common viral and bacterial agents such as influenza, staphalococcus, streptococcus, if the subject is determined to have a loss of smell. Treatments for such conditions are known in the art and include monoclonal antibody therapties, antivirals, antibiotics, antidepressants (eg., rasagiline, selegiline, etc), anti-tremor medications, dopamine promoters, memantine, rivastigmine, galantamine, etc.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g, the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species. e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate. e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects. A subject can be male or female.

A subject can be one who has been previously diagnosed with or identified as suffering from or having a condition in need of treatment (e.g., being unable to detect odor) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. A protein or polypeptide can be produced naturally or in vitro by synthetic means. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. Polypeptides can also undergo maturation or post-translational modification processes that may include, but are not limited to: glycosylation, proteolytic cleavage, lipidization, signal peptide cleavage, pro peptide cleavage, phosphorylation, and such like.

The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. The terms also refer to fragments or variants of the polypeptide that maintain at least 50% of the activity or effect, e.g. odor detection by the full length polypeptide. Conservative substitution variants that maintain the activity of a wildtype protein will include a conservative substitution as defined herein. The identification of amino acids most likely to be tolerant of conservative substitution while maintaining at least 50% of the activity of the wildtype is guided by, for example, sequence alignment with homologs or paralogs from other species. Amino acids that are identical between homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants, fragments, and/or fusion proteins can be tested for activity, for example, by administering the variant to an appropriate animal model of loss of smell as described herein.

In some embodiments, a polypeptide, can be a variant of a sequence described herein. In some embodiments, the variant is a conservative substitution variant. Variants can be obtained by mutations of native nucleotide sequences, for example. A “variant.” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., at least 50% of the wildtype protein. As to amino acid sequences, one of skill will recognize that individual substitutions, deletions or additions to a nucleic acid, peptide, polypeptide, or protein sequence which alters a single amino acid or a small percentage. (i.e., 5% or fewer. e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of the wildtype, e.g., 110%, 125%, 150%, 175%, 200%, 500%, 1000% or more.

One method of identifying amino acid residues which can be substituted is to align, for example, human to a homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence, e.g., a nucleic acid encoding one of those amino acid sequences. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g., BLASTp or BLASTn (available on the world wide web at blast.ncbi.nlm.nih.gov), with default parameters set.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile. Val. Leu, or Ala for one another), or substitution of one polar residue for another (such as between Lys and Arg: Glu and Asp; or Gin and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity. e.g., the activity and/or specificity of a native or reference polypeptide is retained.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure.

Amino acids can be grouped according to similarities in the properties of their side chains (in A. L. Lehninger, in Biochemistry, second ed., pp. 73-75. Worth Publishers. New York (1975)): (1) non-polar: Ala (A), Val (V), Leu (L), Ile (I), Pro (P), Phe (F), Trp (W), Met (M); (2) uncharged polar; Gly (G), Ser(S), Thr (T), Cys (C), Tyr (Y), Asn (N), Gln (Q); (3) acidic: Asp (D), Glu (E); (4) basic; Lys (K), Arg (R), His (H), Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. Typically conservative substitutions for one another also include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (sec. e.g., Creighton, Proteins (1984)).

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example, A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

In some embodiments, a polypeptide can comprise one or more amino acid substitutions or modifications. In some embodiments, the substitutions and/or modifications can prevent or reduce proteolytic degradation and/or prolong half-life of the polypeptide in a subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov. 2002); and/or Fc fragments (Ashkenazi and Chamow, 1997). The references in the foregoing paragraph are incorporated by reference herein in their entireties.

In some embodiments, a polypeptide as described herein can comprise at least one peptide bond replacement. A polypeptide as described herein can comprise one type of peptide bond replacement or multiple types of peptide bond replacements, e.g., 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide as described herein can comprise naturally occurring amino acids commonly found in polypeptides and/or proteins produced by living organisms. e.g. Ala (A), Val (V), Leu (L), Ile (I), Pro (P), Phe (F), Trp (W), Met (M), Gly (G), Ser(S), Thr (T), Cys (C), Tyr (Y), Asn (N), Gln (Q), Asp (D), Glu (E), Lys (K), Arg (R), and His (H). In some embodiments, a polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA).

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., an infectious disease. The term “treating” includes reducing or alleviating at least one adverse effect or symptom of a condition, disease or disorder associated with an infectious disease. Treatment is generally “effective” if one or more symptoms or clinical markers are reduced. Alternatively, treatment is “effective” if the progression of a disease is reduced or halted. That is. “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to a loss of smell or a disease characterized by a loss of smell. In some embodiments of any of the aspects, prophylactic treatment is not prevention of all symptoms or signs of a disease.

As used herein, the term “administering.” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein. “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection: an act of dispensing, mixing, and/or decanting; and/or manipulation of a delivery device or machine.

The term “statistically significant” or “significantly.” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a.” “an.” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation. “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.). The Encyclopedia of Molecular Cell Biology and Molecular Medicine, published by Blackwell Science Ltd., 1999-2012 (ISBN 9783527600908); and Robert A. Meyers (ed.). Molecular Biology and Biotechnology: a Comprehensive Desk Reference, published by VCH Publishers. Inc., 1995 (ISBN 1-56081-569-8); Immunology by Werner Luttmann, published by Elsevier, 2006; Janeway's Immunobiology. Kenneth Murphy. Allan Mowat. Casey Weaver (eds.). W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook. Molecular Cloning: A Laboratory Manual, 4th ed., Cold Spring Harbor Laboratory Press. Cold Spring Harbor, N.Y., USA (2012) (ISBN 1936113414); Davis et al., Basic Methods in Molecular Biology. Elsevier Science Publishing. Inc., New York. USA (2012) (ISBN 044460149X); Laboratory Methods in Enzymology: DNA. Jon Lorsch (ed.) Elsevier, 2013 (ISBN 0124199542); Current Protocols in Molecular Biology (CPMB), Frederick M. Ausubel (ed.). John Wiley and Sons, 2014 (ISBN 047150338X, 9780471503385). Current Protocols in Protein Science (CPPS), John E. Coligan (ed.). John Wiley and Sons. Inc., 2005; and Current Protocols in Immunology (CPI) (John E. Coligan. ADA M Kruisbeck. David H Margulies. Ethan M Shevach. Warren Strobe. (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications: including literature references, issued patents, published patent applications, and co-pending patent applications: cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:

1. A method comprising:
measuring the expression of at least one odor response gene in a first olfactory sensory neuron (OSN) of a first subtype that has been contacted with at least one volatized chemical compound.

2. The method of paragraph 1, wherein the method further comprises measuring the expression of at least one odor response gene in a second OSN that has not been contacted with the at least one volatized chemical compound.

3. The method of any one of the preceding paragraphs, further comprising determining that the first subtype expresses an odorant receptor that binds to the at least one volatized chemical compound if the expression of the at least one odor response gene in the first OSN is different from a reference level of expression in a second OSN that has not been contacted with the at least one volatized chemical compound.

4. A method comprising:
measuring the expression of at least one odor response gene in a first OSN of a first subtype that has been contacted with at least one volatized chemical compound;
measuring expression of at least one odor response gene in a second OSN that has not been contacted with the at least one volatized chemical compound; and
determining that the first subtype expresses an odorant receptor that binds to the at least one volatized chemical compound if the expression of the at least one odor response gene in the first OSN and the second OSN are different.

5. The method of any one of the preceding paragraphs, wherein the at least one odor response gene is selected from the group consisting of:
Tsc22d1, Rcan1, Fosb, Pcdh10, Dusp14, Srxn1, Gm13889, Golga4, Heg1, Cdkn1a, Gpr137c, Zfp655, Ublcp1, Efr3b, Etf1, Srrm4, Epcam, Trp53inp2, Arhgap17, Ric8b, 9530034E10Rik, Oaz2, Ctxn3, Slc38a2, Exoc6b, Nfatc1, Junb, Tle3, Siah2, Fos, Ago2, Arhgef28, Ddx3x, Hspa8, Hnrnph3, Erf, Ak2, Pnrc1, Mafg, D1Ertd622c, Arid5b, Scn3b, Phtf2, Hsph1, Cpeb4, Ubc, Sema7a, Dleu2, Rc3h1, Pcnx4, Btg2, 6430548M08Rik, Selenok, Psmd8, Map9, Cry2, Hspa41, Mcl1, Dnaja1, Fth1, Fam208a, Bmpr2, Zfp608, BC005561, Rab11fip4, Smim13, Ppp2ca, Sod1, Neat1, Brinp2, Tuba4a, Cdc14a, Kdm6b, Uckl1os, Stk40, Dnajb2, Dnajb9, Spsb3, Sub1, Rheb, Manea, Fzd3, Zfand5, Uhrf1bp1l, Cebpb, Wdfy3, Mfge8, Inpp4a, Nab1, Tceal9, Ube2g1, Pcdh7, Eefla1, Tnrc6c, Cd82, BC005537, Sdcbp, Ddx5, Olfm1, Srpk1, M6pr, Braf, Ahi1, Clcn3, Eef2, Rbm26, Arfgef3, Eif5, Tmem150c, Acbp2, Snrk, Bex3, Epb4112, Samd4b, Stard10, Pappa, Grsf1, Ralgds, Arih1, Micu3, Ptges3, Kras, Morf412, Rtp2, Wdr26, Hspa5, Zmynd8, Sv2c, Paip2, Rab39b, Ywhaz, Gpat3, Psen2, Pde4a, Atp2b1, Irs2, Cfap69, Hspa9, Tob1, Hspa14, Pard6a, Mapk4, Nr4a1, Sik3, Epha7, Hagh, Kalrn, Prkar1a, Lmbrd1, Erich3, Sf3b1, Ksr2, Ybx1, Nfix, Hivep1, Mfn2, Elof1, Wdfy2, Emd, Azin1, Wbp1, Nop53, Syt7, Ncoa1, Smarca5, Aes, Rnf150, Plxnb2, Wdsub1, Dyrk4, Epb4111, Pcdh8, Daam1, Prpf4b, Tnrc6a, Rufy3, Ptdss2, Tmem230, Scn3a, Rock1, Dph3, Zbtb7b, Yipf4, Ift74, Ebf1, Wsb2, Rnf182, Arglu1, Ppm11, Ccdc189, Ptov1, Ccdc157, Ppa1, Pnpla8, Rack1, Rsrp1, Ttc9, Hcn2, Ccdc40, Kat2b, Cngb1, Ndufa3, Eefla2, Fmn2, Macrod1, Polr2i, 1700016K19Rik, Jade1, Slcla2, Ralbp1, Eif4c3, Morn2, Trappc21, Scrn1, Rtp1, Phyh, Fetub, Mapre3, Dnajb13, Coa3, Spef1, 1810058124Rik, Cep290, Bbs4, Nin, D430042009Rik, Tex9, Sdc3, Ebf4, Oaz1, Napa, Ttc8, Sdhaf4, Cuta, Commd6, Sfr1, Hcfclr1, Nxnl2, Kcnh3, Trpm7, Glb112, Slc25a39, Nsmf, Pifo, Oscp1, 1110008P14Rik, Egr1, Ttl16, Sem1, Aplg1. Fkbp2, Suclg1, Cidea, Dynlrb2, Ndufa2, Map1a, Sun1, Rufy2, Rnh1, Dtna, Anapc16, Pdhb, A430035B10Rik, Faim2, Ebf2, Ccdc28b, Ttll3, Drc1, Sys1, Prdx5, Palm, Slu7, Dnajc15, Elmod1, Paqr9, Tubb3, Ascc1, Ndufc1, Pon2, Fkbp4, Trnp1, Cldn3, Cby1, Tctex1d2, Slc48a1, Nme5, 1110004E09Rik, Cnppd1, Naa38, Vdac3, Puf60, Cers5, Cbr1, Ift43, Fam217a, Ankrd10, Ube2n, Pfdn6, Mpc2, Ncbp2, Timm13, Lamtor5, Uqcr10, Slc27a2, Arpc51, Adh5, Gkap1, Ndufaf4, Psip1, Omp, Bcl11a, Ift81, 2010107E04Rik, Ubxn1, Ech1, Tnrc6b, Tmpo, Ssbp4, 2410015M20Rik, Mapk8ip2, Naxe, Gm13589, Hdgfl3, Slc22a23, Cacna1h, Slc25a5, Ndufa13, Anp32a, Ndufb10, Lhx2, Cenpx, Rnf220, Ankrd54, Ppdpf, Dtd1, Auh, Mrpl51, Psmd13, Lamtor4, Hist2h2be, Mapkap1, Arhgdig, Ndufv3, Ilkap, Ctsa, Ift27, Lpcat4, Atp50, Ypel3, Gadl1, Msln1, Hint2, Acbd7, Manf, Lamtor2, Ak1, Arl6, Uxs1, Sri, Tomm7, Ubxn6, Cd47, Snrpd3, Ppid, Rsph9, Rbfox2, Pigp, C1qbp, Fmc1, Bcl7a, Krtcap2, Flrt1, Sdhd, Bphl, Sod2, Ndufb7, Prrc2b, Rex1bd, Rab36, Pdrg1, Pfn2, Rarg, B230118H07Rik, Elav13, Mlf1, Eif1, Mett126, Kmt5a, Ndufa5, Ndufb3, Emc8, Bad, Smc3, Dpm3, Ndufa11, Cisd1, Mrpl27, Cldn9, Sec61b, Tpd5212, Polr2c, Ndufa7, Atf5, Gabarap, Srsf3, Psmb3, Bax, Atox1, 2410004P03Rik, Atpla1, Ptprs, Cib1, Hist1h2bc, Atp5k, Hmox2, Fam92b, Chd4, Elob, Kif5a, Tbcb, Ndufa4, Uqcr11, Hras, Atp6v1f, Pam16, Pim3, Lrriq1, Nfu1, Supt4a, Swi5, Mtx2, Faim, Taldo1, Ccdc34, Rbm39, 2210016L21Rik, Dpy30, Tomm22, Pygo1, Mat2b, Mrpl28, Eid1, Snrpe, Atp5j, Ndufs7, Park7, Ndufs4, Cirbp, Polr2f, Fbxo9, Mgst3, Gtf2h5, Mtch1, Mrps24, Actr1b, Sh3bgrl3, Bmpr1a, Pfn1, Tesc, Tmem258, Mrps14, Abhd16a, Vdac2, Nxph3, Polr2g, Them6, Cfap 126, Pitpnc1, Kctd1, and Dmkn.

6. The method of any one of the preceding paragraphs, wherein the at least one odor response gene is selected from the group consisting of:
Tsc22d1, Rcan1, Fosb, Pcdh10, Dusp14, Srxn1, Fos, and Nr4a1.

7. The method of any one of the preceding paragraphs, wherein the at least one odor response gene is selected from the group consisting of:
Tsc22d1, Rcan1, Fosb, Pcdh10, Dusp14, and Srxn1.

8. The method of any one of the preceding paragraphs, wherein the at least one odor response gene is selected from the group consisting of:
Btg2, Egr1, Fos, Fosb, Gm13889, Junb, Nr4a1, Nr4a2, Pcdh10, and Srxn1.

9. The method of any one of the preceding paragraphs, wherein the at least one odor response gene is selected from the group consisting of:
Btg2, Egr1, Fos, Fosb, Gm13889, Junb, Nr4a1, Nr4a2, Pcdh10, Srxn1, Tsc22d1, Rcan1, and Dusp14.

10. The method of any one of the preceding paragraphs, wherein the at least one odor response gene comprises wat least one odor response gene, at least two odor response genes, at least three odor response genes, at least five odor response genes, at least ten odor response genes, at least fifty odor response genes, or at least one hundred odor response genes.

11. The method of any one of preceding paragraphs, further comprising measuring the expression of at least one odorant receptor gene.

12. The method of any one of the preceding paragraphs, wherein the expression of the at least one odor response gene is measured 2 or more hours after contacting the OSN with the at least one volatized chemical compound.

13. The method of any one of the preceding paragraphs, wherein the expression of the at least one odor response gene is measured 3 days or more after contacting the OSN with the at least one volatized chemical compound.

14. The method of any one of the preceding paragraphs, wherein the first subtype is an odorant receptor subtype.

15. The method of any one of the preceding paragraphs, wherein the first subtype expresses one odorant receptor.

16. The method of any one of the preceding paragraphs, wherein the odorant receptor is a human odorant receptor.

17. The method of any one of the preceding paragraphs, wherein the odorant receptor is a murine odorant receptor.

18. The method of paragraph 16, wherein the human odorant receptor is selected from the group consisting of:
human OR family 1, human OR family 2, human OR family 3, human OR family 4, human OR family 5, human OR family 6, human OR family 7, human OR family 8, human OR family 9, human OR family 10, human OR family 11, human OR family 12, human OR family 13, human OR family 14, human OR family 51, human OR family 52, human OR family 55, human OR family 56, and any variant thereof having an amino acid sequence having at least 80% identity with the amino acid sequence of one of said human odorant receptors.

19. The method of any one of the preceding paragraphs, wherein the odorant receptor is selected from the group consisting of:
Olfr 545, Olfr 160, Olfr 17, Olfr 727, Olfr 728, and Olfr 729.

20. The method of paragraph any one of the preceding paragraphs, wherein the odorant receptor is selected from the group consisting of:
Olfr 545, Olfr 160, and Olfr 17.

21. The method of paragraph 17, wherein the murine odorant receptor is selected from the group consisting of:
murine OR family 1, murine OR family 2, murine OR family 3, murine OR family 4, murine OR family 5, murine OR family 6, murine OR family 7, murine OR family 8, murine OR family 9, murine OR family 10-15, murine OR family 16-34.

22. The method of any one of the preceding paragraphs, wherein the first OSN and/or the second OSN expresses at least one olfactory receptor.

23. The method of any one of the preceding paragraphs, wherein the first OSN and/or the second OSN is in vitro during the contacting.

24. The method of any one of the preceding paragraphs, wherein the first OSN and/or the second OSN is in vivo during the contacting.

25. The method of any one of the preceding paragraphs, wherein the contacting the first OSN comprises contacting a subject comprising the first OSN with air comprising the at least one volatilized chemical compound.

26. The method of any one of the preceding paragraphs, wherein the not contacting the second OSN comprises:
occluding the nostrils of a subject comprising the second OSN, or contacting a subject comprising the second OSN with air not comprising the at least one volatilized chemical compound.

27. The method of any one of the preceding paragraphs, wherein the second OSN is not contacted with the at least one volatilized chemical compound for at least two hours prior to the measuring.

28. The method of any one of the preceding paragraphs, wherein measuring the expression comprises measuring the level of one or more mRNAs.

29. The method of paragraph 28, wherein measuring the level of one or more mRNAs comprises measuring the level of the one or more mRNAs in a single cell.

30. The method of paragraph 29, wherein measuring the level of one or more mRNAs comprises single-cell sequencing.

31. The method of any one of the preceding paragraphs, further comprising measuring the expression of at least one odor response gene in a plurality of OSNs contacted with at least one volatilized chemical compound, each of the plurality of OSNs expressing a different odorant receptor.

32. The method of any one of the preceding paragraphs, further comprising measuring the expression of at least one odor response gene in a plurality of OSNs, each of the plurality of OSNs having been contacted with a different at least one volatilized chemical compound.

33. The method of any one of paragraphs 31-32, wherein each of the plurality of OSNs is cultured in different wells of a multi-well plate or different cell culture containers.

34. The method of any one of paragraphs 31-32, wherein the plurality of OSNs are cultured in the same well of a multi-well plate or the same cell culture container.

35. The method of any one of paragraphs 33-34, wherein each well of a multi-well plate or each cell culture container is contacted with the same at least one volatilized chemical compound.

36. The method of any one of the preceding paragraphs, wherein each well of a multi-well plate or cell culture container is contacted with a different at least one volatilized chemical compound.

37. The method of any one of paragraphs 31-32, wherein each of the plurality of OSNs is present in a different subject during the contacting.

38. The method of any one of paragraphs 31-32, wherein the plurality of OSNs are present in the same subject during the contacting.

39. The method of any one of paragraphs 37-38, wherein each subject is contacted with the same at least one volatilized chemical compound.

40. The method of any one of paragraphs 37-38, wherein each subject is contacted with a different at least one volatilized chemical compound.

41. The method of any one of the preceding paragraphs, further comprising obtaining a sample comprising the first OSN and/or second OSN from the nose of a subject after the contacting and before the measuring.

42. The method of any one of the preceding paragraphs, wherein the sample is obtained from the olfactory bulb, the olfactory epithelium, the nerve endings, and/or the nasal cavity.

43. The method of any one of the preceding paragraphs, wherein the sample comprises epithelial cells, endothelial cells, olfactory sensory neurons, goblet cells, cilia, and/or mast cells.

44. The method of any one of the preceding paragraphs, wherein the subject is a mammal.

45. The method of any one of the preceding paragraphs, wherein the subject is a mouse or human.

46. The method of any one of the preceding paragraphs, further comprising comparing the expression of the at least one odor response gene in the first OSN to the expression of the at least one odor response genes in an OSN of the first subtype that has been contacted with a benchmark scent.

47. A method of identifying an odorant receptor ligand, or measuring the strength of an odorant receptor ligand's binding, the method comprising
measuring the expression of at least one odor response gene in a first olfactory sensory neuron (OSN) expressing a first odorant receptor that has been contacted with a candidate odorant receptor ligand; and
determining that the candidate odorant receptor ligand binds the first odorant receptor if the expression of the at least one odor response gene in the first OSN is different from a reference level of expression in OSN expressing the first odorant receptor that has not been contacted with the candidate odorant receptor ligand.

48. The method of paragraph 47, further comprising measuring the expression of at least odor response gene in a plurality of OSNs, each of the plurality of OSNs having been contacted with a different candidate odorant receptor ligand.

49. The method of paragraphs 47 or 48, wherein the magnitude of the difference in expression of the at least one odor response gene in the first OSN from the reference level of expression in an OSN expressing the first odorant receptor that has not been contacted with the candidate odorant receptor ligand correlates to the strength of the binding of the candidate odorant receptor ligand and the odorant receptor.

50. A method comprising
measuring the expression of at least one odor response gene in a first olfactory sensory neuron (OSN) that has been contacted with at least one volatized chemical compound, wherein the first OSN is obtained from or present in a subject; and
determining that the subject has a loss of smell if the expression of the at least one odor response gene in the first OSN is different from the expression in a second OSN obtained from or present in a normal, healthy individual.

51. The method of paragraph 50, wherein the loss of smell indicates the subject has or is at risk of having Parkinson's Disease, Alzheimer's Disease, COVID-19, SARS-COV, MERS-COV, coronavirus, influenza, staphalococcus, and streptococcus.

52. The method of any one of paragraphs 50 or 51, further comprising administering a treatment for Parkinson's Disease, Alzheimer's Disease, COVID-19, SARS-COV, MERS-COV, coronavirus, influenza, staphalococcus, and streptococcus if the subject is determined to have a loss of smell.

The technology described herein is further illustrated by the examples which in no way should be construed as being further limiting.

### EXAMPLES

### Example 1: A Transcriptional Rheostat Couples Past Activity to Future Sensory Responses

### Summary

Animals traversing different environments encounter both stable background stimuli and novel cues, which are thought to be detected by primary sensory neurons and then distinguished by downstream brain circuits. Here it is shown that that each of the ˜1000 olfactory sensory neuron (OSN) subtypes in the mouse harbors a distinct transcriptome whose content is precisely determined by interactions between its odorant receptor and the environment. This transcriptional variation is systematically organized to support sensory adaptation: expression levels of more than 70 genes relevant to transforming odors into spikes continuously vary across OSN subtypes, dynamically adjust to new environments over hours, and accurately predict acute OSN-specific odor responses. The sensory periphery therefore separates salient signals from predictable background via a transcriptional rheostat whose moment-to-moment state reflects the past and constrains the future; these findings show a general model in which structured transcriptional variation within a cell type reflects individual experience.

### Introduction

Sensory adaptation allows neurons and networks to minimize responses to background stimuli, thereby building more efficient neural codes that emphasize surprising or novel information (Attneave, 1954; Barlow, 1961; Benda, 2021; Weber et al., 2019). Rapid (milliseconds to seconds) sensory adaptation via post-translational mechanisms occurs in most sensory neurons, and plays an important role in shaping peripheral responses to highly dynamic stimuli (Kostal et al., 2008; Martelli and Storace, 2021; Moore, 1994). However, it is not clear how sensory neurons adapt at the longer timescales over which animals traverse different environments—and therefore encounter different background stimuli—as is common during a typical circadian cycle.

Activity-dependent transcription evolves over minutes to hours and therefore in principle could support longer-term adaptation (Yap and Greenberg, 2018). For example, mature brain neurons are thought to use the regulated transcription of synaptic proteins and ion channels to homeostatically maintain their firing rates within a narrow target range (Davis, 2006, 2013; Marder, 2011; Marder and Goaillard, 2006; Turrigiano, 2011; Turrigiano, 1999; Turrigiano, 2017). If similar transcriptional mechanisms underlic adaptation in the periphery, then sensory neurons of a given type would be expected to occupy a range of transcriptional states, with the specific state adopted by any given neuron both reflecting its prior activity history and predicting its future responses to sensory inputs (Tyssowski and Gray, 2019). However, the relationship between activity-dependent gene regulation and functional adaptation remains elusive, in part because it has not yet been shown in either peripheral or central neurons that analog changes in activity yield proportional, bidirectional and systematic changes in the expression of genes that tune neural function (Davis, 2013).

There are ˜1000 different olfactory sensory neuron (OSN) subtypes in the mouse, each of which can be identified across individuals based upon its expression of a single odorant receptor (OR) (Monahan and Lomvardas, 2015). ORs are G-protein coupled receptors that transduce odor binding into an influx of calcium, which leads to action potentials. The expressed OR endows each OSN with a specific level of neural activity that depends upon the odors present within a given environment. Although post-translational mechanisms sculpt OSN responses to rapidly fluctuating odor plumes, at longer timescales it is thought that OSNs faithfully report odor-receptor interactions to the brain. For example, OSN responses to odors (as assessed by functional imaging of OSN axons in the olfactory bulb) appear similar in individual mice tested at different times, across different mice, and in mice before and after training to associate a test odorant with a reward (Bozza et al., 2004; Chu et al., 2017; Kato et al., 2012; Rubin and Katz, 1999; Soucy et al., 2009; Spors and Grinvald, 2002; Wachowiak and Cohen, 2001). In contrast, olfactory bulb and cortical neurons adapt to the repeated presentation of an odor on timescales of minutes (Best et al., 2005; Cleland and Linster, 2005; McNamara et al., 2008; Wilson, 1998; Wilson, 2000; Yadon and Wilson, 2005). These findings indicate that ORs confer stable odor response properties upon OSNs, while the brain separates salient odors from background; this idea has pervasively influenced theoretical and computational models of odor coding (Brann and Datta, 2020; Chong et al., 2020; Grabska-Barwińska et al., 2016; Hopfield, 1991; Litwin-Kumar et al., 2017; Schaffer et al., 2018; Teşilcanu et al., 2019; Wilson et al., 2017; Zwieker et al., 2016).

Consistent with this view, transcriptional analyses indicate that the genes expressed by mature OSNs are largely similar across subtypes, with the exception of the ORs themselves, a handful of genes related to dorsoventral position in the olfactory epithelium, and ˜10 axon guidance genes that enable glomerular targeting (Fletcher et al., 2017; Hanchate et al., 2015; Mori and Sakano, 2011; Nakashima et al., 2013; Scholz et al., 2016; Vihani et al., 2020; Wu et al., 2018). This homogeneity indicates that individual OSN subtypes-which vary widely in their level of activity within a given environment-do not adapt their odor responses by actively modulating gene expression. In contrast, bulk RNA and activity measurements that pool across all OSNs in mice have hinted that OSNs may use transcription to compensate for the chronic absence or presence of activity, although results and interpretations have varied across experiments (Barber and Coppola, 2015; Cadiou et al., 2014; Coppola and Waggener, 2012; Coppola et al., 2006; Fischl et al., 2014; Fitzwater and Coppola, 2020; Hagendorf et al., 2009; Kass et al., 2012; Waggener and Coppola, 2007; Wang et al., 1993; Wang et al., 2017). Resolving these discrepancies—and ultimately testing the hypothesis that dynamic gene expression mediates long-term adaptation in sensory neurons—requires asking how odor-evoked activity influences gene expression within particular OSN subtypes; this level of specificity is essential to clarify whether OSN transcriptomes are changed in an OR- and environment-specific manner: whether these changes are limited to small numbers of genes (as appears to be the case in C. elegans (Cho et al., 2016; Juang et al., 2013; L'Etoile et al., 2002)) or organized into a systematic program of gene expression; and whether environment-dependent transcriptional changes actually adapt OR-specific odor responses.

To address these questions the inventors characterized, via single cell RNA sequencing (scSeq), transcriptional variation across the extraordinarily large array of identifiable OSN subtypes. These experiments reveal that each OSN subtype expresses a distinguishable set of transcripts that is stereotyped across mice within a given environment. The main axis of transcriptional variability includes more than 70 functionally-relevant genes that adaptively attenuate or amplify the transformation of odors into spikes. Furthermore, the expression of these genes coordinately and continuously varies across OSN subtypes, is modulated by specific interactions between ORs and the environment, and predicts OSN responses to new odors. Transcriptional variation among OSNs is therefore systematically organized through a rheostat-like mechanism (akin to a balance control on a stereo) whose setting in each OSN is defined by OR-environment interactions. The findings indicate that the olfactory system uses this transcriptional rheostat to proportionally and bidirectionally adapt to persistent background odors—thereby enabling OSNs to engage in a form of sensory predictive coding—before odor information is transmitted to neural circuits responsible for perception. These data reveal that peripheral odor codes are flexible rather than fixed, and support a broad model in which neurons continuously individualize their transcriptomes to facilitate functional adaptation.

### Results

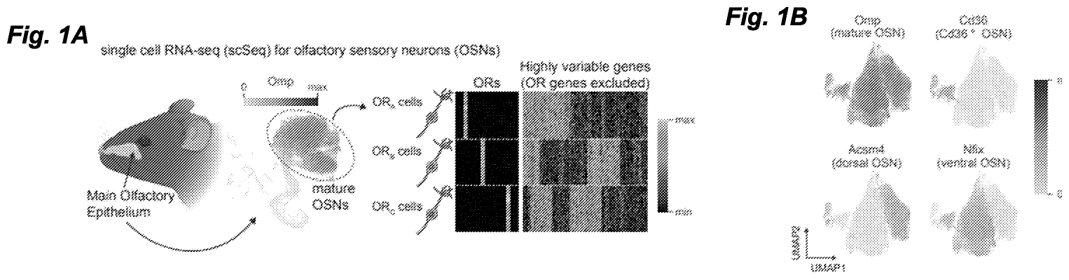

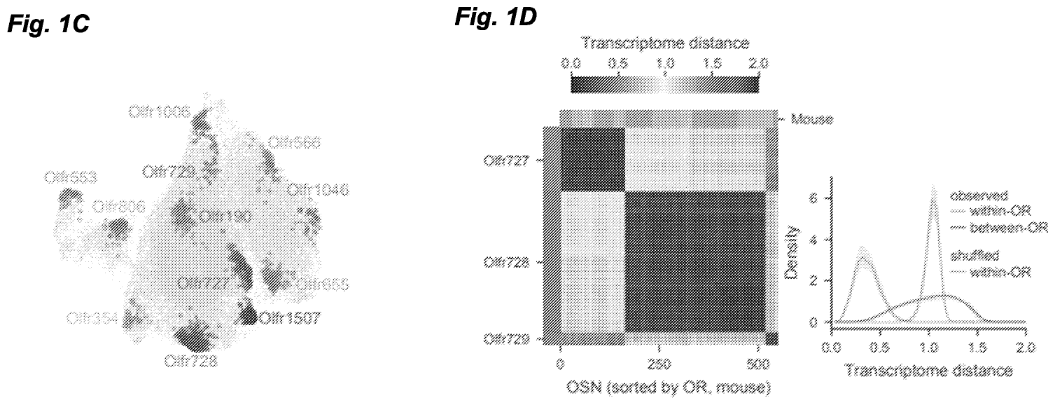

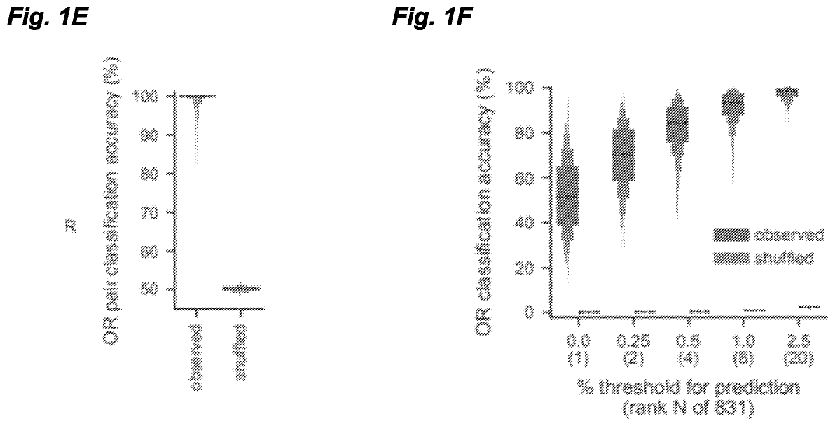

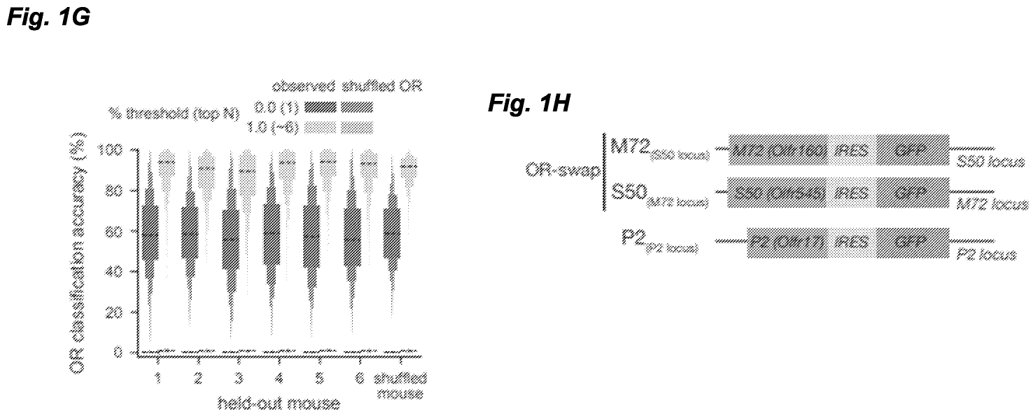

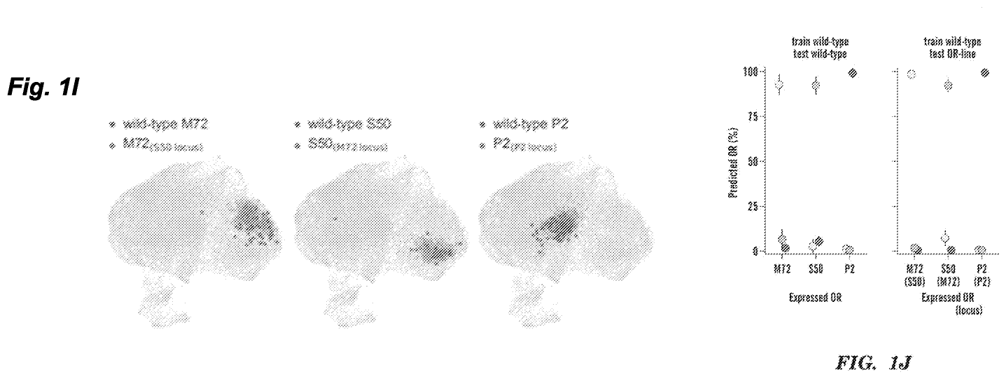

To assess relationships between transcription and function in the peripheral olfactory system scSeq was performed on −770,000 mature mouse OSNs. The initial analysis focused on −40,000 mature OSNs derived from adult mice housed in a typical home cage environment; collectively these OSNs expressed over 1,000 odorant receptors (ORs), with nearly every mature neuron expressing only one OR (FIGS. 1A, 81A-1D). UMAP embeddings of −1.350 highly variable genes (HVGs, here and in all analyses excluding the OR genes themselves, see Methods) revealed that OSNs are partitioned into several broad subtypes related to positional identity and the expression of the lipid receptor CD36 (FIG. 1B).

### OSN Transcriptomes are Stereotyped and Uniquely Specified by the Expressed OR

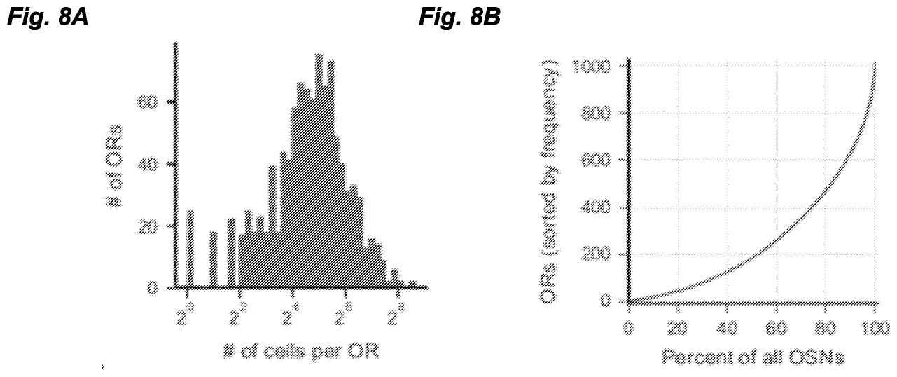

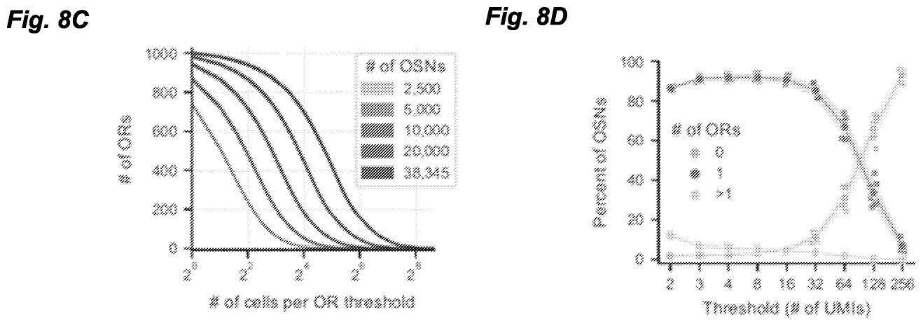

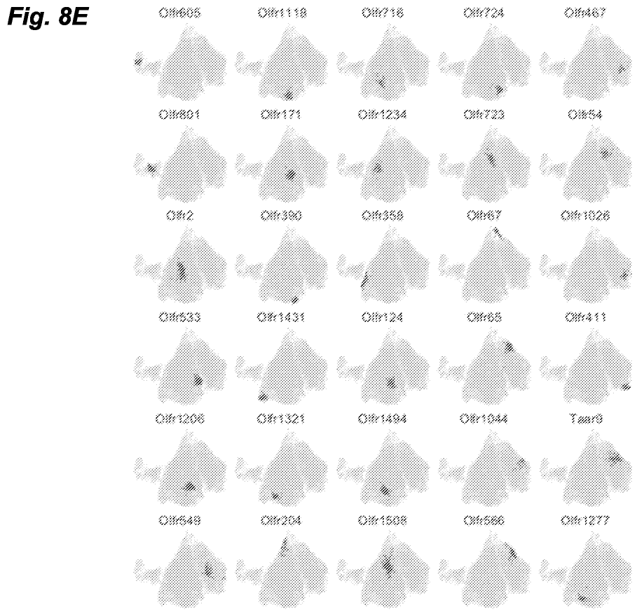

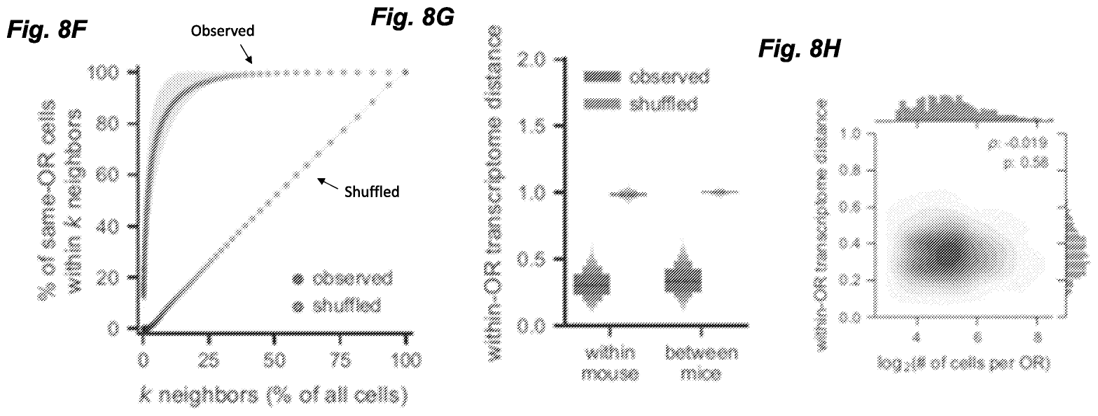

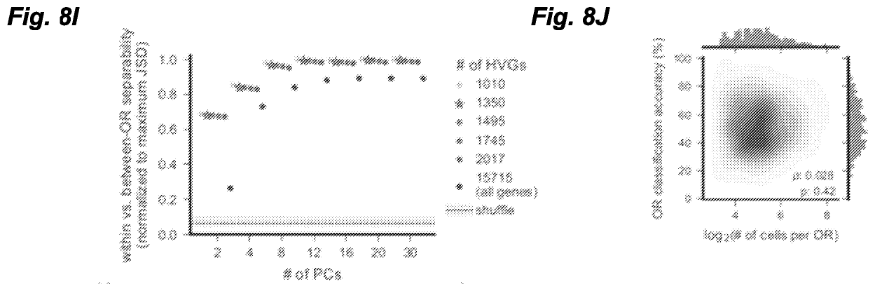

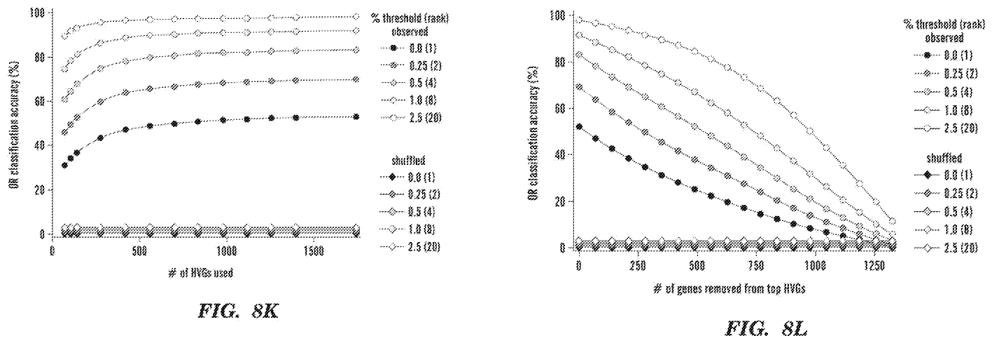

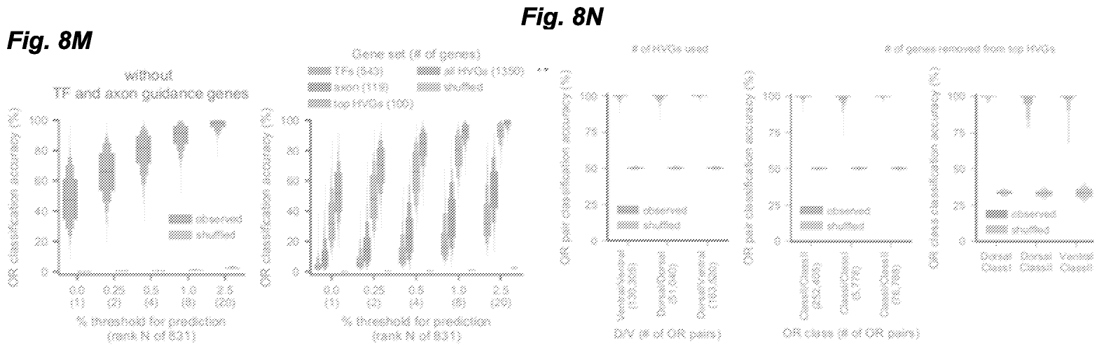

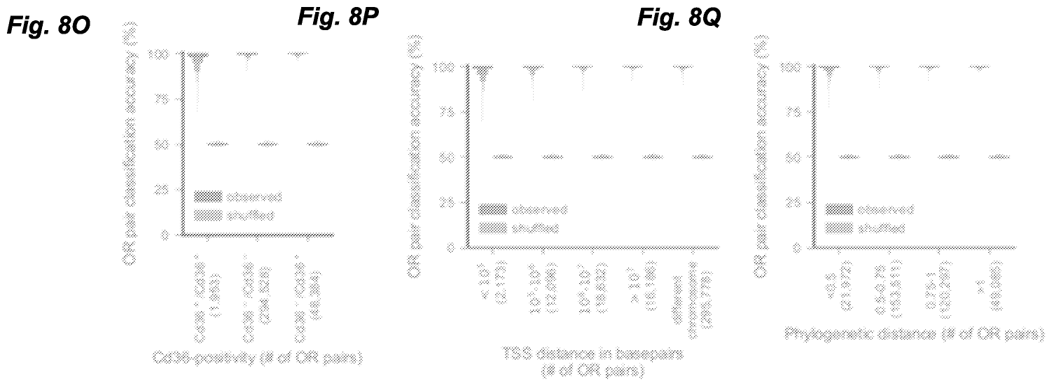

Given the few known transcriptional differences among mature OSNs, it was expected that OR-defined OSN subpopulations would overlap in UMAP space: instead. OSNs expressing each OR were locally clustered and separated from those expressing different ORs, showing that each OSN subtype is transcriptionally unique (FIGS. 1C, 8E). For example, the transcriptomes of OSNs expressing Olfr727 were self-similar, and distinct from OSNs expressing Olfr728 and Olfr729 (FIG. 1D). The transcriptomes of all OR-identified OSN subtypes were distinguishable, regardless of how transcriptome distances were computed or the frequency with which each OR was expressed (FIGS. 1D, 8F-8I). Linear classifiers distinguished OSNs expressing different ORs with near perfect accuracy and predicted the specific expressed OR (of the 831 queried) with >50% accuracy (vs. 0.1% at chance) (FIGS. 1E-1F, 8J). Approximately 1000 highly variable genes were required for maximum classification accuracy, although individual genes contributed marginally to overall performance (FIGS. 8K-L). Importantly, classifiers accurately predicted the identity of the expressed OR in OSNs derived from held-out mice, indicating OR-transcriptome relationships generalize across individuals housed in a similar environment (FIG. 1G).

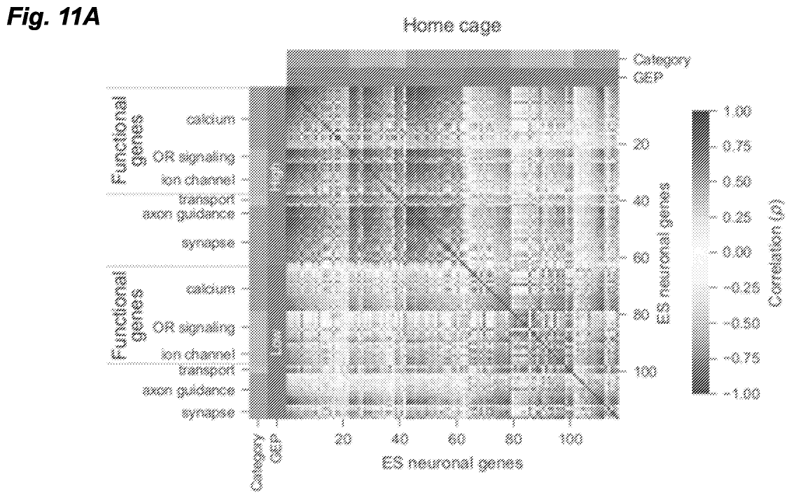

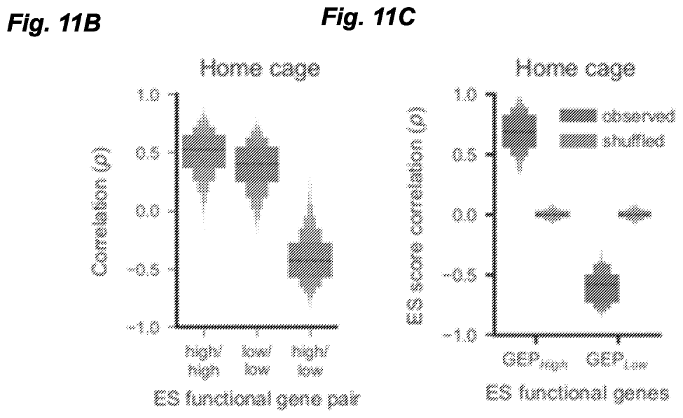

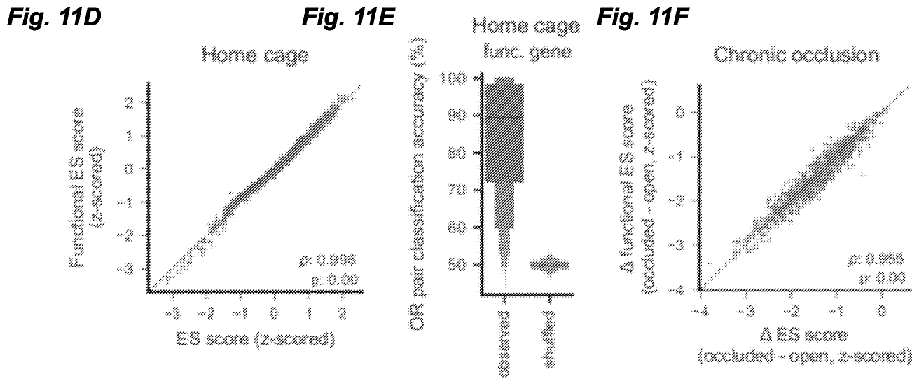

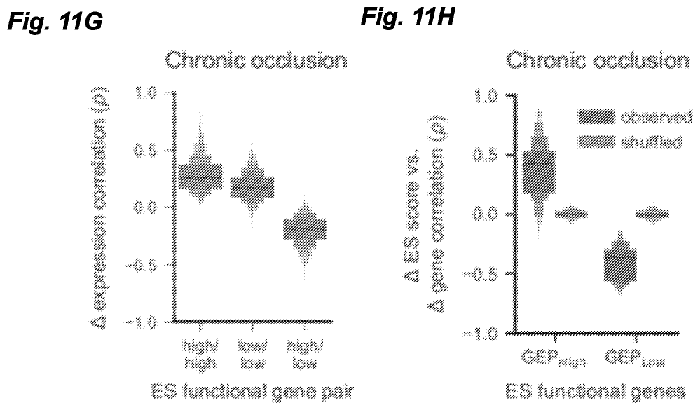

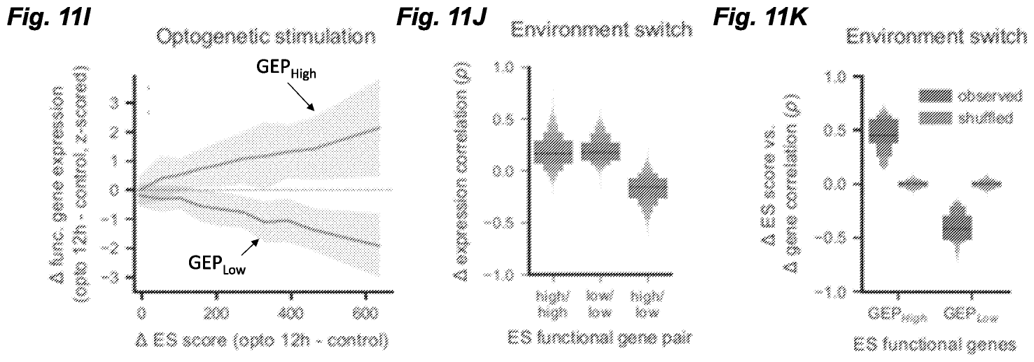

The unexpectedly close relationship between OR expression and OSN transcriptomes could result from shared regulatory mechanisms, or from each OR determining its associated transcriptome. To distinguish these possibilities, scSeq was performed in mice with swapped coding regions for the M72 (Olfr160) and S50 (Olfr545) receptor genes (FIG. 1H). OSNs expressing M72 receptor from the S50 gene locus (M72 (S50 locus)) aligned with wild-type M72 OSNs: likewise. OSNs expressing S50 from the M72 gene locus S50 (M72 locus) mice resembled wild-type S50 OSNs (FIGS. 11-1J). The unique gene expression signature associated with each OSN is therefore largely specified by the expressed OR protein.

OSNs express a limited number of transcription factors and axon guidance genes in an OR-dependent manner during development (Mori and Sakano, 2011; Parrilla et al., 2016). However, genes from these categories were neither required for accurate OR predictions nor were as predictive as either the complete set or the most predictive subset of HVGs (FIG. 8M). Each OSN can also be categorized by the expression of CD36, the specific class of its expressed OR (Class I vs Class II), or the dorsal or ventral location of its associated glomeruli in the olfactory bulb: classifiers accurately distinguished ORs from OSNs within or between each of these categories (FIGS. 8N-8O). Furthermore, classifiers distinguished OSNs independently of the genomic or phylogenetic relationships between ORs (FIGS. 8P-8Q). Therefore, known differences relating to OSN identity or genomic/phylogenetic features of ORs do not explain the transcriptional diversity of mature OSNs.

Decomposing OSN Transcriptomes into Identity and Activity Gene Expression Programs

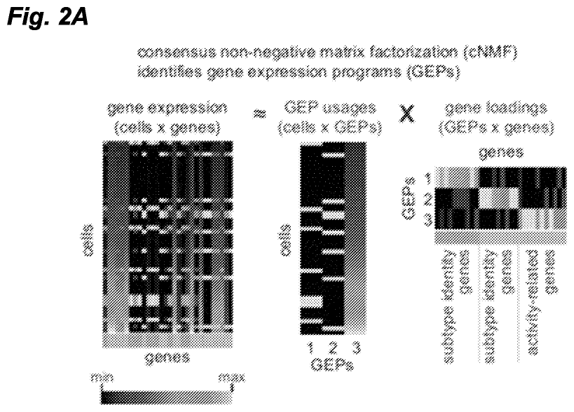

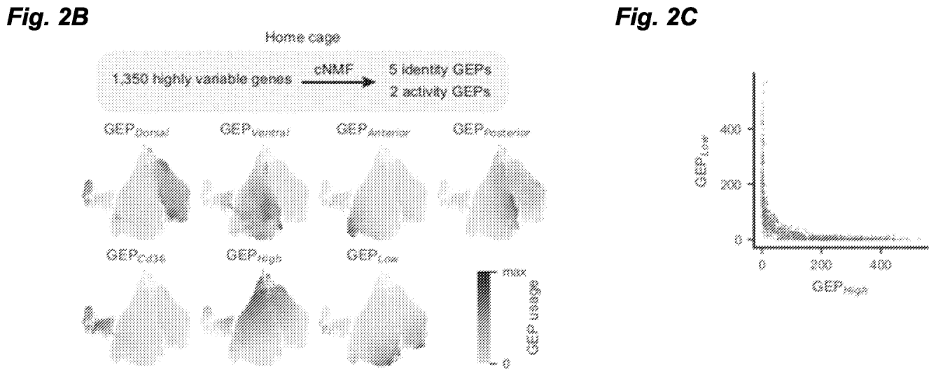

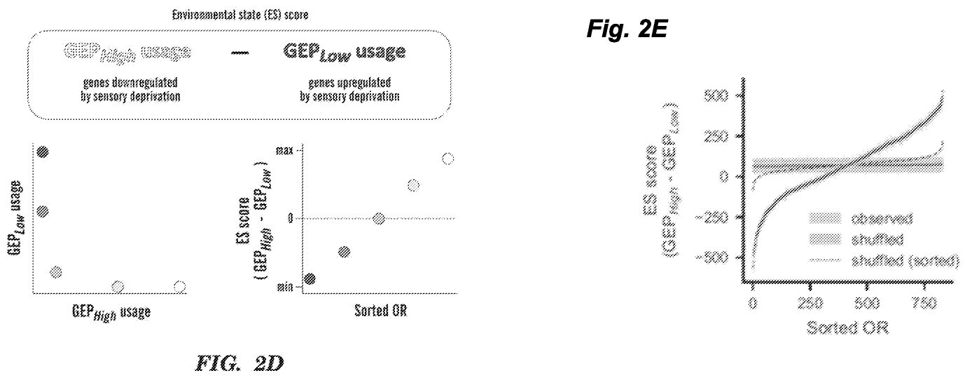

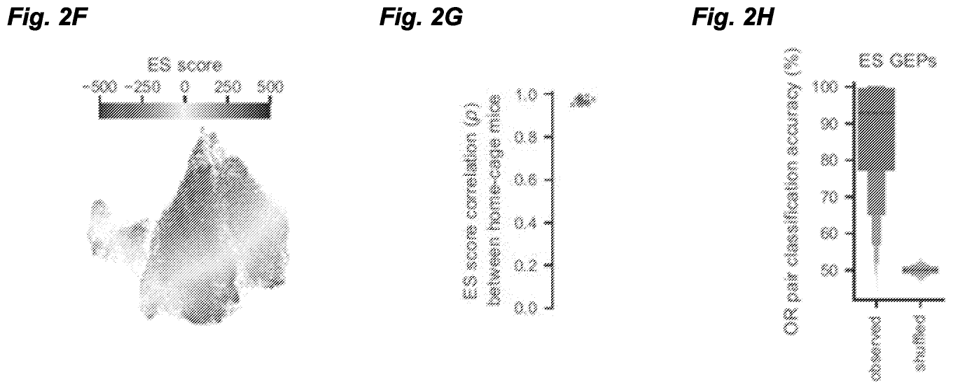

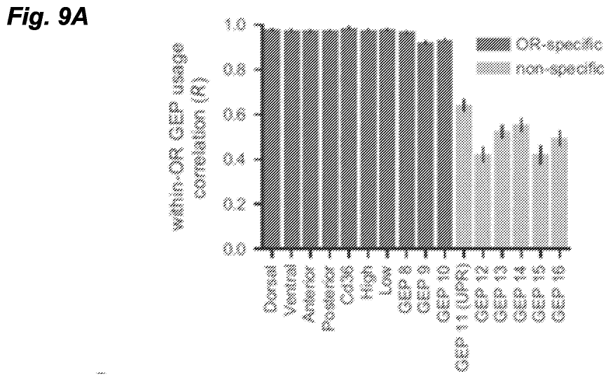

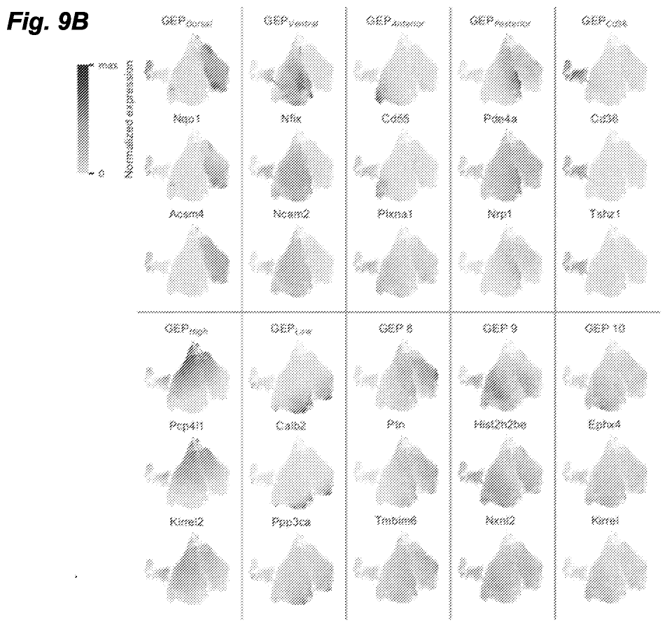

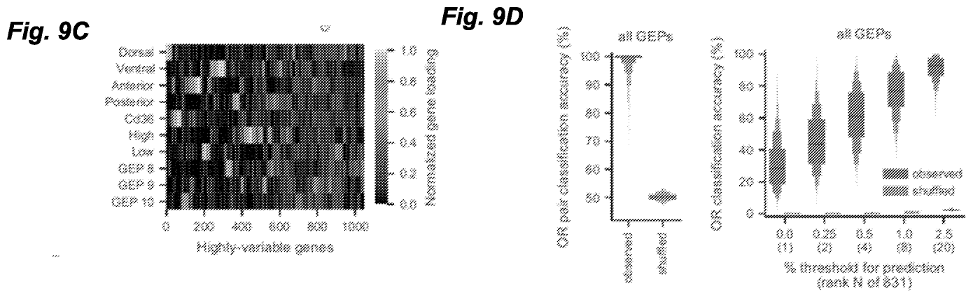

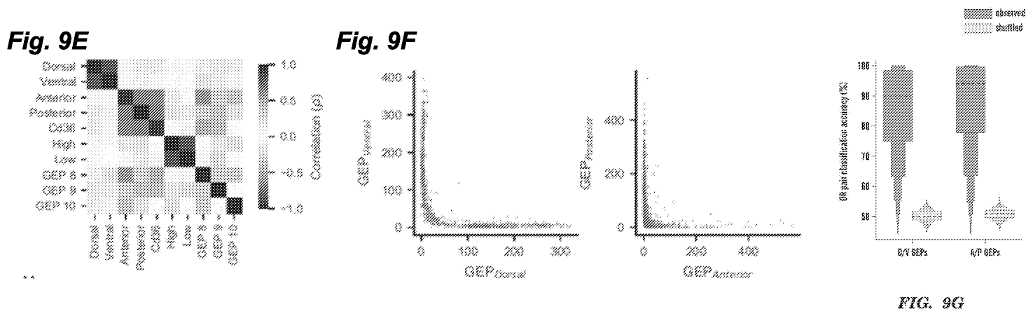

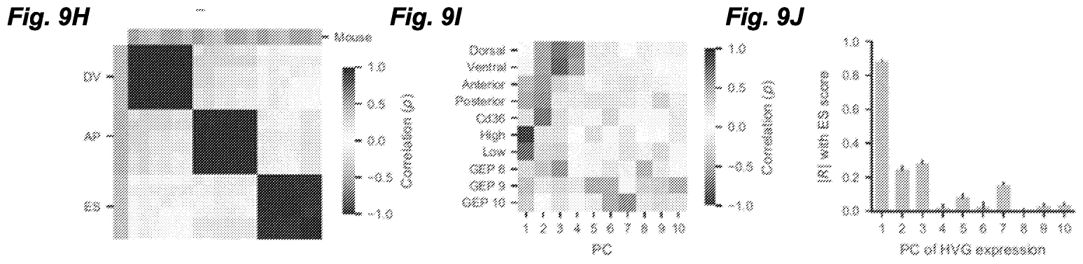

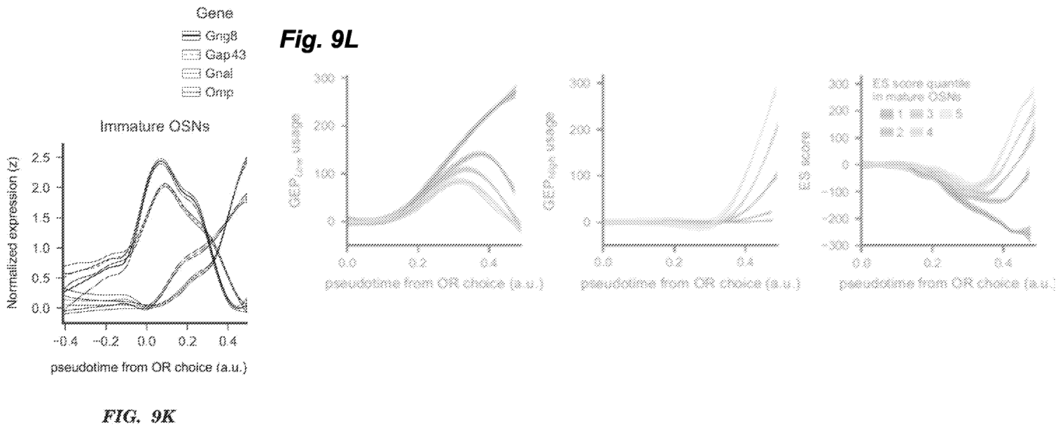

Alternatively, the diversity in OSN transcriptomes might reflect OR-associated differences in neural activity. To test this possibility, consensus non-negative matrix factorization (cNMF) was performed, which decomposes gene expression patterns observed across OSNs into sets of co-expressed genes called gene expression programs (GEPs) (FIG. 2A, see Methods); importantly, cNMF successfully identified both identity-associated and activity-related GEPs in a prior study (Kotliar et al., 2019), cNMF identified 10 GEPs—each composed of 100s of largely non-overlapping genes—that effectively captured OR-driven transcriptional variability, whose usages varied across OSNs in an OR-specific manner, and which accurately predicted the expressed OR in each OSN (FIGS. 2B and 9A-9D).

Of the 10 identified GEPs, the seven whose constituent genes indicated putative functions were focused on (FIG. 9E). Five GEPs encoded known aspects of OSN identity (dorsal, ventral, anterior, posterior, and CD36-positivity. (FIGS. 9B, 9F-9H). The other two GEPs, which are referred to as GEPHigh and GEPLow, included genes associated with neural activity in OSNs: GEPHigh included genes downregulated by chronic sensory deprivation (e.g., S100a5, Pep411, and Kirrel2) while GEPLow included genes upregulated after chronic sensory deprivation (e.g., Calb2, Kirrel3, and Ppp3cal) (Fischl et al., 2014; Wang et al., 2017). Each OSN primarily used either GEPLow or GEPHigh, and the usage of each GEP varied continuously across OSN subtypes (FIGS. 2C, 9B).

It was hypothesized that persistent interactions between each OR and the wide variety of odors in a given environment (like the food, bedding and semiochemical odors present in a typical home cage) might explain the continuous variation of GEPLow or GEPHigh across OSN subtypes. To explore this idea, a metric was developed (simply the difference between GEPHigh and GEPLow) that is provisionally refer to as the environmental state(ES) score, (FIG. 2D). ES scores for OSNs derived from mice housed in the home cage varied continuously across OSN subtypes; however, OR-specific ES scores were remarkably consistent across mice and enabled accurate predictions of OR identity (FIGS. 2E-2H). Both ES scores and GEPHigh and GEPLow usages aligned with the first principal component of the OSN transcriptomes (FIGS. 9I-9J). GEPHigh and GEPLow genes were not expressed in immature OSNs but their expression increased and diversified after the onset of OR expression (FIGS. 9K-9L).

### OSN Transcriptomes Systematically Reflect Environment-Dependent Activity

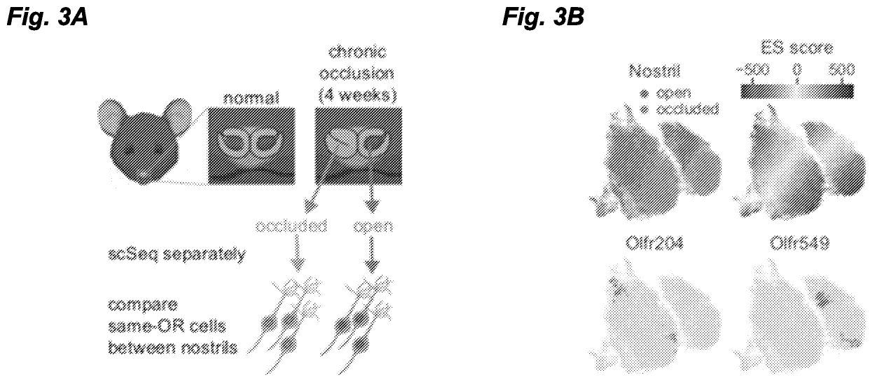

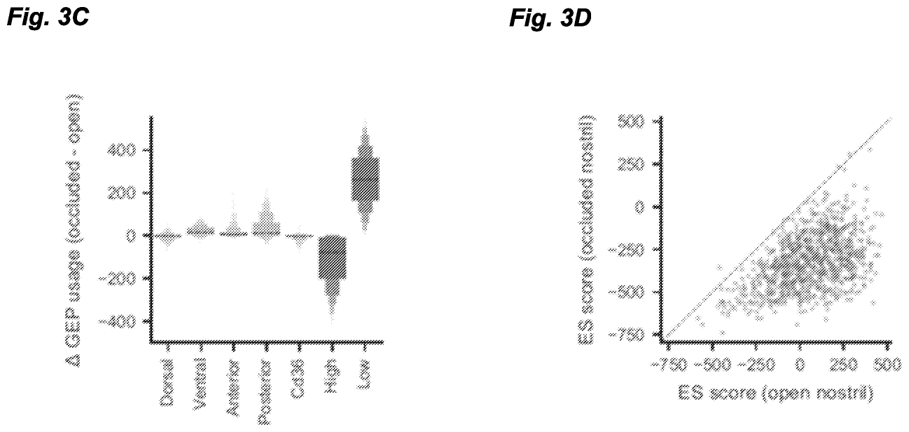

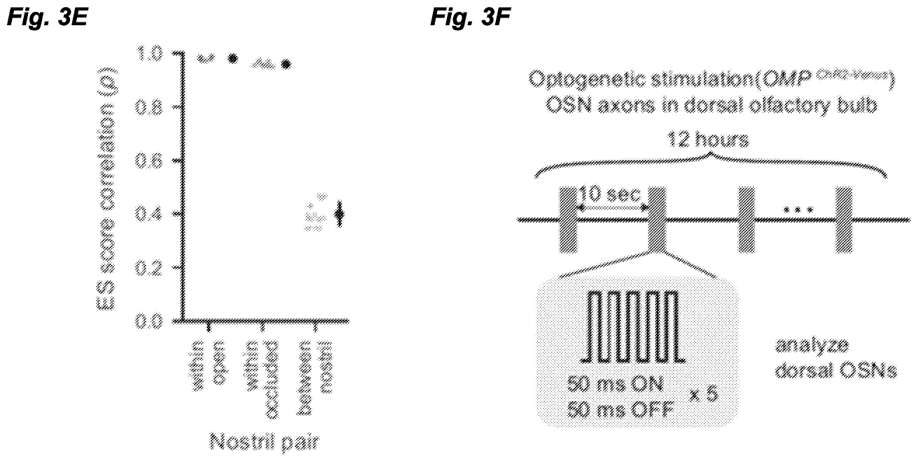

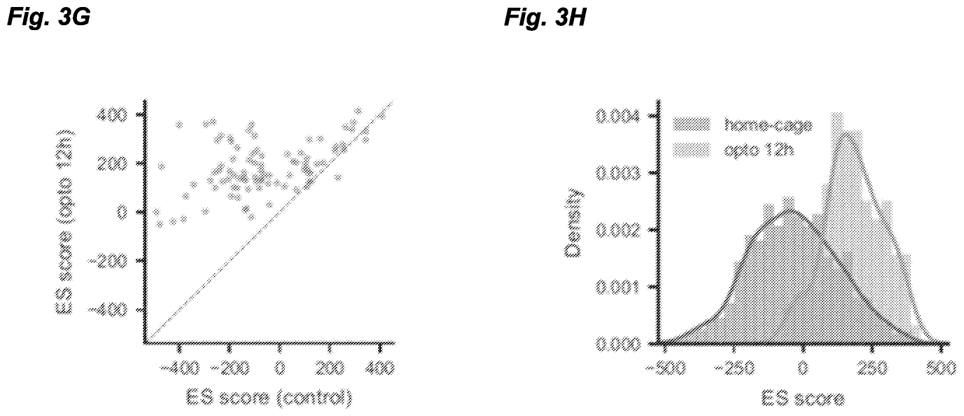

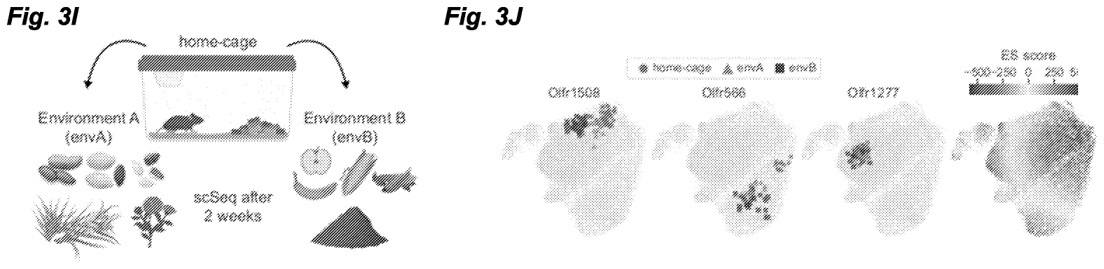

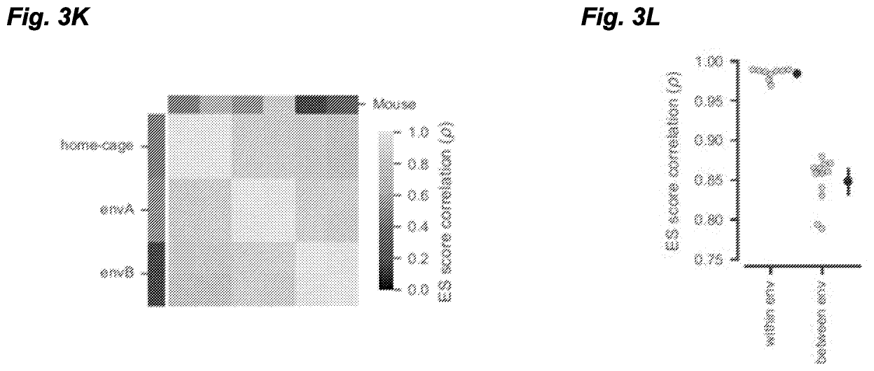

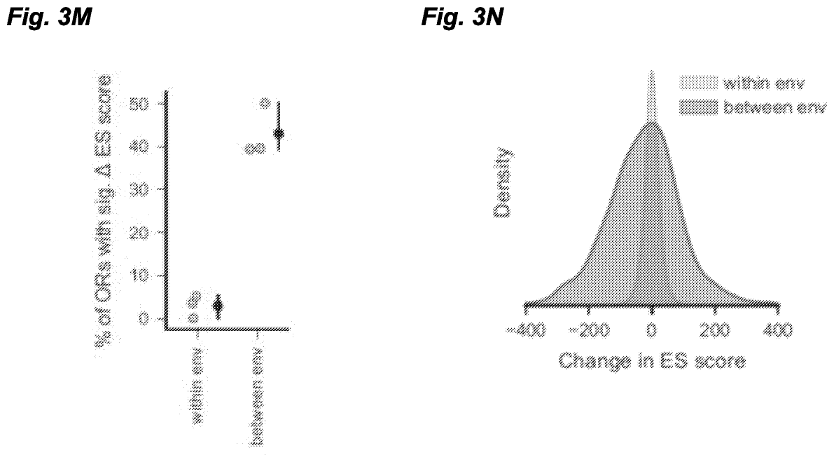

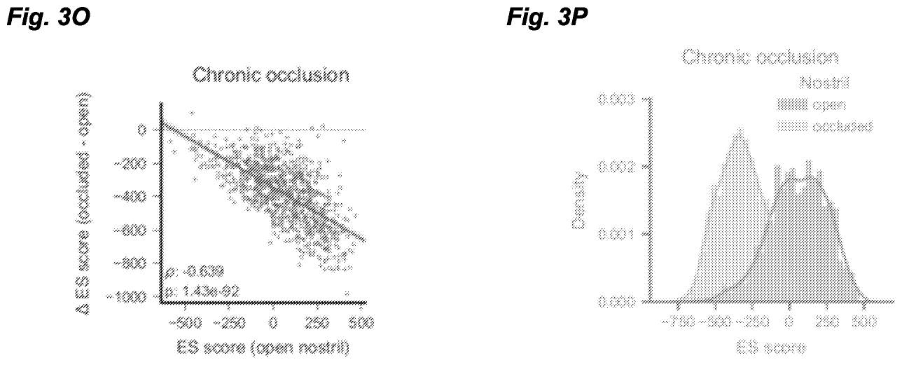

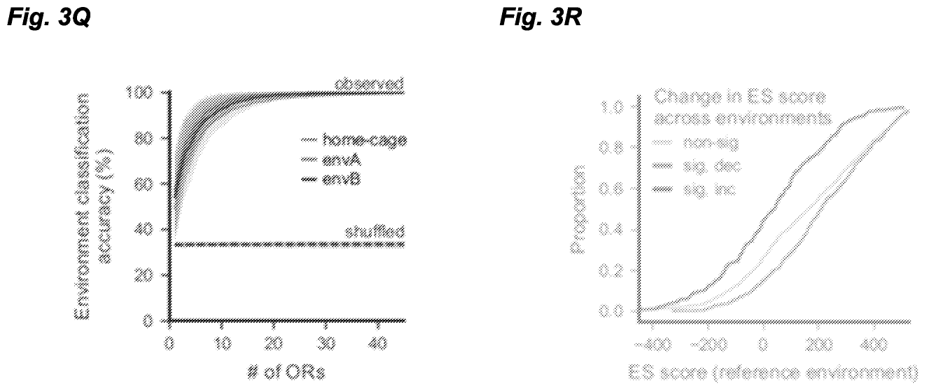

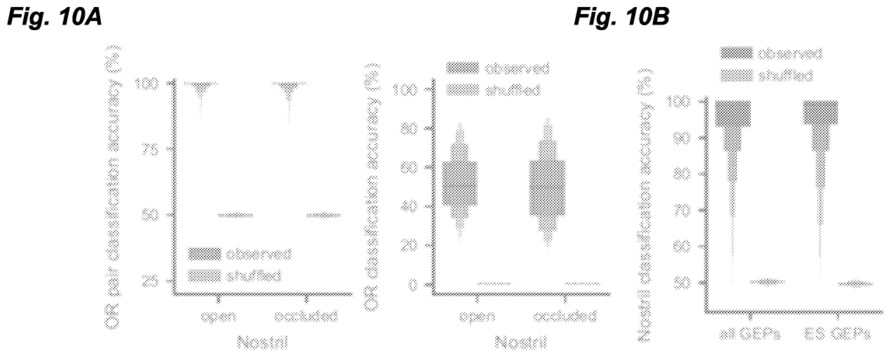

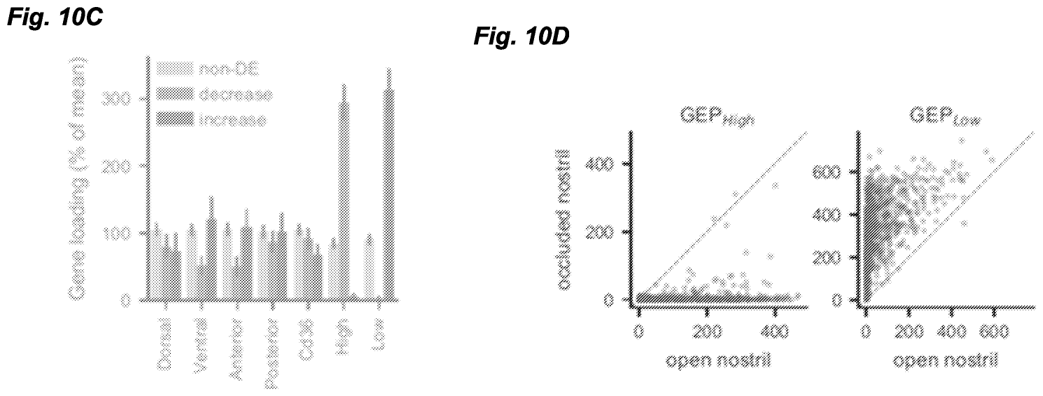

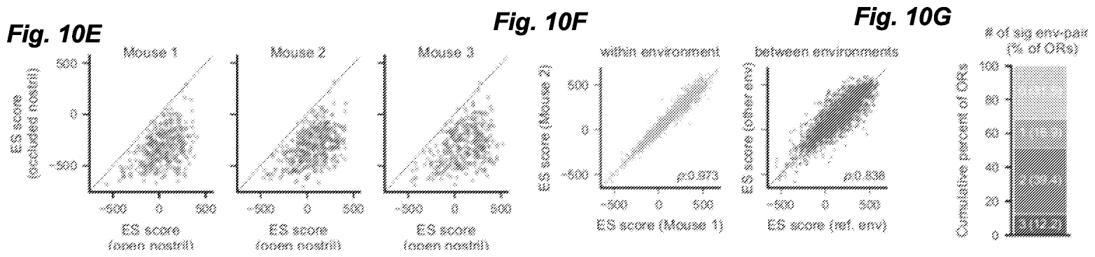

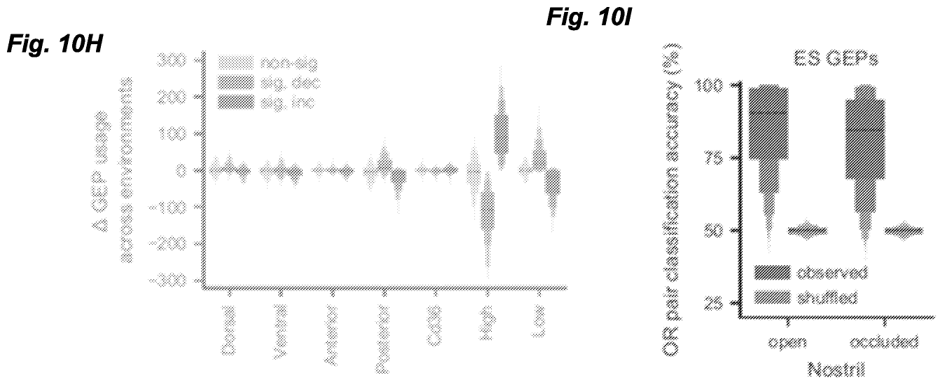

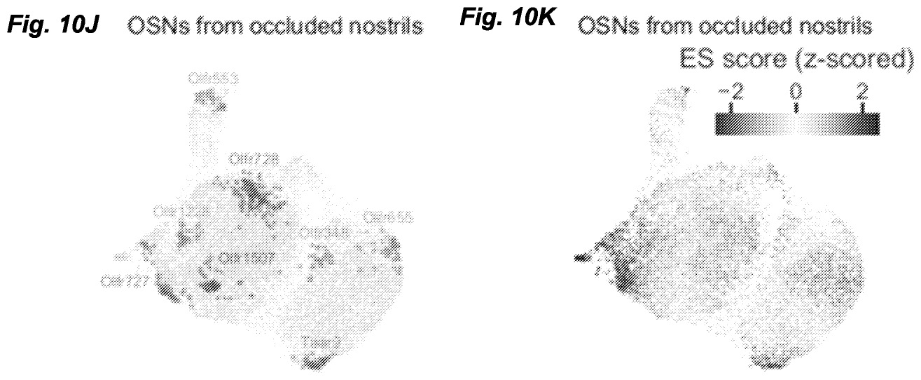

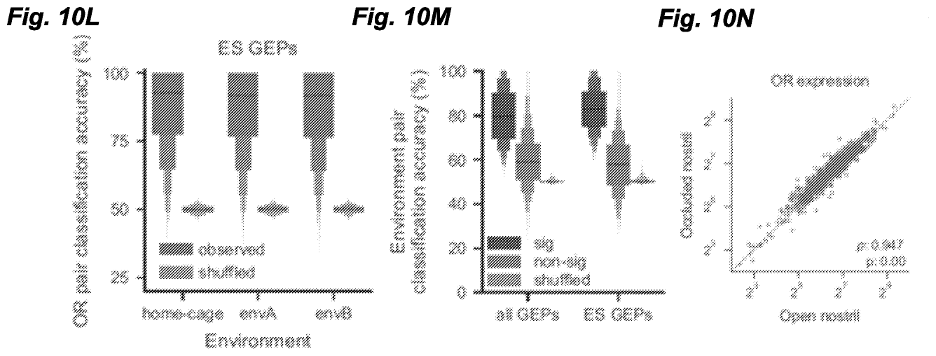

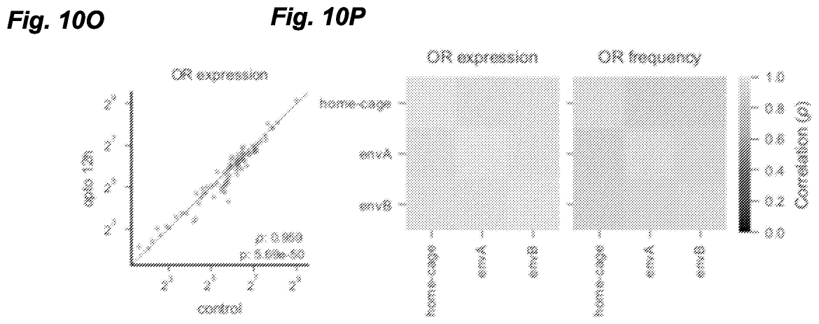

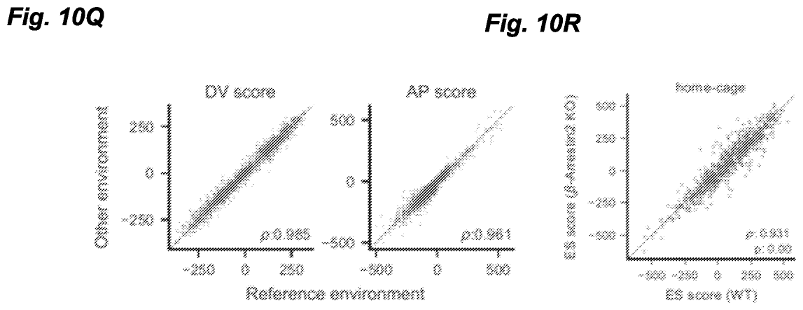

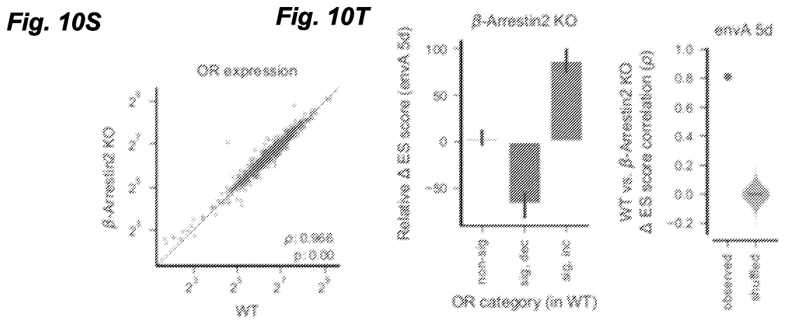

Consistent with the hypothesis that GEPHigh and GEPLow genes, and thus ES scores, are sensitive to the chronic activity state of each OSN, artificially lowering (via nares occlusion for a month) or raising (via pulsed optogenetic stimulation for 12 hours) OSN activity systematically decreased or increased ES scores, respectively (FIGS. 3A-3H, 10A-10E). To address whether OSN ES scores are sensitive to chronic activity levels under more physiological conditions. OSN gene expression was compared between mice housed in either a regular home cage or in two distinct naturalistic odor environments (FIG. 3I). ES scores shifted significantly and bidirectionally in ˜45% of OSN subtypes across environments, consistent with specific odor environments differentially ligating or disengaging distinct ORs (FIGS. 3J-3N, 10F-10H).

Three additional lines of evidence argue that ES scores reflect specific interactions between each OR and each environment. First, occlusion-dependent changes in ES scores for each OR-defined OSN subtype were negatively correlated with the ES score in the open nostril (FIG. 3O). ES scores after occlusion were sufficiently diverse to enable accurate predictions of OR identity; this diversity might reflect a variety of OR-specific spontaneous activity patterns (FIGS. 3P, 10I-10K). Second, after switching environments. ES scores for each OSN subtype were consistent across mice and accurately predicted the specific environment in which each mouse was housed (FIGS. 3Q, 10L-10M). Finally. OSN subtypes whose ES scores increased across odor environments tended to have relatively low initial ES scores, and conversely those that fell tended to have relatively high initial ES scores (FIG. 3R).

Several alternative explanations were ruled out for environment-dependent changes in OSN ES scores. Odor-dependent modulation in OR expression levels or β-Arresting-mediated OR endocytosis could, in principle, influence ES scores (Ibarra-Soria et al., 2017; Mashukova et al., 2006; von der Weid et al., 2015). However, across all three of the experimental manipulations. OR gene expression and the usage of identity-related GEPs remained constant (FIGS. 3C, 10C, 10H, 10N-10Q). Furthermore. β-Arresting knockout mice exhibited largely normal ES scores that adapted to a new environment (FIGS. 10R-10T). Taken together, these results reveal that specific interactions between each OR and the environment differentially drive activity in each OSN, which in turn specifies the particular level at which GEPHigh and GEPLow genes are expressed. The results also demonstrate that, under physiological conditions and in realistic odor environments, odors and air chronically engage the entire OSN array.

### Structured Correlations and Flexibility in Genes Influencing Odor-Activity Coupling

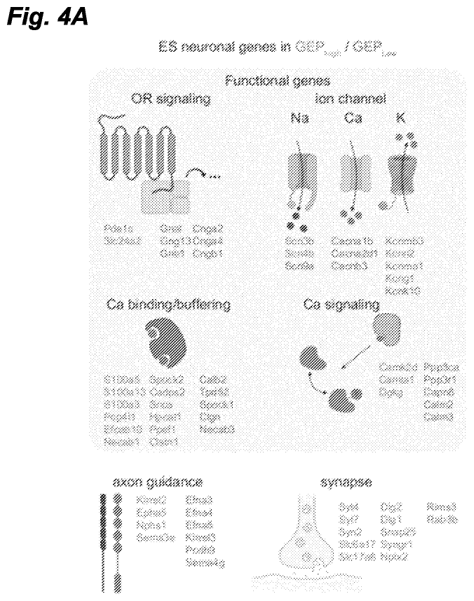

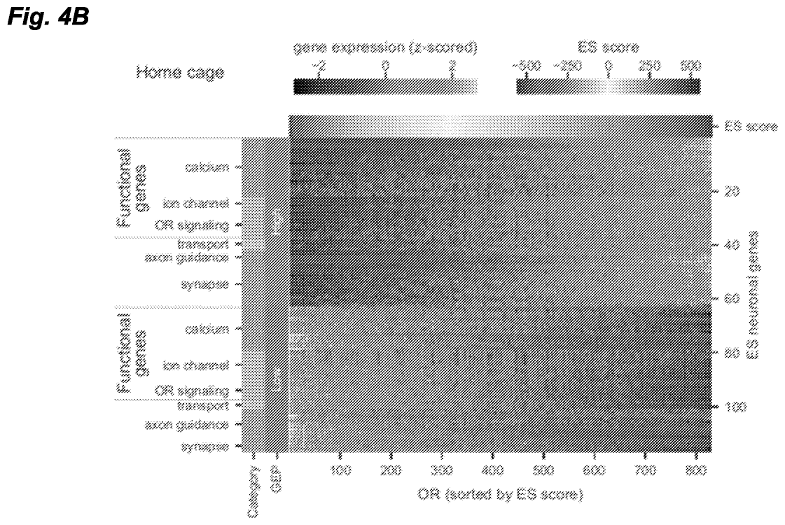

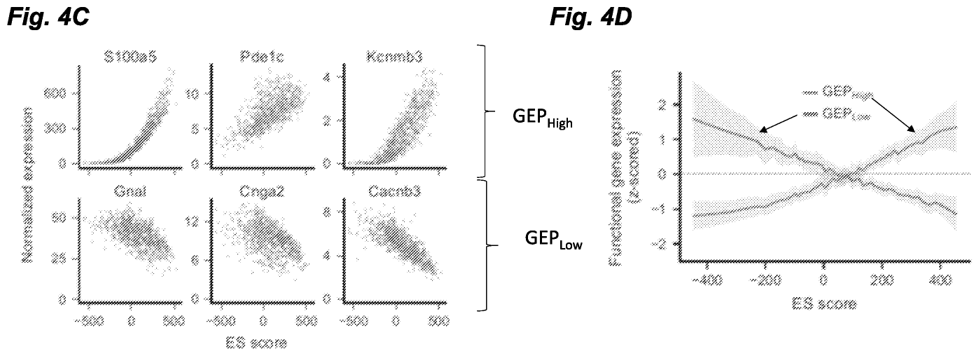

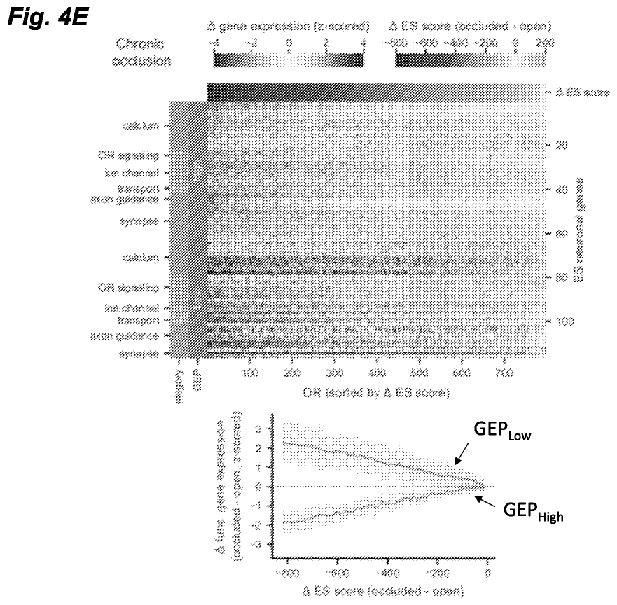

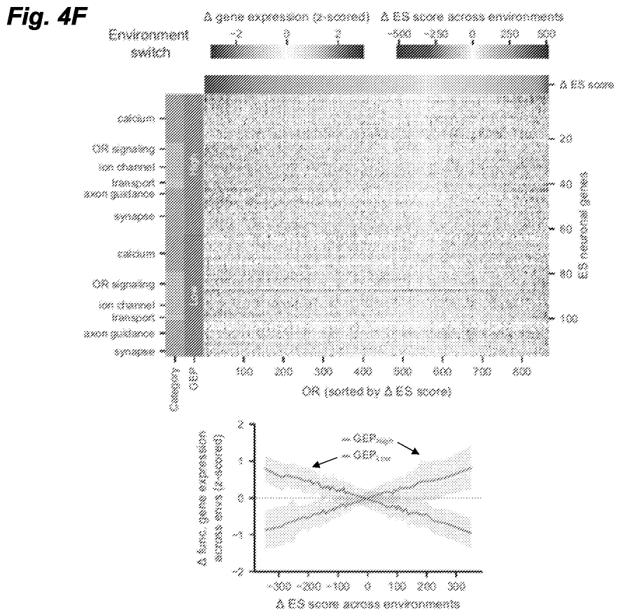

What functions might environment-dependent gene expression confer upon mature OSNs? Inspection revealed that GEPHigh and GEPLow include 73 genes with known or likely roles in shaping OSN sensory responses (e.g., calcium homeostasis. OR signaling, and intrinsic excitability), and an additional −40 genes putatively involved in axon guidance or synaptic transmission (FIG. 4A). The expression of these genes smoothly co-varied across OSN subtypes and exhibited structured correlations and anticorrelations with other GEPHigh and GEPLow genes (FIGS. 4B, 11A-11C). Like the ES score itself, the expression of “functional” genes (here and throughout, defined as the set of 73 GEPHigh and GEPLow genes relevant to sensory responses) both distinguished all OSNs and systematically fell after naris occlusion (FIGS. 11D-11F). The observed pattern of functional gene expression in each OSN indicated a role in compensating for ongoing activity: the expression of genes that likely attenuate odor responses was higher in OSNs with high ES scores than in those with low ES scores, whereas genes that likely amplify responses showed the opposite trend (FIGS. 4C-4D). Furthermore, persistently changing OSN activity by chronic occlusion, optogenetic stimulation, and switching environments caused functional gene expression to change bidirectionally in an OR-dependent manner (FIGS. 4E-4F and 11G-11K).

### Act-Seq Quantifies Odor-Evoked OSN Activation

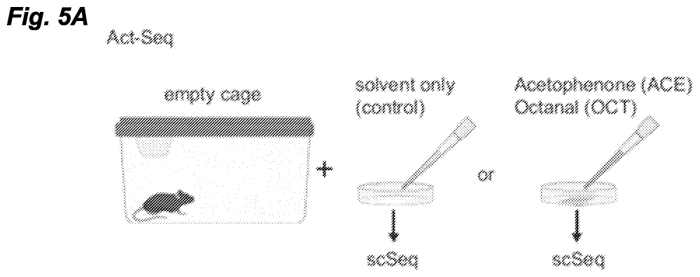

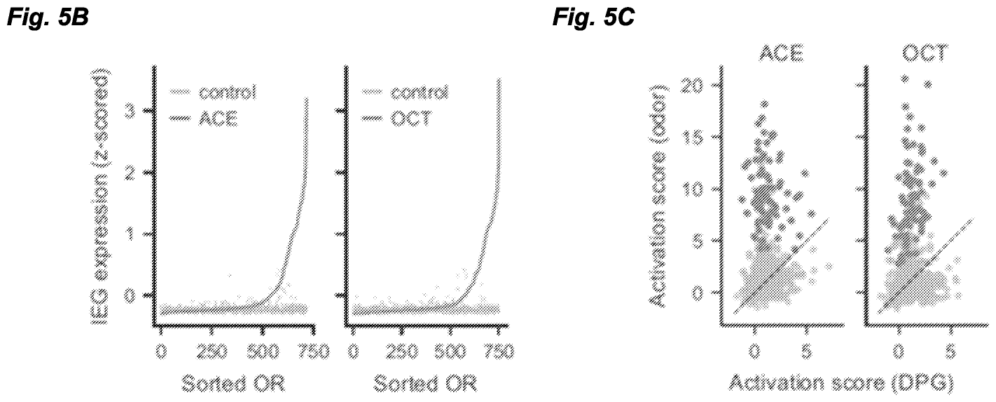

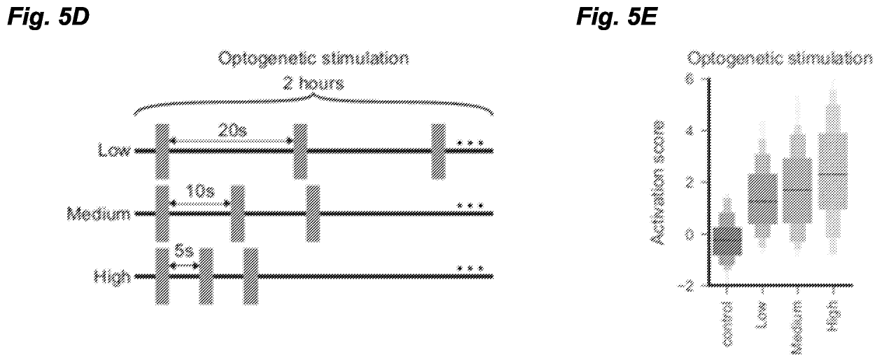

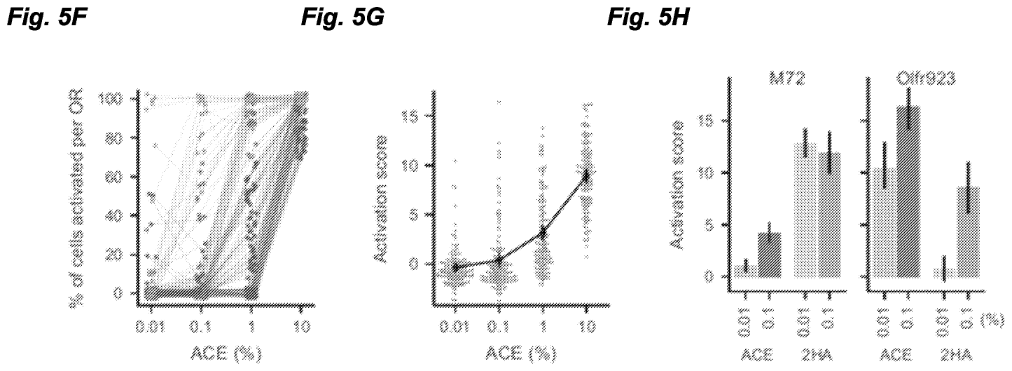

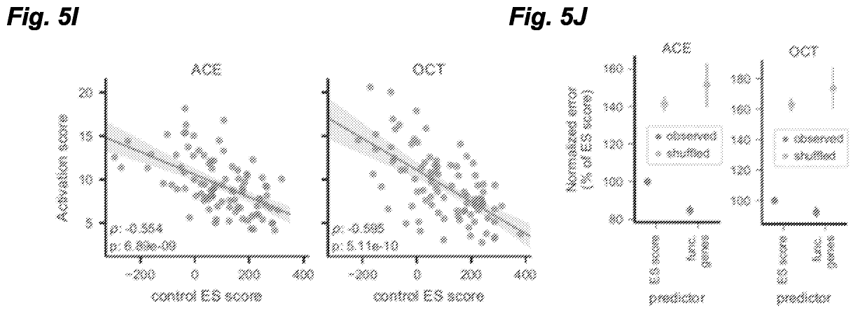

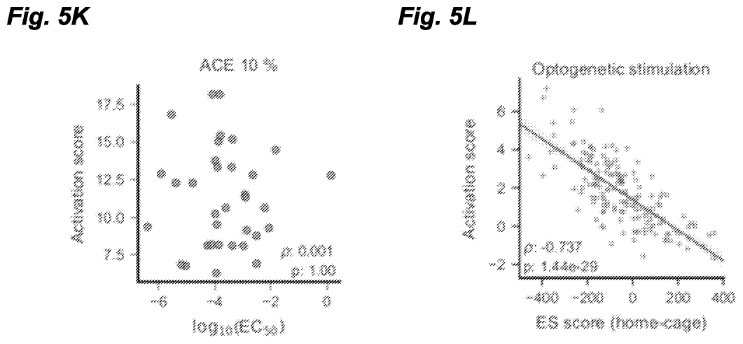

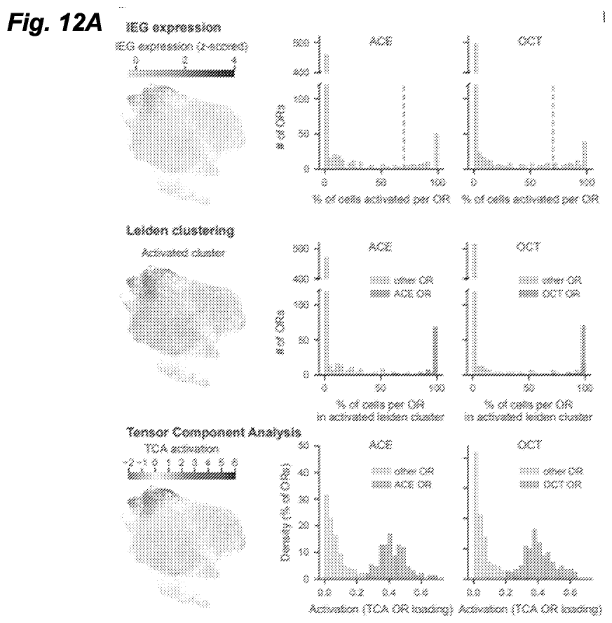

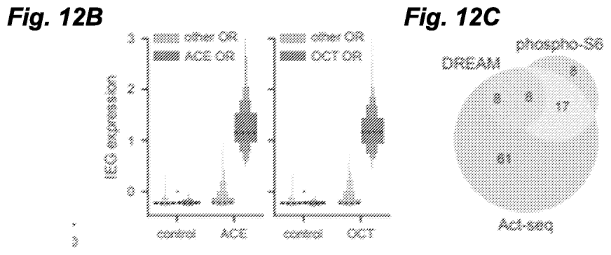

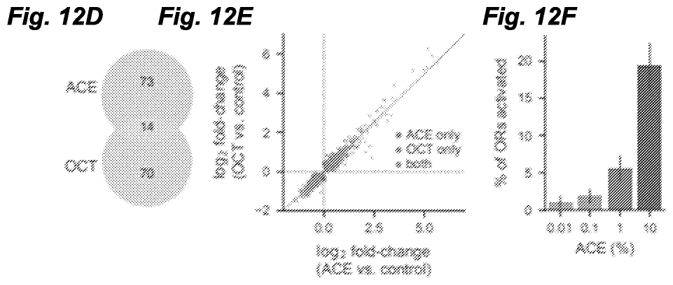

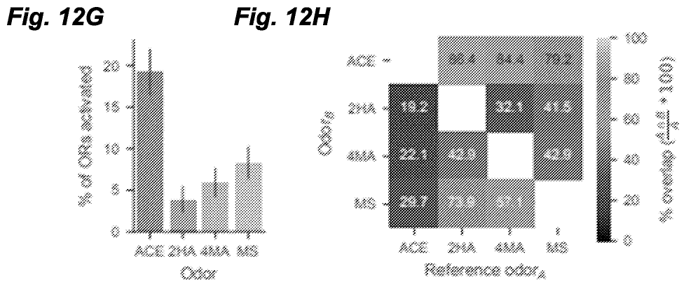

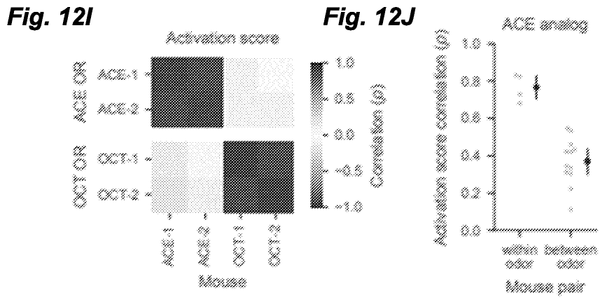

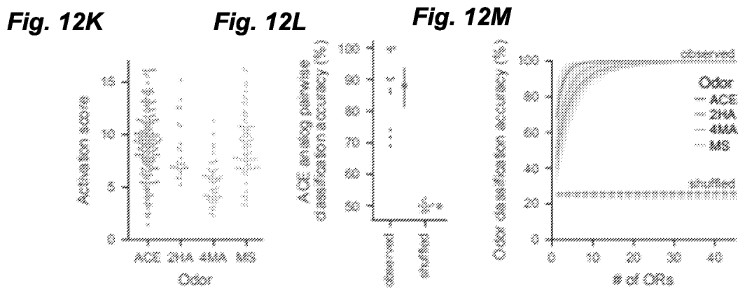

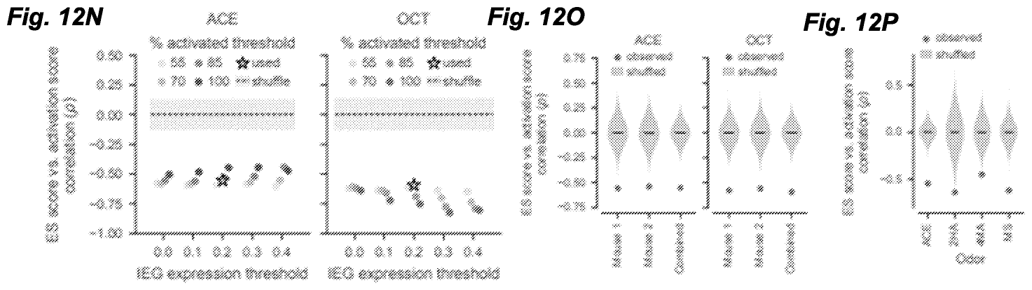

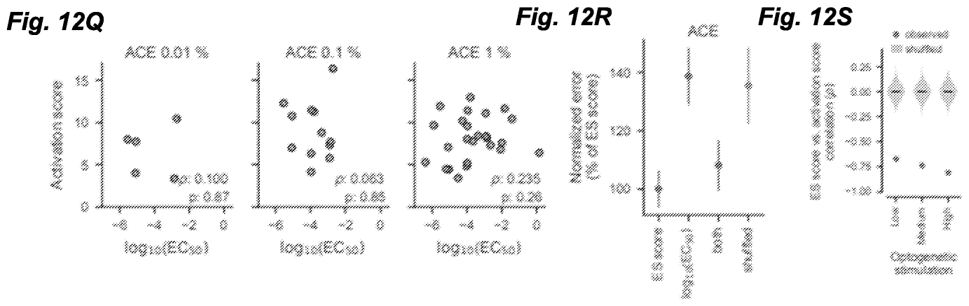

To test the possibility that the continuous variation in functional gene expression uniquely adapts the odor responses of each OR-defined OSN subtype to each environment, the olfactory epithelium was subjected to Act-Seq, a scSeq variant in which neural activity is read out as a rapid change in gene expression (Wu et al., 2017). Act-Seq reliably identified a subset of OSNs (and therefore ORs) that acutely responded after a two-hour exposure to the volatile odorants acetophenone and octanal, as assessed by immediate early gene (IEG) expression (with similar results obtained using unsupervised analysis of odor-evoked transcriptional changes: FIGS. 5A-5B and 12A, see Methods). Act-Seq identified a subset of ORs (−12-13% of total ORs) activated by either acetophenone or octanal; these included most known responsive ORs for each odor, as well as many new acetophenone and octanol receptors (FIGS. 12B-12D) (Bozza et al., 2002; Jiang et al., 2015; Repicky and Luctje. 2009; von der Weid et al., 2015).

Acetophenone and octanal elicited similar acute transcriptional changes across many genes (FIG. 12E). Therefore an analog measure was developed of odor response (referred to herein as an activation score) to quantitatively summarize information from the ˜500 genes (including IEGs) that were reliably and rapidly changed by odor exposure (and which lacked strong ES score correlations. FIG. 5C, see Methods). To test whether activation scores capture meaningful variation in activity OSNs were subjected to optogenetic stimulation, which revealed a tonic relationship between stimulation frequency and activation scores (FIG. 5D-5E). Similarly, increasing concentrations of acetophenone tonically increased both the number of ORs activated by acetophenone and the activation scores of acetophenone-responsive OSNs (FIGS. 5F-5G, 12F).

To test whether activation scores also capture differences in binding affinities between different ligands for the same receptor. 2-hydroxyacetophenone (2-HA) was used which, relative to acetophenone, binds more strongly to the M72 receptor (Arncodo et al., 2018; Zhang et al., 2012). Act-Seq revealed that 2-HA indeed elicits higher activation scores than acetophenone in OSNs expressing M72 (FIG. 5H). Conversely, activation scores for OSNs expressing Olfr923, a high affinity acetophenone receptor, were higher for acetophenone than 2-HA (Hu et al., 2020; Jiang et al., 2015). Furthermore. 2-HA, acetophenone, and two related odors each elicited a broad range of OR- and odor-specific activation scores that were stereotyped across mice and accurately predicted the specific odor each mouse experienced (FIGS. 12G-12M). Taken together, these results demonstrate that Act-Seq can identify the OSN subtypes (and thus the ORs) that can bind to and respond to any odorant, and that the activation score affords a quantitative transcriptional measure of the degree to which a given OSN has responded to an odor in vivo.

### OSN Transcriptomes Predict Acute Odor Responses

Consistent with the hypothesis that the transcriptome of each OSN shapes its acute odor responses. ES scores were negatively correlated with odor activation: the higher the ES score for a given acetophenone-responsive OSN subtype (identified across experiments via its OR), the lower its acute, acetophenone-evoked response (FIGS. 51, 12N-12O). This negative correlation was also observed in OSNs responding to octanal and to the three acetophenone-related odors (FIGS. 51, 12O-12P). The 73 functional genes were as effective as the ES score at predicting acute OSN odor responses, but the in vitro EC50 of each acetophenone OR was unable to support in vivo odor response predictions (FIGS. 5J-5K, 12Q-12R) (Jiang et al., 2015; Saito et al., 2009). Interestingly. OSN activation scores following two hours of acute optogenetic stimulation were also negatively correlated with control ES scores, indicating that the ability of light to transcriptionally activate each OSN also depends on the expression levels of functional genes (FIGS. 5L, 12S).

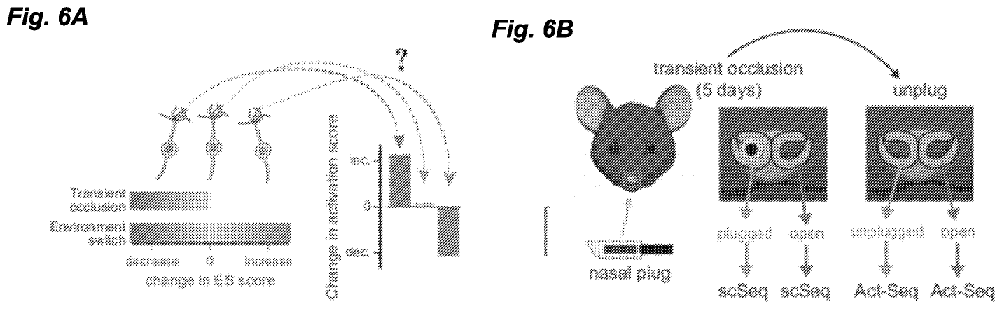

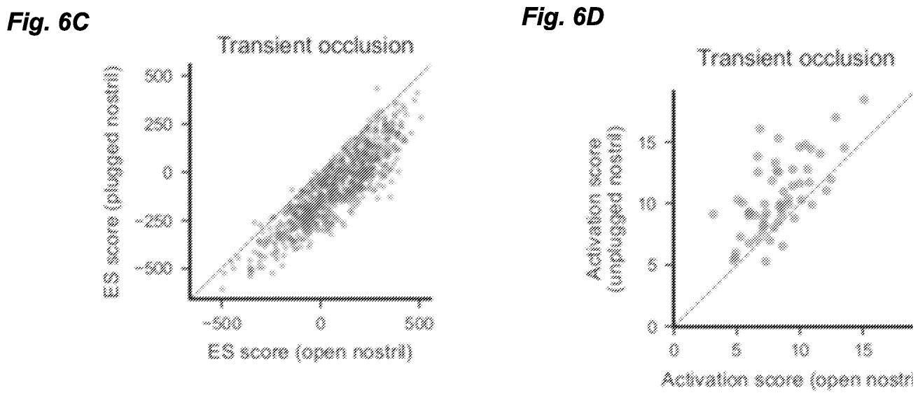

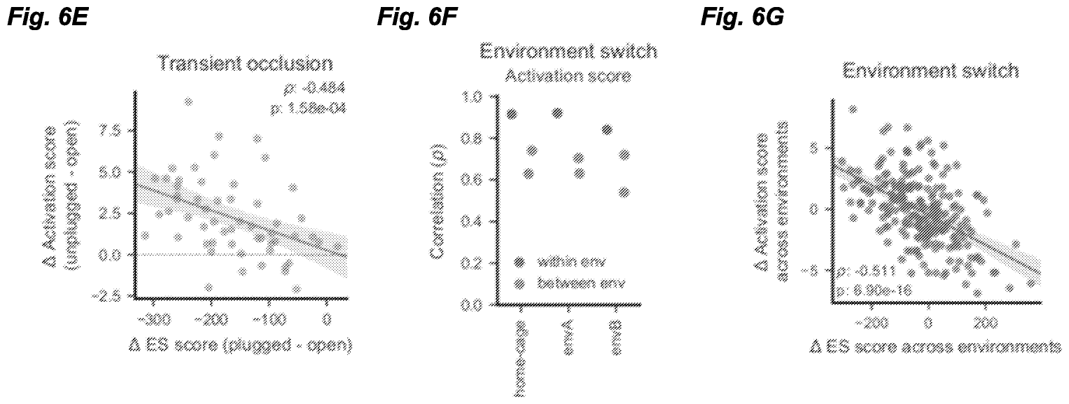

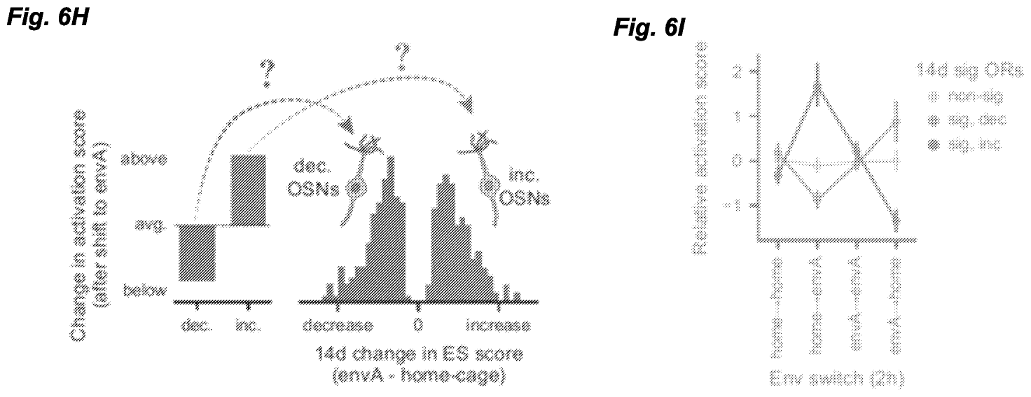

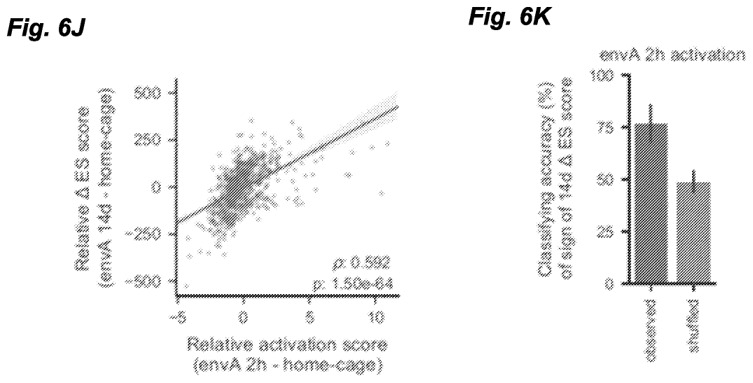

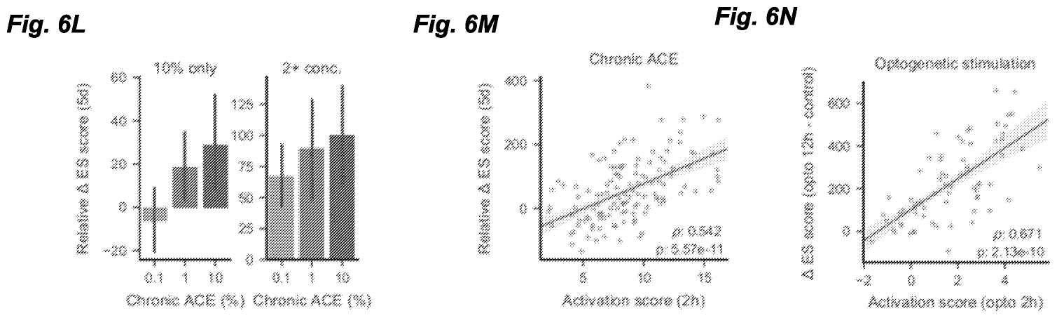

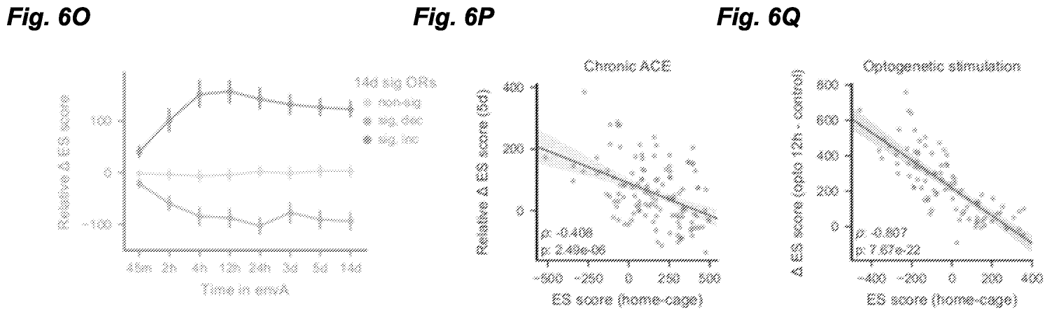

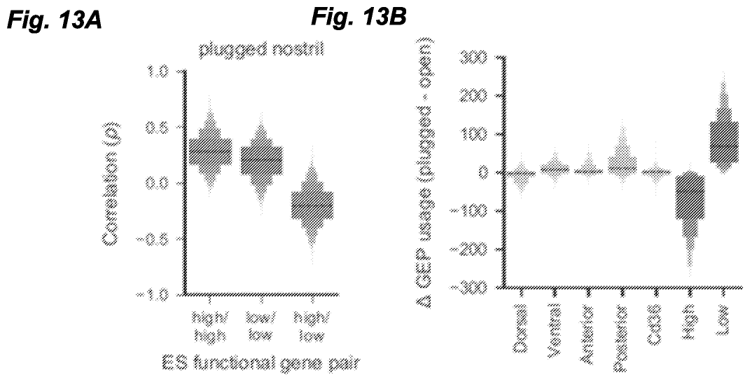

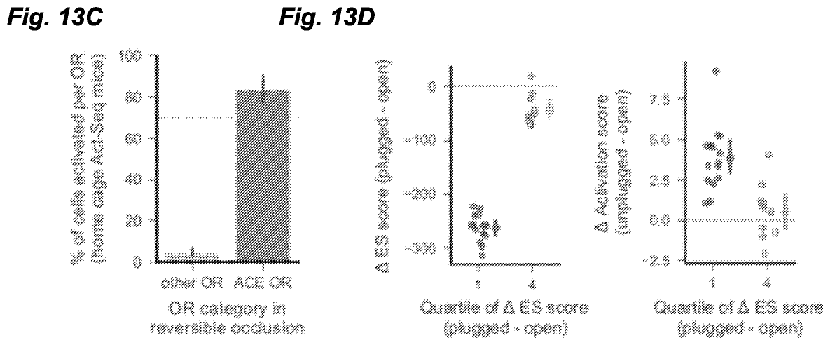

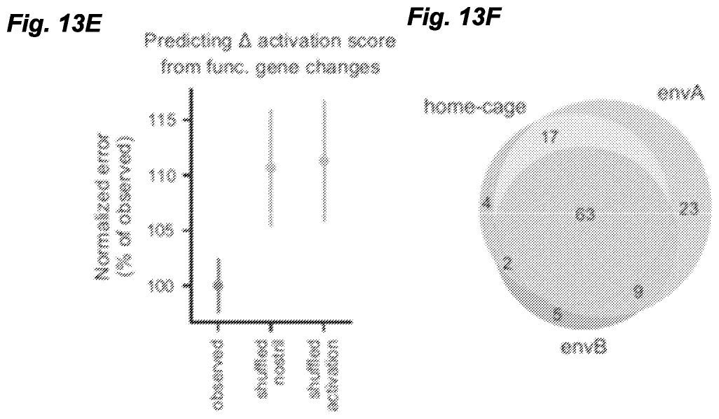

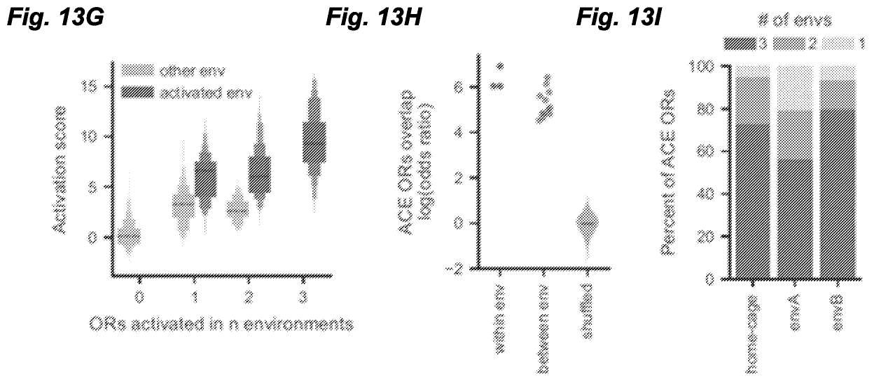

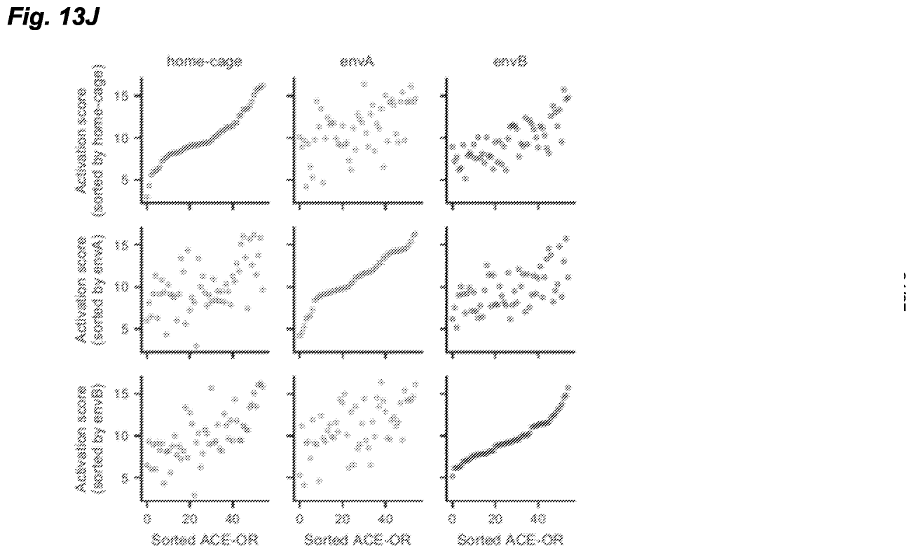

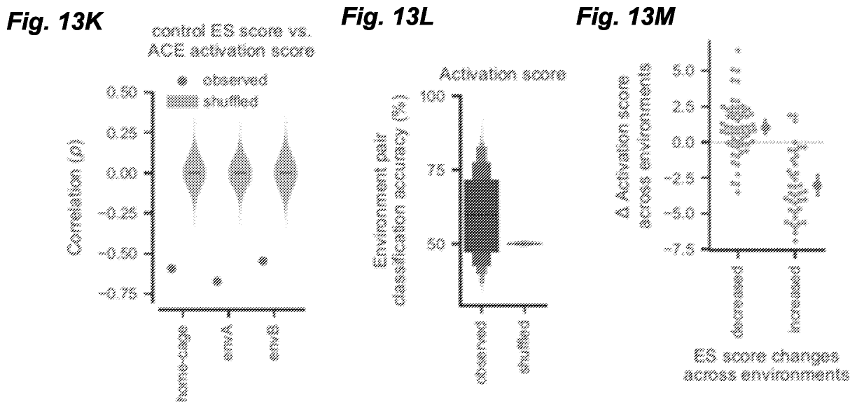

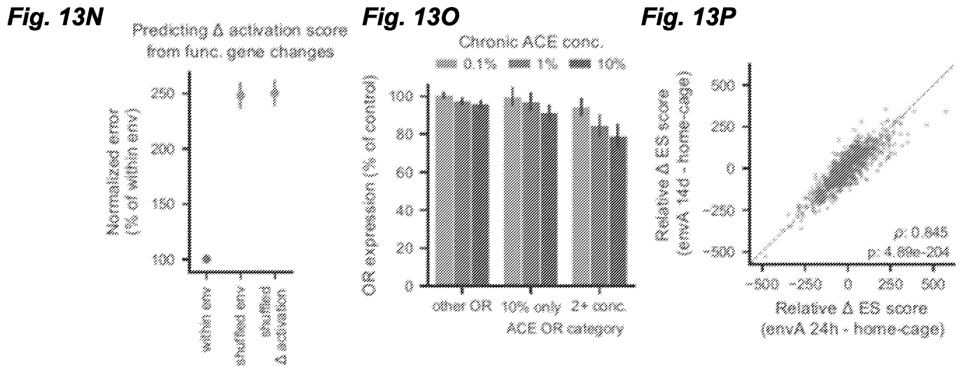

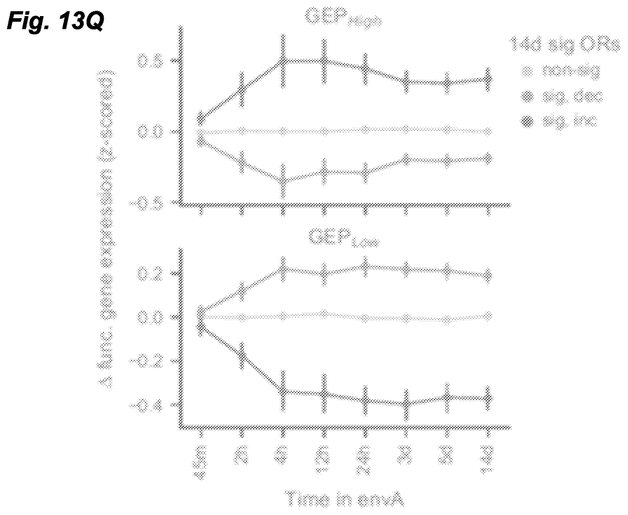

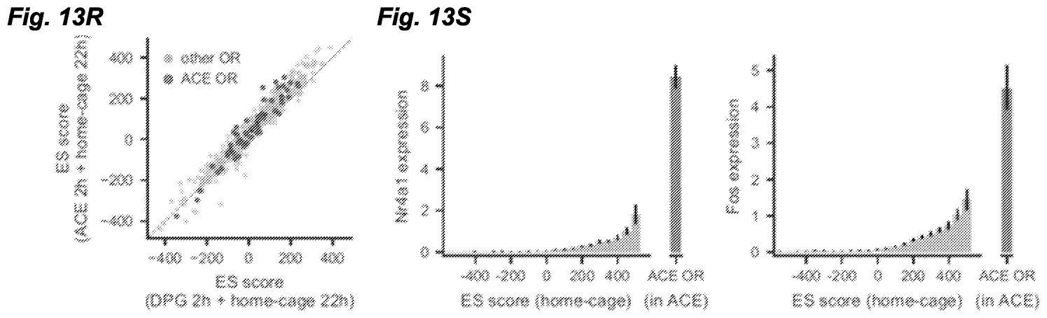

If functional gene expression indeed plays a causal role in determining odor responses, then experimentally raising or lowering ES scores should predictably change acute odor responses (FIG. 6A). This hypothesis was tested by lowering OSN activity via transient unilateral naris occlusion using removable plugs, and then performing Act-Seq after acute odor exposure (FIG. 6B). Transient occlusion reduced ES scores in an OR-specific manner but did not change the correlation structure between functional genes (FIGS. 6C, 13A-13B). Similar sets of ORs were activated by acetophenone in control conditions and after occlusion, but acetophenone receptor-expressing OSNs derived from occluded nostrils exhibited higher activation scores (FIGS. 6D, 13C). Critically, the occlusion-dependent changes in ES scores and functional gene expression predicted the occlusion-dependent changes in odor-evoked activation (FIGS. 6E, 13D-13E).

Similarly, switching mice from the home cage into a new odor environment (which bidirectionally modulates ES scores (FIGS. 3I-3N)), altered acetophenone-driven OSN activation scores in an environment-dependent manner (FIGS. 6F, 13F-13I); these scores were sufficiently different across environments and reliable across mice that they could be used to identify the environment in which a given mouse was housed (FIGS. 6F, 13J-13L). Activation scores for acetophenone-responsive OSN subtypes were also negatively correlated with ES scores within a given environment, and the bidirectional changes in ES scores or functional gene expression following environment shifts predicted the corresponding change in the acetophenone response for each OSN subtype (FIGS. 6G, 13M-13N). Together, these results demonstrate that odor-evoked activation is not determined solely by the affinities of odors for their receptors, but rather is flexibly tuned in each OSN by functional genes, which adapt OSNs in an OR-specific manner to the specific contents of each odor environment.

### Acute Odor Responses Predict Future Changes in Functional Gene Expression

It was hypothesized how different environments induce changes in OSN transcriptomes, and that acute activity triggered by new odor environments sculpts long term functional gene expression (FIG. 6H). For OSN subtypes whose ES scores changed significantly across environments, switching between different environments recruited changes in OSN activation scores, while activation was negligible in mice moved between similar environments, demonstrating that OSN activation captures salient changes in the odor environment (FIG. 6I). Furthermore. OSN-specific activation scores measured two hours after an environment shift predicted OSN ES score changes measured after two weeks (FIGS. 6J-6K). Similarly. OSN activation scores observed after acute two-hour exposure to acetophenone or optogenetic stimulation predicted future ES score changes after chronic acetophenone exposure or prolonged optogenetic stimulation, respectively (FIGS. 6L-6N, 13O). These data demonstrate that ES scores adjust proportionally to acute perturbations in activity. Interestingly, much of the long-term change in ES scores and functional genes was apparent four hours after switching environments, indicating that ES scores integrate activity on timescales of hours (FIGS. 6O, 13P-13Q). In contrast, transient two-hour exposure of mice to acetophenone did not change ES scores after 24 hours (FIG. 13R), indicating that the ongoing presence of odors is required for persistent changes in functional gene expression.

It was observed that the long-term ES score changes observed after chronic acetophenone exposure could be predicted by the baseline ES score of each acetophenone-responsive OSN subtype in the home cage: a similar relationship was observed following optogenetic stimulation (FIGS. 6P-6Q). Given that baseline ES scores also determine acute responses after odor or light stimulation (FIGS. 5I, 5L), these results are consistent with odor-evoked activity and ES scores being linked together in a closed loop. It was noted that OSNs with high ES scores exhibited some residual IEG expression in the home cage (FIG. 13S). This finding indicates that transcription-based adaptation in OSNs compensates for but does not fully eliminate activity driven by background odors, indicating that under normal circumstances OSNs are continuously re-adapting to their environment.

### Odor Environments Influence Odor Codes Accessed by the Brain

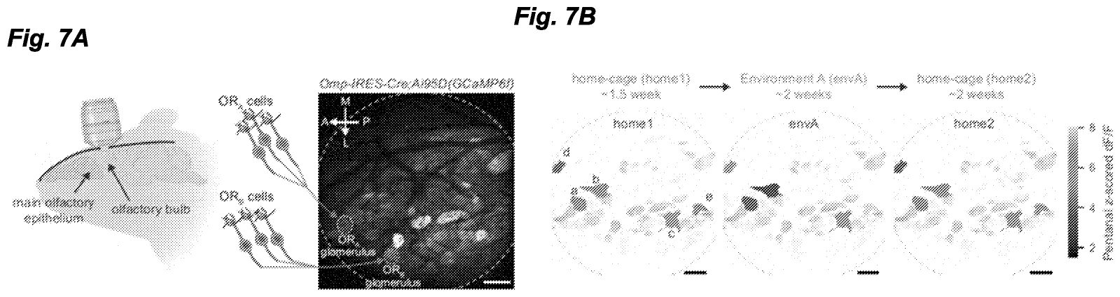

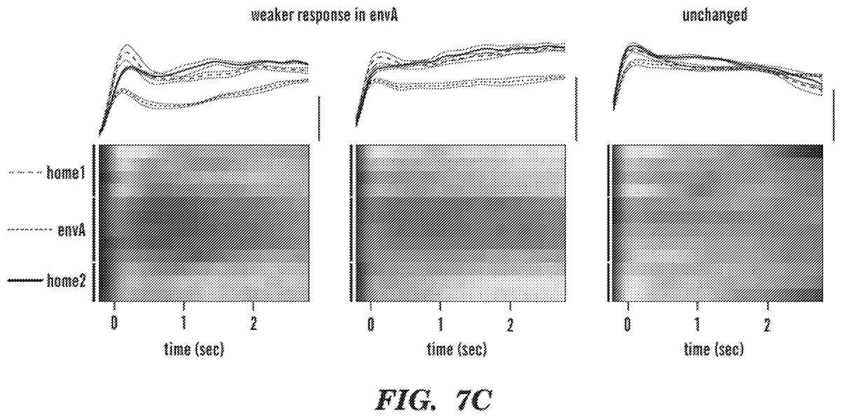

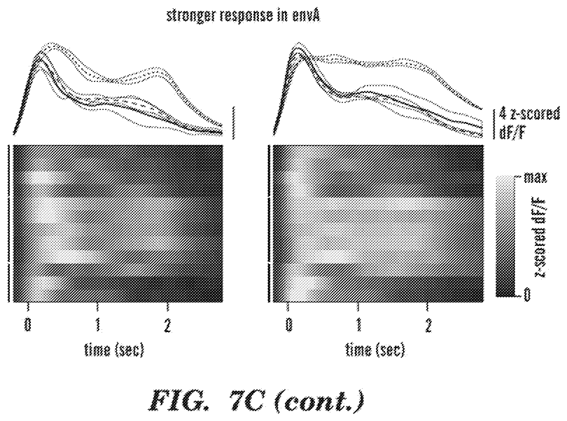

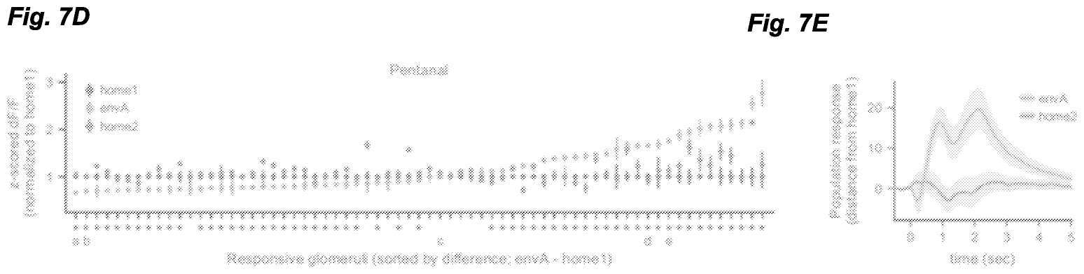

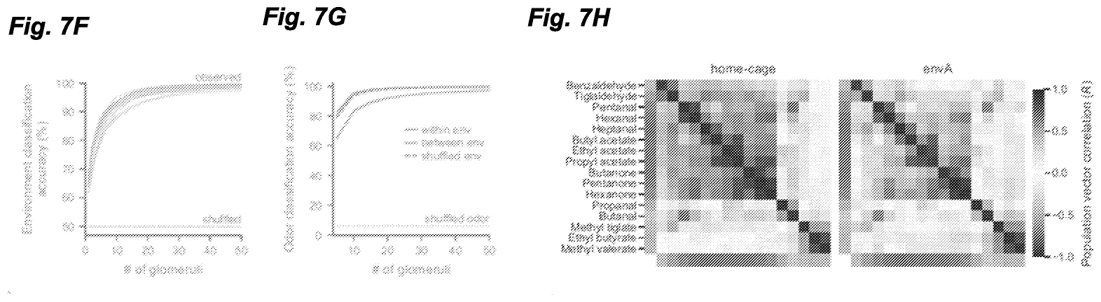

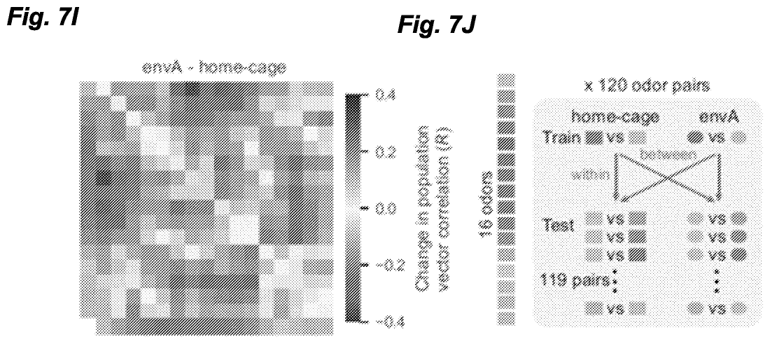

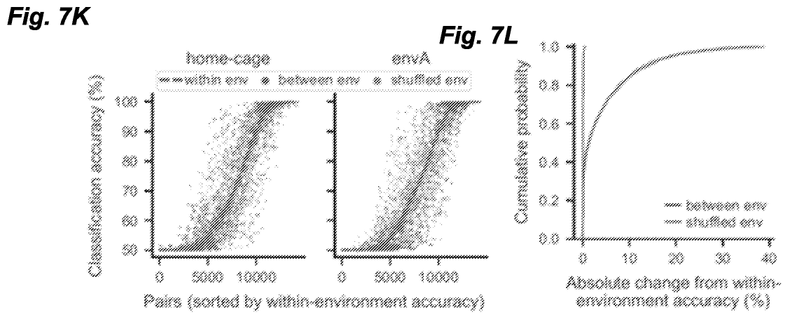

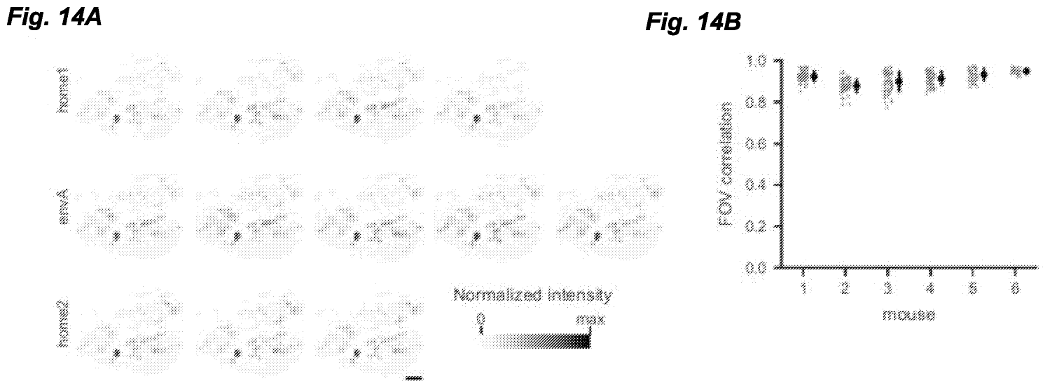

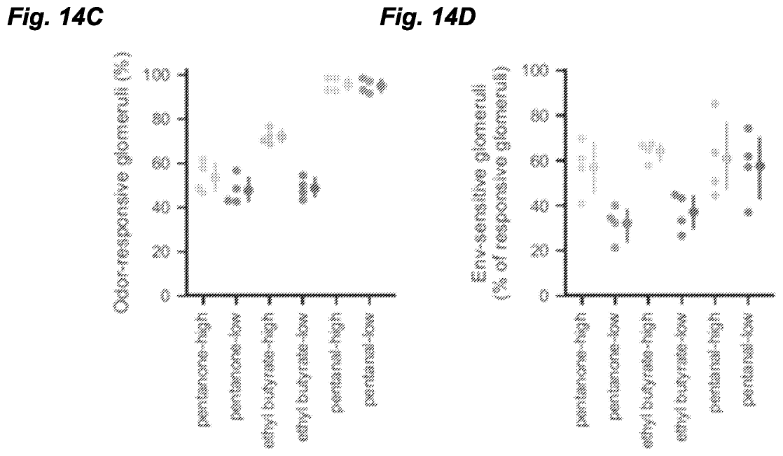

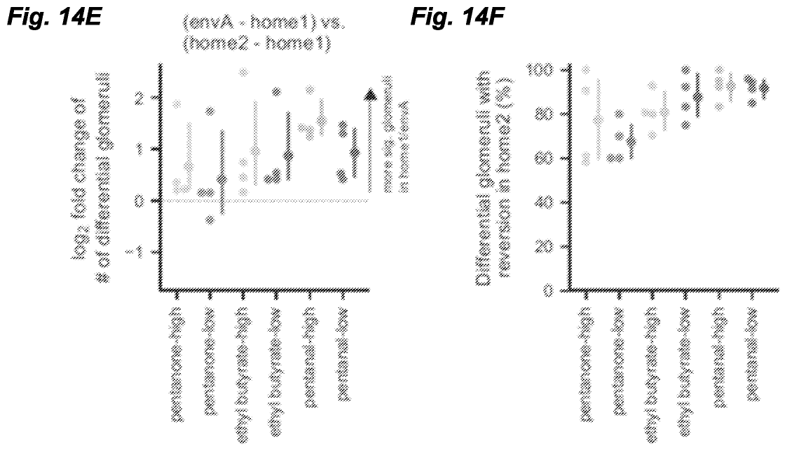

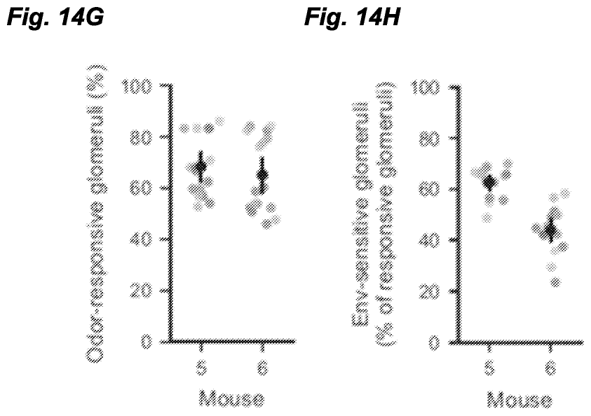

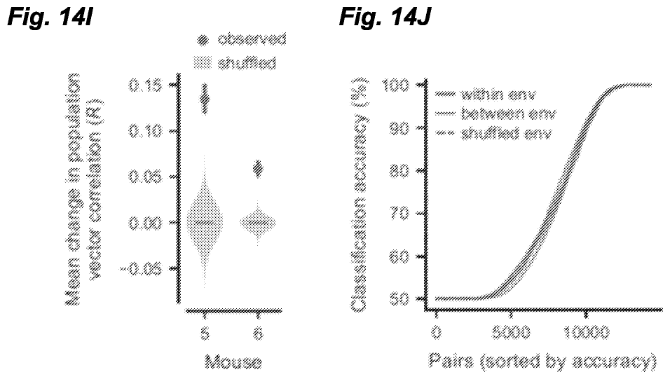

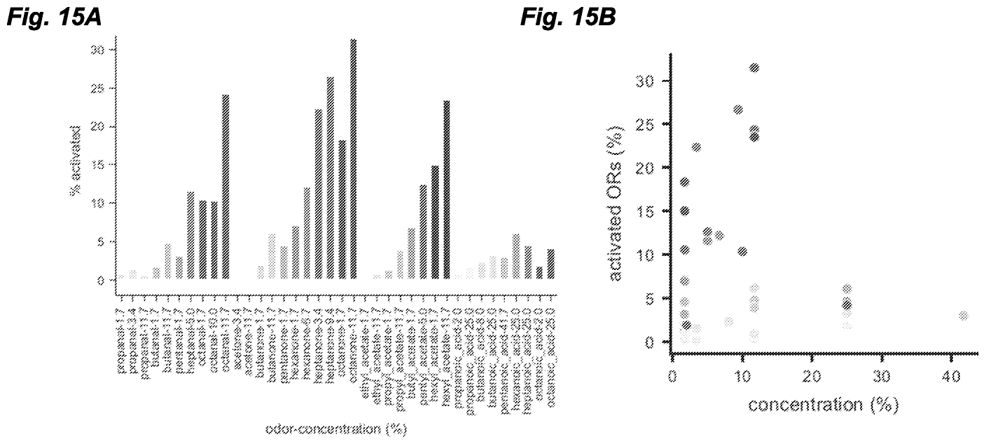

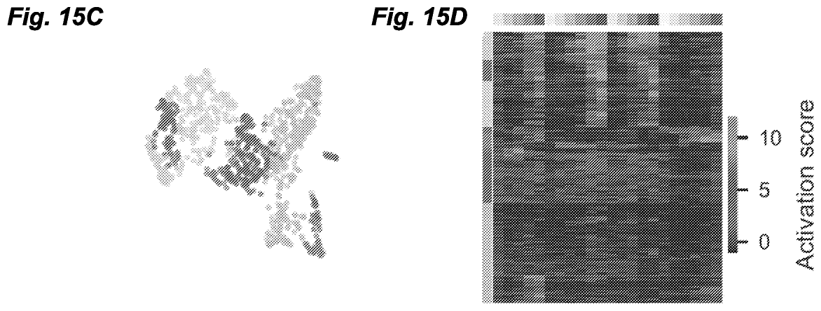

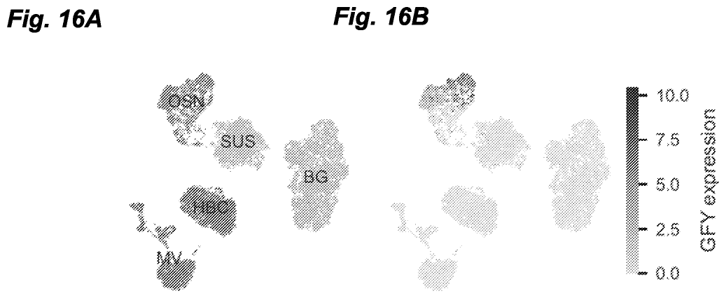

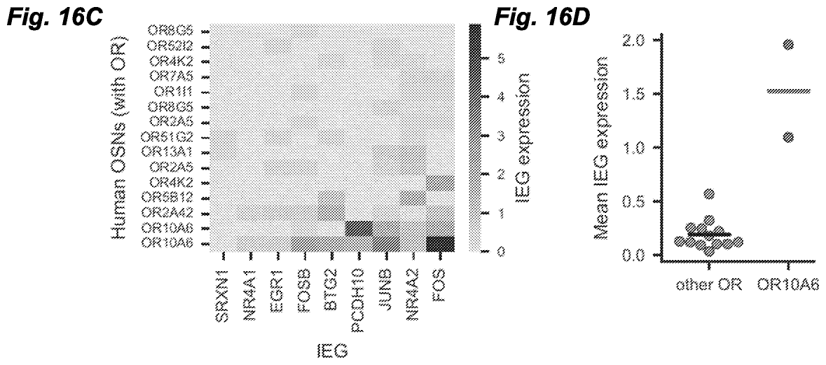

To test whether the ambient environment also shapes odor codes in the brain, we performed presynaptic functional imaging of OSN axons in the olfactory bulb (FIG. 7A). OSN axons expressing the same OR converge on one or more insular structures known as glomeruli; imaging pre-synaptic OSN neural activity in olfactory bulb glomeruli can therefore characterize odor responses across different OSN subtypes. Responses were imaged to a panel of purified odors before, during, and after a two-week period in which mice were housed in one of the alternative odor environments described above (FIGS. 7B, 14A-14B). Response amplitudes to the same odor stimulus changed bidirectionally in ˜50% of glomeruli after mice were switched from the standard home cage into a new environment, consistent with prior work exploring the influence of a single odor on subsequent responses (Kass et al., 2016); these environment-dependent changes were consistent across both mice and odorants, and reverted when mice were later returned to standard home cages (FIGS. 7C-7E, 14C-14F).

To determine how environmental history influences odor coding, responses were imaged to a panel of 16 odors that broadly activated dorsal glomeruli (FIG. 14G). A large fraction of glomeruli exhibited environment-specific odor response amplitudes, and linear decoders for each odor accurately distinguished between environments (FIGS. 7F and 14H). Odor identity could also be accurately predicted based upon glomerular responses within any given environment; however, classifiers trained in one environment performed worse across environments (FIG. 7G). Quantification of pairwise correlations in glomerular odor responses revealed that relationships for many odor pairs were modulated across environments, while close odor relationships were often preserved (FIGS. 7H-7I, 14I); similarly, classification analysis designed to quantify the relational structure in odor responses revealed diffuse changes in odor relationships across odor environments (FIG. 7J-7L, 14J). Taken together, these data demonstrate that changes in OSN transcriptomes evoked by complex naturalistic environments are paralleled by pervasive environment-dependent changes in both pre-synaptic glomerular odor responses and population-level odor codes in the brain.

### DISCUSSION

OSNs are thought to primarily communicate information about odor-OR interactions, which is used by the brain to facilitate odor perception (Buck. 1996; Cleland and Linster. 2005; de March et al., 2020; Firestein. 2001; Imai et al., 2010; Sullivan et al., 1995; Touhara. 2002). Here it is found that each of the ˜1000 OR-defined OSN subtypes in the mouse olfactory system adapt to the environment through a common mechanism: a transcriptional rheostat. This rheostat is composed of more than 70 genes relevant to OSN function, whose expression levels vary continuously across OSNs in an OR-dependent manner and adapt dynamically as mice traverse distinct environments. The specific position adopted by this rheostat (i.e., the OR-specific pattern of functional gene expression) predicts the odor response amplitude of a given OSN: furthermore, acute odor responses triggered by novel odor environments predict future patterns of functional gene expression (i.e., the setting of the rheostat).

These observations indicate a closed regulatory loop in which analog changes in environment-driven activity yield proportional transcriptional changes that predictably influence future neural responses to odors. Thus, rather than faithfully reporting the extent of odor-OR interactions, the peripheral olfactory system uses gene expression to instantiate expectation, thereby building odor codes that are personalized by each animal's experience. While fast, post-translational mechanisms for sensory adaptation have been observed across modalities—and many circuit-level mechanisms have been characterized that adapt central sensory responses—the data reveal a systematic, large-scale adaptive transcriptional program that operates at the level of sensory neurons themselves (Benda. 2021; Burns and Baylor. 2001; Fettiplace and Ricci. 2003; Kadohisa and Wilson. 2006; Martelli and Storace. 2021; Wark et al., 2007; Zufall and Leinders-Zufall, 2000).

The data demonstrate that the OR repertoire is densely activated by airflow and odors in our environments, indicating that every OSN must contend with some degree of chronic activation. Because experience bidirectionally alters ES gene expression on timescales of hours, transcription-mediated adaptation helps to center OSNs in their dynamic range as animals traverse different odor contexts, or as odor environments evolve during circadian cycles. As such, the adaptive mechanism identified here serves a distinct purpose from those that rapidly truncate responses to odor filaments (and which support e.g., odor-guided navigation), and from central habituation mechanisms that sparsen odor representations on the minutes-long timescale but cannot restore information that is lost when OSNs operate outside their dynamic range (Kadohisa and Wilson, 2006; Kostal et al., 2008; Lecoq et al., 2009; Martelli and Storace. 2021; Moore. 1994; Nagel and Wilson, 2011; Wilson, 2009).

Transcription factors and axon guidance genes appear insufficient to effectively distinguish OSN transcriptomes or predict OR expression. Although known identity markers have been thought to only assign OSNs to broad dorsal or ventral domains (Bozza et al., 2009; Kobayakawa et al., 2007; Tan and Xie, 2018), the dorsal, ventral, anterior and posterior GEPs are sufficiently diverse and organized to support OR predictions (FIGS. 9F-9H, 10Q). These observations show a fine gradation in OSN cell identities organized across the two main anatomical axes (Bozza et al., 2009; Miyamichi et al., 2005; Pacifico et al., 2012). The usages of these identity-related GEPs and OR expression levels remained largely constant across all our experimental manipulations, indicating that functional genes are uniquely sensitive to the environment. Although the mechanisms that render ES genes sensitive to neural activity—and which coordinate expression levels across ES genes-remain to be determined, mature OSNs express several transcription factors (including Ebf1 and Lhx2) that in principle could be sensitive to activity: the prominent role of heterochromatin in regulating OR expression indicates that epigenetic mechanisms involving chromatin or DNA modifications might also coordinate ES gene expression (Monahan and Lomvardas. 2015; Monahan et al., 2017).

There is a close conceptual relationship between the bidirectional sensory adaptation we describe here and firing rate homeostasis (FRH), the process through which neurons in a network maintain stable firing rates by adjusting both synaptic weights and intrinsic neural properties (Davis. 2006, 2013; Marder. 2011; Marder and Goaillard. 2006; Turrigiano, 2011; Turrigiano. 1999; Turrigiano. 2008, 2017). Invertebrate neurons of a given type with identical firing properties can exhibit significant variation in ion channel expression, demonstrating that there are many transcriptional means to achieving the same functional end (Goldman et al., 2001; Marder. 2011; O'Leary et al. 2013; Schulz et al., 2007). In contrast, the experiments in OSNs identify a deterministic and proportional interdependence between environment-dependent activity and functional gene expression patterns. It has been proposed that structured correlations in the expression of functional genes can be used to define different cell types (O'Leary et al., 2013; Schulz et al., 2007); OSNs, whose functional genes exhibit near-perfect expression correlations along a continuum of activity levels, clearly meet this operational definition.

scSeq analysis reveals a startling diversity of transcriptomes associated with neurons of a single putative type (Kim et al., 2019; Li et al., 2017; Tasic et al., 2018). Together, the findings—enabled by the ability to query gene expression and activity in ˜1000 different OSN subtypes across experiments—are consistent with a general model in which neurons systematically modulate their transcriptomes to continuously adapt to their inputs.

### EXPERIMENTAL MODEL AND SUBJECT DETAILS

### Mice

C57BL/6J. OMP-IRES-GEP. Ai95D (GCaMP6f) and β-arrestin2 knock out mice were obtained from Jackson Laboratory (stock number 000664, 006667, 028865, 011130). “OR-swap” mice (M72 (S50 locus) and S50 (M72 locus)) were maintained in the Bozza laboratory (and are available from Jackson Laboratory with stock numbers 006715, 006714) (Bozza et al., 2009). P2-IRES-GFP mice (P2 (P2 locus)) were obtained from the Lomvardas laboratory (and are available from Jackson Laboratory with stock number 006669) (Feinstein and Mombaerts, 2004). OMP-IRES-Cre mice were obtained from the Axel laboratory (Eggan et al., 2004). OMP-ChR2 (H134R)-Venus mice were maintained in the Bozza laboratory and obtained from the Rinberg laboratory (Li et al., 2014). Mice of either sex between 6-16 weeks-old were used for experiments. Mouse husbandry and experiments were performed following institutional and federal guidelines and were approved by Harvard Medical School's Institutional Animal Care and Use Committee.

### Method Details

### Chronic Naris Occlusion

To identify gene expression programs sensitive to ongoing activity from environmental odorants, 7 day old mice were anesthetized on ice, and one of the two nostrils was occluded by cautery, as previously described (Fischl et al., 2014). This age was chosen to minimize deficits in axon targeting that occur from manipulating activity levels at earlier post-natal timepoints (Ma et al., 2014). Unilateral naris occlusion was confirmed using a dissection microscope. After ˜1 month, mice were used for scSeq experiments. Cells isolated from occluded and open nostrils were processed and analyzed separately.

### Odor Exposure (Act-Seq)

Act-Seq (Wu et al., 2017) was performed following odor exposure to quantify odor-evoked responses in each OSN subtype (defined by their expressed OR) across the entire OR repertoire. Mice were habituated to a reversed light cycle for at least for 1 week before odor exposure, transferred to a new disposable cage with regular bedding/food and kept overnight in a satellite animal facility. On the experimental day, each mouse was first transferred to a new empty disposable cage during the dark cycle and habituated for 20 minutes. Empty cages were used to avoid any activation by odorants present within the environments in which the mice were housed. Odors were added to a piece of filter paper (100 μL of odorant) in a 35 mm petri dish and a cotton ball (200 μL of odorant) and placed in each cage. After 2 hours, mice were euthanized, and dissociation was performed as described below. Odors used in this study were as follows. Dipropylene glycol (DPG, control solvent), and 10% of acetophenone, octanal, 2-hydroxy acetophenone, 4-methyl acetophenone, and methyl salicylate. As a control, mice were exposed to DPG alone for 30 min. This control condition was used to account for any drift in transcriptomes during the overnight housing and habituation periods prior to odor exposure and designed to capture the transcriptome in the state it would have been in the odor-exposed animals prior to odor exposure. The 30 min DPG control was used for all Act-Seq experiments, except following transient naris occlusion (see below); in the Act-Seq experiments our results did not depend upon the specific control condition we used, which likely reflects the relative stability of the transcriptome for each OSN subtype. Act-Seq was also performed using lower concentrations of acetophenone (1.0, 0.1, 0.01%) and 2-hydroxy acetophenone (0.1%, 0.01%). All odor dilutions were made with DPG, and all odors and DPG were obtained from Sigma. To assess the persistence of any transcriptional changes observed as a result of odor exposure, a cohort of mice was exposed to acetophenone or DPG for 2 hours, transferred to new clean regular home cages, and subjected to scSeq after an additional 22 hours.

### Transient Naris Occlusion

Because odor responses in occluded OSNs cannot be examined in the chronic occlusion experiment due to permanent cautery, transient unilateral naris occlusion was performed using removable nasal plugs. Adult mice (6-12 weeks old) were anesthetized by isoflurane and removal nasal plugs were inserted into one of the two nostrils, as previously described (Galliano et al., 2021; Kass et al., 2012). The occlusion was confirmed by measuring airflow from the occluded nostril via thermocouple. After 5 days, a subset of mice was used for scSeq experiment directly (control mice) to assess the effects of naris occlusion on changes in gene expression. Under brief anesthesia by isoflurane, the remaining mice were unplugged and transferred to a new empty disposable cage containing either DPG alone or 10% acetophenone. After 2 hours, mice were subjected to scSeq. Each of the two nostrils was used separately in both control (open and plugged) and odor-exposed mice (open and unplugged). Mice whose nostrils did not experience airflow after unplugged, as assessed transcriptionally, were excluded from any analyses.

### Environment Switch Experiments

To examine environment-dependent changes in gene expression and their effects on odor-evoked responses. Act-Seq was performed 2 weeks after environment switches. Adult mice (6-12 weeks old) were group-housed in the regular home-cage environment for at least one week before the transfer to their novel environments. Individual mice were transferred to a new disposable cage containing materials for a novel environment in the satellite animal facility. Two different novel environments were used, with each containing non-overlapping contents; Environment A (paper bedding, hay, dried flowers (such as marigold), peanuts, seeds (e.g., sunflower seeds, pumpkin seeds), puffed rice), and Environment B (garden soil, aspen shavings, coconut husks, dried fruits (e.g., berries, mango, pineapple), dried vegetables (e.g., green peas, corn, bell peppers), millet, fresh fruits (banana, apple, peach), corn flakes). Mice were singly-housed throughout the entire 2 weeks and were transferred to newly prepared environmental cages every 12 hours to minimize the contributions of any murine odors, which are likely the predominant odorants present within standard home-cage housing environments. After 2 weeks, individual mice were transferred to new empty disposable cages, and Act-Seq using acetophenone was performed as above.

To assess the dynamics of gene expression changes in response to novel environments, mice were housed overnight in a new disposable cage with regular bedding/food in a satellite animal facility. On the experimental day, each mouse was first transferred to Environment A cages and subjected to scSeq after 45 minutes. 2, 4, 12, 24 hours, or 3, 5, 14 days (with cages refreshed every 12 hours for durations longer than 12 hours). To account for any non-specific effects resulting from transferring mice from the animal facility to disposable cages within the satellite facility, data from mice transferred and housed in new disposable cages with regular bedding and food overnight were used as controls for downstream analyses of the effects of Environment A on gene expression.

For β-arrestin2 knock out mice, scSeq was performed on cohorts of mice that were either housed in home cages, housed in new disposable cages with regular bedding and food overnight and then transferred to Environment A cages for 5 days, or housed in new disposable cages with regular bedding and food overnight as controls, as described above. Each of the three knock out datasets was compared to its respective dataset from wild-type mice housed in the same condition.

To determine whether OSN activation reflects salient differences between environments, one set of mice were housed in a new disposable cage with regular bedding/food overnight in a satellite animal facility and then transferred to Environment A for 5 days. On the experimental day, mice were transferred to either a new Environment A cage or a home cage for two hours before being subjected to scSeq. An additional cohort of mice was housed in the home cage. The day before the experiment, they were transferred to a new disposable cage with regular bedding/food overnight in a satellite animal facility. On the experimental day they were transferred to a new home cage for two hours before being subjected to scSeq. These three conditions were compared to mice transferred to Environment A cages for 2 hours as part of the time course described above, thus generating all four combinations of environmental switches between mice housed in Environment A and home cages.

### Chronic Acetophenone Exposure

To compare the effects of chronic odor exposure with the acute activation observed via Act-Seq, scSeq was performed after exposure to DPG alone or 0.1, 1. 10% of acetophenone (diluted by DPG) for 5 days. Adult mice were individually housed in regular home cages in a satellite animal facility overnight, then two cotton balls, each of which was soaked with 300 μL of DPG or diluted acetophenone solution, were added into each cage. The odorized cotton balls were replaced every 12 hours and cages were replaced every 2 days.

### Optogenetic Stimulation

To characterize quantitative changes in gene expression after activation of OSNs. OMP-ChR2 (H134R)-Venus mice were subject to optogenetic activation and analyzed via scSeq. Mice (N=14) were anesthetized with 2% isoflurane and injected with bupivacaine (1.25 mg/kg) under the scalp. An incision was then made to expose the dorsal skull and a scalpel (Aspen Surgical 372615 Bard-Parker) was used to thin the bone overlying the right olfactory bulb, after which cyanoacrylate glue (Loctite Glass Gluc) was applied over the thinned bone. A blue LED (470 nm, Lumileds LXZI-PB01, Digikey) was soldered to a two-pin connector (Millmax, Digikey ED8450-ND) and manually placed over the bulb. A titanium head bar was then placed over the caudal end of the skull, and the whole dorsal surface was covered with dental cement (Metabond, Parkell). After the dental cement dried, a layer of black nail polish was applied to minimize the leak of blue light during stimulation. Postoperative care included a subcutaneous injection of buprenorphine SR (1 mg/kg, given 1 hour prior to surgery start) and carprofen (5 mg/kg) administered through drinking water. Before experiments, mice were tethered to a cable connecting the LED to a microcontroller (Teensy 3.2. Adafruit) that provided a custom stimulation pattern. The LED emitted 100 mW at 470 nm, measured with a light meter (Thorlabs PM100D).

For chronic activation. OSNs were activated by 5 pulses (50 msec on. 50 msec off cycle) with a 10 second inter-pulse interval for 12 hours before being subjected to scSeq. For acute activation. OSNs were activated by the same cycle as above with a 5, 10, or 20 second inter-pulse interval for 2 hours (denoted as High, Medium, and Low, respectively) and then subjected to scSeq. For both chronic and acute activation, control mice underwent the same surgery and tethering, but were implanted with a dummy connector and did not receive LED stimulation. Home cage control samples for optogenetic experiments were mice that underwent the same surgery but were subjected to scSeq directly from the home cage without any optogenetic stimulation. Because the LED was placed over the right olfactory bulb, only the right nostril was dissociated and used for scSeq for all optogenetic stimulation and control experiments.

Preparation of Single Cell Suspensions for scSeq

The main olfactory epithelium was dissected in Earle's Balanced Salt Solution (EBSS, pre-treated with carbogen for at least 5 minutes before each use, Worthington), then transferred to a round-bottomed glass dish containing 750 μL of papain solution (one vial of Papain (Worthington) dissolved in 5 mL of EBSS and then equilibrated 10 minutes at 37° C.) and 100 μL of DNase solution (one vial of DNase-I (Worthington) dissolved in 500 μL of EBSS). Bone was removed from the epithelium under a dissecting microscope and the resulting epithelial tissue was placed in a 5 mL tube (Becton Dickinson) with an additional 1.75 mL of Papain solution and 200 μL of DNase-I solution and rocked gently for-60 minutes at 37° C. The tissue was then gently triturated with a 5 mL pipette 10-15 times, passed through a 40 μm cell strainer (Becton Dickinson), and washed with 1 mL of Hibernate-A medium containing 10% Fetal Bovine Serum (FBS. GIBCO). Filtered cells were transferred to a new 5 mL tube and centrifuged 5 minutes at 300×g. The supernatant was decanted, the cells were washed once with 4 mL Hibernate-A containing 10% FBS and resuspended in 1 mL Hibernate-A containing 0.2% FBS. Importantly, each of the solutions above contained transcriptional inhibitors (5 μg/mL of Actinomycin D, 10 μg/mL of Anisomycin and 10 μM of Triptolide, all obtained from Sigma) for all experiments, especially the Act-Seq experiments, except where noted.

### Fluorescence Activated Cell Sorting

Dissociated cells were stained with propidium iodide (final concentration 1.65 μg/mL) and subjected to FACS to remove dead cells and obvious doublets. For fluorescent reporter-expressing mouse lines (OMP-IRES-GFP. P2 (P2 locus), M72 (S50 locus), S50 (M72 locus), OMP-ChR2 (H134R)-Venus), fluorescence positive and negative cells were sorted separately using a FITC filter. Cells were sorted into Hibernate-A containing 5% FBS (and 0.1× concentration of transcriptional inhibitors), centrifuged for 5 minutes at 300 g, and resuspended with PBS. For samples from OMP-IRES-GFP and OMP-ChR2 (H134R)-Venus mice, fluorescence positive cells were used for single cell RNA-seq (scSeq) experiments. For samples from OR lines (P2 (P2 locus), M72 (S50 locus) and S50 (M72 locus)), fluorescence positive and negative cells were combined for scSeq experiments.

### Single Cell Library Preparation

Single cell RNA-seq library were prepared from the single cell suspensions via the chromium single cell gene expression system (chromium single cell 3′ reagents and GEM v2, v3 or v3.1 dual index, 10× genomics), using the default protocols provided by 10× genomics. Each replicate, other than GFP-positive ones, was loaded at a concentration predicted to yield 10,000 cells, for which the expected multiplet rate is-8.0%.

### Sequencing

Sequencing library fragments were examined using the Agilent High Sensitivity DNA kit (Agilent) and quantified via qPCR by the KAPA library quantification kit (Roche). NextSeq and NovaSeq platforms were used for sequencing libraries. 75 cycle High output kit was used for NextSeq (Read1=26 cycle. Index (17)=8 cycle. Read2=58 cycle). Full flowcells of 100 cycle SP/S1/S2 kits, full flowcells or single lanes of 200 cycle S4 kit were used for NovaSeq (minimum read lengths: Read1=28 cycle. Index (17)=8 cycle. Read2=89 cycle for v2 and v3 kits and Read1=28 cycle. Index (17)=10 cycle. Index (15)=10 cycle. Read2=77 cycle for v3.1 dual index kits).

Demultiplexed fastq files were generated by mkfastq function in Cell Ranger software.

Generating gene expression matrix (raw UMI count) from sequencing data Demultiplexed fastq files were aligned to the mouse reference genome mm 10 (Ensembl 93) and converted into gene expression matrices using the 10× Genomics Cell Ranger software (version 2.2.0 (v2 and v3 samples) or 4.0.0 (v3.1 samples)) with two key modifications. First, despite the fact that the 10× genomic platforms have relatively high rates of intronic priming (La Manno et al., 2018). Cell Ranger by default considers multi-mapped reads that map to a single exonic locus as well as to non-exonic (e.g. intronic) locations as being uniquely mapped to that gene (and modifies the MAPQ scores accordingly), even when the read may have better alignment to the non-exonic loci. These multimapped reads (typically 510% of all reads) were filtered out by removing any read in the bam file with a MM:i:1 tag. Second, the output BAM files from Cell Ranger also contained many reads that were uniquely mapped to separate genes despite sharing the same cell barcode and unique molecular identifier (UMI), which is biologically implausible given that the cell barcode and UMI should uniquely identify each individual transcript. This could be either the result of UMI collision or misalignment. In support of the latter possibility, different reads of individual UMIs were often each uniquely aligned to multiple related genes, such as the large family of olfactory receptors (ORs). To avoid double-counting UMIs and inflating the numbers of genes. ORs, or UMIs detected in each cell, all ambiguous UMIs that mapped to multiple genes were removed from the BAM file with custom scripts, using samtools and pysam. The gene expression matrix was then recomputed by counting the number of distinct UMIs for each cell barcode for each gene.

Extracting Mature Olfactory Sensory Neurons from all Cells

Mature OSNs were identified using an iterative subclustering procedure via the Scanpy python package (Wolf et al., 2018). In each cell, the UMIs were total-count normalized and scaled by 10,000 (TPT normalized). Variable genes that were overdispersed relative to their mean were identified using the SPRING gene filtering function “filter genes” with parameters (90, 3, 10; see (Weinreb et al., 2018)). Ribosomal, mitochondrial, and OR genes were excluded from the variable genes. To identify mature OSNs, the gene expression of the set of variable genes was log-normalized, and for each gene, the residuals from linear regression models using the total number of UMIs and percent of UMIs for mitochondrial genes as predictors were then scaled via z-scoring and reduced to a smaller number of dimensions via principal component analysis (PCA). Cells were clustered using the top 35 PCs via the Leiden algorithm (resolution=1.2). Clusters containing mature OSNs were identified based on their expression of known mature OSN marker genes (e.g. Omp, Stoml3, Cnga2, Adcy3), the presence of cells expressing OR genes, as well as the absence of immature OSN marker genes or genes found in non-neuronal cells (e.g. Sox11, Gap43, Chr2, Clqb, Aqp3). A small cluster containing the non-canonical Gucy2d (GCD) and Gucylb2 expressing neurons was routinely identified but not used for any analyses in this paper. Clusters containing dying cells with low numbers of total counts, low numbers of genes, and high percentages of mitochondrial genes were removed. This clustering procedure typically revealed a cluster of OSNs that had higher than average total counts (often close to double the mean of other OSN clusters) and the majority of cells in such clusters often expressed multiple ORs; these cells are likely OSN-OSN doublets and were not considered further. Similarly, clusters containing mixtures of OSN and non-neuronal markers were also likely doublets between OSNs and other cell types and were not considered further. Using only the mature OSN clusters that passed this initial filtering step (˜60-65% of cells), the above procedure (starting from variable gene identification but using only 20 PCs) was repeated multiple times to remove any additional immature or unhealthy OSNs (with low counts or >10% of total counts in mitochondrial genes) until stable results were obtained. Importantly, although immature OSNs (e.g. Gap43+ and weakly Omp+) routinely express OR genes and other mature OSN markers, these OSNs were excluded by the conservative subclustering procedure described above so that any comparisons between cells expressing the same OR would not be confounded by any differences in OSN maturity.

### OR Gene Annotation (Including Functional or Pseudogenes, Class I or II, Dorsal or Ventral, and CD36-Positive or -Negative), OR Protein Similarity, and OSN Positional Subtypes

The Ensembl database (Release 93) was used for OR gene annotation. Functional OR genes were identified as genes that are associated with GO term “olfactory receptor activity” (GO: 0004984); the TAAR family of receptors was also included (GO: 0001594. GO: 1990081). To identify the OR class for each mouse OR, phylogenetic analysis was performed using all mouse and zebrafish OR proteins in MEGA7 with default parameters. As previously described. OR proteins cluster into two main phylogenetic clusters (Zhang and Firestein. 2002). The ORs that clustered together with zebrafish ORs are the class I ORs and the remaining ORs are class II ORs. To compare OR protein similarity, the pairwise phylogenetic distance matrix between OR protein sequences was calculated by taking the cophenetic distance of the phylogenetic tree of all ORs. For each pair of ORs, the cophenetic distance is equal to the height of a dendrogram in which the two branches of the phylogenetic tree containing both ORs first merge into a single cluster. Unsupervised graph-based clustering of cells identified a cluster of OSNs expressing Cd36, as previously described (Oberland et al., 2015; Xavier et al., 2016). ORs were identified as Cd36-positive if the majority of OSNs expressing that OR were found within the Cd36-positive clusters. OSNs were categorized as dorsal or ventral based on their expression of known dorsal (Nqo1, Acsm4) and ventral (Ncam2, Nfix) marker genes. ORs expressed in dorsal or ventral OSNs were referred to as dorsal or ventral ORs. With the exception of three known ventral class I ORs, all other class I ORs are dorsal, whereas OSNs expressing individual class II OR expressed either dorsal or ventral marker genes. The OR genes are located in gene clusters across most chromosomes in the genome: OR genes whose transcription start sites (TSS) were within 3 megabases of any other OR gene were considered part of the same gene cluster (Monahan et al., 2017).

### Identification of the OR Expressed in Each OSN

Of the 1172 identified functional ORs, an OR was considered expressed in any given OSN if at least 3 UMIs were detected (except for the experiments using the 10×v2 chemistry, where a threshold of 2 UMIs was used). Mature OSNs expressing single ORs were identified based on the OR that they expressed, and all cells expressing the same OR were considered as the same OSN subtype for downstream analyses. The vast majority of mature OSNs expressed a single OR (Figure S1D). OSNs expressing multiple ORs at high levels typically had higher numbers of total counts and were likely cell doublets that remained even after the previous filtering steps. OSN expressing multiple ORs with higher expression for a single OR gene also sometimes expressed other OR genes at low levels, as seen during development (Hanchate et al., 2015), or due to contamination from ambient RNA or errors in gene alignment. Regardless, any OSN expressing cither zero or multiple OR genes was not considered further for any downstream analyses. OR frequency was defined empirically based on the fraction of all mature OSNs expressing a given OR. The expression level for each OR was determined based on the normalized expression (see below) of that OR gene within the OSNs singly-expressing that OR. In the “OR-swap” mice, in which the coding sequence for each OR was knocked into the locus of the other, cells expressing swapped ORs were identified based on the gene counts for the locus rather than the expressed OR (e.g. in M72 (S50 locus) mice. OR transcripts that originated from the S50 locus indicate the expression of M72 swap cells while OSNs expressing M72 from the endogenous locus were not considered and vice versa for S50 (M72 locus) mice) due to fact that only coding sequences were swapped whereas the 3′ UTR for the original OR remain and are detected given the 3′ bias of the 10× data that arises from the use of oligo (dT) primers. The median number of UMIs/genes detected per mature OSNs and number of mature OSNs used for each replicate are summarized.

### Accounting for OR Frequency

ORs were expressed at a wide range of frequencies, spanning multiple orders of magnitude (Figure S1A-B). For instance, given that the 200 OR genes with the highest frequencies are expressed in ˜50% of all OSNs, if standard algorithms like PCA are applied on all OSNs at once, they would weight ORs expressed in 100 cells 100× more than an OR expressed in a single cell, and the resulting top PCs would capture the axes of transcriptional variation that distinguished highly-expressed ORs rather than all OSN subtypes. Similarly, classifiers trained on all cells would more easily distinguish highly-expressed ORs (from their higher influence on the PCs, increased presence in training data, and higher likelihood of accurate predictions by chance), and one could achieve high levels of overall prediction accuracy even with models that were unable to distinguish among the majority of ORs. Therefore, to account for OR frequency, most analyses were performed using equal numbers of OSNs per OR by subsampling from the population of OSNs that expressed the set of ORs each detected in at least a certain number of cells (e.g. at least 4-10, as specified, depending on the analysis). This subsampling procedure was performed 1,000 times and results were summarized for each OSN subtype (as defined by the expressed OR) across the 1,000 restarts. Analyses at the OSN subtype level were performed by first averaging across all OSNs expressing the same OR within a given condition.

### Normalization and Scaling

The filtered mature OSN datasets containing only cells expressing single ORs were renormalized, starting again from the raw UMI counts for each gene. Normalization was performed by dividing the counts for each gene in each cell by the total counts across all genes (excluding mitochondrial and ribosomal genes, as well as highly-expressed lncRNAs and lincRNAs like Malat1 and Gm42418, and sex-specific transcripts like Xist) in that cell and multiplying by 10,000 to yield tags per ten thousand (TPT) normalized data. Normalized expression in the figures and throughout refers to TPT-normalized data, where a value of 1 indicates on average 1 UMI for that gene per 10,000 UMIs. For analyses that required scaled data (e.g. PCA and classification), the logarithm of the TPT-normalized data was used (log (TPT+1)). Scaling via z-scoring was performed for each gene, using means and standard deviations identified by taking the mean of the values from 100 restarts containing equal numbers of cells per OR. The home-cage data consisted of six replicates distributed across 2 batches: separate scaling was used for each batch, but similar results were obtained by scaling both batches together. For the “OR-swap” experiments, the means and standard deviations were fit using only cells from wild-type mice collected with the same 10×v2 chemistry platform, and these were applied to the “OR-swap” and P2 (P2 locus) OSNs. In the chronic occlusion data, the means and standard deviations were recomputed using data from both nostrils. In the environment switch experiments, the gene scaling was recomputed using data from all environments.

### Dataset Integration

Importantly, besides scaling and normalizing the gene expression data, no methods for batch correction or dataset integration were performed for any dataset. The mature OSNs were readily identified and separately isolated from each dataset (obviating the need for any additional alignment of cell types across datasets), and batch effects were rarely observed when comparing the normalized expression between datasets (apart from changes in the levels of mitochondrial and ribosomal genes, which were excluded from normalization procedures and downstream analyses). This analytical choice was deliberate, as data integration procedures are affected by changes in OSN composition (due to differences in the set and frequency of ORs detected across datasets and the wide range of OR frequencies), can modify the transcriptomes by averaging and leaking data across cells (which may express different ORs), and could make OSNs transcriptomes more or less similar to each other in a manner that would hinder and bias downstream comparisons between cells expressing the same OR across experimental conditions. For instance, in the Act-Seq experiments, our expectation was that a small fraction of OSNs would exhibit difference in their transcriptomes upon being activated by odorants; however, dataset integration procedures typically attempt to normalize such differences, and thus tend to make control OSNs appear more activated and odor-exposed OSNs appear less activated. Some datasets were collected with different 10× kits (v2, v3, and v3.1); as these kits differ in their sensitivities, each sample was compared with appropriate control samples collected with the same platform.

### Gene Selection

A consensus set of genes whose expression varied across mature OSNs were identified and used as the basis of most downstream analyses where transcriptomes were compared across OSNs and conditions. After using the OR genes to identify the OR expressed in each cell, the OR genes were not considered further, and were subsequently removed from all downstream analyses. Mitochondrial and ribosomal genes, and the lncRNAs mentioned above were also excluded. Using the home-cage data, the population of OSNs that expressed 831 ORs in at least 10 cells was used. To identify genes whose expression varied across cells expressing different ORs. 10 cells were selected for each OR and the F-score between the scaled gene expression and OR identity was computed for each gene, using the set of ˜15.000 genes that were expressed in at least 0.2% of OSNs. This procedure was performed 1000 times and genes that were consistently strongly significant (Benjamini-Hochberg FDR-corrected p-value≤1×10⁻⁷ for at least 90% of restarts) were considered highly-variable genes (HVGs). This procedure identified a set of 1350 non-OR genes that were used for downstream analyses. Varying thresholds, such as the number of restarts to consider a gene as a HVG, yielded different numbers of HVGs but gave consistent results with respect to other analyses (Figure S1I).

### Principal Component Analysis

Principal component analysis (PCA) was performed using equal numbers of cells for each OR to avoid any OR having undue weights on any PC based on its frequency of expression. All versions of PCA shown used the scaled expression of the set of 1350 HVGs, and these HVGs were reduced to the top 20 PCs: however, stable results were obtained with different numbers of HVGs and PCs (Figure S1I). After fitting PCA in this manner using equal numbers of cells for each OR the fitted gene loadings (V^{T}) were applied to the scaled HVG expression for all cells to obtain PCA scores for the remaining cells. In analyses in which this subsampling procedure was repeated across restarts. PCA was fit on the subset of cells sampled for each restart. For UMAP visualization, the results from a single restart are shown (although qualitatively similar restarts were obtained across restarts). In the “OR-swap” experiments. PCA was fit using only OSNs from wild-type mice (with equal numbers of cells per OR) and then applied to the cells from the “OR-swap” and P2 (P2 locus) mouse lines. In the chronic occlusion experiments. PCA was refit using equal numbers of cells per each OR for each nostril, and in the environment switch experiments. PCA was fit using equal cells for each OR from each of the DPG-exposed control mice from each of the three environments.

### Visualization Via UMAP

UMAP was used solely for visualization purposes (McInnes et al., 2018). To generate UMAP visualizations, the gene expression of each cell was further reduced from 20 PCs to 2 dimensions using the Uniform Manifold Approximation and Projection (UMAP) technique with parameters n_neighbors=25, min_dist=0.6, and metric=“euclidean”. As for PCA. UMAP embeddings were fit using balanced numbers of cells for each OR (and only for wild-type OSNs in the “OR-swap” experiments), and these embeddings were then used to transform to the PCA scores for all cells of a given dataset into the same UMAP space. UMAP was fit using 1000 epochs and a learning rate of 0.25 to improve the stability of this transformation step. The UMAP embeddings were refit separately using the PCA scores from each dataset, yielding embeddings that differed qualitatively in appearance yet retained similar features (dorsal, ventral, and (′d36-positive OSNs were separated, the main axis was activity-dependent, cells expressing the same OR were locally clustered). UMAP and PCA were also refit using only cells from the occluded nostrils to determine if OSN subtypes cluster based on intrinsic activity in the absence of odor-evoked activity.

### Transcriptome Distance

To measure the similarity of cells based on their gene expression, the transcriptome distance was defined as the cosine distance (1-cosine similarity) between the PC scores of pairs of OSNs. The cosine distance is bounded between 0 (an angle of 0° indicating similar transcriptomes). 1 (an angle of 90° indicating orthogonality) and 2 (an angle of 180° indicating opposing transcriptomes).

Equal numbers of cells (10) were selected for each OR (of the 831 ORs detected in at least 10 cells in the home-cage dataset). PCA was refit based on the scaled HVG expression of these 8310 cells and the pairwise transcriptome distance matrix was then evaluated. Within-OR distances we calculated, for each OR, as the median pairwise distance between all pairs of cells sharing that OR. For between-OR distances, the median pairwise distance between all pairs of ORs was calculated. Then, since each OR is more similar and dissimilar to specific subsets of other ORs, for each OR, the distribution of between-OR distances were summarized across all pairs of ORs sharing that OR. Starting from subsampling OSNs, the within- and between-OR distances were recomputed across 1000 restarts, and the distribution of within-OR distances across ORs was compared to the distribution of pairwise distances between each OR and all other ORs. On each restart, the OR labels were also shuffled across cells, and within-OR distances were recomputed (and were close to 1 indicating that the transcriptomes of random cells were dissimilar). Similar results were also obtained using the Euclidean and Correlation distance metrics. To evaluate whether these distributions of within- and between-OR transcriptome distances remained separable (even though distances increase with increasing dimensionality) across various choices of gene sets and numbers of PCs, the Jensen-Shannon divergence was computed by comparing the discretized versions of these two distributions on each restart (with 111 evenly spaced bins between 0 and 2) and comparing the entropy of the average distribution to that of the average of the entropics of each individual distribution.

k-Nearest Neighbor Analysis

To determine whether cells sharing the same OR are clustered locally within gene expression space, a k-nearest neighbor (KNN) approach was used. For each of 1,000 restarts, using the pairwise distance matrix between cells described above, for each cell for each OR, the fraction of all the other cells that share the same OR within k neighbors was computed empirically for progressively larger groups of k neighbors. The fraction of same-OR cells found within k neighbors was averaged for each OR across restarts and was then summarized across the entire set of 831 ORs. The local clustering of cells sharing the same-OR was not apparent when shuffling the OR labels across the cells from each restart.

### Classification of ORs Expressed in Home Cage Data

### Classification Pipeline

In all instances, classification was performed in a cross-validated manner using linear classifiers fit on equal numbers of cells for each OR. Equal numbers of cells were randomly selected for each OR (10 cells for each of the 831 ORs expressed in at least 10 OSNs in the home-cage data). For both pairwise and OR identity classification, the number of cross-validation folds was chosen to be equal to the number of subsampled cells (10) for each OR. For each fold, one cell expressing each OR was held out and the remained cells were used to train linear support vector machine (SVM) models (SVC with kernel=“linear”, decision_function_shape=“one-vs-one”, and regularization parameter C=0.03). The hyperparameters for the classification pipeline were identified via a random search, but similar results were obtained across a range of hyperparameters. The input to the classifiers was the scaled gene expression (excluding any OR, ribosomal, or mitochondrial genes), using all genes (˜13,000) that were expressed in at least 0.5% of all cells. During each cross-validation fold, feature selection was performed by keeping the top 10% of genes with the highest F-score with the OR labels of the training fold cells. Importantly, although this is the same procedure used for HVG selection described above and the 10% threshold was designed to yield a similar number of genes, because feature selection was performed in a cross-validated manner, the resulting set of 1,300 genes identified for each fold may differ slightly from the set of HVGs used for other analyses). Next, the dimensionality of these 1,300 genes was further reduced to 20 dimensions with linear transformations via PCA and Linear Discriminant Analysis (LDA), also using only the cells in the training fold. Importantly, in addition to using equal numbers of cells per OR (to make predictions at similar chance levels for each OR), the feature selection. PCA and LDA transformations, and SVM decision boundaries were all fit using only the data in the training folds and then applied to the held-out cells in the test fold to generate predictions of the expressed OR in each held-out cell. This classification pipeline therefore avoids data leakage (which could aid in the prediction of OR identities and potentially bias results) between training and test folds.

### Classification Between OR Pairs

Pairwise classification was performed using the same classification pipeline. For each training fold, as described above, the gene sets. PCA and LDA transformations were fit using all cells in the training fold, and then applied to transform all held-out cells into the same 20D subspace. Then, using the training data, separate SVM models were fit for each pair of ORs (831C2=344.865 pairs) and these decisions boundaries were applied to the held-out cells of that OR pair to predict which OR within that pair each held-out cell expressed. This cross-validation procedure was repeated for each cross-validation fold, and the mean classification accuracy was computed for each OR pair. The entire pairwise classification procedure (starting from subsampling OSNs) was repeated across 1,000 restarts (yielding in total 344.865×10×1,000=3.45 billion models). The mean accuracy for each OR pair was summarized across the 1000 restarts, and the distributions of classifications accuracies were shown for the 344.865 OR pairs. The median prediction accuracy was 100% (compared to a chance accuracy of 50%), indicating that in the majority of OR pairs, the OR identity for every OSN of that OR pair was correctly predicted in each of the 1,000 restarts. The same classification procedure was performed separately for both nostrils for the 436 ORs detected in at least 10 cells in each nostril in the chronic occlusion data.

### Classification of OR Identity Across all ORs

The same cross-validation and classification procedure was used to predict which OR (out of 831) was expressed in each held-out cell. “One-vs-one” SVM models were first fit on the PCA and LDA-transformed gene expression of all OR pairs in the training set and then these transformations and models were applied to each cell in the test fold: this procedure was repeated for each fold. The “decision function” from this procedure thus returned for each held-out cell the predictions from the model fitted on each pair of ORs in the respective training fold, yielding a matrix of 8310 cells×344.865 models that indicates for each of the 344.865 “one-vs-one” binary pairwise classifiers which OR within that pair was more likely to be expressed. Predictions were generated using the standard voting procedure: for each cell, for each OR, the number of times that the subset of 831 pairwise models containing that OR predicted that specific OR (rather than the other OR in each pair) as more likely to be expressed were summed. The OR with the highest number of “+1” votes (out of a maximum possible 831) was predicted as the OR that was most likely to be expressed in a given cell. Since this procedure calculated the total number of votes for each OR for each cell, it also generated the likelihood (based on the ranks of the number of “+1” votes for each OR) any given OR was expressed in any given cell. Cells whose OR identity was correctly predicted were those in which the actual OR expressed in that cell was the one that received the most votes (or on rare occasions tied for the most votes).

Given that correctly predicting the exact OR (out of 831) of each held-out cell given only 9 training cells expressing the same OR and 7470 (830×9) cells expressing other ORs is a difficult task (with chance accuracy 1/831), the frequency in which the OR expressed in each held-out cell was within the top-N % of predicted ORs was also calculated using the ranks of the number of votes for each OR described above. In this case, predictions were considered as accurate at increasing percent thresholds (e.g. 0.25%=the actual OR was within the top 2 ORs with the most votes and 1%=within the top 8 ORs out of 831). For both the exact and top-N % classification procedures, the classification accuracy for each OR was summarized across the 10 cells expressing that OR in each restart and the accuracy for each OR was then averaged across 1000 restarts yielding the average classification accuracy for each of the 831 ORs, which are shown in the figures. Across the 831 ORs, the median classification accuracy was-50%. Given the balanced number of cells per OR, classification accuracy did not depend on OR frequency (Figure S1J) and was at chance levels for classifiers fit on the same data in each restart but with shuffled OR labels (1/831=0.1% for the exact predictions and 1% for the top-1% predictions). This same OR identity classification procedure was performed separately for both nostrils for the 436 ORs detected in at least 10 cells in each nostril in the chronic occlusion data.

Classification with Different Numbers of Genes

OR identity classification was performed as described above, but the % threshold for the F-score feature selection procedure of the classification pipeline was modified to assess the classification performance with varying numbers of genes. First, the high threshold was progressively increased from 0.5 to 12.5% of the −13.000 expressed genes (compared to the default of 10%). Next, starting from the default threshold of 10%, the most informative genes were progressively excluded by increasing the low threshold in 0.5% intervals such that the top 0.5%, then the top 1%, were excluded until only genes in the top 9.5-10% remained. 10 restarts were performed for each gene threshold, and the mean accuracy across ORs was calculated.

Classification with Different Gene Sets

OR identity classification was performed as above for the different gene sets, using genes from each set that were expressed in at least 0.5% of cells. These gene sets included the set of 543 mouse transcription factors detected in the OSNs (Aibar et al., 2017), and a set of 119 axon guidance genes (defined as known cell adhesion molecules with the following

prefixes: Slit, Robo, Epha, Ephb, Efn, Nrp, Sema, Plx, Kirrel, Pcdh, Cdh, Tenm; using these prefixes was important because common GO terms for axon guidance genes also include OSN-specific transcription factors like Lhx2 and Bcl11b and other genes like ion channels that are not directly involved in axon guidance). As a comparison, classification was performed using only the top 0.75% of highly variable genes based on their F-score (rather than the default used above of 10%, thus yielding −100 rather than 1,300 genes). Optimal hyperparameters for feature selection (based on their F-score in the training folds, as before) for the percent of genes from each gene set to keep, as well as the number of dimensions for PCA and LDA and SVM regularization strength were identified via randomized searches for each gene set. Classification was also performed after removing all transcription factors and axon guidance genes (and increasing the F-score threshold to 10.5% to again use the top −1,300 genes). Models trained with only axon guidance genes or transcription factors performed substantially worse than those with the top 100 F-score genes, and excluding all axon guidance genes and transcription factors genes had little effect on classification accuracy (Figure S1M).

### Classification Generalization Across Mice

Pairwise and OR identity classification was performed with the same pipeline described above. Rather than just generalizing across held-out cells (which may have been from either the same or other mice), here classification was performed by leaving out all of the OSNs from single mouse, while the remaining 5 out of 6 mice were used for training to fit the classification pipeline (gene selection, PCA and LDA transformations, and the SVM decision boundaries). For each mouse, ORs found in at least 10 cells in the 5 training mice and at least 4 cells in the test mouse were used, and classification accuracies for each OR in the test mouse were evaluated across 1,000 restarts. Similar accuracies were observed when shuffling mouse identities, demonstrating that models successful generalize across mice. Generalization performance was at chance levels when trained on models with shuffled OR labels.

### Classification of OR Identity in “OR-Swap” Mice.

Classification was performed for the “OR-swap” mice using the PCs fit on the set of 1350 HVGs. The top 10 PCs were used as inputs to “one-vs-one” SVM linear classifiers trained to distinguish between M72, S50, and P2. Using the 42/18/84 cells for M72/S50/P2 in the wild-type data, the models were trained using 15-fold cross validation (subsampling 15 cells for each OR) to evaluate their ability to distinguish between wild-type OSNs expressing the three ORs. Models fit on only the 15 wild-type cells subsampled for each OR also successfully generalized to accurately predict the OR identities in the more than 500 held-out cells for each OR from the “OR-swap” and P2 (P2 locus) mouse lines. Classification accuracy was summarized across 1,000 restarts.

### Classification of OR Classes

Classification of OR class was performed at the OSN subtype level (by taking the means of all the OSNs expressing each OR) between the 504/207/105 ventral class II, dorsal class II, and dorsal class I ORs. For each of 1,000 restarts. 80 OSN subtypes were selected for each OR type and classification was performed using 10-fold cross-validation with a pipeline that used linear SVMs (SVC with kernel=“linear”, decision_function_shape=“one-vs-one”, and regularization parameter C=0.03) fit on the top 10 PCs in each training fold to predict the OR class of the held-out ORs. Classification accuracies were at chance performance (33.3%) upon shuffling the OR type labels across OSN subtypes.

Consensus Non-Negative Matrix Factorization (cNMF)

Non-negative Matrix Factorization (NMF) was used to decompose the transcriptomes of each OSN into a smaller set of interpretable factors. NMF decomposes the gene expression matrix (cells×HVGs) into a combination of two matrices (W*H), one containing gene loadings (H) for each factor and another containing the usages (W) of these factors in each cell (FIG. 2A). In contrast to PCA, these NMF factors often represent biologically meaningful and interpretable gene sets, and therefore were referred to as gene expression programs (GEPs). Because NMF is non-deterministic, a recently-described consensus NMF (cNMF) strategy was used, which has successfully found identity- and activity-related GEPs in a range of scSeq datasets (Kotliar et al., 2019). TPT-normalized data rather than raw counts (as used previously) were used as inputs to the cNMF procedure, since biological variation in the number of counts per cell was not expected given that all cells were mature OSNs. Each gene was scaled by its standard deviation using the standard deviations described above.

In brief, as previously described (Kotliar et al., 2019), cNMF applies NMF multiple times and, after excluding outliers (based on their distance to nearest neighbors, density_threshold=0.3), clusters the gene loadings across restarts with k-means clustering and uses the median of these clusters as the new gene loadings. In each of 20 restarts in which 10 cells were resampled for each of the 831 ORs. NMF was refit 150 times across a range of factors. A rank 18 decomposition, which balanced the tradeoff between reconstruction error (as measured by the Frobenius norm) and the consistency of the identified GEPs across restarts (as measured by the silhouette score), was used for the analyses presented here, but similar GEPs were observed with factorizations of other ranks. To match factors across restarts, the 54.000×54.000 distance matrix (18×150×20) was subject to k-means clustering using the value of k (19) that maximized the silhouette score. Clusters for stable factors should have close to 3,000 members (20×150). For each of the 19 clusters, the median loading across cluster members for each gene were used as the consensus gene loadings for that GEP. Three of these 19 clusters either appeared in few restarts or had non-zero usages only in small numbers of cells and were not considered further. For the 16 remaining GEPs, the loadings were normalized to sum to 1 for each GEP. Using this set of 16 GEPs as the new H matrix, the usages of these GEPs for each OSN were calculated by reapplying NMF while keeping the gene loadings constant (with update_H=False).

### Identification of OR-Specific GEPs, the Environmental State (ES) GEPs, and Calculation of the Environmental State (ES) Score

Individual GEPs may explain variation across ORs, allowing for accurate classification of OR identities, or they may explain other, independent, aspects of variation. To distinguish between these possibilities, across 10,000 restarts 10 cells were randomly subsampled for each OR, and the correlation between the mean GEP usages for cells expressing each OR (vectors of length 831 for each of the 16 GEPs) were compared between the first and second half of cells for each OR. The 10 GEPs with consistently-high correlations (R>0.9) were considered “OR-specific”, and each had higher gene loadings in a restricted set of HVGs (Figure S2A-B).

GEPs were identified based on manual inspection of genes with high loadings and high correlations with the usages for that GEP: four GEPs included known dorsal, ventral, anterior or posterior identity marker genes, while one GEP was specific to genes associated with CD36 positivity. One GEP contained genes like Ddit3 (CHOP) that are associated with ER stress and the unfolded protein response, but this GEP was not identified as OR-specific in our mature OSN dataset. GEPs that were not OR-specific (GEPs 11-16) and whose means across cells expressing the same OR are therefore not meaningful, or GEPs whose functions were unable to be identified based on their gene loadings (GEPs 8-10, which also had usages that were weakly correlated with other GEPs) were not considered for downstream analyses.

Two of the OR-specific GEPs encoded many genes associated with neural activity in OSNs (reviewed in (Wang et al., 2017)). One included S100a5, Pcp411, and Kirrel2 (which are downregulated by chronic sensory deprivation), while another included Calb2, Kirrel3, Ppp3ca (which are upregulated after chronic sensory deprivation: see FIG. 4C). These two GEPs were referred to as GEPHigh and GEPLow. The usages of GEPHigh and GEPLow were anti-correlated and expressed in a mutually-exclusive manner across a continuous range of values (FIGS. 2 and S2). The difference in their usages was therefore summarized as a single metric to define a single continuous axis of gene expression. This difference (GEPHigh-GEPLow) was referred to as the Environmental State (ES) score, and GEPHigh and GEPLow were collectively referred to as ES GEPs, given the hypothesis that variation in ES GEP usages and thus ES scores across OSNs expressing different ORs is a consequence of the differential contribution of environmental odors on the activity levels and expression of ES-related genes across OSNs.

Among the identity GEPs, GEPDorsal and GEPVentral, as well as GEPAnterior and GEPPosterior also varied smoothly and in a similar mutually-exclusive manner across ORs; their differences in usages were therefor also summarized into single metrics (DV score=GEPDorsal-GEPVentral and AP score=GEPAnterior-GEPPosterior) to capture the continuous variation across these 3 independent axes (DV. AP, and ES: see Figure S2). The DV scores were distributed bimodally, and OSNs whose DV score=GEPDorsal-GEPVentral was at least 40 were considered as dorsal OSNs, yielding consistent results as the manual gene-based identification of dorsal OSNs described above.

### Identification of Neuronal and Functional Genes in Environmental State (ES) GEPs

Genes associated with each of GEPHigh and GEPLow were identified by two criteria: 1) the gene was within the top 200 genes with the highest loading for that GEP and 2) the expression of that gene was correlated with the GEP usage on OR-by-OR basis in the home cage data (the Spearman's rank correlation between the vectors of GEP means and gene means across the 831 ORs was 0.25). This approach identified 168 and 177 genes in GEPHigh and GEPLow, respectively. The potential functions/ontologies of these genes were manually annotated, and 63 and 54 “neuronal” genes whose known or proposed function in neurons were identified for the two ES GEPs. These neuronal genes were categorized according to their putative roles in either calcium homeostasis (calcium binding, calcium signaling, inositol phosphatide related) ion transfer (ion channel, ion pump/transporter), axon guidance (axon guidance, cell adhesion), synapse (synapse, secretory peptide), or protein transport (ER/golgi/cilia related). The remaining genes with high loadings and correlations with the ES GEPs had similar patterns of gene expression in OSNs as the above genes, but their functions in OSNs remain unannotated. The “neuronal” genes were further restricted to include only the “functional” gene categories that are predicted to directly impact OSN odor responses, and which included the 73 genes that were part of the calcium homeostasis, ion transfer and OR signaling categories. Of note, while many known components of OR signaling are included in this list, the expression of Ano2 and Adcy3 remained constant across OSN subtypes and did not meet the above correlation thresholds (p<0.2 for GEPHigh. GEPLow, and the ES score for both genes). Furthermore, although Omp was weakly correlated with the ES score (p=0.36), because it was not part of the HVGs it did not contribute to the usages of any GEP.

Applying cNME from Home-Cage Data to Other Data Sets

Although cNMF gene loadings were identified using the home-cage data, they were subsequently applied to other datasets to thus compare, on an OR-by-OR basis, changes in gene expression and GEP usages across experimental conditions. For all datasets, the TPT-normalized HVG expression from that dataset was scaled by the standard deviations from the home-cage data that were used when initially fitting cNMF. Using the fixed set of gene loadings (H), new usages (W) were solved by refitting NMF (update_H=False). To compare GEP usages across conditions, the mean GEP usage in that condition was calculated for each OSN subtype for all cells expressing that OR, and then differences between conditions were computed for each OSn subtype; distributions depict the change across OSN subtypes.

### Comparisons of ES Scores Across Mice and Conditions

Changes in ES scores were evaluated by comparing the ES scores for each OSN subtype across experimental conditions. For chronic and transient occlusion ES scores from occluded nostrils were compared to those from open nostrils. For environment switches ES scores were compared for each OSN subtype between pairs of environments. For optogenetic stimulation, the change in ES scores was compared between mice housed in the home cages and those that either were or were not optogenetically-stimulated. In the chronic acetophenone experiments. ES scores were compared between mice that were chronically exposed to various concentrations of acetophenone and those that were exposed to the control solvent DPG. The changes in ES scores and z-scored functional gene expression across conditions for each OSN subtype are described. The relative ES score change for each OSN subtype was calculated by subtracting the mean across all OSN subtypes, to normalize for any global changes across conditions that were present across the population of OSNs.

The Spearman's rank correlation was also used to compare the consistency of ES scores in OSNs expressing the same OR across datasets. ORs identified in each dataset were compared, and the correlation coefficient was calculated on an OR-by-OR basis between the vectors of mean usages for each OSN subtype in each dataset (where each value is the mean ES score across all OSNs that express that OR in each dataset), On-diagonal values for pairwise correlation matrices were calculated via bootstrapping, by resampling with replacement the ES scores for each OSN subtype from the OSNs expressing that OR and taking the mean of the pairwise correlations (of the OR-by-OR ES score means) between all pairs (10,000) from 100 bootstrap restarts.

In all experiments, identity-related GEP usages were stable for each OR across conditions (p>0.95), whereas the ES GEPs and ES scores moved predictably with changes in activity. To assess how the change in ES scores related to the overall change in transcriptomes across conditions, equal numbers of cells were subsampled for each OR, and the difference in the mean ES score for each OR was correlated with the PCs of the \ HVG vs OR matrix (the difference in mean expression of each HVG between cells expressing each OR in each condition). In the chronic occlusion, optogenetic stimulation, and environment switch experiments, the changes in ES scores were strongly correlated (p>0.8) with the top PC of this matrix, and thus well aligned with the main axis of overall gene expression changes. Given that ES scores are also aligned with the top PC of the transcriptomes in the home cage, these data together indicate that activity-regulated ES genes define a main axis of transcriptional variation, and experimental manipulation causes restricted changes in gene expression along this axis.

Identifying ORs with Significant ES Score Changes Across Datasets

ORs with significant changes in their associated ES scores across datasets were identified empirically using the d-prime metric, which measured the separability of the ES score distributions for each OSN subtype. Within each OSN subtype, the d-prime statistic was calculated by taking the difference in the mean ES scores for each condition and dividing by the square-root of the average of the variance in the two conditions. This observed d-prime statistic was compared to the distribution of resulting d-prime values from permuting the condition labels across all cells of a given OSN subtype 10,000 times. P-values were calculated empirically, corrections for multiple comparisons were performed via the Benjamini-Hochberg FDR procedure, and FDR-corrected p-values≤0.01 were considered as statistically significant. ORs with significant shifts in the chronic occlusion and environment switch experiments had mean d-prime values of 5.63 and 2.50, respectively, indicating excellent separability. OSN subtypes with significant ES score changes were separated into those whose mean ES scores increased and decreases based on the sign of the difference in mean ES scores across datasets. In the environment switch experiments, each pair of environments was analyzed separately, but results are concatenated across the 3 environment pairs (and therefore in plots depicting OSN subtypes there are 3 datapoints for OSN subtypes identified in all 3 environments).

Classification of OR Identity with NMF GEPs

Pairwise OR classification and classification of OR identity was performed using the same cross-validation and balanced subsampling procedures described above. However, rather than performing gene selection and dimensionality reduction during training, the direct input to the linear SVM classifiers was the GEP usages for each OSN for either all 10 OR-specific GEPs or only the two ES GEPs, as specified. Classification was performed using the 831 ORs found in at least 10 cells in the home-cage data, the 436 ORs found in at least 10 cells in both nostrils in the chronic occlusion data, and in the 488/496/522 ORs found in at least 8 cells in home-cage, environment A and environment B datasets from the environment switch experiments. Despite using only 2 or 10 dimensions to distinguish the entire set of ORs, classification accuracies were above chance for all datasets, and at chance levels upon shuffling OR labels for each OSN.

### Classification of Nostril and Environment Identities Using the NMF GEPs and ES Scores

Rather than identifying the OR expressed in each cell, linear SVM classifiers were also used to test whether OSNs expressing the same OR were distinguishable across datasets based on their GEP usages. Separate classifiers were used for each OSN subtype and k-fold cross-validation was used, with k set to be equal to the numbers of cells subsampled per condition. As expected, classification accuracy was significantly above chance for the chronic occlusion and environment switch experiments, especially for the ORs with significant ES shifts (but ORs with smaller but non-significant shifts were still discriminable at above-chance levels). Classification performance was similar using all 10 GEPs or only the 2 ES GEPs, and at chance levels when the dataset label was permuted across OSNs for each of 1,000 restarts, further demonstrating that the main change across conditions is restricted to changes in the ES score axis.

Classification of environment identity was also performed across subpopulations of OSN subtypes, using a minimum distance classification procedure. Across 1,000 restarts, six OSNs were subsampled for each OSN subtype for each environment (from the 477 subtypes present with enough cells in all environments), and five OSNs were used for training. The mean ES score for each subtype for each of the three environments was calculated (yielding three training vectors containing the mean ES scores across the population of 477 subtypes). The ES scores of the held-out cells for each subtype were similarly concatenated into three test vectors. For each of the 1,000 restarts, subpopulations of the OSN subtypes (containing 1-50 subtypes) were randomly sub-selected 1,000 times and the training vector for that subpopulation with the minimum distance to the same subpopulation from each test vector was the predicted environment label: predictions were considered accurate if the training vector with the minimum distance came from the same environment as the test vector. Accuracies were summarized across restarts, and classification performance was at chance levels when the environment labels were shuffled across training cells for each OSN subtype.

Modified ES Scores with HVG Subsets

Modified versions of the ES GEPs were constructed to assess the contributions of individual genes within the ES GEPs. Although many genes have low loadings for each GEP and a restricted subset have higher loadings, each GEP was defined based on their loadings across the entire set of 1350 HVGs. To test whether the 73 functional genes could substitute for the entire set of HVGs, the loadings for all of the remaining HVGs were set to 0, the total loadings for each GEP were rescaled to sum to 1 across the 73 functional genes, and this new gene loading (H) matrix was used to calculate updated usages (W) by reapplying NMF (with update_H=False).

The “functional” ES score, constructed again as GEPHigh-GEPLow from the new set of usages was highly correlated (p>0.99) with the ES score from all HVGs, decreased as expected upon occlusion, and could distinguish between OSNs expressing different pairs of ORs using the classification procedure described above, thus indicating that the “functional” genes and their expression can substitute for the entire set of HVGs (Figure S4). We also observed that the levels of some of the activation genes varied slightly across environments. To assess the contribution of these activation genes on ES scores and on the ES scores changes measured after environmental shifts, the loadings for the 169 activation genes that were part of the HVGs were set to 0 for all GEPs and the GEP usage and ES score was recalculated as described above. The ES scores and changes in ES scores calculated using all HVGs or excluding the activation genes were highly correlated (p>0.99), indicating that activation genes (whose expression primarily changes during periods of acute activation) do not account for the significant shifts in ES scores observed across environments.

### Identification and Pseudotime Analysis of Immature OSNs

Pseudotime analysis was performed on the immature OSNs identified from mice housed in the home cages. The Leiden clustering procedure that was used to identify mature OSNs also identified clusters of immature OSNs identified, which were used for pseudotime analysis. Diffusion pscudotime (n_des=10) was calculated in Scanpy on the nearest neighbor graph (n_pcs=10, n_neighbors=10), and normalized to the lowest pseudotime values of immature OSNs that had undergone OR choice and were expressing ORs. To compare gene expression patterns and GEP usages in immature OSNs expressing given ORs with those of their respective mature OSN subtype, only immature OSNs that had chosen and expressed ORs were used. Gene expression and GEP usage as a function of pseudotime were evaluated using linear generalized additive models (GAMs) in pyGAM. A linear GAM was fit using 15 basis splines (lambda=1) for individual genes and fit using 50 splines (lambda=5) for GEP usages, and the mean and 95% confidence intervals of the mean were evaluated across a grid of 500 evenly-spaced pseudotime values. The mature ES scores across OSN subtypes were discretized into five quantiles, and separate GAMs were fit using the immature OSNs expressing ORs from each mature ES score quantile.

### Differential Expression Testing

Given the wide variation in gene expression across OSNs in the home-cage condition, differential expression testing was performed at the OR-by-OR level to identify genes that consistently changed across multiple OSN subtypes. Differential expression (DE) testing was performed using ORs found in at least 6 cells in each dataset to balance the trade-off between the number of OSN subtypes and within-subtype variance. Testing was performed via the Wilcoxon signed-rank test (comparing, for each gene, the differences between means for each OSN subtype across conditions). P-values were corrected for multiple comparisons across genes via the Benjamini-Hochberg FDR procedure.

For chronic occlusion, the DE genes with the most significant changes were defined as those among the top 500 genes with the lowest p-values that had consistent changes across ORs (same sign of change in at least 50% of the 673 OSN subtypes detected in at least 6 cells in each nostril and absolute value of the median log 2-fold change across OSN subtypes of at least 0.5); however, consistent results were obtained across a wide range of thresholds given that most DE genes changed in the majority of the OSN subtypes. As expected, genes that increased or decreased during occlusion had high loadings for GEPLow and GEPHigh,
respectively. DE testing via the Wilcoxon signed-rank test was also performed to identify genes that were acutely responsive to odor exposure.

For each odor, the same DE testing procedure was performed separately for activated and non-activated OSN subtypes (as defined based on the percentage of activated cells expressing that OR; see below). To exclude the small subsets of genes that changed indiscriminately across all OSN subtypes regardless of whether they had been activated by odor, genes whose p-value was higher for non-activated ORs or those whose mean change in expression across OSN subtypes was less than 1.5 times higher in the activated ORs than non-activated ORs were removed and not considered for downstream analyses.

### Identification of Activated Cells and ORs by Act-Seq

OSN subtypes that were responsive to odorants were identified via a two-step process. First, individual OSNs (as defined by the expressed OR) were determined as activated based on their mean z-scored immediate early genes (IEG) expression. A set of 10 IEGs were used (Btg2, Egr1, Fos, Fosb, Gm13889, Junb, Nr4a1, Nr4a2, Pcdh10, and Srxn1). These genes were reliably and strongly induced in odor-exposed mice and, unlike many of the activity-dependent genes that are part of the ES GEPs and whose expression varies widely across OSNs expressing different ORs, these IEGs had consistently low expression across OSNs in control mice (average expression −0.15 in the control conditions and average log 2 fold-change of −5 in the odor conditions). The set of 10 IEGs was used since even though all of the IEGs were specific to acute activation by odors, they were still only detected in a minority of cells due to the sparse nature of scSeq. The means and standard deviations for IEG z-scoring were fit on the TPT-normalized IEG expression (without the log transformation due to the sparsity of expression) across 100 restarts containing equal numbers of subsampled cells for each OR from mice that were exposed to either acetophenone or octanal. OSNs whose mean z-scored expression was above 0.2 (−15% of OSNs in odor-exposed mice but less than 2% of OSNs in control mice exposed to the DPG solvent alone).

Second, as expected, the distribution of the percent of OSNs per OSN subtype was bimodal, with a majority of OSN subtypes showing low percentages of activated OSNs with a smaller subset showing high percentages of activated cells. Using the ORs detected in at least 4 cells in both control and odor-exposed conditions, activated ORs were defined as those in which less than 20% of cells expressing that OR were activated in the control conditions and more than 70% of cells were activated in the odor-exposed conditions, with the additional requirement of non-zero expression of at least 4 IEGs (to avoid false positives from OSNs activated by semiochemicals in the empty cages used for Act-Seq or those that strongly induced a small number of IEGs). Given that the set of ORs and the number of cells expressing each OR differed across datasets, odor-responsive OSN subtypes were re-evaluated for each dataset. Overall, odor-responsive OSN subtypes that were detected in multiple datasets were consistently identified as odor-responsive, though a small fraction of them (and thus their expressed ORs) were identified as activated in some datasets but only partially activated in others. These results are likely due to both the consequences of experimental manipulations, which were designed to affect odor responses, as well as the relatively conservative thresholds used above to identify ORs as activated.

For the environment switch experiments and the Act-Seq experiments using the four acetophenone-related odors, activated ORs were identified as described above, except using an IEG threshold of −0.05 because of the higher levels of IEG expression in the 10×v3.1 data. For the acetophenone concentration series experiment, activated ORs were either identified for each concentration, or the ORs identified as activated at 10% were also examined at lower concentrations, as specified in the text. In the optogenetic stimulation experiments, no IEG thresholds were used to identify activated cells, and instead all dorsal OSN subtypes were used for downstream analyses. In the chronic acetophenone exposure experiments, the set of acetophenone-responsive ORs identified via the acute Act-Seq experiments were used.

### Quantifying the Magnitude of Odor Responses Via the Activation Score

Although IEG induction could identify activated OSNs in a binary manner, the sparse nature of the IEGs made them less suitable for measuring differences in the degree of activation. Therefore, a larger set of odor-responsive genes (from the DE genes identified above) were used to construct an analog metric for activation and therefore compare

differences in the amount of activation across OSN subtypes (as defined by their expressed ORs). The set of “activation” genes used for this metric satisfied the following criteria 1) significant responses (FDR≤1×10⁻³) to both acetophenone and octanal, and 2) average TPT-expression of at least 0.8 in activated ORs (such that the metric wouldn't be biased by the presence of zeros). Acetophenone and octanal induced similar changes in gene expression in their respective responsive OSN subtypes. Of the ˜500 genes passing these criteria, the small number of genes that had high (p>0.4) correlations with the ES score in the control mice were removed, leaving a set of 472 “activation” genes that were used for all Act-Seq experiments.

Consistent with activation score genes generally not being differentially used in the home cage, the majority of these activation genes were not part of the 1350 HVGs in the home-cage data, and thus do not contribute to any GEP usages. The remaining 169 activation genes that were also considered as HVGs often had weak loadings across multiple GEPs. Only 16 of these 169 genes had high loadings for GEPHigh, and removing all activation genes from the HVGs and recomputing GEP usages yielded similar results. Together, these results indicate that activation gene expression is specific to the acute effects of odor exposure and does not contribute significantly to baseline variation in transcriptional identities or ES scores across OSN subtypes. To summarize the effects of odor activation across this set of activation genes, the TPT-expression of these activation genes were z-scored (using means and standard deviations for these genes in the control, acetophenone, and octanal datasets, identified by subsampling equal numbers of cells across all ORs expressed in at least 6 cells across 100 restarts). Next, using the combined set of activated acetophenone- and octanal-responsive OSN subtypes. PCA was performed on the differences in z-scored activation gene expression between control and odor conditions. The top PC of this activation gene delta matrix was kept, and the activation score was defined as the product of the weights for this top PC and the difference in z-scored activation gene expression between odor and control conditions.

Thus, the activation score summarizes for each OSN subtype the overall movement of the activation genes between control and odor conditions along this main activation axis. Although the activation score weights were fit using the combined set of acetophenone- and octanal-responsive OSN subtypes expressed in at least 6 cells for stability purposes, all subsequent Act-Seq results are shown using the larger sets of ORs detected in at least 4 cells in both control and odor conditions. To obtain activation scores for OSN subtypes from other datasets (such as those in the transient occlusion and environment switch experiments), the activation score was evaluated in a similar manner by multiplying the activation PC weights and the delta activation gene expression (odor-control) for each OSN subtype. Only activated OSNs from the odor-exposed mice were used for calculating the activation score. The weights were kept constant across experiments, but three separate versions of the gene scaling was used for the ACE and OCT Act-Seq experiments, the transient occlusion experiments, and all other Act-Seq and environment switch experiments, respectively, to account for changes in the gene means due to differences in 10× kit versions and sequencing depth across these datasets. In all experiments, activation scores were higher in odor-exposed and odor-responsive OSN subtypes. Across replicates from the same condition. OSN subtypes had consistent levels of activation, demonstrating that the activation score successfully captures differences in the amount of activation in each condition across OSNs expressing different ORs.

### Unsupervised Methods for Identifying Activated Cells and ORs

Two additional complementary methods were used to identify activated cells and measure activation in the ACE and OCT Act-Seq experiments. First unsupervised graph-based Leiden clustering (resolution=0.6) was fit on the top 20 PCs from the scaled HVGs (where PCA was fit using equal numbers of cells from control, acetophenone, and octanal mice and applied to all cells). A single cluster bore the hallmarks of activated cells, and primarily contained cells from the odor condition with increased IEG expression. Additionally, nearly all of the OSNs from odor-exposed mice that expressed activated ORs, as identified via the IEG approach described above, were part of this activated cluster.

Second, activated ORs and weights on genes affected by odor exposure were simultaneously identified via unsupervised tensor component analysis (TCA). Nonnegative tensor decomposition was fit using the hierarchical alternating least-squares algorithm (ncp_hals) in the tensor tools python package (Williams et al., 2018), and the tensor of conditions×activation genes×OSN subtypes was reduced with a rank 4 decomposition into 4 rank-1 tensors. One of these tensors represented activation and had 1) high weights in the odor but not control condition. 2) high weights for activation genes affected by acute odor exposure (like Pcdh10 and Fos), as well as 3) high weights for the odor-responsive OSN subtypes identified above based on their IEG expressions: furthermore, the TCA weights for the activation factor each OSN subtype was correlated with the mean activation score for that subtype (p>0.85 for both ACE and OCT).

### Comparison of ES Scores and Activation Scores for Act-Seq Experiments

ES scores were compared to the activation scores for each OSN subtype to test how OSN transcriptional variation affects odor responses. All correlations were calculated between the ES scores in control mice and the activation scores from separate odor-exposed mice, using the set of odor-responsive OSN subtypes detected in a least 4 cells in both control and odor conditions. Comparisons were made at the OSN subtype level, between the mean ES score from the control condition and that OSN subtype's activation score. When comparing the change in ES score and change in activation scores across datasets, only OSN subtypes present in at least 4 cells in both control and odor conditions for each dataset being compared were used.

Predictions of Activation Scores from Control ES Scores and Functional Gene Expression Via Linear Regression Models

Cross-validated linear regression models were constructed using the TPT-normalized expression of the functional genes in the control mice as predictors of the activation score in odor-exposed mice. Regularization was performed using elastic-net regularized linear models, where the optimal hyperparameters (alpha, the 11-ratio, and standardization of regressors) were identified through grid searches for each dataset. 5-fold cross-validation was used, and model performance was evaluated via the mean squared error (MSE) between predicated and observed activation scores from each test fold. This cross-validation procedure was repeated 1,000 times and the distribution of observed MSEs was compared to the predictions obtained when the same models were trained and tested on data in which the activation scores were permuted across OSN subtypes: this shuffling procedure represents performance similar to that of null models that predict the mean activation score for all subtypes. Reported errors were normalized as a percentage of the observed data to facilitate comparisons across datasets. For a subset of datasets, the predictions of the activation score from the functional genes were also compared to predictions from ordinary least squares (OLS) linear regression models that used the control ES score as a single predictor. Predictions of the changes in activation scores were evaluated in a similar way to those of the activation score, except the changes in functional gene expression for each OSN subtype were used as predictors. In addition to shuffling the change in activation score across OSN subtypes, these models were also evaluated on data where the condition label was permuted across OSNs for each OSN subtype before calculating the changes in the functional gene expression for that subtype.

Classification of Odors and Environments from Activation Scores

Classification of odor identity for the acetophenone analogs based on activation scores was performed using a minimum distance procedure in a similar manner to classification of environmental identity via ES scores. The input was the set of 106 OSN subtypes whose ORs were activated by at least one of the four acetophenone-related odors and were detected in at least four OSNs for each odor (since few ORs were present and activated in enough cells across all four datasets: but see below). Across 1,000 restarts, three OSNs per odor were used for training and the fourth was used for testing. The mean activation score for each subtype for each odor was calculated. For each of the 1,000 restarts, subpopulations of the OSN subtypes were randomly sub-selected 1,000 times and predictions were considered accurate if, across the subpopulation of activation scores, the training vector with the minimum distance for each test vector shared the same odor label. Predictions were at chance levels upon shuffling odor labels across cells for each OSN subtype. The same classification procedure was also used for each pair of acetophenone odors, but the input was the set of ORs activated by both analogs for each pair to determine if the pattern of activation scores across OSN subtypes can distinguish between odors, even for ORs activated by both odors.

Classification of environments using OSN activation scores after odor exposure as predictors was performed using cross-validated linear SVM classifiers. Activation scores were computed for each OSN (rather than the OSN subtype as described above) by applying the activation gene weights to the difference in expression between the activation gene expression in each cell in the odor condition and that of the mean of its respective OSN subtype in the control condition. Classification was performed as before by subsampling equal numbers of cells per OR on each of 1,000 restarts, and prediction accuracies were at chance levels when the OR labels were shuffled across OR pairs or the environment pair labels were shuffled across the OSNs expression each OR.

### In Vitro Odor-Receptor Interaction Metrics

The set of activated ORs identified via Act-Seq were compared to those identified via the DREAM and phospho-S6 immunoprecipitation methods (Jiang et al., 2015; von der Weid et al., 2015). In vitro EC50 values to acetophenone for its receptors were obtained from (Jiang et al., 2015; Saito et al., 2009). Only ORs from these prior papers that were also identified in the Act-Seq experiments described above were used for comparisons. The 717 ORs detected in four or more OSNs in both DPG and acetophenone-exposed animals included 16/22 DREAM and 25/49 phospho-S6 ACE ORs. ORs that were not identified by Act-Seq were also not identified in more than 4 OSNs in these experiments, indicating that these receptors might be “missing” because they are expressed in particularly small numbers of OSNs: note that this observation raises the possibility that these receptors are also near the noise floor in the DREAM and pS6 methods, since these approaches rely on bulk sequencing approaches that are also likely affected by OR frequency. In contrast to the relationship observed between the control ES score and activation scores, there was no relationship between in vitro EC50 values and activation scores from any concentration of ACE and OLS linear regression models were unable to predict activation scores from the EC50 values alone.

### Comparisons of Acute and Long-Term ES Changes Upon Environment Shifts

OSN specific activation scores were measured after mice were shifted from the home-cage to environment A (envA). The activation score at these timepoints was then compared to the two-week changes in the ES score (as measured as the difference in the ES score between home-cage and env-housed mice in the environment switch experiments). The activation score was calculated by applying the activation gene weights to the activation gene deltas between mice from each timepoint and control mice housed overnight. The activation scores were normalized by subtracting the mean activation score across OSN subtypes, to account for any non-specific changes in activation across the entire population (e.g. due to changes in odor sampling that may occur upon the transfer to new environment cages). The resulting “relative activation scores” were compared between OSN subtypes with significant increases and decreases in their ES scores after two weeks in envA, for subtypes present in at least 4 cells in all 4 conditions (home-cage and envA after two-weeks, as well as in the overnight control data and in the envA two hour dataset). As expected. OSN subtypes with significant increases/decreases in their ES scores after two weeks showed increased/decreased activation (relative to the mean) at each timepoint. In contrast, the opposite effects were observed for mice switched from envA back to home cages, and little activation was observed between mice switched to cages of the same environment.

The correlation between the activation scores at two hours and the two-week ES score shifts was evaluated using all OSN subtypes, and the two-week ES score shifts were also compared to the change in the ES score after 24 hours. Predictions of the sign of the two-week ES score based on the activation score at each timepoint was performed using 10-fold cross-validated linear SVMs (fit at the OSN subtype level using a random subset of 80 of the OSN subtypes whose ES scores significantly increased and 80 that decreased after two-weeks for classification on each of 1,000 restarts). Classification accuracy was consistently above chance levels but was at chance levels upon shuffling the sign of the two-week ES score change for each restart.

### Craniotomy and Cranial Window for Chronic Olfactory Bulb Imaging

All surgical procedures were performed in accordance with the guidelines provided by IACUC at Harvard Medical School. Prior to surgery. 4-6 weeks old Omp-IRES-Cre: Ai951) (GCaMP6f) mice, were subcutaneously injected with the analgesic Buprenorphine-SR (1 mg/kg) and anesthetized 1-2 hours later with isoflurane (3% induction. 1-2% maintenance). The skin overlying the left olfactory bulb was retracted and the underlying periosteum was scraped off with a scalpel. The skull was subsequently cleaned with saline and allowed to dry. A 2.5 mm circular craniotomy was made using a biopsy punch (Integra Miltex) taking care not to damage the underlying dura. Gelfoam saturated with cold saline was placed over the craniotomy to reduce swelling and suppress minor bleeds. A custom cranial window was constructed using two circular coverslips (Warner Instruments, 3 mm. #1 thickness). The first coverslip was ground down to 2.5 mm in diameter using a diamond scribe and subsequently bonded to the second 3 mm coverslip using Norland 71 optical epoxy. With the smaller coverslip in contact with the dura, the exposed edges of the 3 mm coverslip were gently depressed until flush with the surrounding skull and secured in place with glass glue (Loctite. P.N. 233841). This “top hat” design ensured a tight seal around the craniotomy. For head-fixation, a custom titanium head plate was positioned over the skull and, using a manual manipulator, aligned to be coplanar with the cranial window. Following alignment, the head-plate was secured to the skull using dental cement (C&B Metabond. Parkell) and the animal was returned to the home cage for recovery. The supplementary analgesic Carprofen (5 mg/kg) was provided in drinking water for 4 days following surgery. Throughout the 1-2 weeks prior to chronic imaging, the craniotomy was regularly inspected for any changes in clarity. Animals with excessive overgrowth of dura and skull tissue were not imaged and were removed from the study. In order to keep the cranial window clear of debris between imaging sessions, the dorsal surface of the skull was covered with silicone sealant (Kwik-Cast. World Precision Instruments).

### Odor Delivery

A 23-valve olfactometer was used to present odors, as previously described (Island Motion Systems, see (Pashkovski et al., 2020)). In brief, custom Arduino software was used to control valve opening and closing, thereby enabling switching between odor vials and the blank vial. This software also controlled the output of two mass flow controllers (MFC). The first MFC delivered a constant carrier flow at 0.75 L min⁻¹ of purified air into a common channel: the second MFC supplied a constant flow at 0.25 L min⁻¹ of clean air that was injected into an odor vial (see below) and then merged with the carrier flow 1 inch (2.54 cm) in front of the mouse's nose. A larger exhaust fan drew air from the cage enclosing the imaging rig to prevent any cross-contamination between odors. Monomolecular odors were diluted in Mineral oil (Sigma), and this vapor-phase concentration was further diluted 1:4 by the carrier airflow. Odor presentations lasted for two seconds and were interleaved by 30 seconds of blank (Mineral oil) delivery. The order of presentation of odors was pseudo-randomized for each experiment, such that on any given trial, odors were presented once in no predictable order.

### In Vivo Olfactory Bulb Imaging

Chronic imaging of the dorsal surface of the olfactory bulb was performed after recovery from the surgery (1-2 weeks) under ketamine (100 mg/kg) and xylazine (10 mg/kg) induced anesthesia, to specifically isolate responses in OSN axons with less modulation from local interneurons and long-range cortical feedback. Depth of anesthesia was assessed every 15 minutes via the toc-pinch reflex and intraperitoneal ketamine was administered at 1/3 concentration of the induction dose for anesthesia maintenance. Multiphoton imaging was performed at 60 Hz using a 16 kHz resonant galvo scanner (Cambridge Technologies) with a chameleon laser tuned to 920 nm delivered to 30-50 mW of excited power at the front end of the objective (10× Olympus XLUMPLFL10XW-SP lens. NA 0.6), as previously described (Pashkovski et al. 2020). Emitted fluorescence was detected using Hamamatsu H10770PA-40 PMTs. Scanimage 5 was used for hardware control and image data acquisition: WaveSurfer was used for recording of acquisition, odor and frame triggers, as well as sniffing data in a subset of mice. To obtain consistent field of views across sessions. 0.5 mg of Texas-Red conjugated Dextran (3000 MW. D3328 Thermo Fisher) dye was injected subcutaneously, and the field of view was aligned using both red and green channels based on patterns of vasculature and glomeruli, respectively (using the first session as a template). Animals were monitored throughout each imaging session for any drift in the field of view across trials.

### Experimental Designs for Olfactory Bulb Imaging Across Environment Shifts

Initial experiments consisted of a cohort of four mice imaged across multiple environment shifts. Each imaging session consisted of at least 8 trials, in which each of the 7 odors were presented. The odors used included 3 odors that activate broad sets of the dorsal glomeruli and which were presented at both a high/low concentration (pentanone (25/2.5%), ethyl butyrate (2.5/0.25%), and pentanal (25/2.5%)) as well as a 7th blank odorant (Mineral oil). When mice were not being imaged, they were housed in either regular home-cage or Environment A (envA) cages, consisting of the same contents as used for the scSeq experiments. Throughout the course of the experiment, mice were sequentially housed in the regular home cages (home1), envA cages (envA), and then regular home cages again (home2), 4-6 sessions were obtained for home1, 4-6 sessions were obtained for envA (across 14 days of housing in envA), and 2-3 sessions were obtained for home2 (across an additional 14 days of housing in the regular home-cage environments).

In an additional cohort of two mice, the responses to a broader panel of 16 odors (to facilitate decoding analyses) that densely activated the dorsal olfactory bulb were imaged. Each session consisted of at least 6 trials, in which a blank odor and each of the 16 odors (2.5% of Pentanal, Pentanone, Ethyl butyrate, Heptanal, Hexanal, Propanal, Butanal, Butanone, Hexanone, Ethyl acetate, Propyl acetate, Butyl acetate, Benzaldehyde, Tiglaldehyde, Methyl tiglate, Methyl valerate, all of which were obtained from Sigma except for Methyl tiglate, which was obtained from Thermo Fisher) were presented pseudo-randomly. Throughout the course of the experiment, mice were sequentially housed in the regular home cages (home-cage) and envA cages (envA).

### Registration, ROI Detection and Signal Extraction for Olfactory Bulb Imaging Data

For alignment of the FOV across sessions, baseline-subtracted images were downsampled (from 60 Hz to 10 Hz) and the mean image from each session was used to register the field of view across imaging sessions. Registration across sessions was performed using an iterative approach to align each session to a common template session. First, the optimal geometric transformation that maximized the overall cross correlation between mean images was identified using OpenCV (findTransformECC with warp_mode=MOTION_HOMOGRAPHY and gaussFiltSize=1). Next, local deformations were further aligned with Dipy (SymmetricDiffcomorphicRegistration with metric-CCMetric, level_iters=[250, 100, 50, 25], and inv_iter=50). The resulting procedure gave an invertible warping for each session that was subsequently applied to all frames from that session. Throughout a session, the field of view was stable, and no motion correction was applied: therefore, after registration, glomeruli were stably aligned across all trials and sessions. The z-depth of the image plane was aligned for every imaging session based on the specific pattern of vasculature and glomeruli found in each mouse, and this single plane was imaged throughout each session. Figure S7B shows that FOVs are stable across sessions and environments, as assessed by the pairwise enhanced correlation coefficient between mean images of the entire FOVs (computeECC function in OpenCV). Regions of interest (ROIs, glomeruli) were identified via Suite2p (skipping the registration and deconvolution steps) from the entire set of registered baseline-subtracted and down-sampled images from all sessions (Pachitariu et al., 2017). ROIs that covered multiple glomeruli or masks glomeruli split into multiple ROIs were manually corrected. Using the set of identified ROIs, fluorescence traces were manually extracted from the raw movies from each session by applying the session-specific warping described above to each movie and then taking the dot product of the fluorescence in the aligned movies with the pixel weights of each ROI mask. Neuropil subtraction was not performed since annuli from each glomerulus would overlap with other glomeruli and there was little background fluorescence outside of activated glomeruli.

### Analysis of GCaMP Signals

### Normalization of GCaMP Signals

For each trial and glomerulus, GCaMP signals were baseline-subtracted and normalized relative to their baseline (dF/F). The 10th percentile of the ten seconds prior to each odor presentation was considered as the baseline, dF/F values were interpolated to 50 Hz and smoothed by convolving with a gaussian filter (length=21, standard deviation=5). Because of the long duration of the initial cohort, in which mice were imaged across multiple environment shifts over more than a month, dF/F traces were z-scored for each trial: dF/F values were not z-scored for the later cohort, which was imaged for a shorter duration across a single environment shift.

### Identifying Odor-Responsive Glomeruli

Responsive glomeruli were identified separately for each environment. Responsive glomeruli were defined as those whose mean response from 0.0 to 3.0 seconds after odor valve onset was significantly higher than that of the prior 10 seconds (p≤1×10⁻³, via the Wilcoxon signed-rank test and adjusted via the Holm-Bonferroni method across odors for each glomerulus) and whose mean z-scored dF/F was greater than 0.5 (or mean dF/F was greater than 0.125 for the second cohort).

### Aligning Responses to the Population Mean

Responses were aligned across trials using the population mean to account for the variability in response onset present because odors were given in open loop without controlling for sniffing. The set of glomeruli that responded in at least two environments with a positive mean odor response were used for aligning odor responses across trials. The population mean was computed across these responsive glomeruli for each trial and the population onset was defined as the first timepoint in which the population exceeded a threshold (0.75 for z-scored dF/F data and 0.25 for dF/F); traces were aligned across trials by shifting them based on the identified population onset. This alignment procedure increased the correlation of the odor response across trials for each odor, indicating that responses are more reliable once aligned to population, rather than odor, onset. Sniffing was also recorded with a nasal pressure sensor (Honeywell AWM3100V) in a subset of mice to further assess the performance of this alignment procedure. Sniffing signals were uncorrelated across trials when using the odor valve times but were correlated across trials when using the shifts identified via the population onset. After alignment, the population response reliably increased following inspiration, further indicating that this alignment procedure helps to correct to variability in timing of odor responses across trials due to sniffing. After alignment relative to the population mean, responsive glomeruli were re-identified used the same thresholds and procedure described above, except now using the first 3 seconds after population onset, and responsive glomeruli kept for downstream analyses were those that were identified as responsive in at least two environments, and whose response to a given odor was a least twice that of the response to the blank.

### Identifying Environment-Sensitive Glomeruli

For the responsive glomeruli for each odor, the mean response amplitudes for each trial (the mean dF/F or z-scored dF/F values in the first 3 seconds following population onset) were averaged across trials within each environment. Glomeruli whose odor response amplitude differed across environment pairs were identified via permutation testing. The observed differences in means across environments was compared to a null distribution calculated by following the same procedure after permuting environment labels across trials 100,000 times. P-values were calculated empirically for each glomerulus-odor pair and were adjusted pairs by the Benjamini-Hochberg FDR procedure. Pairs whose FDR-corrected p-value was ≤0.01 were considered as significantly different across environments. Across mice and odors. 20-80% of responsive glomeruli demonstrated significant shifts in response amplitudes across the shift from home-cage to envA. In contrast, fewer glomerular-odor pairs were significantly different between home1 and home2. Glomerular responses were considered as reverted if the response amplitudes in home2 were more similar to home1 than envA. While one would naively expect reversions via regression to the mean if data were independent, no glomeruli were identified as environmentally sensitive when environmental labels were shuffled before calculating the observed difference, indicating that the observed differences in response amplitudes between home1 and envA (as well as those between envA and home2) are not the results of spurious partitioning of responses. Together, these results indicate that a large fraction of glomeruli responsive to a given odor are stable within environments but vary across environments.

### Population Level Analysis

Population odor responses were analyzed using pseudo-populations of glomeruli imaged across mice. Pentanal elicited the broadest responses across the population of glomeruli and was therefore used to assess how the population response differed between environments. To test whether differences at the population level in the pentanal responses in envA compared to home1 were reverted in home2, an “environmental axis” that separated the home1 and envA responses was constructed. For each mouse, two testing sessions were held out for both home1 and envA, and the remaining sessions were used for training, and the response at each timepoint in these training sessions was averaged across all training trials. PCA was fit on the population responses in the training data to reduce the dimensionality of the glomerular population to 10 dimensions, and the mean response was calculated in 30 overlapping windows in time (each 2 seconds long starting from −0.2 s and each subsequent one offset by 0.05 s from odor onset). The differences across the 10 PCs between the response in envA and home1 was calculated for each window, and the weights were averaged across the 30 windows and then normalized by their L-2 norm. This procedure thus returns the projection vector designed to maximally separate the odor responses in the two environments. To test whether this projection generalized to held-out sessions, the PCA transformation was applied to the mean response in the held-out sessions for home1 and envA, as well as to the home2 sessions. The difference between the population responses along this environment axis between the held-out home1 and envA session sessions was compared to the difference between the home2 and held-out home1 sessions. This cross-validation procedure was repeated across 1,000 restarts, and the mean and standard deviations across restarts were evaluated. Across all restarts, the response in envA readily diverged after odor onset, whereas the response in home2 remained similar to that in home1 at all timepoints after odor onset.

Pairwise Odor Correlations within and Across Environments

Pairwise correlations for each odor pair were calculated for each environment using the population vectors of mean odor responses (averaged across the first 3 seconds following population onset). For each odor pair, the union of responsive glomeruli was used. The pairwise correlation distance between all trials for each odor was computed and the median across trial pairs was computed for each odor pair. This same procedure also applied to pairs of the same odor, and the on-diagonal of the resulting correlation matrix represents the median across trials pairs and demonstrates the stability of the population response to the same odor across trials. The difference in correlation matrices across environments was computed, and the observed difference in the mean absolute change in pairwise correlations of the lower triangle (excluding the diagonal) was compared to a null distribution of means from shuffling the environment identity of each trial across 10,000 shuffles. For both mice, the observed difference was greater than that observed in any shuffle. Correlation matrices are shown after hierarchical clustering odors using Ward's method.

### Classifying Environment and Odor Identities Via Glomerular Populations

### Environment Identity Classification

Classification of environment identity was performed for each odor separately using the responsive glomeruli for each odor. Classification was performed using pseudo-populations of glomeruli combined across mice, using the mean response for each glomerulus across the first 3 seconds following population onset. For each of 100 restarts. 18 trials were randomly chosen for each mouse for each environment and were split into 12 training and 6 test trials. Glomerular responses were permuted across trials within each mouse and then concatenated across mice to generate pseudo-trials for model training and testing. On each restart, subpopulations of glomeruli were randomly selected 100 times, thus yielding 10,000 models. Standardization across trials for each glomerulus via z-scoring and linear SVM classifiers (regularization parameter C=0.1) were both fit on the training data and applied to predict the environment label of the held-out test pseudo-trials, and the accuracy was summarized for each subpopulation across restarts. Performance was a chance levels upon shuffling the environment labels of the training and test data.

### Odor Identity Classification and Classification Generalization

Odor identity was predicted in a similar manner, using the mean responses across the first 3 seconds following population onset in pseudo-populations of glomeruli. For each of 100 restarts, for each environment, 6 test trials (containing the responses to each of the 16 odors) were randomly held out, and the glomeruli were randomly permuted across test trials 10 times to generate pseudo-populations for 60 test pseudo-trials for each of the 16 odors. The remaining training trials were randomly subsampled from each environment to yield 24 training trials for each odor. On each restart, subpopulations of glomeruli were randomly selected 20 times, thus yielding 2.000 (20×100) models for each threshold. A classification pipeline was fit on the training data, consisting of standardization across trials for each glomerulus via z-scoring, reducing the dimensionality via PCA (n_components=5 when the number of glomeruli was <10, else n_components=10), and fitting linear SVM classifiers (“one-vs-one” configuration with regularization parameter C=0.5), and then applied to the test data. The training data from each environment and the test data from held-out trials from each environment were each used for training and testing, thus generating all four combinations (2×2) of training and testing data from either the same or different environments. Classification performance was summarized across restarts for each threshold for each of the within-environment and between-environment training/testing combinations and compared to 1) performance when environment labels were shuffled across the training trials and across the test trials from each environment and 2) performance when the odor labels were shuffled. Classification performance quickly saturated with increasing number of glomeruli, was higher for held-out trials from the same environment, and was at chance performance when odor labels were shuffled.

Odor generalization was performed to determine how the changes in pairwise odor correlations observed across environments impacted odor coding. The same pseudo-populations were constructed for training and test trials from each environment as described above for odor decoding. The entire pseudo-population of glomeruli was used without subsampling. The training and test data was first transformed with the z-scoring and PCA transformation (n_components=10) described above and fit on the training data. However, rather than fitting a single classifier to all trials from all odors, a separate classifier was fit on each odor pair in the training data (16C2=120 pairs) and applied to the remaining odor pairs (119) in the test data, thus yielding 120×119=14.280 predictions. This same procedure was repeated for each of the four within- and between-environment training/testing combinations, and the entire procedure was repeated when the environment labels were shuffled across the training trials and across the test trials from each environment. Classification accuracy from classifiers trained and tested on different odor pairs from the same environment (within-environment) was sorted across the 14.280 pairs and the accuracy for each pair in the within-environment condition was compared to that of the between-environment and shuffled-environment conditions. Although the overall distribution of classification accuracies was similar for each of the three conditions, the mean absolute difference between within-environment and between-environment accuracies for each pair was greater for the observed, rather than the shuffled environmental labels, further demonstrating that pairwise relationships between odors are altered across environments.

### Software Used for Data Analysis

Single-cell analyses were performed in python (versions 3.6-3.8) using the Scanpy package (Wolf et al., 2018) as well as custom-written scripts using the open-source python scientific stack (pysam, SciPy, NumPy, scikit-learn, umap-learn, pandas, statsmodels, matplotlib, seaborn, numba, dask). cNMF was performed using modified versions of the code described in (Kotliar et al., 2019) and accessed on the world wide web at github.com/dylkot/cNMF. Imaging data was analyzed using additional packages, including OpenCV and Suite2p (Pachitariu et al., 2017). Scripts to replicate data analyses are available on the world wide web at github.com/dattalab/Tsukahara_Brann_OSN.

### QUANTIFICATION AND STATISTICAL ANALYSIS

Hypothesis testing was performed using non-parametric statistical tests, including the Mann-Whitney U Test and the Wilcoxon signed-rank test, except in the cases where p-values were calculated empirically using resampling-based permutation tests. Distributions were compared via the Kolmogorov-Smirnov (KS) test. Experiments with multiple factors were tested with either a two-way ANOVA or with the Jonckheere-Terpstra trend test (calculated empirically via 10,000 permutations), a non-parametric extension of the Kruskal-Wallis test to evaluate trends across groups. Statistical tests that were performed for multiple genes, ORs, GEPs, glomeruli, and odors were corrected for multiple comparisons using the Benjamini-Hochberg FDR Procedure or the Holm-Bonferroni method, as specified in the text. The results of statistical tests are listed in the figure legends. The precision of sample statistics and regression trend lines were evaluated using bootstrapping, and, except where noted, plots and error bars depict the mean and the 95% confidence intervals of the mean across 1,000-10,000 bootstraps. Results from classification, regression, and other analyses were repeated across 1,000 restarts, using balanced subsamples of the data in analyses at the OSN-level to account for differences in OR frequencies. Throughout the paper, a non-parametric version of the box plot (also known as a letter-value plot) was used to represent multiple quantiles and tails of large distributions of data (e.g. to summarize across OSN subtypes or sets of ORs or OR pairs) in an agnostic way that does require setting bandwidth parameters as in violin plots or kernel density estimates. Like a conventional box plot, the largest box represents the interquartile range (25-75 percentile) and the median (dotted-line). Subsequent boxes recursively represent exponentially-smaller quantiles (the 12.5-25 and 75-87.5 percentiles, then 6.25-12.5 and 87.5-93.75 percentiles, then 3.125-6.25 and 93.75-96.875 percentiles, and so forth).

### REFERENCES

Aibar, S., Gonzalez-Blas, C. B., Moerman, T., Huynh-Thu, V. A., Imrichova, H., Hulselmans, G., Rambow, F., Marine, J.-C., Geurts, P., Aerts, J. et al. (2017). SCENIC: single-cell regulatory network inference and clustering. Nature Methods 14, 1083-1086.
Arncodo, E. M., Penikis, K. B., Rabinowitz, N., Licata, A., Cichy, A., Zhang, J., Bozza, T., and Rinberg, D. (2018). Stimulus dependent diversity and stereotypy in the output of an olfactory functional unit. Nat Commun 9, 1347.
Attneave, F. (1954). Some informational aspects of visual perception. Psychol Rev 61, 183-193. Barber, C. N., and Coppola, D. M. (2015). Compensatory plasticity in the olfactory epithelium: age, timing, and reversibility. Journal of Neurophysiology 114, 2023-2032.
Barlow, H. (1961). Possible principles underlying the transformation of sensory messages. In Sensory Communication (Cambridge, MA: MIT press), pp. 217-234.
Benda, J. (2021). Neural adaptation. Current Biology 37, R110-R116.
Best, A. R., Thompson, J. V., Fletcher, M. L., and Wilson, D. A. (2005). Cortical metabotropic glutamate receptors contribute to habituation of a simple odor-evoked behavior. Journal of Neuroscience 25, 2513-2517.
Bozza, T., Feinstein, P., Zheng, C., and Mombaerts, P. (2002). Odorant receptor expression defines functional units in the mouse olfactory system. Journal of Neuroscience 22, 3033-3043.
Bozza, T., McGann, J. P., Mombaerts, P., and Wachowiak, M. (2004). In vivo imaging of neuronal activity by targeted expression of a genetically encoded probe in the mouse. Neuron 42, 9-21.
Bozza, T., Vassalli, A., Fuss, S., Zhang, J.-J., Weiland, B., Pacifico, R., Feinstein, P., and Mombaerts, P. (2009). Mapping of class I and class II odorant receptors to glomerular domains by two distinct types of olfactory sensory neurons in the mouse. Neuron 61, 220-233.
Brann, D. H., and Datta, S. R. (2020). Finding the Brain in the Nose. Annual Review of Neuroscience 43, 277-295.
Buck, L. B. (1996). Information coding in the vertebrate olfactory system. Annual Review of Neuroscience 19, 517-544.
Burns, M. E., and Baylor, D. A. (2001). Activation, deactivation, and adaptation in vertebrate photoreceptor cells. Annual Review of Neuroscience 24, 779-805.
Cadiou, H., Aoude, I., Tazir, B., Molinas, A., Fenech, C., Meunier, N., and Grosmaitre, X. (2014). Postnatal Odorant Exposure Induces Peripheral Olfactory Plasticity at the Cellular Level. Journal of Neuroscience 34, 4857-4870.
Cho, C. E., Brueggemann, C., Noelle, D. L., and Bargmann, C. I. (2016). Parallel encoding of sensory history and behavioral preference during Caenorhabditis elegans olfactory learning. Elife.
Chong, E., Moroni, M., Wilson, C., Shoham, S., Panzeri, S., and Rinberg, D. (2020). Manipulating synthetic optogenetic odors reveals the coding logic of olfactory perception. Science 368.
Chu, M. W., Li, W. L., and Komiyama, T. (2017). Lack of Pattern Separation in Sensory Inputs to the Olfactory Bulb during Perceptual Learning. eNeuro 4.
Cleland. T. A., and Linster, C. (2005). Computation in the olfactory system. Chemical Senses 30, 801-313.
Coppola, D. M., and Waggener, C. T. (2012). The effects of unilateral naris occlusion on gene expression profiles in mouse olfactory mucosa. J Mol Neurosci 47, 604-618.
Coppola, D. M., Waguespack, A. M., Recms, M. R., Butman, M. L., and Cherry, J. A. (2006). Naris occlusion alters transductory protein immunoreactivity in olfactory epithelium. Histol Histopathol 21, 487-501.
Davis, G. W. (2006). Homeostatic control of neural activity: from phenomenology to molecular design. Annual Review of Neuroscience 29, 307-323.
Davis, G. W. (2013). Homeostatic signaling and the stabilization of neural function. Neuron 80, 718-728.
de March, C. A., Titlow, W. B., Sengoku, T., Brcheny, P., Matsunami, H., and Mcclintock, T. S. (2020). Modulation of the combinatorial code of odorant receptor response patterns in odorant mixtures. Mol Cell Neurosci 104, 103469.
Dey, S., Chamero, P., Pru, J. K., Chien, M.-S., Ibarra-Soria, X., Spencer, K. R., Logan, D. W., Matsunami, H., Peluso, J. J., and Stowers, L. (2015). Cyclic Regulation of Sensory Perception by a Female Hormone Alters Behavior. Cell 161, 1334-1344.
Eggan, K., Baldwin, K., Tackett, M., Osborne, J., Gogos, J., Chess, A., Axel, R., and Jaenisch, R. (2004). Mice cloned from olfactory sensory neurons. Nature 428, 44-49.
Feinstein, P., and Mombaerts, P. (2004). A contextual model for axonal sorting into glomeruli in the mouse olfactory system. Cell 117, 817-831.
Fettiplace, R., and Ricci, A. J. (2003). Adaptation in auditory hair cells. Curr Opin Neurobiol/3, 446-451.
Firestein, S. (2001). How the olfactory system makes sense of scents. Nature 4/3, 211-218. Fischl, A. M., Heron, P. M., Stromberg, A. J., and Mcclintock, T. S. (2014). Activity-dependent genes in mouse olfactory sensory neurons. Chemical Senses 39, 439-449.
Fitzwater, E., and Coppola, D. M. (2020). Olfactory Deprivation and Enrichment: An Identity of Opposites? Chemical Senses 46.
Fletcher, R. B., Das, D., Gadye, L., Street, K. N., Baudhuin, A., Wagner, A., Cole, M. B., Flores, Q., Choi, Y. G., Yosef, N. et al. (2017). Deconstructing Olfactory Stem Cell Trajectories at Single-Cell Resolution. Cell Stem Cell 20, 817-830.e818.
Galliano, E., Hahn, C., Browne, L. P., R Villamayor, P., Tufo, C., Crespo, A., and Grubb, M. S. (2021). Brief Sensory Deprivation Triggers Cell Type-Specific Structural and Functional Plasticity in Olfactory Bulb Neurons. Journal of Neuroscience 47, 2135-2151.
Goldman, M. S., Golowasch, J., Marder, E., and Abbott, L. F. (2001). Global structure, robustness, and modulation of neuronal models. Journal of Neuroscience 21, 52295238.
Gouwens, N. W., Sorensen, S. A., Baftizadeh, F., Budzillo, A., Lec, B. R., Jarsky, T., Alfiler, L., Baker, K., Barkan, E., Berry, K. et al. (2020). Integrated Morphoelectric and Transcriptomic Classification of Cortical GABAergic Cells. Cell 183, 935-953.c919.
Grabska-Barwińska, A., Barthelme, S., Beck, J., Mainen, Z. F., Pouget, A., and Latham, P. E. (2016). A probabilistic approach to demixing odors. Nature Neuroscience 20, 98-106.
Hagendorf, S., Fluegge, D., Engelhardt, C., and Spehr, M. (2009). Homeostatic control of sensory output in basal vomeronasal neurons: activity-dependent expression of ether-à-go-go-related gene potassium channels. Journal of Neuroscience 29, 206-221.
Hanchate, N. K., Kondoh, K., Lu, Z., Kuang, D., Ye, X., Qiu, X., Pachter, L., Trapnell, C., and Buck, L. B. (2015). Single-cell transcriptomics reveals receptor transformations during olfactory neurogenesis. Science 350, 1251-1255.
Hopfield, J. J. (1991). Olfactory computation and object perception. Proceedings of the National Academy of Sciences 88, 6462-6466.
Hu, X. S., Ikegami, K., Vihani, A., Zhu, K. W., Zapata, M., de March, C. A., Do, M., Vaidya, N., Kucera, G., Bock, C., et al. (2020). Concentration-Dependent Recruitment of Mammalian Odorant Receptors. eNeuro 7.
Ibarra-Soria. X., Nakahara, T. S., Liluc, J., Jiang. Y., Trimmer, C., Souza, M. A., Netto, P. H., Ikegami, K., Murphy, N. R., Kusma, M., et al. (2017). Variation in olfactory neuron repertoires is genetically controlled and environmentally modulated. Elife 6, 1083. Imai, T., Sakano, H., and Vosshall, L. B. (2010). Topographic Mapping—The Olfactory System. Cold Spring Harbor Perspectives in Biology 2.
Jiang, Y., Gong, N. N., Hu, X. S., Ni, M. J., Pasi, R., and Matsunami, H. (2015). Molecular profiling of activated olfactory neurons identifies odorant receptors for odors in vivo. Nature Neuroscience, 1-11.
Juang. B.-T., Gu, C., Starnes, L., Palladino, F., Goga, A., Kennedy, S., and L'Etoile, N. D. (2013). Endogenous nuclear RNAi mediates behavioral adaptation to odor. Cell 154, 1010-1022. Kadohisa. M., and Wilson, D. A. (2006). Olfactory cortical adaptation facilitates detection of odors against background. Journal of Neurophysiology 95, 1888-1896.
Kass, M. D., Pottackal, J., Turkel, D. J., and McGann, J. P. (2012). Changes in the Neural Representation of Odorants After Olfactory Deprivation in the Adult Mouse Olfactory Bulb. Chemical Senses 38, 77-89.
Kato, H. K., Chu, M. W., Isaacson, J. S., and Komiyama, T. (2012). Dynamic sensory representations in the olfactory bulb: modulation by wakefulness and experience. Neuron 76, 962-975.
Kim, D.-W., Yao, Z., Graybuck, L. T., Kim, T.-K., Nguyen, T. N., Smith, K. A., Fong, O., Yi, L., Koulena. N., Pierson, N., et al. (2019). Multimodal Analysis of Cell Types in a Hypothalamic Node Controlling Social Behavior. Cell 179, 713-728.c717.
Kim, E. J., Zhang, Z., Huang. L., Ito-Cole, T., Jacobs, M. W., Juavinett, A. L., Senturk, G., Hu, M., Ku. M., Ecker, J. R., et al. (2020). Extraction of Distinct Neuronal Cell Types from within a Genetically Continuous Population. Neuron 107, 274-282.e276.
Kostal, L., Lansky, P., and Rospars, J.-P. (2008). Efficient olfactory coding in the pheromone receptor neuron of a moth. PLOS Computational Biology 4, e1000053.
Kotliar, D., Veres, A., Nagy, M. A., and Tabrizi, S. (2019). Identifying gene expression programs of cell-type identity and cellular activity with single-cell RNA-Seq. Elife.
L'Etoile, N. D., Coburn, C. M., Eastham, J., Kistler, A., Gallegos, G., and Bargmann, C. I. (2002). The cyclic GMP-dependent protein kinase EGL-4 regulates olfactory adaptation in C. elegans. Neuron 36, 1079-1089.
La Manno, G., Soldatov, R., Zeisel, A., Braun, E., Hochgerner, H., Petukhov, V., Lidschreiber, K., Kastriti, M. E., Lönnerberg. P., Furlan, A., et al. (2018). RNA velocity of single cells. Nature 560, 494-498.
Lecoq, J., Tiret, P., and Charpak, S. (2009). Peripheral adaptation codes for high odor concentration in glomeruli. Journal of Neuroscience 29, 3067-3072.
Li. A., Gire, D. H., Bozza. T., and Restrepo, D. (2014). Precise detection of direct glomerular input duration by the olfactory bulb. Journal of Neuroscience 34, 16058-16064.
Li, H., Horns, F., Wu, B., Xic, Q., Li, J., Li. T., Luginbuhl, D. J., Quake, S. R., and Luo, L. (2017). Classifying Drosophila Olfactory Projection Neuron Subtypes by Single-Cell RNA Sequencing. Cell 171, 1206-1220.
Litwin-Kumar, A., Harris. K. D., Axel. R., Sompolinsky. H., and Abbott. L. F. (2017). OptimalDegrees of Synaptic Connectivity. Neuron 93, 1153-1164.
Ma, L., Wu. Y., Qiu. Q., Scheerer. H., Moran. A., and Yu. C. R. (2014). A Developmental Switch of Axon Targeting in the Continuously Regenerating Mouse Olfactory System. Science 344, 194-197.
Marder, E. (2011). Variability, compensation, and modulation in neurons and circuits. Proceedings of the National Academy of Sciences 108 Suppl 3, 15542-15548.
Marder, E., and Goaillard. J.-M. (2006). Variability, compensation and homeostasis in neuron and network function. Nature Reviews Neuroscience 7, 563-574.
Martelli, C., and Storace. D. A. (2021). Stimulus Driven Functional Transformations in the Early Olfactory System, Frontiers in Cellular Neuroscience/5, 684742.
Mashukova, A., Spehr. M., Hatt, H., and Neuhaus, E. M. (2006), beta-Arresting-Mediated Internalization of Mammalian Odorant Receptors. Journal of Neuroscience 26, 9902-9912.
McInnes, L., Healy. J., and Melville, J. (2018), UMAP: Uniform Manifold Approximation and Projection for Dimension Reduction, arXivorg stat.ML.
McNamara, A. M., Magidson, P. D., Linster. C., Wilson, D. A., and Cleland. T. A. (2008). Distinct neural mechanisms mediate olfactory memory formation at different timescales. Learn Mem 15, 117-125.
Miyamichi, K., Serizawa. S., Kimura, H. M., and Sakano, H. (2005). Continuous and overlapping expression domains of odorant receptor genes in the olfactory epithelium determine the dorsal/ventral positioning of glomeruli in the olfactory bulb. Journal of Neuroscience 25, 3586-3592.
Monahan, K., and Lomvardas, S. (2015). Monoallelic expression of olfactory receptors. Annual Review of Cell and Developmental Biology 37, 721-740.
Monahan, K., Schieren, I., Cheung, J., Mumbey-Wafula. A., Monuki, E. S., and Lomvardas, S. (2017), Cooperative interactions enable singular olfactory receptor expression in mouse olfactory neurons. Elife 6, c28620.
Moore, P. A. (1994). A model of the role of adaptation and disadaptation in olfactory receptor neurons: implications for the coding of temporal and intensity patterns in odor signals. Chemical Senses 19, 71-86.
Mori, K., and Sakano, H. (2011). How is the olfactory map formed and interpreted in the mammalian brain? Annual Review of Neuroscience 34, 467-499.
Nagel, K. I., and Wilson, R. I. (2011). Biophysical mechanisms underlying olfactory receptor neuron dynamics. Nature Neuroscience/4, 208-216.
Nakashima, A., Takeuchi, H., Imai, T., Saito, H., Kiyonari, H., Abe, T., Chen, M., Weinstein, L. S., Yu, C. R., Storm, D. R., et al. (2013). Agonist-independent GPCR activity regulates anterior-posterior targeting of olfactory sensory neurons. Cell 154, 1314-1325.
Ng, R., Salem, S. S., Wu, S.-T., Wu, M., Lin, H.-H., Shepherd, A. K., Joiner, W. J., Wang, J. W., and Su, C.-Y. (2019). Amplification of Drosophila Olfactory Responses by a DEG/ENaC Channel. Neuron 104, 947-959.e945.
O'Leary, T., Williams, A. H., Caplan, J. S., and Marder, E. (2013). Correlations in ion channel expression emerge from homeostatic tuning rules. Proceedings of the National Academy of Sciences 110, E2645-2654.
Oberland, S., Ackels, T., Gaab, S., Pelz, T., Spehr, J., Spehr, M., and Neuhaus, E. M. (2015). CD36 is involved in oleic acid detection by the murine olfactory system. Frontiers in Cellular Neuroscience 9, 366.
Pachitariu, M., Stringer, C., Dipoppa, M., Schröder, S., Rossi, L. F., Dalgleish, H., Carandini, M., and Harris, K. D. (2017). Suite2p: beyond 10,000 neurons with standard two-photon microscopy. bioRxiv.
Pacifico, R., Dewan, A., Cawley, D., Guo, C., and Bozza, T. (2012). An olfactory subsystem that mediates high-sensitivity detection of volatile amines. Cell Reports 2, 76-88.
Parrilla, M., Chang, I., Degl'Innocenti, A., and Omura, M. (2016). Expression of homeobox genes in the mouse olfactory epithelium. The Journal of Comparative Neurology 524, 2713-2739.
Pashkovski, S. L., Iurilli, G., Brann, D., Chicharro, D., Drummey, K., Franks, K., Panzeri, S., and Datta, S. R. (2020). Structure and flexibility in cortical representations of odour space. Nature 583, 253-258.
Repicky, S. E., and Luetje, C. W. (2009). Molecular receptive range variation among mouse odorant receptors for aliphatic carboxylic acids. Journal of Neurochemistry 109, 193-202. Rubin, B. D., and Katz, L. C. (1999). Optical imaging of odorant representations in the mammalian olfactory bulb. Neuron 23, 499-511.
Saito, H., Chi, Q., Zhuang, H., Matsunami, H., and Mainland, J. D. (2009). Odor Coding by a Mammalian Receptor Repertoire. Science Signaling 2, ra9-ra9.
Scala, F., Kobak, D., Bernabucci, M., Bernaerts, Y., Cadwell, C. R., Castro, J. R., Hartmanis, L., Jiang. X., Laturnus, S., Miranda, E., et al. (2021). Phenotypic variation of transcriptomic cell types in mouse motor cortex. Nature 598, 144-150.
Schaffer, E. S., Stettler, D. D., Kato, D., Choi, G. B., Axel, R., and Abbott, L. F. (2018). Odor Perception on the Two Sides of the Brain: Consistency Despite Randomness. Neuron 98, 736-742.
Scholz, P., Kalbe, B., Jansen, F., Altmueller, J., Becker, C., Mohrhardt, J., Schreiner, B., Gisselmann, G., Hatt, H., and Osterloh, S. (2016). Transcriptome Analysis of Murine Olfactory Sensory Neurons during Development Using Single Cell RNA-Seq. Chemical Senses 41, 313-323.
Schulz, D. J., Goaillard, J.-M., and Marder, E. E. (2007). Quantitative expression profiling of identified neurons reveals cell-specific constraints on highly variable levels of gene expression. Proceedings of the National Academy of Sciences 104, 13187-13191.
Soucy, E. R., Albeanu, D. F., Fantana, A. L., Murthy, V. N., and Meister, M. (2009). Precision and diversity in an odor map on the olfactory bulb. Nature Neuroscience 12, 210-220.
Spors, H., and Grinvald, A. (2002). Spatio-temporal dynamics of odor representations in the mammalian olfactory bulb. Neuron 34, 301-315.
Sullivan, S. L., Ressler, K. J., and Buck, L. B. (1995). Spatial patterning and information coding in the olfactory system. Current Opinion in Genetics & Development 5, 516-523.
Tasic. B., Yao, Z., Graybuck, L. T., Smith, K. A., Nguyen, T. N., Bertagnolli, D., Goldy, J., Garren, E., Economo, M. N., Viswanathan, S., et al. (2018). Shared and distinct transcriptomic cell types across neocortical areas. Nature 563, 72-78.
Teşileanu, T., Cocco. S., Monasson, R., and Balasubramanian, V. (2019). Adaptation of olfactory receptor abundances for efficient coding. Elife 8, 308.
Touhara. K. (2002). Odor discrimination by G protein-coupled olfactory receptors. Microsc Res Tech 58, 135-141.
Turrigiano, G. (2011). Too many cooks? Intrinsic and synaptic homeostatic mechanisms in cortical circuit refinement. Annual Review of Neuroscience 34, 89-103.
Turrigiano, G. G. (1999). Homeostatic plasticity in neuronal networks: the more things change, the more they stay the same. Trends Neurosci 22, 221-227.
Turrigiano, G. G. (2008). The self-tuning neuron: synaptic scaling of excitatory synapses. Cell 135, 422-435.
Turrigiano, G. G. (2017). The dialectic of Hebb and homeostasis. Philosophical Transactions of the Royal Society B: Biological Sciences 372.
Tyssowski, K. M., and Gray, J. M. (2019). The neuronal stimulation-transcription coupling map. Curr Opin Neurobiol 59, 87-94.
Vihani, A., Hu, X. S., Gundala, S., Koyama, S., Block, E., and Matsunami, H. (2020).
Semiochemical responsive olfactory sensory neurons are sexually dimorphic and plastic. Elife 9. von der Weid, B., Rossier, D., Lindup, M., Tuberosa, J., Widmer, A., Col, J. D., Kan, C., Carleton, A., and Rodriguez, I. (2015). Large-scale transcriptional profiling of chemosensory neurons identifies receptor-ligand pairs in vivo. Nature Neuroscience, 1-11.
Wachowiak, M., and Cohen, L. B. (2001). Representation of odorants by receptor neuron input to the mouse olfactory bulb. Neuron 32, 723-735.
Waggener, C. T., and Coppola, D. M. (2007). Naris occlusion alters the electro-olfactogram: evidence for compensatory plasticity in the olfactory system. Neurosci Lett 427, 112-116. Wang, H. W., Wysocki, and Gold, G. H. (1993). Induction of olfactory receptor sensitivity in mice.
Science 260, 998-1000.
Wang, Q., Titlow, W. B., Mcclintock, D. A., Stromberg, A. J., and Mcclintock, T. S. (2017). Activity-Dependent Gene Expression in the Mammalian Olfactory Epithelium. Chemical Senses 42, 611-624.
Wark, B., Lundstrom, B. N., and Fairhall, A. (2007). Sensory adaptation. Curr Opin Neurobiol 17, 423-429.
Weber, A. I., Krishnamurthy, K., and Fairhall, A. L. (2019). Coding Principles in Adaptation. Annual Review of Vision Science 5, 427-449.
Weinreb, C., Wolock, S., and Klein, A. M. (2018). SPRING: a kinetic interface for visualizing high dimensional single-cell expression data. Bioinformatics 34, 1246-1248.
Williams, A. H., Kim, T. H., Wang, F., Vyas, S., Ryu, S. I., Shenoy, K. V., Schnitzer, M., Kolda, T. G., and Ganguli, S. (2018). Unsupervised Discovery of Demixed, Low-Dimensional Neural Dynamics across Multiple Timescales through Tensor Component Analysis. Neuron 98, 1099-1115.
Wilson, C. D., Serrano, G. O., Koulakov, A. A., and Rinberg, D. (2017). A primacy code for odor identity. Nat Commun 8, 1477.
Wilson, D. A. (1998). Habituation of odor responses in the rat anterior piriform cortex. Journal of Neurophysiology 79, 1425-1440.
Wilson, D. A. (2000). Comparison of odor receptive field plasticity in the rat olfactory bulb and anterior piriform cortex. Journal of Neurophysiology 84, 3036-3042.
Wilson, D. A. (2009). Olfaction as a model system for the neurobiology of mammalian short-term habituation. Neurobiology of Learning and Memory 92, 199-205.
Wolf, F. A., Angerer, P., and Theis, F. J. (2018). SCANPY: large-scale single-cell gene expression data analysis. Genome Biology 19, 15.
Wu, Y., Ma, L., Duyck, K., Long, C. C., Moran, A., Scheerer, H., Blanck, J., Peak, A., Box, A., Perera, A., et al. (2018). A Population of Navigator Neurons Is Essential for Olfactory Map Formation during the Critical Period. Neuron 100, 1066-1082.
Wu, Y. E., Pan, L., Zuo, Y., Li, X., and Hong, W. (2017). Detecting Activated Cell Populations Using Single-Cell RNA-Seq. Neuron 96, 313-329.
Xavier, A. M., Ludwig, R. G., Nagai, M. H., de Almeida, T. J., Watanabe, H. M., Hirata, M. Y., Rosenstock, T. R., Papes, F., Malnic, B., and Glezer, I. (2016). CD36 is expressed in a defined subpopulation of neurons in the olfactory epithelium. Scientific Reports 6, 25507.
Yadon, C. A., and Wilson, D. A. (2005). The role of metabotropic glutamate receptors and cortical adaptation in habituation of odor-guided behavior. Learn Mem 12, 601-605.
Yap, E.-L., and Greenberg, M. E. (2018). Activity-Regulated Transcription: Bridging the Gap between Neural Activity and Behavior. Neuron 100, 330-348.
Zhang, J., Huang, G., Dewan, A., Feinstein, P., and Bozza, T. (2012). Uncoupling stimulus specificity and glomerular position in the mouse olfactory system. Mol Cell Neurosci 51, 79-88.
Zhang, X., and Firestein, S. (2002). The olfactory receptor gene superfamily of the mouse. Nature Neuroscience 5, 124-133.
Zufall, F., and Leinders-Zufall, T. (2000). The cellular and molecular basis of odor adaptation. Chemical Senses 25, 473-481.
Zwicker, D., Murugan, A., and Brenner, M. P. (2016). Receptor arrays optimized for natural odor statistics. Proceedings of the National Academy of Sciences 113, 5560-5575.

1. A method comprising:
measuring the expression of at least one odor response gene in a first olfactory sensory neuron (OSN) of a first subtype that has been contacted with at least one volatized chemical compound.

2. The method of claim 1, wherein the method further comprises measuring the expression of at least one odor response gene in a second OSN that has not been contacted with the at least one volatized chemical compound.

3. (canceled)

4. A method comprising:
measuring the expression of at least one odor response gene in a first OSN of a first subtype that has been contacted with at least one volatized chemical compound;
measuring expression of at least one odor response gene in a second OSN that has not been contacted with the at least one volatized chemical compound; and
determining that the first subtype expresses an odorant receptor that binds to the at least one volatized chemical compound if the expression of the at least one odor response gene in the first OSN and the second OSN are different.

5. The method of claim 1, wherein
the at least one odor response gene is selected from the group consisting of: Tsc22d1, Rcan1, Fosb, Pcdh10, Dusp14, Srxn1, Gm13889, Golga4, Heg1, Cdkn1a, Gpr137c, Zfp655, Ublcp1, Efr3b, Etf1, Srrm4, Epcam, Trp53inp2, Arhgap17, Ric8b, 9530034E10Rik, Oaz2, Ctxn3, Slc38a2, Exoc6b, Nfatc1, Junb, Tle3, Siah2, Fos, Ago2, Arhgef28, Ddx3x, Hspa8, Hnmnph3, Erf, Ak2, Pnrc1, Mafg, D1Ertd622e, Arid5b, Scn3b, Phtf2, Hsph1, Cpeb4, Ubc, Sema7a, Dleu2, Rc3h1, Pcnx4, Btg2, 6430548M08Rik, Selenok, Psmd8, Map9, Cry2, Hspa41, Mcl1, Dnaja1, Fth1, Fam208a, Bmpr2, Zfp608, BC005561, Rab11fip4, Smim13, Ppp2ca, Sod1, Neat1, Brinp2, Tuba4a, Cdc14a, Kdm6b, Uckl1os, Stk40, Dnajb2, Dnajb9, Spsb3, Sub1, Rheb, Manea, Fzd3, Zfand5, Uhrf1bp11, Cebpb, Wdfy3, Mfge8, Inpp4a, Nab1, Tceal9, Ube2g1, Pcdh7, Eef1a1, Tnrc6c, Cd82, BC005537, Sdcbp, Ddx5, Olfm1, Srpk1, M6pr, Braf, Ahi1, Clcn3, Eef2, Rbm26, Arfgef3, Eif5, Tmem150c, Aebp2, Snrk, Bex3, Epb4112, Samd4b, Stard10, Pappa, Grsf1, Ralgds, Arih1, Micu3, Ptges3, Kras, Morf412, Rtp2, Wdr26, Hspa5, Zmynd8, Sv2c, Paip2, Rab39b, Ywhaz, Gpat3, Psen2, Pde4a, Atp2b1, Irs2, Cfap69, Hspa9, Tob1, Hspa14, Pard6a, Mapk4, Nr4a1, Sik3, Epha7, Hagh, Kalrn, Prkar1a, Lmbrd1, Erich3, Sf3b1, Ksr2, Ybx1, Nfix, Hivep1, Mfn2, Elof1, Wdfy2, Emd, Azin1, Wbp1, Nop53, Syt7, Ncoa1, Smarca5, Aes, Rnf150, Plxnb2, Wdsub1, Dyrk4, Epb4111, Pcdh8, Daam1, Prpf4b, Tnrc6a, Rufy3, Ptdss2, Tmem230, Scn3a, Rock1, Dph3, Zbtb7b, Yipf4, Ift74, Ebf1, Wsb2, Rnf182, Arglu1, Ppm11, Ccdc189, Ptov1, Ccdc157, Ppa1, Pnpla8, Rack1, Rsrp1, Ttc9, Hcn2, Ccdc40, Kat2b, Cngb1, Ndufa3, Eefla2, Fmn2, Macrod1, Polr2i, 1700016K19Rik, Jade1, Slcla2, Ralbp1, Eif4e3, Morn2, Trappc21, Scrn1, Rtp1, Phyh, Fetub, Mapre3, Dnajb13, Coa3, Spef1, 1810058124Rik, Cep290, Bbs4, Nin, D430042009Rik, Tex9, Sdc3, Ebf4, Oaz1, Napa, Ttc8, Sdhaf4, Cuta, Commd6, Sfr1, Hcfclr1, Nxn12, Kcnh3, Trpm7, Glb112, Slc25a39, Nsmf, Pifo, Oscp1, 1110008P14Rik, Egr1, Ttl16, Sem1, Aplg1, Fkbp2, Suclg1, Cidea, Dynlrb2, Ndufa2, Map1a, Sun1, Rufy2, Rnh1, Dtna, Anapc16, Pdhb, A430035B10Rik, Faim2, Ebf2, Ccdc28b, Ttl13, Drc1, Sys1, Prdx5, Palm, Slu7, Dnajc15, Elmod1, Paqr9, Tubb3, Ascc1, Ndufc1, Pon2, Fkbp4, Trnp1, Cldn3, Cby1, Tctex1d2, Slc48a1, Nme5, 1110004E09Rik, Cnppd1, Naa38, Vdac3, Puf60, Cers5, Cbr1, Ift43, Fam217a, Ankrd10, Ube2n, Pfdn6, Mpc2, Ncbp2, Timm13, Lamtor5, Uqcr10, Slc27a2, Arpc51, Adh5, Gkap1, Ndufaf4, Psip1, Omp, Bcl11a, Ift81, 2010107E04Rik, Ubxn1, Ech1, Tnrc6b, Tmpo, Ssbp4, 2410015M20Rik, Mapk8ip2, Naxe, Gm13589, Hdgf13, Slc22a23, Cacna1h, Slc25a5, Ndufa13, Anp32a, Ndufb10, Lhx2, Cenpx, Rnf220, Ankrd54, Ppdpf, Dtd1, Auh, Mrp151, Psmd13, Lamtor4, Hist2h2be, Mapkap1, Arhgdig, Ndufv3, Ilkap, Ctsa, Ift27, Lpcat4, Atp50, Ypel3, Gadl1, Msln1, Hint2, Acbd7, Manf, Lamtor2, Ak1, Arl6, Uxs1, Sri, Tomm7, Ubxn6, Cd47, Snrpd3, Ppid, Rsph9, Rbfox2, Pigp, C1qbp, Fmc1, Bcl7a, Krtcap2, Flrt1, Sdhd, Bphl, Sod2, Ndufb7, Prrc2b, Rex1bd, Rab36, Pdrg1, Pfn2, Rarg, B230118H07Rik, Elavl3, Mlf1, Eif1, Mettl26, Kmt5a, Ndufa5, Ndufb3, Emc8, Bad, Smc3, Dpm3, Ndufa11, Cisd1, Mrp127, Cldn9, Sec61b, Tpd5212, Polr2c, Ndufa7, Atf5, Gabarap, Srsf3, Psmb3, Bax, Atox1, 2410004P03Rik, Atpla1, Ptprs, Cib1, Hist1h2bc, Atp5k, Hmox2, Fam92b, Chd4, Elob, Kif5a, Tbcb, Ndufa4, Uqcr11, Hras, Atp6vlf, Pam16, Pim3, Lrriq1, Nfu1, Supt4a, Swi5, Mtx2, Faim, Taldo1, Ccdc34, Rbm39, 2210016L21Rik, Dpy30, Tomm22, Pygo1, Mat2b, Mrpl28, Eid1, Snrpe, Atp5j, Ndufs7, Park7, Ndufs4, Cirbp, Polr2f, Fbxo9, Mgst3, Gtf2h5, Mtch1, Mrps24, Actr1b, Sh3bgrl3, Bmpr1a, Pfn1, Tesc, Tmem258, Mrps14, Abhd16a, Vdac2, Nxph3, Polr2g, Them6, Cfap126, Pitpnc1, Kctd1, and Dmkn.

6. The method of claim 1, wherein the at least one odor response gene is selected from the group consisting of:
Tsc22d1, Rcan1, Fosb, Pcdh10, Dusp14, Srxn1, Fos, and Nr4a1.

7. The method of claim 1, wherein the at least one odor response gene is selected from the group consisting of:
Tsc22d1, Rcan1, Fosb, Pcdh10, Dusp14, and Srxn1.

8. The method of claim 1, wherein the at least one odor response gene is selected from the group consisting of:
Btg2, Egr1, Fos, Fosb, Gm13889, Junb, Nr4a1, Nr4a2, Pcdh10, and Srxn1.

9. The method of claim 1, wherein the at least one odor response gene is selected from the group consisting of:
Btg2, Egr1, Fos, Fosb, Gm13889, Junb, Nr4a1, Nr4a2, Pcdh10, Srxn1, Tsc22d1, Rcan1, and Dusp14.

10. (canceled)

11. The method of claim 1, further comprising measuring the expression of at least one odorant receptor gene.

12.-17. (canceled)

18. The method of claim 11, wherein the human odorant receptor is selected from the group consisting of:
human OR family 1, human OR family 2, human OR family 3, human OR family 4, human OR family 5, human OR family 6, human OR family 7, human OR family 8, human OR family 9, human OR family 10, human OR family 11, human OR family 12, human OR family 13, human OR family 14, human OR family 51, human OR family 52, human OR family 55, human OR family 56, murine OR family 1, murine OR family 2, murine OR family 3, murine OR family 4, murine OR family 5, murine OR family 6, murine OR family 7, murine OR family 8, murine OR family 9, murine OR family 10-15, murine OR family 16-34, and any variant thereof having an amino acid sequence having at least 80% identity with the amino acid sequence of one of said odorant receptors.

19. The method of claim 11, wherein the odorant receptor is selected from the group consisting of:
Olfr 545, Olfr 160, Olfr 17, Olfr 727, Olfr 728, and Olfr 729.

20. The method of claim 11, wherein the odorant receptor is selected from the group consisting of:
Olfr 545, Olfr 160, and Olfr 17.

21.-22. (canceled)

23. The method of claim 1, wherein the first OSN and/or the second OSN is in vitro during the contacting.

24. The method of claim 1, wherein the first OSN and/or the second OSN is in vivo during the contacting.

25. The method of claim 1, wherein the contacting the first OSN comprises contacting a subject comprising the first OSN with air comprising the at least one volatilized chemical compound.

26. The method of claim 2, wherein the not contacting the second OSN comprises:
occluding the nostrils of a subject comprising the second OSN, or
contacting a subject comprising the second OSN with air not comprising the at least one volatilized chemical compound.

27. (canceled)

28. The method of claim 1, wherein measuring the expression comprises measuring the level of one or more mRNAs.

29.-41. (canceled)

42. The method of claim 1, wherein the sample is obtained from the olfactory bulb, the olfactory epithelium, the nerve endings, and/or the nasal cavity.

43. The method of claim 1, wherein the sample comprises epithelial cells, endothelial cells, olfactory sensory neurons, goblet cells, cilia, and/or mast cells.

44.-49. (canceled)

50. A method comprising
measuring the expression of at least one odor response gene in a first olfactory sensory neuron (OSN) that has been contacted with at least one volatized chemical compound, wherein the first OSN is obtained from or present in a subject; and
determining that the subject has a loss of smell if the expression of the at least one odor response gene in the first OSN is different from the expression in a second OSN obtained from or present in a normal, healthy individual.

51.-52. (canceled)
